SEQUENCE LISTING (134 sequences)

SEQ ID NO: 1 - DNA, 2080 bp, unidentified
  source 1..2080: mol_type = genomic DNA; note = Varicella Zoster Virus
tcaagcttttggaccctcgtacagaagctaatacgactcactatagggaaataagagagaaaagaagagtaagaagaaatataagagccaccatggggacagtgaataagccggttgtgggcgtgcttatgggctttgggattattaccggtacattacgaattaccaatccagtgcgcgccagtgtgctgcgttacgacgactttcacattgacgaggataagctggatactaacagcgtgtacgaaccttattaccactcagatcatgccgaatcaagctgggttaatagaggagaaagcagccgaaaagcctacgaccacaactcaccttatatttggcccagaaacgattatgacggtttcctggaaaacgcacatgaacaccatggagtctacaaccaaggcaggggaatcgacagtggcgagcgtcttatgcagccaacacagatgtcggcacaggaggatctcggtgatgacaccggcatacacgtgattcccacattaaacggcgacgacagacataagatcgtcaatgtggatcagcgtcagtatggggatgtctttaaaggcgatttgaatccaaagccccaaggacagagactgatcgaggtctctgtagaagaaaatcaccccttcactttgcgcgctccaatccagaggatttacggggtgcgttataccgaaacttggagtttcttgccgtcactgacgtgtacgggggatgccgcccccgcaatccagcacatctgtctgaaacacaccacatgctttcaggacgtggttgtggatgtggattgcgcggaaaacacaaaagaagaccaactcgccgaaatcagctatcgttttcagggtaaaaaagaggccgaccaaccgtggattgttgtgaatacgagcacgctcttcgatgagcttgaactcgatcccccggaaatcgagcctggggttctaaaagtgttgaggaccgagaagcagtacctcggggtttatatctggaatatgagaggctccgatggcacctctacctacgcaacgtttctggttacctggaagggagacgagaagacacggaatccaacgcccgctgtgacccctcagcctaggggagccgaattccacatgtggaactatcactcccatgtattcagtgtgggtgacactttcagcctggccatgcacctgcagtataagattcacgaggcacccttcgacctcctgctggagtggttgtacgtacctattgatcccacttgtcagcccatgcgcctgtactccacttgcttgtaccaccccaatgcaccacagtgtctatcacacatgaactccgggtgtacctttacttcaccccatcttgcccagcgggtcgccagcacagtgtatcagaactgtgagcatgctgacaactatactgcttattgcctcggaatatcccatatggagccaagcttcgggctcatactgcacgatggtggtacgacactcaagttcgtggacacccccgaaagcctttctggcttgtacgtgttcgtggtctacttcaatggacatgtggaggcagtggcttacacagtggtttcgacagttgatcactttgtaaatgccattgaggaacgcggcttcccgcctacagcgggccagccccctgcgacaacaaaaccaaaagagattacgcccgttaatcctgggactagtccattgctgaggtatgccgcctggactggcggtctggcggccgtggtacttctgtgtttagtcatatttctgatctgtaccgctaaacgtatgcgggtcaaggcttaccgtgttgacaagtctccttacaatcagtcaatgtactatgcaggactccctgttgacgatttcgaagactcagagagtacagacacagaagaagaattcggaaacgctataggtggctctcacggaggtagctcgtatacagtgtacatcgataaaaccagatgataataggctggagcctcggtggccatgcttcttgccccttgggcctccccccagcccctcctccccttcctgcacccgtacccccgtggtctttgaataaagtctgagtgggcggc

SEQ ID NO: 2 - RNA, 1276 nt, unidentified
  source 1..1276: mol_type = genomic RNA; note = Varicella Zoster Virus
tcaagcttttggaccctcgtacagaagctaatacgactcactatagggaaataagagagaaaagaagagtaagaagaaatataagagccaccatgtttttaatccaatgtttgatatcggccgttatattttacatacaagtgaccaacgctttgatcttcaagggcgaccacgtgagcttgcaagttaacagcagtctcacgtctatccttattcccatgcaaaatgataattatacagagataaaaggacagcttgtctttattggagagcaactacctaccgggacaaactatagcggaacactggaactgttatacgcggatacggtggcgttttgtttccggtcagtacaagtaataagatacgacggatgtccccggattagaacgagcgcttttatttcgtgtaggtacaaacattcgtggcattatggtaactcaacggatcggatatcaacagagccggatgctggtgtaatgttgaaaattaccaaaccgggaataaatgatgctggtgtgtatgtacttcttgttcggttagaccatagcagatccaccgatggtttcattcttggtgtaaatgtatatacagcgggctcgcatcacaacattcacggggttatctacacttctccatctctacagaatggatattctacaagagccctttttcaacaagctcgtttgtgtgatttacccgcgacacccaaagggtccggtacctccctgtttcaacatatgcttgatcttcgtgccggtaaatcgttagaggataacccttggttacatgaggacgttgttacgacagaaactaagtccgttgttaaggaggggatagaaaatcacgtatatccaacggatatgtccacgttacccgaaaagtcccttaatgatcctccagaaaatctacttataattattcctatagtagcgtctgtcatgatcctcaccgccatggttattgttattgtaataagcgttaagcgacgtagaattaaaaaacatccaatttatcgcccaaatacaaaaacaagaaggggcatacaaaatgcgacaccagaatccgatgtgatgttggaggccgccattgcacaactagcaacgattcgcgaagaatcccccccacattccgttgtaaacccgtttgttaaatagtgataataggctggagcctcggtggccatgcttcttgccccttgggcctccccccagcccctcctccccttcctgcacccgtacccccgtggtctttgaataaagtctgagtgggcggc

SEQ ID NO: 3 - DNA, 1897 bp, synthetic construct
  source 1..1897: mol_type = other DNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttgatgataataggctggagcctcggtggccatgcttcttgccccttgggcctccccccagcccctcctccccttcctgcacccgtacccccgtggtctttgaataaagtctgagtgggcggc

SEQ ID NO: 4 - DNA, 1867 bp, synthetic construct
  source 1..1867: mol_type = other DNA
tcaagcttttggaccctcgtacagaagctaatacgactcactatagggaaataagagagaaaagaagagtaagaagaaatataagagccaccatggaaaccccggcgcagctgctgtttctgctgctgctgtggctgccggataccaccggctccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttgatgataataggctggagcctcggtggccatgcttcttgccccttgggcctccccccagcccctcctccccttcctgcacccgtacccccgtggtctttgaataaagtctgagtgggcggc

SEQ ID NO: 5 - DNA, 1933 bp, synthetic construct
  source 1..1933: mol_type = other DNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtgatgataataggctggagcctcggtggccatgcttcttgccccttgggcctccccccagcccctcctccccttcctgcacccgtacccccgtggtctttgaataaagtctgagtgggcggc

SEQ ID NO: 6 - DNA, 1933 bp, synthetic construct
  source 1..1933: mol_type = other DNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgatgataataggctggagcctcggtggccatgcttcttgccccttgggcctccccccagcccctcctccccttcctgcacccgtacccccgtggtctttgaataaagtctgagtgggcggc

SEQ ID NO: 7 - DNA, 2083 bp, synthetic construct
  source 1..2083: mol_type = other DNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggacgccgaagccgccgatgccgaagaagagtttggtaacgcgattggagggagtcacgggggttcgagttacacggtgtatatagataagacccggtgatgataataggctggagcctcggtggccatgcttcttgccccttgggcctccccccagcccctcctccccttcctgcacccgtacccccgtggtctttgaataaagtctgagtgggcggc

SEQ ID NO: 8 - DNA, 2083 bp, synthetic construct
  source 1..2083: mol_type = other DNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtatggcgctggccttccagtggacgatttcgaggacgccgaagccgccgatgccgaagaagagtttggtaacgcgattggagggagtcacgggggttcgagttacacggtgtatatagataagacccggtgatgataataggctggagcctcggtggccatgcttcttgccccttgggcctccccccagcccctcctccccttcctgcacccgtacccccgtggtctttgaataaagtctgagtgggcggc

SEQ ID NO: 9 - RNA, 2080 nt, synthetic construct
  source 1..2080: mol_type = other RNA
tcaagcttttggaccctcgtacagaagctaatacgactcactatagggaaataagagagaaaagaagagtaagaagaaatataagagccaccatggggacagtgaataagccggttgtgggcgtgcttatgggctttgggattattaccggtacattacgaattaccaatccagtgcgcgccagtgtgctgcgttacgacgactttcacattgacgaggataagctggatactaacagcgtgtacgaaccttattaccactcagatcatgccgaatcaagctgggttaatagaggagaaagcagccgaaaagcctacgaccacaactcaccttatatttggcccagaaacgattatgacggtttcctggaaaacgcacatgaacaccatggagtctacaaccaaggcaggggaatcgacagtggcgagcgtcttatgcagccaacacagatgtcggcacaggaggatctcggtgatgacaccggcatacacgtgattcccacattaaacggcgacgacagacataagatcgtcaatgtggatcagcgtcagtatggggatgtctttaaaggcgatttgaatccaaagccccaaggacagagactgatcgaggtctctgtagaagaaaatcaccccttcactttgcgcgctccaatccagaggatttacggggtgcgttataccgaaacttggagtttcttgccgtcactgacgtgtacgggggatgccgcccccgcaatccagcacatctgtctgaaacacaccacatgctttcaggacgtggttgtggatgtggattgcgcggaaaacacaaaagaagaccaactcgccgaaatcagctatcgttttcagggtaaaaaagaggccgaccaaccgtggattgttgtgaatacgagcacgctcttcgatgagcttgaactcgatcccccggaaatcgagcctggggttctaaaagtgttgaggaccgagaagcagtacctcggggtttatatctggaatatgagaggctccgatggcacctctacctacgcaacgtttctggttacctggaagggagacgagaagacacggaatccaacgcccgctgtgacccctcagcctaggggagccgaattccacatgtggaactatcactcccatgtattcagtgtgggtgacactttcagcctggccatgcacctgcagtataagattcacgaggcacccttcgacctcctgctggagtggttgtacgtacctattgatcccacttgtcagcccatgcgcctgtactccacttgcttgtaccaccccaatgcaccacagtgtctatcacacatgaactccgggtgtacctttacttcaccccatcttgcccagcgggtcgccagcacagtgtatcagaactgtgagcatgctgacaactatactgcttattgcctcggaatatcccatatggagccaagcttcgggctcatactgcacgatggtggtacgacactcaagttcgtggacacccccgaaagcctttctggcttgtacgtgttcgtggtctacttcaatggacatgtggaggcagtggcttacacagtggtttcgacagttgatcactttgtaaatgccattgaggaacgcggcttcccgcctacagcgggccagccccctgcgacaacaaaaccaaaagagattacgcccgttaatcctgggactagtccattgctgaggtatgccgcctggactggcggtctggcggccgtggtacttctgtgtttagtcatatttctgatctgtaccgctaaacgtatgcgggtcaaggcttaccgtgttgacaagtctccttacaatcagtcaatgtactatgcaggactccctgttgacgatttcgaagactcagagagtacagacacagaagaagaattcggaaacgctataggtggctctcacggaggtagctcgtatacagtgtacatcgataaaaccagatgataataggctggagcctcggtggccatgcttcttgccccttgggcctccccccagcccctcctccccttcctgcacccgtacccccgtggtctttgaataaagtctgagtgggcggc

SEQ ID NO: 10 - protein, 623 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 11 - RNA, 1869 nt, synthetic construct
  source 1..1869: mol_type = other RNA
atggggacagtgaataagccggttgtgggcgtgcttatgggctttgggattattaccggtacattacgaattaccaatccagtgcgcgccagtgtgctgcgttacgacgactttcacattgacgaggataagctggatactaacagcgtgtacgaaccttattaccactcagatcatgccgaatcaagctgggttaatagaggagaaagcagccgaaaagcctacgaccacaactcaccttatatttggcccagaaacgattatgacggtttcctggaaaacgcacatgaacaccatggagtctacaaccaaggcaggggaatcgacagtggcgagcgtcttatgcagccaacacagatgtcggcacaggaggatctcggtgatgacaccggcatacacgtgattcccacattaaacggcgacgacagacataagatcgtcaatgtggatcagcgtcagtatggggatgtctttaaaggcgatttgaatccaaagccccaaggacagagactgatcgaggtctctgtagaagaaaatcaccccttcactttgcgcgctccaatccagaggatttacggggtgcgttataccgaaacttggagtttcttgccgtcactgacgtgtacgggggatgccgcccccgcaatccagcacatctgtctgaaacacaccacatgctttcaggacgtggttgtggatgtggattgcgcggaaaacacaaaagaagaccaactcgccgaaatcagctatcgttttcagggtaaaaaagaggccgaccaaccgtggattgttgtgaatacgagcacgctcttcgatgagcttgaactcgatcccccggaaatcgagcctggggttctaaaagtgttgaggaccgagaagcagtacctcggggtttatatctggaatatgagaggctccgatggcacctctacctacgcaacgtttctggttacctggaagggagacgagaagacacggaatccaacgcccgctgtgacccctcagcctaggggagccgaattccacatgtggaactatcactcccatgtattcagtgtgggtgacactttcagcctggccatgcacctgcagtataagattcacgaggcacccttcgacctcctgctggagtggttgtacgtacctattgatcccacttgtcagcccatgcgcctgtactccacttgcttgtaccaccccaatgcaccacagtgtctatcacacatgaactccgggtgtacctttacttcaccccatcttgcccagcgggtcgccagcacagtgtatcagaactgtgagcatgctgacaactatactgcttattgcctcggaatatcccatatggagccaagcttcgggctcatactgcacgatggtggtacgacactcaagttcgtggacacccccgaaagcctttctggcttgtacgtgttcgtggtctacttcaatggacatgtggaggcagtggcttacacagtggtttcgacagttgatcactttgtaaatgccattgaggaacgcggcttcccgcctacagcgggccagccccctgcgacaacaaaaccaaaagagattacgcccgttaatcctgggactagtccattgctgaggtatgccgcctggactggcggtctggcggccgtggtacttctgtgtttagtcatatttctgatctgtaccgctaaacgtatgcgggtcaaggcttaccgtgttgacaagtctccttacaatcagtcaatgtactatgcaggactccctgttgacgatttcgaagactcagagagtacagacacagaagaagaattcggaaacgctataggtggctctcacggaggtagctcgtatacagtgtacatcgataaaaccaga

SEQ ID NO: 12 - RNA, 2141 nt, synthetic construct
  source 1..2141: mol_type = other RNA
ggggaaataagagagaaaagaagagtaagaagaaatataagagccaccatggggacagtgaataagccggttgtgggcgtgcttatgggctttgggattattaccggtacattacgaattaccaatccagtgcgcgccagtgtgctgcgttacgacgactttcacattgacgaggataagctggatactaacagcgtgtacgaaccttattaccactcagatcatgccgaatcaagctgggttaatagaggagaaagcagccgaaaagcctacgaccacaactcaccttatatttggcccagaaacgattatgacggtttcctggaaaacgcacatgaacaccatggagtctacaaccaaggcaggggaatcgacagtggcgagcgtcttatgcagccaacacagatgtcggcacaggaggatctcggtgatgacaccggcatacacgtgattcccacattaaacggcgacgacagacataagatcgtcaatgtggatcagcgtcagtatggggatgtctttaaaggcgatttgaatccaaagccccaaggacagagactgatcgaggtctctgtagaagaaaatcaccccttcactttgcgcgctccaatccagaggatttacggggtgcgttataccgaaacttggagtttcttgccgtcactgacgtgtacgggggatgccgcccccgcaatccagcacatctgtctgaaacacaccacatgctttcaggacgtggttgtggatgtggattgcgcggaaaacacaaaagaagaccaactcgccgaaatcagctatcgttttcagggtaaaaaagaggccgaccaaccgtggattgttgtgaatacgagcacgctcttcgatgagcttgaactcgatcccccggaaatcgagcctggggttctaaaagtgttgaggaccgagaagcagtacctcggggtttatatctggaatatgagaggctccgatggcacctctacctacgcaacgtttctggttacctggaagggagacgagaagacacggaatccaacgcccgctgtgacccctcagcctaggggagccgaattccacatgtggaactatcactcccatgtattcagtgtgggtgacactttcagcctggccatgcacctgcagtataagattcacgaggcacccttcgacctcctgctggagtggttgtacgtacctattgatcccacttgtcagcccatgcgcctgtactccacttgcttgtaccaccccaatgcaccacagtgtctatcacacatgaactccgggtgtacctttacttcaccccatcttgcccagcgggtcgccagcacagtgtatcagaactgtgagcatgctgacaactatactgcttattgcctcggaatatcccatatggagccaagcttcgggctcatactgcacgatggtggtacgacactcaagttcgtggacacccccgaaagcctttctggcttgtacgtgttcgtggtctacttcaatggacatgtggaggcagtggcttacacagtggtttcgacagttgatcactttgtaaatgccattgaggaacgcggcttcccgcctacagcgggccagccccctgcgacaacaaaaccaaaagagattacgcccgttaatcctgggactagtccattgctgaggtatgccgcctggactggcggtctggcggccgtggtacttctgtgtttagtcatatttctgatctgtaccgctaaacgtatgcgggtcaaggcttaccgtgttgacaagtctccttacaatcagtcaatgtactatgcaggactccctgttgacgatttcgaagactcagagagtacagacacagaagaagaattcggaaacgctataggtggctctcacggaggtagctcgtatacagtgtacatcgataaaaccagatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 13 - RNA, 2050 nt, synthetic construct
  source 1..2050: mol_type = other RNA
tcaagcttttggaccctcgtacagaagctaatacgactcactatagggaaataagagagaaaagaagagtaagaagaaatataagagccaccatggagactcccgctcagctactgttcctcctgctcctttggctgcctgatactacaggctctgttttgcggtacgacgactttcacatcgatgaggacaagctcgacactaatagcgtgtatgagccctactaccattcagatcacgccgagtcctcttgggtgaacaggggtgaaagttctaggaaagcctatgatcacaacagcccttatatttggccacggaatgattacgacggatttctcgaaaatgcccacgagcatcacggagtgtacaaccagggccgtggaatcgactctggggagagattgatgcaacctacacagatgagcgcccaggaagatctcggggatgatacaggaattcacgttatccctacattaaacggagatgaccgccacaaaatcgtcaatgtcgatcaaagacagtatggagatgtgttcaaaggcgatctcaaccctaagccgcagggccagagactcattgaggtgtctgtcgaagagaaccaccctttcactctgcgcgctcccattcagagaatctatggagttcgctatacggagacttggtcattccttccttccctgacatgcaccggagacgccgcccctgccattcagcacatatgcctgaaacataccacctgtttccaggatgtggtggttgatgttgattgtgctgaaaataccaaggaagaccaactggccgagattagttaccggttccaagggaaaaaggaagccgaccagccatggattgtggttaatacaagcactctgttcgatgagctcgagctggatccccccgagatagaacccggagttctgaaagtgctccggacagaaaaacaatatctgggagtctacatatggaacatgcgcggttccgatgggacctccacttatgcaacctttctcgtcacgtggaagggagatgagaaaactaggaatcccacacccgctgtcacaccacagccaagaggggctgagttccatatgtggaactatcatagtcacgtgtttagtgtcggagatacgttttcattggctatgcatctccagtacaagattcatgaggctcccttcgatctgttgcttgagtggttgtacgtcccgattgacccgacctgccagcccatgcgactgtacagcacctgtctctaccatccaaacgctccgcaatgtctgagccacatgaactctgggtgtactttcaccagtccccacctcgcccagcgggtggcctctactgtttaccagaactgtgagcacgccgacaactacaccgcatactgcctcggtatttctcacatggaaccctccttcggactcatcctgcacgatgggggcactaccctgaagttcgttgatacgccagaatctctgtctgggctctatgttttcgtggtctacttcaatggccatgtcgaggccgtggcctatactgtcgtttctaccgtggatcattttgtgaacgccatcgaagaacggggattcccccctacggcaggccagccgcctgcaaccaccaagcccaaggaaataacaccagtgaaccctggcacctcacctctcctaagatatgccgcgtggacagggggactggcggcagtggtgctcctctgtctcgtgatctttctgatctgtacagccaagaggatgagggtcaaggcttatagagtggacaagtccccctacaatcagtcaatgtactacgccggccttcccgttgatgattttgaggattccgagtccacagatactgaggaagagttcggtaacgctataggcggctctcacgggggttcaagctacacggtttacattgacaagacacgctgataataggctggagcctcggtggccatgcttcttgccccttgggcctccccccagcccctcctccccttcctgcacccgtacccccgtggtctttgaataaagtctgagtgggcggc

SEQ ID NO: 14 - protein, 613 aa, synthetic construct
METPAQLLFLLLLWLPDTTGSVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 15 - RNA, 1839 nt, synthetic construct
  source 1..1839: mol_type = other RNA
atggagactcccgctcagctactgttcctcctgctcctttggctgcctgatactacaggctctgttttgcggtacgacgactttcacatcgatgaggacaagctcgacactaatagcgtgtatgagccctactaccattcagatcacgccgagtcctcttgggtgaacaggggtgaaagttctaggaaagcctatgatcacaacagcccttatatttggccacggaatgattacgacggatttctcgaaaatgcccacgagcatcacggagtgtacaaccagggccgtggaatcgactctggggagagattgatgcaacctacacagatgagcgcccaggaagatctcggggatgatacaggaattcacgttatccctacattaaacggagatgaccgccacaaaatcgtcaatgtcgatcaaagacagtatggagatgtgttcaaaggcgatctcaaccctaagccgcagggccagagactcattgaggtgtctgtcgaagagaaccaccctttcactctgcgcgctcccattcagagaatctatggagttcgctatacggagacttggtcattccttccttccctgacatgcaccggagacgccgcccctgccattcagcacatatgcctgaaacataccacctgtttccaggatgtggtggttgatgttgattgtgctgaaaataccaaggaagaccaactggccgagattagttaccggttccaagggaaaaaggaagccgaccagccatggattgtggttaatacaagcactctgttcgatgagctcgagctggatccccccgagatagaacccggagttctgaaagtgctccggacagaaaaacaatatctgggagtctacatatggaacatgcgcggttccgatgggacctccacttatgcaacctttctcgtcacgtggaagggagatgagaaaactaggaatcccacacccgctgtcacaccacagccaagaggggctgagttccatatgtggaactatcatagtcacgtgtttagtgtcggagatacgttttcattggctatgcatctccagtacaagattcatgaggctcccttcgatctgttgcttgagtggttgtacgtcccgattgacccgacctgccagcccatgcgactgtacagcacctgtctctaccatccaaacgctccgcaatgtctgagccacatgaactctgggtgtactttcaccagtccccacctcgcccagcgggtggcctctactgtttaccagaactgtgagcacgccgacaactacaccgcatactgcctcggtatttctcacatggaaccctccttcggactcatcctgcacgatgggggcactaccctgaagttcgttgatacgccagaatctctgtctgggctctatgttttcgtggtctacttcaatggccatgtcgaggccgtggcctatactgtcgtttctaccgtggatcattttgtgaacgccatcgaagaacggggattcccccctacggcaggccagccgcctgcaaccaccaagcccaaggaaataacaccagtgaaccctggcacctcacctctcctaagatatgccgcgtggacagggggactggcggcagtggtgctcctctgtctcgtgatctttctgatctgtacagccaagaggatgagggtcaaggcttatagagtggacaagtccccctacaatcagtcaatgtactacgccggccttcccgttgatgattttgaggattccgagtccacagatactgaggaagagttcggtaacgctataggcggctctcacgggggttcaagctacacggtttacattgacaagacacgc

SEQ ID NO: 16 - RNA, 2111 nt, synthetic construct
  source 1..2111: mol_type = other RNA
ggggaaataagagagaaaagaagagtaagaagaaatataagagccaccatggagactcccgctcagctactgttcctcctgctcctttggctgcctgatactacaggctctgttttgcggtacgacgactttcacatcgatgaggacaagctcgacactaatagcgtgtatgagccctactaccattcagatcacgccgagtcctcttgggtgaacaggggtgaaagttctaggaaagcctatgatcacaacagcccttatatttggccacggaatgattacgacggatttctcgaaaatgcccacgagcatcacggagtgtacaaccagggccgtggaatcgactctggggagagattgatgcaacctacacagatgagcgcccaggaagatctcggggatgatacaggaattcacgttatccctacattaaacggagatgaccgccacaaaatcgtcaatgtcgatcaaagacagtatggagatgtgttcaaaggcgatctcaaccctaagccgcagggccagagactcattgaggtgtctgtcgaagagaaccaccctttcactctgcgcgctcccattcagagaatctatggagttcgctatacggagacttggtcattccttccttccctgacatgcaccggagacgccgcccctgccattcagcacatatgcctgaaacataccacctgtttccaggatgtggtggttgatgttgattgtgctgaaaataccaaggaagaccaactggccgagattagttaccggttccaagggaaaaaggaagccgaccagccatggattgtggttaatacaagcactctgttcgatgagctcgagctggatccccccgagatagaacccggagttctgaaagtgctccggacagaaaaacaatatctgggagtctacatatggaacatgcgcggttccgatgggacctccacttatgcaacctttctcgtcacgtggaagggagatgagaaaactaggaatcccacacccgctgtcacaccacagccaagaggggctgagttccatatgtggaactatcatagtcacgtgtttagtgtcggagatacgttttcattggctatgcatctccagtacaagattcatgaggctcccttcgatctgttgcttgagtggttgtacgtcccgattgacccgacctgccagcccatgcgactgtacagcacctgtctctaccatccaaacgctccgcaatgtctgagccacatgaactctgggtgtactttcaccagtccccacctcgcccagcgggtggcctctactgtttaccagaactgtgagcacgccgacaactacaccgcatactgcctcggtatttctcacatggaaccctccttcggactcatcctgcacgatgggggcactaccctgaagttcgttgatacgccagaatctctgtctgggctctatgttttcgtggtctacttcaatggccatgtcgaggccgtggcctatactgtcgtttctaccgtggatcattttgtgaacgccatcgaagaacggggattcccccctacggcaggccagccgcctgcaaccaccaagcccaaggaaataacaccagtgaaccctggcacctcacctctcctaagatatgccgcgtggacagggggactggcggcagtggtgctcctctgtctcgtgatctttctgatctgtacagccaagaggatgagggtcaaggcttatagagtggacaagtccccctacaatcagtcaatgtactacgccggccttcccgttgatgattttgaggattccgagtccacagatactgaggaagagttcggtaacgctataggcggctctcacgggggttcaagctacacggtttacattgacaagacacgctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 17 - RNA, 1897 nt, synthetic construct
  source 1..1897: mol_type = other RNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttgatgataataggctggagcctcggtggccatgcttcttgccccttgggcctccccccagcccctcctccccttcctgcacccgtacccccgtggtctttgaataaagtctgagtgggcggc

SEQ ID NO: 18 - protein, 561 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTA

SEQ ID NO: 19 - RNA, 1686 nt, synthetic construct
  source 1..1686: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttga

SEQ ID NO: 20 - RNA, 1958 nt, synthetic construct
  source 1..1958: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttgatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 21 - RNA, 1867 nt, synthetic construct
  source 1..1867: mol_type = other RNA
tcaagcttttggaccctcgtacagaagctaatacgactcactatagggaaataagagagaaaagaagagtaagaagaaatataagagccaccatggaaaccccggcgcagctgctgtttctgctgctgctgtggctgccggataccaccggctccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttgatgataataggctggagcctcggtggccatgcttcttgccccttgggcctccccccagcccctcctccccttcctgcacccgtacccccgtggtctttgaataaagtctgagtgggcggc

SEQ ID NO: 22 - protein, 551 aa, synthetic construct
METPAQLLFLLLLWLPDTTGSVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTA

SEQ ID NO: 23 - RNA, 1656 nt, synthetic construct
  source 1..1656: mol_type = other RNA
atggaaaccccggcgcagctgctgtttctgctgctgctgtggctgccggataccaccggctccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttga

SEQ ID NO: 24 - RNA, 1928 nt, synthetic construct
  source 1..1928: mol_type = other RNA
ggggaaataagagagaaaagaagagtaagaagaaatataagagccaccatggaaaccccggcgcagctgctgtttctgctgctgctgtggctgccggataccaccggctccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggcttgatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 25 - RNA, 2083 nt, synthetic construct
  source 1..2083: mol_type = other RNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggacgccgaagccgccgatgccgaagaagagtttggtaacgcgattggagggagtcacgggggttcgagttacacggtgtatatagataagacccggtgatgataataggctggagcctcggtggccatgcttcttgccccttgggcctccccccagcccctcctccccttcctgcacccgtacccccgtggtctttgaataaagtctgagtgggcggc

SEQ ID NO: 26 - protein, 623 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDAEAADAEEEFGNAIGGSHGGSSYTVYIDKTR

SEQ ID NO: 27 - RNA, 1872 nt, synthetic construct
  source 1..1872: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggacgccgaagccgccgatgccgaagaagagtttggtaacgcgattggagggagtcacgggggttcgagttacacggtgtatatagataagacccggtga

SEQ ID NO: 28 - RNA, 2144 nt, synthetic construct
  source 1..2144: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggacgccgaagccgccgatgccgaagaagagtttggtaacgcgattggagggagtcacgggggttcgagttacacggtgtatatagataagacccggtgatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 29 - RNA, 2083 nt, synthetic construct
  source 1..2083: mol_type = other RNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtatggcgctggccttccagtggacgatttcgaggacgccgaagccgccgatgccgaagaagagtttggtaacgcgattggagggagtcacgggggttcgagttacacggtgtatatagataagacccggtgatgataataggctggagcctcggtggccatgcttcttgccccttgggcctccccccagcccctcctccccttcctgcacccgtacccccgtggtctttgaataaagtctgagtgggcggc

SEQ ID NO: 30 - protein, 623 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYGAGLPVDDFEDAEAADAEEEFGNAIGGSHGGSSYTVYIDKTR

SEQ ID NO: 31 - RNA, 1872 nt, synthetic construct
  source 1..1872: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtatggcgctggccttccagtggacgatttcgaggacgccgaagccgccgatgccgaagaagagtttggtaacgcgattggagggagtcacgggggttcgagttacacggtgtatatagataagacccggtga

SEQ ID NO: 32 - RNA, 2144 nt, synthetic construct
  source 1..2144: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtatggcgctggccttccagtggacgatttcgaggacgccgaagccgccgatgccgaagaagagtttggtaacgcgattggagggagtcacgggggttcgagttacacggtgtatatagataagacccggtgatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 33 - RNA, 1933 nt, synthetic construct
  source 1..1933: mol_type = other RNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtgatgataataggctggagcctcggtggccatgcttcttgccccttgggcctccccccagcccctcctccccttcctgcacccgtacccccgtggtctttgaataaagtctgagtgggcggc

SEQ ID NO: 34 - protein, 573 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDK

SEQ ID NO: 35 - RNA, 1722 nt, synthetic construct
  source 1..1722: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtga

SEQ ID NO: 36 - RNA, 1994 nt, synthetic construct
  source 1..1994: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtgatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 37 - RNA, 1933 nt, synthetic construct
  source 1..1933: mol_type = other RNA
tcaagcttttggaccctcgtacagaagctaatacgactcactatagggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgatgataataggctggagcctcggtggccatgcttcttgccccttgggcctccccccagcccctcctccccttcctgcacccgtacccccgtggtctttgaataaagtctgagtgggcggc

SEQ ID NO: 38 - protein, 573 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAARVDK

SEQ ID NO: 39 - RNA, 1722 nt, synthetic construct
  source 1..1722: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtga

SEQ ID NO: 40 - RNA, 1994 nt, synthetic construct
  source 1..1994: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 41 - DNA, 2050 bp, synthetic construct
  source 1..2050: mol_type = other DNA
tcaagcttttggaccctcgtacagaagctaatacgactcactatagggaaataagagagaaaagaagagtaagaagaaatataagagccaccatggagactcccgctcagctactgttcctcctgctcctttggctgcctgatactacaggctctgttttgcggtacgacgactttcacatcgatgaggacaagctcgacactaatagcgtgtatgagccctactaccattcagatcacgccgagtcctcttgggtgaacaggggtgaaagttctaggaaagcctatgatcacaacagcccttatatttggccacggaatgattacgacggatttctcgaaaatgcccacgagcatcacggagtgtacaaccagggccgtggaatcgactctggggagagattgatgcaacctacacagatgagcgcccaggaagatctcggggatgatacaggaattcacgttatccctacattaaacggagatgaccgccacaaaatcgtcaatgtcgatcaaagacagtatggagatgtgttcaaaggcgatctcaaccctaagccgcagggccagagactcattgaggtgtctgtcgaagagaaccaccctttcactctgcgcgctcccattcagagaatctatggagttcgctatacggagacttggtcattccttccttccctgacatgcaccggagacgccgcccctgccattcagcacatatgcctgaaacataccacctgtttccaggatgtggtggttgatgttgattgtgctgaaaataccaaggaagaccaactggccgagattagttaccggttccaagggaaaaaggaagccgaccagccatggattgtggttaatacaagcactctgttcgatgagctcgagctggatccccccgagatagaacccggagttctgaaagtgctccggacagaaaaacaatatctgggagtctacatatggaacatgcgcggttccgatgggacctccacttatgcaacctttctcgtcacgtggaagggagatgagaaaactaggaatcccacacccgctgtcacaccacagccaagaggggctgagttccatatgtggaactatcatagtcacgtgtttagtgtcggagatacgttttcattggctatgcatctccagtacaagattcatgaggctcccttcgatctgttgcttgagtggttgtacgtcccgattgacccgacctgccagcccatgcgactgtacagcacctgtctctaccatccaaacgctccgcaatgtctgagccacatgaactctgggtgtactttcaccagtccccacctcgcccagcgggtggcctctactgtttaccagaactgtgagcacgccgacaactacaccgcatactgcctcggtatttctcacatggaaccctccttcggactcatcctgcacgatgggggcactaccctgaagttcgttgatacgccagaatctctgtctgggctctatgttttcgtggtctacttcaatggccatgtcgaggccgtggcctatactgtcgtttctaccgtggatcattttgtgaacgccatcgaagaacggggattcccccctacggcaggccagccgcctgcaaccaccaagcccaaggaaataacaccagtgaaccctggcacctcacctctcctaagatatgccgcgtggacagggggactggcggcagtggtgctcctctgtctcgtgatctttctgatctgtacagccaagaggatgagggtcaaggcttatagagtggacaagtccccctacaatcagtcaatgtactacgccggccttcccgttgatgattttgaggattccgagtccacagatactgaggaagagttcggtaacgctataggcggctctcacgggggttcaagctacacggtttacattgacaagacacgctgataataggctggagcctcggtggccatgcttcttgccccttgggcctccccccagcccctcctccccttcctgcacccgtacccccgtggtctttgaataaagtctgagtgggcggc

SEQ ID NO: 42 - protein, 354 aa, synthetic construct
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPENLLIIIPIVASVMILTAMVIVIVISVKRRRIKKHPIYRPNTKTRRGIQNATPESDVMLEAAIAQLATIREESPPHSVVNPFVK

SEQ ID NO: 43 - RNA, 1065 nt, synthetic construct
  source 1..1065: mol_type = other RNA
atgtttttaatccaatgtttgatatcggccgttatattttacatacaagtgaccaacgctttgatcttcaagggcgaccacgtgagcttgcaagttaacagcagtctcacgtctatccttattcccatgcaaaatgataattatacagagataaaaggacagcttgtctttattggagagcaactacctaccgggacaaactatagcggaacactggaactgttatacgcggatacggtggcgttttgtttccggtcagtacaagtaataagatacgacggatgtccccggattagaacgagcgcttttatttcgtgtaggtacaaacattcgtggcattatggtaactcaacggatcggatatcaacagagccggatgctggtgtaatgttgaaaattaccaaaccgggaataaatgatgctggtgtgtatgtacttcttgttcggttagaccatagcagatccaccgatggtttcattcttggtgtaaatgtatatacagcgggctcgcatcacaacattcacggggttatctacacttctccatctctacagaatggatattctacaagagccctttttcaacaagctcgtttgtgtgatttacccgcgacacccaaagggtccggtacctccctgtttcaacatatgcttgatcttcgtgccggtaaatcgttagaggataacccttggttacatgaggacgttgttacgacagaaactaagtccgttgttaaggaggggatagaaaatcacgtatatccaacggatatgtccacgttacccgaaaagtcccttaatgatcctccagaaaatctacttataattattcctatagtagcgtctgtcatgatcctcaccgccatggttattgttattgtaataagcgttaagcgacgtagaattaaaaaacatccaatttatcgcccaaatacaaaaacaagaaggggcatacaaaatgcgacaccagaatccgatgtgatgttggaggccgccattgcacaactagcaacgattcgcgaagaatcccccccacattccgttgtaaacccgtttgttaaatag

SEQ ID NO: 44 - RNA, 1337 nt, synthetic construct
  source 1..1337: mol_type = other RNA
ggggaaataagagagaaaagaagagtaagaagaaatataagagccaccatgtttttaatccaatgtttgatatcggccgttatattttacatacaagtgaccaacgctttgatcttcaagggcgaccacgtgagcttgcaagttaacagcagtctcacgtctatccttattcccatgcaaaatgataattatacagagataaaaggacagcttgtctttattggagagcaactacctaccgggacaaactatagcggaacactggaactgttatacgcggatacggtggcgttttgtttccggtcagtacaagtaataagatacgacggatgtccccggattagaacgagcgcttttatttcgtgtaggtacaaacattcgtggcattatggtaactcaacggatcggatatcaacagagccggatgctggtgtaatgttgaaaattaccaaaccgggaataaatgatgctggtgtgtatgtacttcttgttcggttagaccatagcagatccaccgatggtttcattcttggtgtaaatgtatatacagcgggctcgcatcacaacattcacggggttatctacacttctccatctctacagaatggatattctacaagagccctttttcaacaagctcgtttgtgtgatttacccgcgacacccaaagggtccggtacctccctgtttcaacatatgcttgatcttcgtgccggtaaatcgttagaggataacccttggttacatgaggacgttgttacgacagaaactaagtccgttgttaaggaggggatagaaaatcacgtatatccaacggatatgtccacgttacccgaaaagtcccttaatgatcctccagaaaatctacttataattattcctatagtagcgtctgtcatgatcctcaccgccatggttattgttattgtaataagcgttaagcgacgtagaattaaaaaacatccaatttatcgcccaaatacaaaaacaagaaggggcatacaaaatgcgacaccagaatccgatgtgatgttggaggccgccattgcacaactagcaacgattcgcgaagaatcccccccacattccgttgtaaacccgtttgttaaatagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 45 - protein, 623 aa, Homo sapiens; identical to SEQ ID NO: 10

SEQ ID NO: 46 - protein, 354 aa, Homo sapiens; identical to SEQ ID NO: 42

SEQ ID NO: 47 - protein, 556 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAWTGGLAAVVLLCLVIFLICTA

SEQ ID NO: 48 - protein, 574 aa, Homo sapiens
MKRIQINLILTIACIQLSTESQPTPVSITELYTSAATRKPDPAVAPTSAATRKPDPAVAPTSAATRKPDPAVAPTSAATRKPDPAVAPTSAATRKPDPAVAPTSAATRKPDPAVAPTSAASRKPDPAVAPTSAASRKPDPAVAPTSAASRKPDPAANTQHSQPPFLYENIQCVHGGIQSIPYFHTFIMPCYMRLTTGQQAAFKQQQKTYEQYSLDPEGSNITRWKSLIRPDLHIEVWFTRHLIDPHRQLGNALIRMPDLPVMLYSNSADLNLINNPEIFTHAKENYVIPDVKTTSDFSVTILSMDATTEGTYIWRVVNTKTKNVISEHSITVTTYYRPNITVVGDPVLTGQTYAAYCNVSKYYPPHSVRVRWTSRFGNIGKNFITDAIQEYANGLFSYVSAVRIPQQKQMDYPPPAIQCNVLWIRDGVSNMKYSAVVTPDVYPFPNVSIGIIDGHIVCTAKCVPRGVVHFVWWVNDSPINHENSEITGVCDQNKRFVNMQSSCPTSELDGPITYSCHLDGYPKKFPPFSAVYTYDASTYATTFSVVAVIIGVISILGTLGLIAVIATLCIRCCS

SEQ ID NO: 49 - protein, 159 aa, Homo sapiens
MASHKWLLQIVFLKTITIAYCLHLQDDTPLFFGAKPLSDVSLIITEPCVSSVYEAWDYAAPPVSNLSEALSGIVVKTKCPVPEVILWFKDKQMAYWTNPYVTLKGLAQSVGEEHKSGDIRDALLDALSGVWVDSTPSSTNIPENGCVWGADRLFQRVCQ

SEQ ID NO: 50 - protein, 435 aa, Homo sapiens
MGTQKKGPRSEKVSPYDTTTPEVEALDHQMDTLNWRIWIIQVMMFTLGAVMLLATLIAASSEYTGIPCFYAAVVDYELFNATLDGGVWSGNRGGYSAPVLFLEPHSVVAFTYYTALTAMAMAVYTLITAAIIHRETKNQRVRQSSGVAWLVVDPTTLFWGLLSLWLLNAVVLLLAYKQIGVAATLYLGHFATSVIFTTYFCGRGKLDETNIKAVANLRQQSVFLYRLAGPTRAVFVNLMAALMAICILFVSLMLELVVANHLHTGLWSSVSVAMSTFSTLSVVYLIVSELILAHYIHVLIGPSLGTLVACATLGTAAHSYMDRLYDPISVQSPRLIPTTRGTLACLAVFSVVMLLLRLMRAYVYHRQKRSRFYGAVRRVPERVRGYIRKVKPAHRNSRRTNYPSQGYGYVYENDSTYETDREDELLYERSNSGWE

SEQ ID NO: 51 - protein, 841 aa, Homo sapiens
MFALVLAVVILPLWTTANKSYVTPTPATRSIGHMSALLREYSDRNMSLKLEAFYPTGFDEELIKSLHWGNDRKHVFLVIVKVNPTTHEGDVGLVIFPKYLLSPYHFKAEHRAPFPAGRFGFLSHPVTPDVSFFDSSFAPYLTTQHLVAFTTFPPNPLVWHLERAETAATAERPFGVSLLPARPTVPKNTILEHKAHFATWDALARHTFFSAEAIITNSTLRIHVPLFGSVWPIRYWATGSVLLTSDSGRVEVNIGVGFMSSLISLSSGPPIELIVVPHTVKLNAVTSDTTWFQLNPPGPDPGPSYRVYLLGRGLDMNFSKHATVDICAYPEESLDYRYHLSMAHTEALRMTTKADQHDINEESYYHIAARIATSIFALSEMGRTTEYFLLDEIVDVQYQLKFLNYILMRIGAGAHPNTISGTSDLIFADPSQLHDELSLLFGQVKPANVDYFISYDEARDQLKTAYALSRGQDHVNALSLARRVIMSIYKGLLVKQNLNATERQALFFASMILLNFREGLENSSRVLDGRTTLLLMTSMCTAAHATQAALNIQEGLAYLNPSKHMFTIPNVYSPCMGSLRTDLTEEIHVMNLLSAIPTRPGLNEVLHTQLDESEIFDAAFKTMMIFTTWTAKDLHILHTHVPEVFTCQDAAARNGEYVLILPAVQGHSYVITRNKPQRGLVYSLADVDVYNPISVVYLSRDTCVSEHGVIETVALPHPDNLKECLYCGSVFLRYLTTGAIMDIIIIDSKDTERQLAAMGNSTIPPFNPDMHGDDSKAVLLFPNGTVVTLLGFERRQAIRMSGQYLGASLGGAFLAVVGFGIIGWMLCGNSRLREYNKIPLT

SEQ ID NO: 52 - protein, 661 aa, Homo sapiens
MFYEALKAELVYTRAVHGFRPRANCVVLSDYIPRVACNMGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 53 - protein, 868 aa, Homo sapiens
MFVTAVVSVSPSSFYESLQVEPTQSEDITRSAHLGDGDEIREAIHKSQDAETKPTFYVCPPPTGSTIVRLEPTRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 54 - protein, 340 aa, Homo sapiens
MQALGIKTEHFIIMCLLSGHAVFTLWYTARVKFEHECVYATTVINGGPVVWGSYNNSLIYVTFVNHSTFLDGLSGYDYSCRENLLSGDTMVKTAISTPLHDKIRIVLGTRNCHAYFWCVQLKMIFFAWFVYGMYLQFRRIRRMFGPFRSSCELISPTSYSLNYVTRVISNILLGYPYTKLARLLCDVSMRRDGMSKVFNADPISFLYMHKGVTLLMLLEVIAHISSGCIVLLTLGVAYTPCALLYPTYIRILAWVVVCTLAIVELISYVRPKPTKDNHLNHINTGGIRGICTTCCATVMSGLAIKCFYIVIFAIAVVIFMHYEQRVQVSLFGESENSQKH

SEQ ID NO: 55 - protein, 87 aa, Homo sapiens
MGSITASFILITMQILFFCEDSSGEPNFAERNFWHASCSARGVYIDGSMITTLFFYASLLGVCVALISLAYHACFRLFTRSVLRSTW

SEQ ID NO: 56 - protein, 18 aa, synthetic construct
MDWTWILFLVAAATRVHS

SEQ ID NO: 57 - protein, 20 aa, synthetic construct
METPAQLLFLLLLWLPDTTG

SEQ ID NO: 58 - protein, 6 aa, synthetic construct
AEAADA

SEQ ID NO: 59 - protein, 6 aa, synthetic construct
SESTDT

SEQ ID NO: 60 - RNA, 1885 nt, synthetic construct
  source 1..1885: mol_type = other RNA
gggaaataagagagaaaagaagagtaagaagaaatataagagccaccatgggcaccgtgaacaagcccgtcgtgggcgtgctgatgggcttcggcatcatcaccggcaccctgcggatcaccaatcctgtgcgggccagcgtgctgagatacgacgacttccacatcgacgaggacaagctggacaccaacagcgtgtacgagccctactaccacagcgaccacgccgagagcagctgggtcaacagaggcgagtccagccggaaggcctacgaccacaacagcccctacatctggccccggaacgactacgacggcttcctggaaaatgcccacgagcaccacggcgtgtacaaccagggcagaggcatcgacagcggcgagagactgatgcagcccacccagatgagcgcccaggaagatctgggcgacgacaccggcatccacgtgatccctaccctgaacggcgacgaccggcacaagatcgtgaacgtggaccagcggcagtacggcgacgtgttcaagggcgacctgaaccccaagccccagggacagcggctgattgaggtgtccgtggaagagaaccaccccttcaccctgagagcccctatccagcggatctacggcgtgcgctataccgagacttggagcttcctgcccagcctgacctgtactggcgacgccgctcctgccatccagcacatctgcctgaagcacaccacctgtttccaggacgtggtggtggacgtggactgcgccgagaacaccaaagaggaccagctggccgagatcagctaccggttccagggcaagaaagaggccgaccagccctggatcgtcgtgaacaccagcaccctgttcgacgagctggaactggaccctcccgagatcgaacccggggtgctgaaggtgctgcggaccgagaagcagtacctgggagtgtacatctggaacatgcggggcagcgacggcacctctacctacgccaccttcctcgtgacctggaagggcgacgagaaaacccggaaccctacccctgccgtgacccctcagcctagaggcgccgagtttcacatgtggaattaccacagccacgtgttcagcgtgggcgacaccttctccctggccatgcatctgcagtacaagatccacgaggcccctttcgacctgctgctggaatggctgtacgtgcccatcgaccctacctgccagcccatgcggctgtactccacctgtctgtaccaccccaacgcccctcagtgcctgagccacatgaatagcggctgcaccttcaccagccctcacctggctcagagggtggccagcaccgtgtaccagaattgcgagcacgccgacaactacaccgcctactgcctgggcatcagccacatggaacccagcttcggcctgatcctgcacgatggcggcaccaccctgaagttcgtggacacccctgagtccctgagcggcctgtacgtgttcgtggtgtacttcaacggccacgtggaagccgtggcctacaccgtggtgtccaccgtggaccacttcgtgaacgccatcgaggaacggggcttccctccaactgctggacagcctcctgccaccaccaagcccaaagaaatcacccctgtgaaccccggcaccagcccactgctgcgctatgctgcttggacaggcggactggctgctgtggtgctgctgtgcctcgtgattttcctgatctgcaccgccaagcggatgagagtgaaggccgccagagtggacaagtgataataggctggagcctcggtggccatgcttcttgccccttgggcctccccccagcccctcctccccttcctgcacccgtacccccgtggtctttgaataaagtctgagtgggcggc

SEQ ID NO: 61 - protein, 573 aa, synthetic construct; identical to SEQ ID NO: 38

SEQ ID NO: 62 - RNA, 1719 nt, synthetic construct
  source 1..1719: mol_type = other RNA
atgggcaccgtgaacaagcccgtcgtgggcgtgctgatgggcttcggcatcatcaccggcaccctgcggatcaccaatcctgtgcgggccagcgtgctgagatacgacgacttccacatcgacgaggacaagctggacaccaacagcgtgtacgagccctactaccacagcgaccacgccgagagcagctgggtcaacagaggcgagtccagccggaaggcctacgaccacaacagcccctacatctggccccggaacgactacgacggcttcctggaaaatgcccacgagcaccacggcgtgtacaaccagggcagaggcatcgacagcggcgagagactgatgcagcccacccagatgagcgcccaggaagatctgggcgacgacaccggcatccacgtgatccctaccctgaacggcgacgaccggcacaagatcgtgaacgtggaccagcggcagtacggcgacgtgttcaagggcgacctgaaccccaagccccagggacagcggctgattgaggtgtccgtggaagagaaccaccccttcaccctgagagcccctatccagcggatctacggcgtgcgctataccgagacttggagcttcctgcccagcctgacctgtactggcgacgccgctcctgccatccagcacatctgcctgaagcacaccacctgtttccaggacgtggtggtggacgtggactgcgccgagaacaccaaagaggaccagctggccgagatcagctaccggttccagggcaagaaagaggccgaccagccctggatcgtcgtgaacaccagcaccctgttcgacgagctggaactggaccctcccgagatcgaacccggggtgctgaaggtgctgcggaccgagaagcagtacctgggagtgtacatctggaacatgcggggcagcgacggcacctctacctacgccaccttcctcgtgacctggaagggcgacgagaaaacccggaaccctacccctgccgtgacccctcagcctagaggcgccgagtttcacatgtggaattaccacagccacgtgttcagcgtgggcgacaccttctccctggccatgcatctgcagtacaagatccacgaggcccctttcgacctgctgctggaatggctgtacgtgcccatcgaccctacctgccagcccatgcggctgtactccacctgtctgtaccaccccaacgcccctcagtgcctgagccacatgaatagcggctgcaccttcaccagccctcacctggctcagagggtggccagcaccgtgtaccagaattgcgagcacgccgacaactacaccgcctactgcctgggcatcagccacatggaacccagcttcggcctgatcctgcacgatggcggcaccaccctgaagttcgtggacacccctgagtccctgagcggcctgtacgtgttcgtggtgtacttcaacggccacgtggaagccgtggcctacaccgtggtgtccaccgtggaccacttcgtgaacgccatcgaggaacggggcttccctccaactgctggacagcctcctgccaccaccaagcccaaagaaatcacccctgtgaaccccggcaccagcccactgctgcgctatgctgcttggacaggcggactggctgctgtggtgctgctgtgcctcgtgattttcctgatctgcaccgccaagcggatgagagtgaaggccgccagagtggacaag

SEQ ID NO: 63 - RNA, 1990 nt, synthetic construct
  source 1..1990: mol_type = other RNA
gggaaataagagagaaaagaagagtaagaagaaatataagagccaccatgggcaccgtgaacaagcccgtcgtgggcgtgctgatgggcttcggcatcatcaccggcaccctgcggatcaccaatcctgtgcgggccagcgtgctgagatacgacgacttccacatcgacgaggacaagctggacaccaacagcgtgtacgagccctactaccacagcgaccacgccgagagcagctgggtcaacagaggcgagtccagccggaaggcctacgaccacaacagcccctacatctggccccggaacgactacgacggcttcctggaaaatgcccacgagcaccacggcgtgtacaaccagggcagaggcatcgacagcggcgagagactgatgcagcccacccagatgagcgcccaggaagatctgggcgacgacaccggcatccacgtgatccctaccctgaacggcgacgaccggcacaagatcgtgaacgtggaccagcggcagtacggcgacgtgttcaagggcgacctgaaccccaagccccagggacagcggctgattgaggtgtccgtggaagagaaccaccccttcaccctgagagcccctatccagcggatctacggcgtgcgctataccgagacttggagcttcctgcccagcctgacctgtactggcgacgccgctcctgccatccagcacatctgcctgaagcacaccacctgtttccaggacgtggtggtggacgtggactgcgccgagaacaccaaagaggaccagctggccgagatcagctaccggttccagggcaagaaagaggccgaccagccctggatcgtcgtgaacaccagcaccctgttcgacgagctggaactggaccctcccgagatcgaacccggggtgctgaaggtgctgcggaccgagaagcagtacctgggagtgtacatctggaacatgcggggcagcgacggcacctctacctacgccaccttcctcgtgacctggaagggcgacgagaaaacccggaaccctacccctgccgtgacccctcagcctagaggcgccgagtttcacatgtggaattaccacagccacgtgttcagcgtgggcgacaccttctccctggccatgcatctgcagtacaagatccacgaggcccctttcgacctgctgctggaatggctgtacgtgcccatcgaccctacctgccagcccatgcggctgtactccacctgtctgtaccaccccaacgcccctcagtgcctgagccacatgaatagcggctgcaccttcaccagccctcacctggctcagagggtggccagcaccgtgtaccagaattgcgagcacgccgacaactacaccgcctactgcctgggcatcagccacatggaacccagcttcggcctgatcctgcacgatggcggcaccaccctgaagttcgtggacacccctgagtccctgagcggcctgtacgtgttcgtggtgtacttcaacggccacgtggaagccgtggcctacaccgtggtgtccaccgtggaccacttcgtgaacgccatcgaggaacggggcttccctccaactgctggacagcctcctgccaccaccaagcccaaagaaatcacccctgtgaaccccggcaccagcccactgctgcgctatgctgcttggacaggcggactggctgctgtggtgctgctgtgcctcgtgattttcctgatctgcaccgccaagcggatgagagtgaaggccgccagagtggacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 64 - RNA, 1885 nt, synthetic construct
  source 1..1885: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

SEQ ID NO: 65 - protein, 573 aa, synthetic construct; identical to SEQ ID NO: 38

SEQ ID NO: 66 - RNA, 1719 nt, synthetic construct
  source 1..1719: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaag

SEQ ID NO: 67 - RNA, 1990 nt, synthetic construct
  source 1..1990: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 68 - RNA, 1885 nt, synthetic construct
  source 1..1885: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

SEQ ID NO: 69 - protein, 573 aa, synthetic construct; identical to SEQ ID NO: 38

SEQ ID NO: 70 - RNA, 1719 nt, synthetic construct
  source 1..1719: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaag

SEQ ID NO: 71 - RNA, 1990 nt, synthetic construct
  source 1..1990: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 72 - RNA, 1885 nt, synthetic construct
  source 1..1885: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

SEQ ID NO: 73 - protein, 573 aa, synthetic construct; identical to SEQ ID NO: 38

SEQ ID NO: 74 - RNA, 1719 nt, synthetic construct
  source 1..1719: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaag

SEQ ID NO: 75 - RNA, 1990 nt, synthetic construct
  source 1..1990: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 76 - RNA, 1885 nt, synthetic construct
  source 1..1885: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

SEQ ID NO: 77 - protein, 573 aa, synthetic construct; identical to SEQ ID NO: 38

SEQ ID NO: 78 - RNA, 1719 nt, synthetic construct
  source 1..1719: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaag

SEQ ID NO: 79 - RNA, 1990 nt, synthetic construct
  source 1..1990: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 80 - RNA, 1885 nt, synthetic construct
  source 1..1885: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

SEQ ID NO: 81 - protein, 573 aa, synthetic construct; identical to SEQ ID NO: 38

SEQ ID NO: 82 - RNA, 1719 nt, synthetic construct
  source 1..1719: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaag

SEQ ID NO: 83 - RNA, 1990 nt, synthetic construct
  source 1..1990: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 84 - RNA, 1885 nt, synthetic construct
  source 1..1885: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

SEQ ID NO: 85 - protein, 573 aa, synthetic construct; identical to SEQ ID NO: 38

SEQ ID NO: 86 - RNA, 1719 nt, synthetic construct
  source 1..1719: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaag

SEQ ID NO: 87 - RNA, 1990 nt, synthetic construct
  source 1..1990: mol_type = other RNA
gggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 88 - RNA, 1885 nt, synthetic construct
  source 1..1885: mol_type = other RNA
gggaaataagagagaaaagaagagtaagaagaaatataagagccaccatggggacagttaataaacctgtggtgggcgtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggattcttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagcttcttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgttcttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacggaggcaccacgttaaagtttgtagatacacccgagagtttgtcgggattatacgtctttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacgcccgtaaaccccggaacgtcaccacttctacgatatgccgcatggaccggagggcttgcagcagtagtacttttatgtctcgtaatattcttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

SEQ ID NO: 89 - protein, 573 aa, synthetic construct; identical to SEQ ID NO: 38

SEQ ID NO: 90 - RNA, 1719 nt, synthetic construct
  source 1..1719: mol_type = other RNA
atggggacagttaataaacctgtggtgggcgtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggattcttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagcttcttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgttcttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacggaggcaccacgttaaagtttgtagatacacccgagagtttgtcgggattatacgtctttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacgcccgtaaaccccggaacgtcaccacttctacgatatgccgcatggaccggagggcttgcagcagtagtacttttatgtctcgtaatattcttaatctgtacggctaaacgaatgagggttaaagccgccagggtagacaag

SEQ ID NO: 91 - RNA, 1990 nt, synthetic construct
  source 1..1990: mol_type = other RNA
gggaaataagagagaaaagaagagtaagaagaaatataagagccaccatggggacagttaataaacctgtggtgggcgtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggattcttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagcttcttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgttcttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacggaggcaccacgttaaagtttgtagatacacccgagagtttgtcgggattatacgtctttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacgcccgtaaaccccggaacgtcaccacttctacgatatgccgcatggaccggagggcttgcagcagtagtacttttatgtctcgtaatattcttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 92 - RNA, 2141 nt, unidentified; identical to SEQ ID NO: 12
  source 1..2141: mol_type = genomic RNA; note = Varicella Zoster Virus

SEQ ID NO: 93 - RNA, 2111 nt, synthetic construct; identical to SEQ ID NO: 16
  source 1..2111: mol_type = other RNA

SEQ ID NO: 94 - RNA, 1958 nt, synthetic construct; identical to SEQ ID NO: 20
  source 1..1958: mol_type = other RNA

SEQ ID NO: 95 - RNA, 1928 nt, synthetic construct; identical to SEQ ID NO: 24
  source 1..1928: mol_type = other RNA

SEQ ID NO: 96 - RNA, 2144 nt, synthetic construct; identical to SEQ ID NO: 28
  source 1..2144: mol_type = other RNA

SEQ ID NO: 97 - RNA, 2144 nt, synthetic construct; identical to SEQ ID NO: 32
  source 1..2144: mol_type = other RNA

SEQ ID NO: 98 - RNA, 1994 nt, synthetic construct; identical to SEQ ID NO: 36
  source 1..1994: mol_type = other RNA

SEQ ID NO: 99 - RNA, 1994 nt, synthetic construct; identical to SEQ ID NO: 40
  source 1..1994: mol_type = other RNA

SEQ ID NO: 100 - RNA, 1337 nt, synthetic construct; identical to SEQ ID NO: 44
  source 1..1337: mol_type = other RNA

SEQ ID NO: 101 - RNA, 1991 nt, synthetic construct
  source 1..1991: mol_type = other RNA
ggggaaataagagagaaaagaagagtaagaagaaatataagagccaccatgggcaccgtgaacaagcccgtcgtgggcgtgctgatgggcttcggcatcatcaccggcaccctgcggatcaccaatcctgtgcgggccagcgtgctgagatacgacgacttccacatcgacgaggacaagctggacaccaacagcgtgtacgagccctactaccacagcgaccacgccgagagcagctgggtcaacagaggcgagtccagccggaaggcctacgaccacaacagcccctacatctggccccggaacgactacgacggcttcctggaaaatgcccacgagcaccacggcgtgtacaaccagggcagaggcatcgacagcggcgagagactgatgcagcccacccagatgagcgcccaggaagatctgggcgacgacaccggcatccacgtgatccctaccctgaacggcgacgaccggcacaagatcgtgaacgtggaccagcggcagtacggcgacgtgttcaagggcgacctgaaccccaagccccagggacagcggctgattgaggtgtccgtggaagagaaccaccccttcaccctgagagcccctatccagcggatctacggcgtgcgctataccgagacttggagcttcctgcccagcctgacctgtactggcgacgccgctcctgccatccagcacatctgcctgaagcacaccacctgtttccaggacgtggtggtggacgtggactgcgccgagaacaccaaagaggaccagctggccgagatcagctaccggttccagggcaagaaagaggccgaccagccctggatcgtcgtgaacaccagcaccctgttcgacgagctggaactggaccctcccgagatcgaacccggggtgctgaaggtgctgcggaccgagaagcagtacctgggagtgtacatctggaacatgcggggcagcgacggcacctctacctacgccaccttcctcgtgacctggaagggcgacgagaaaacccggaaccctacccctgccgtgacccctcagcctagaggcgccgagtttcacatgtggaattaccacagccacgtgttcagcgtgggcgacaccttctccctggccatgcatctgcagtacaagatccacgaggcccctttcgacctgctgctggaatggctgtacgtgcccatcgaccctacctgccagcccatgcggctgtactccacctgtctgtaccaccccaacgcccctcagtgcctgagccacatgaatagcggctgcaccttcaccagccctcacctggctcagagggtggccagcaccgtgtaccagaattgcgagcacgccgacaactacaccgcctactgcctgggcatcagccacatggaacccagcttcggcctgatcctgcacgatggcggcaccaccctgaagttcgtggacacccctgagtccctgagcggcctgtacgtgttcgtggtgtacttcaacggccacgtggaagccgtggcctacaccgtggtgtccaccgtggaccacttcgtgaacgccatcgaggaacggggcttccctccaactgctggacagcctcctgccaccaccaagcccaaagaaatcacccctgtgaaccccggcaccagcccactgctgcgctatgctgcttggacaggcggactggctgctgtggtgctgctgtgcctcgtgattttcctgatctgcaccgccaagcggatgagagtgaaggccgccagagtggacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 102 - RNA, 1991 nt, synthetic construct
  source 1..1991: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 103 - RNA, 1991 nt, synthetic construct
  source 1..1991: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 104 - RNA, 1991 nt, synthetic construct
  source 1..1991: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 105 - RNA, 1991 nt, synthetic construct
  source 1..1991: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 106 - RNA, 1991 nt, synthetic construct
  source 1..1991: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 107 - RNA, 1991 nt, synthetic construct
  source 1..1991: mol_type = other RNA
ggggaaataagagagaaaagaagagtaagaagaaatataagagccaccatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 108 - RNA, 1965 nt, synthetic construct
  source 1..1965: mol_type = other RNA
gagaagaaatataagagccaccatggggacagttaataaacctgtggtgggcgtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaggcgtacgatcataactcaccttatatatggccacgtaatgattatgatggattcttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataagattgtaaatgtggaccaacgtcaatacggtgacgtgtttaaaggagatcttaatccaaagccccaaggccaaagactcattgaggtgtcagtggaagagaatcacccgtttactttacgcgcaccgattcagcggatttatggagtccggtacaccgagacttggagcttcttgccgtcattaacctgtacgggagacgcagcgcccgccatccagcatatatgtttaaagcatacaacatgctttcaagacgtggtggtggatgtggattgcgcggagaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgttcttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacggaggcaccacgttaaagtttgtagatacacccgagagtttgtcgggattatacgtctttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacgcccgtaaaccccggaacgtcaccacttctacgatatgccgcatggaccggagggcttgcagcagtagtacttttatgtctcgtaatattcttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 109 - protein, 24 aa, synthetic construct
MLGSNSGQRVVFTILLLLVAPAYS

SEQ ID NO: 110 - protein, 17 aa, synthetic construct
MKCLLYLAFLFIGVNCA

SEQ ID NO: 111 - protein, 15 aa, synthetic construct
MWLVSLAIVTACAGA

SEQ ID NO: 112 - RNA, 1729 nt, synthetic construct
  source 1..1729: mol_type = other RNA
tcaagcttttggaccctcgtacagaagctaatacgactcactatagggaaataagagagaaaagaagagtaagaagaaatataagagccaccatggcacaagtcattaatacaaacagcctgtcgctgttgacccagaataacctgaacaaatcccagtccgcactgggcactgctatcgagcgtttgtcttccggtctgcgtatcaacagcgcgaaagacgatgcggcaggacaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgcgaatggtactaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatattgatttaaaagaaatcagctctaaaacactgggacttgataagcttaatgtccaagatgcctacaccccgaaagaaactgctgtaaccgttgataaaactacctataaaaatggtacagatcctattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttggcaactgcggaagctacagctattcggggaacggccactataacccacaaccaaattgctgaagtaacaaaagagggtgttgatacgaccacagttgcggctcaacttgctgcagcaggggttactggcgccgataaggacaatactagccttgtaaaactatcgtttgaggataaaaacggtaaggttattgatggtggctatgcagtgaaaatgggcgacgatttctatgccgctacatatgatgagaaaacaggtgcaattactgctaaaaccactacttatacagatggtactggcgttgctcaaactggagctgtgaaatttggtggcgcaaatggtaaatctgaagttgttactgctaccgatggtaagacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggttcagaaccgtttcaactccgctatcaccaacctgggcaataccgtaaataacctgtcttctgcccgtagccgtatcgaagattccgactacgcaaccgaagtctccaacatgtctcgcgcgcagattctgcagcaggccggtacctccgttctggcgcaggcgaaccaggttccgcaaaacgtcctctctttactgcgttgataataggctggagcctcggtggccatgcttcttgccccttgggcctccccccagcccctcctccccttcctgcacccgtacccccgtggtctttgaataaagtctgagtgggcggc

SEQ ID NO: 113 - RNA, 1518 nt, synthetic construct
  source 1..1518: mol_type = other RNA
atggcacaagtcattaatacaaacagcctgtcgctgttgacccagaataacctgaacaaatcccagtccgcactgggcactgctatcgagcgtttgtcttccggtctgcgtatcaacagcgcgaaagacgatgcggcaggacaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgcgaatggtactaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatattgatttaaaagaaatcagctctaaaacactgggacttgataagcttaatgtccaagatgcctacaccccgaaagaaactgctgtaaccgttgataaaactacctataaaaatggtacagatcctattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttggcaactgcggaagctacagctattcggggaacggccactataacccacaaccaaattgctgaagtaacaaaagagggtgttgatacgaccacagttgcggctcaacttgctgcagcaggggttactggcgccgataaggacaatactagccttgtaaaactatcgtttgaggataaaaacggtaaggttattgatggtggctatgcagtgaaaatgggcgacgatttctatgccgctacatatgatgagaaaacaggtgcaattactgctaaaaccactacttatacagatggtactggcgttgctcaaactggagctgtgaaatttggtggcgcaaatggtaaatctgaagttgttactgctaccgatggtaagacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggttcagaaccgtttcaactccgctatcaccaacctgggcaataccgtaaataacctgtcttctgcccgtagccgtatcgaagattccgactacgcaaccgaagtctccaacatgtctcgcgcgcagattctgcagcaggccggtacctccgttctggcgcaggcgaaccaggttccgcaaaacgtcctctctttactgcgt

SEQ ID NO: 114 - RNA, 1790 nt, synthetic construct
  source 1..1790: mol_type = other RNA
ggggaaataagagagaaaagaagagtaagaagaaatataagagccaccatggcacaagtcattaatacaaacagcctgtcgctgttgacccagaataacctgaacaaatcccagtccgcactgggcactgctatcgagcgtttgtcttccggtctgcgtatcaacagcgcgaaagacgatgcggcaggacaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgcgaatggtactaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatattgatttaaaagaaatcagctctaaaacactgggacttgataagcttaatgtccaagatgcctacaccccgaaagaaactgctgtaaccgttgataaaactacctataaaaatggtacagatcctattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttggcaactgcggaagctacagctattcggggaacggccactataacccacaaccaaattgctgaagtaacaaaagagggtgttgatacgaccacagttgcggctcaacttgctgcagcaggggttactggcgccgataaggacaatactagccttgtaaaactatcgtttgaggataaaaacggtaaggttattgatggtggctatgcagtgaaaatgggcgacgatttctatgccgctacatatgatgagaaaacaggtgcaattactgctaaaaccactacttatacagatggtactggcgttgctcaaactggagctgtgaaatttggtggcgcaaatggtaaatctgaagttgttactgctaccgatggtaagacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggttcagaaccgtttcaactccgctatcaccaacctgggcaataccgtaaataacctgtcttctgcccgtagccgtatcgaagattccgactacgcaaccgaagtctccaacatgtctcgcgcgcagattctgcagcaggccggtacctccgttctggcgcaggcgaaccaggttccgcaaaacgtcctctctttactgcgttgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 115 - protein, 506 aa, synthetic construct
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDPITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGAITAKTTTYTDGTGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 116 - protein, 698 aa, synthetic construct
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRGGGGSGGGGSMMAPDPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIKNSISTEWSPCSVTCGNGIQVRIKPGSANKPKDELDYENDIEKKICKMEKCSSVFNVVNS

SEQ ID NO: 117 - protein, 692 aa, synthetic construct
MMAPDPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIKNSISTEWSPCSVTCGNGIQVRIKPGSANKPKDELDYENDIEKKICKMEKCSSVFNVVNSRPVTMAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 118 - protein, 13 aa, unidentified
  source 1..13: note = Salmonella typhimurium
LQRVRELAVQSAN

SEQ ID NO: 119 - protein, 4 aa, synthetic construct
AYRV

SEQ ID NO: 120 - protein, 4 aa, synthetic construct
AARV

SEQ ID NO: 121 - protein, 4 aa, synthetic construct
YAGL

SEQ ID NO: 122 - protein, 4 aa, synthetic construct
SSTT

SEQ ID NO: 123 - RNA, 2080 nt, unidentified; identical to SEQ ID NO: 9
  source 1..2080: mol_type = genomic RNA; note = Varicella Zoster Virus

SEQ ID NO: 124 - RNA, 1276 nt, unidentified; identical to SEQ ID NO: 2
  source 1..1276: mol_type = genomic RNA; note = Varicella Zoster Virus

SEQ ID NO: 125 - RNA, 1897 nt, synthetic construct; identical to SEQ ID NO: 17
  source 1..1897: mol_type = other RNA

SEQ ID NO: 126 - RNA, 1867 nt, synthetic construct; identical to SEQ ID NO: 21
  source 1..1867: mol_type = other RNA

SEQ ID NO: 127 - RNA, 1933 nt, synthetic construct; identical to SEQ ID NO: 33
  source 1..1933: mol_type = other RNA

SEQ ID NO: 128 - RNA, 1933 nt, synthetic construct; identical to SEQ ID NO: 37
  source 1..1933: mol_type = other RNA

SEQ ID NO: 129 - RNA, 2083 nt, synthetic construct; identical to SEQ ID NO: 25
  source 1..2083: mol_type = other RNA

SEQ ID NO: 130 - RNA, 2083 nt, synthetic construct; identical to SEQ ID NO: 29
  source 1..2083: mol_type = other RNA

SEQ ID NO: 131 - RNA, 2050 nt, synthetic construct; identical to SEQ ID NO: 13
  source 1..2050: mol_type = other RNA

SEQ ID NO: 132 - protein, 4 aa, synthetic construct
GAGL

SEQ ID NO: 133 - RNA, 1844 nt, synthetic construct
  source 1..1844: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgataatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgatgataataggctggagcctcggtggccatgcttcttgccccttgggcctccccccagcccctcctccccttcctgcacccgtacccccgtggtctttgaataaagtctgagtgggcggc

SEQ ID NO: 134 - RNA, 1838 nt, synthetic construct
  source 1..1838: mol_type = other RNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaagcatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccacccgagattgaaccgggtgtcttgaaagtacttcggacagagaaacaatacttgggtgtgtacatttggaacatgcgcggctccgatggtacgtctacctacgccacgtttttggtcacctggaaaggggatgagaagacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcagaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagccgccagggtagacaagtgataataggctggagcctcggtggccatgcttcttgccccttgggcctccccccagcccctcctccccttcctgcacccgtacccccgtggtctttgaataaagtctgagtgggcggc

(19) United States
(12) Patent Application Publication
(10) Pub. No.: US 2025/0188132 A1
(43) Pub. Date: Jun. 12, 2025

(54) NUCLEIC ACID VACCINES FOR VARICELLA ZOSTER VIRUS (VZV)

(71) Applicant: ModernaTX, Inc., Cambridge, MA (US)

(72) Inventors: Giuseppe Ciaramella, Sudbury, MA (US); Sayda Mahgoub Elbashir, Cambridge, MA (US)

(73) Assignee: ModernaTX, Inc., Cambridge, MA (US)

(21) Appl. No.: 18/927,762

(22) Filed: Oct. 25, 2024

Related U.S. Application Data
(63) Continuation of application No. 18/365,948, filed on Aug. 4, 2023.
Application No. 18/365,948 is a continuation of application No. 18/176,014, filed on Feb. 28, 2023, now abandoned.
Application No. 18/176,014 is a division of application No. 15/767,587, filed on Oct. 10, 2018, now Pat. No. 11,643,441.
(60) Provisional application No. 62/335,348, filed on May 12, 2016.
(60) Provisional application No. 62/247,697, filed on Oct. 28, 2015.
(60) Provisional application No. 62/245,234, filed on Oct. 22, 2015.
(60) Provisional application No. 62/245,031, filed on Oct. 22, 2015.

(51) Int. Cl. C07K 14/005; A61K 9/00; A61K 9/51; A61K 39/00; A61K 39/25

(52) U.S. Cl. CPC C07K 14/005; A61K 9/0019; A61K 9/5123; A61K 39/25; A61K 2039/53; A61K 2039/54; A61K 2039/545; A61K 2039/575; A61K 2039/70; C12N 2710/16722; C12N 2710/16734; C12N 2710/16771

(57) ABSTRACT

Aspects of the disclosure relate to nucleic acid vaccines. The vaccines include at least one RNA polynucleotides having an open reading frame encoding at least one varicella zoster virus (VZV) antigen. Methods for preparing and using such vaccines are also described.

### RELATED APPLICATIONS

This application is a continuation of U.S. application Ser. No. 18/365,948, filed Aug. 4, 2023, which is a continuation of U.S. application Ser. No. 18/176,014, filed Feb. 28, 2023, which is a divisional application of U.S. application Ser. No. 15/767,587, filed Oct. 10, 2018, which is a national stage filing under 35 U.S.C. § 371 of international patent application number PCT/US2016/058297, filed Oct. 21, 2016, which claims the benefit under 35 U.S.C. § 119 (e) of U.S. provisional application No. 62/245,234, filed Oct. 22, 2015, U.S. provisional application No. 62/247,697, filed Oct. 28, 2015, U.S. provisional application No. 62/335,348, filed May 12, 2016, and U.S. provisional application No. 62/245,031, filed Oct. 22, 2015, each of which is incorporated by reference herein in its entirety.

### REFERENCE TO AN ELECTRONIC SEQUENCE LISTING

The contents of the electronic sequence listing (M137870014US07-SUBSEQ-HCL.xm1; Size: 305,358 bytes; and Date of Creation: Nov. 5, 2024) is herein incorporated by reference in its entirety.

### BACKGROUND

Varicella is an acute infectious disease caused by varicella zoster virus (VZV). Varicella zoster virus is one of eight herpesviruses known to infect humans and vertebrates. VZV is also known as chickenpox virus, varicella virus, zoster virus, and human herpesvirus type 3 (HHV-3). VZV only affects humans, and commonly causes chickenpox in children, teens and young adults and herpes zoster (shingles) in adults (rarely in children). The primary VZV infection, which results in chickenpox (varicella), may result in complications, including viral or secondary bacterial pneumonia. Even when the clinical symptoms of chickenpox have resolved, VZV remains dormant in the nervous system of the infected person (virus latency) in the trigeminal and dorsal root ganglia. In about 10-20% of cases, VZV reactivates later in life, travelling from the sensory ganglia back to the skin where it produces a disease (rash) known as shingles or herpes zoster. VZV can also cause a number of neurologic conditions ranging from aseptic meningitis to encephalitis. Other serious complications of VZV infection include postherpetic neuralgia, Mollaret's meningitis, zoster multiplex, thrombocytopenia, myocarditis, arthritis, and inflammation of arteries in the brain leading to stroke, myelitis, herpes ophthalmicus, or zoster sine herpete. In rare instances, VZV affects the geniculate ganglion, giving lesions that follow specific branches of the facial nerve. Symptoms may include painful blisters on the tongue and car along with one sided facial weakness and hearing loss.

Varicella cases have declined 97% since 1995, mostly due to vaccination. However, an estimated 500,000 to 1 million episodes of herpes zoster (shingles) occur annually in just the United States. The lifetime risk of herpes zoster is estimated to be at least 32%, with increasing age and cellular immunosuppression being the most important risk factors. In fact, it is estimated that 50% of persons living until the age of 85 will develop herpes zoster.

A live attenuated VZV Oka strain vaccine is available and is marketed in the United States under the trade name VARIVAX® (Merck). A similar, but not identical, VZV vaccine is marketed globally as VARILRIX® (GlaxoSmithKline). Since its approval in 1995, it has been added to the recommended vaccination schedules for children in Australia, the United States, and several other countries. In 2007, the Advisory Committee on Immunization Practices (ACIP) recommended a second dose of vaccine before school entry to ensure the maintenance of high levels of varicella immunity. In 2001-2005, outbreaks were reported in schools with high varicella vaccination coverage, indicating that even in settings where most children were vaccinated and the vaccine performed as expected, varicella outbreaks could not be prevented with the one-dose vaccination policy. As a result, two-dose vaccination is the adopted protocol; however, even with two doses of vaccine, there are reported incidences of breakthrough varicella. Furthermore, varicella vaccination has raised concerns that the immunity induced by the vaccine may not be lifelong, possibly leaving adults vulnerable to more severe disease as the immunity from their childhood immunization wanes.

In 2005, the FDA approved the combined live attenuated combination measles-mumps-rubella-varicella (MMRV) vaccine PROQUAD™ (Merck) for use in persons 12 months to 12 years in age. While the attenuated measles, mumps, and rubella vaccine viruses in MMRV are identical and of equal titer to those in the MMR vaccine, the titer of Oka/Merck VZV is higher in MMRV vaccine than in single-antigen varicella vaccine.

In 2006, the United States Food and Drug Administration approved ZOSTAVAX® (Merck) for the prevention of shingles (herpes zoster) in persons 60 years or older (currently 50-59 years of age is approved). ZOSTAVAX® contains the same Oka/Merck varicella zoster virus used in the varicella and MMRV vaccines, but at a much higher titer (>10-fold higher viral dose) than that present in both of these vaccines, as the concentrated formulation is designed to elicit an immune response in older adults whose immunity to VZV wanes with advancing age.

Although the varicella vaccine has been shown to be safe in healthy individuals, there is evidence that immunity to VZV infection conferred by the vaccine wanes over time, rendering the vaccinated individuals susceptible to shingles, a more serious condition. In addition, there have been reports that individuals have developed chicken pox or shingles from the varicella vaccination. The vaccine may establish a latent infection in neural ganglia, which can then reactivate to cause herpes zoster.

Moreover, live attenuated virus is not suitable for all subjects, including pregnant women and persons with moderate or severe acute illnesses. Also, varicella is not suitable or approved for immunocompromised patients, including persons with immunosuppression due to leukemia, lymphoma, generalized malignancy, immune deficiency disease or immunosuppressive therapy. Likewise, persons with moderate or severe cellular immunodeficiency resulting from infection with human immunodeficiency virus (HIV) including those diagnosed with acquired immunodeficiency syndrome (AIDS) should not receive the varicella vaccine. Thus, despite the high risk of morbidity and mortality associated with herpes zoster in immunocompromised individuals, this population is not eligible for vaccination with a live attenuated vaccine, such as ZOSTAVAX®.

There are one million cases of herpes zoster in the U.S. each year. An estimated $1 billion is spent annually on direct medical costs for herpes zoster in the US and treatment for herpes zoster is not always effective or available.

Deoxyribonucleic acid (DNA) vaccination is one technique used to stimulate humoral and cellular immune responses to foreign antigens, such as VZV antigens. The direct injection of genetically engineered DNA (e.g., naked plasmid DNA) into a living host results in a small number of host cells directly producing an antigen, resulting in a protective immunological response. With this technique, however, comes potential problems, including the possibility of insertional mutagenesis, which could lead to the activation of oncogenes or the inhibition of tumor suppressor genes.

### SUMMARY

Provided herein is a ribonucleic acid (RNA) vaccine that builds on the knowledge that RNA (e.g., messenger RNA (mRNA)) can safely direct the body's cellular machinery to produce nearly any protein of interest, from native proteins to antibodies and other entirely novel protein constructs that can have therapeutic activity inside and outside of cells. The varicella zoster virus (VZV) RNA vaccines of the present disclosure may be used to induce a balanced immune response against VZV comprising both cellular and humoral immunity, without many of the risks associated with attenuated virus vaccination.

The RNA (e.g., mRNA) vaccines may be utilized in various settings depending on the prevalence of the infection or the degree or level of unmet medical need. The RNA (e.g., mRNA) vaccines may be utilized to treat and/or prevent a VZV of various genotypes, strains, and isolates. The RNA (e.g., mRNA) vaccines have superior properties in that they produce much larger antibody titers and produce responses earlier than commercially available anti-viral therapeutic treatments. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Various VZV amino acid sequences encompasses by the present disclosure are provided in Tables 1-9. RNA (e.g., mRNA) vaccines as provided herein may include at least one RNA polynucleotide encoding at least one of the VZV glycoproteins provided in Table 1, or a fragment, homolog (e.g., having at least 80%, 85%, 90%, 95%, 98% or 99% identity) or variant or derivative thereof.

Some embodiments of the present disclosure provide VZV vaccines that include at least one ribonucleic acid (RNA) polynucleotide having an open reading frame encoding at least one VZV antigenic polypeptide or an immunogenic fragment or epitope thereof. Some embodiments of the present disclosure provide VZV vaccines that include at least one RNA polynucleotide having an open reading frame encoding two or more VZV antigenic polypeptides or an immunogenic fragment or epitope thereof. Some embodiments of the present disclosure provide VZV vaccines that include two or more RNA polynucleotides having an open reading frame encoding two or more VZV antigenic polypeptides or immunogenic fragments or epitopes thereof.

In some embodiments, an antigenic polypeptide is a VZV glycoprotein. For example, a VZV glycoprotein may be VZV gE, gI, gB, gH, gK, gL, gC, gN, or gM or an immunogenic fragment or epitope thereof. In some embodiments, the antigenic polypeptide is a VZV gE polypeptide. In some embodiments, the antigenic polypeptide is a VZV gI polypeptide. In some embodiments, the antigenic polypeptide is a VZV gB polypeptide. In some embodiments, the antigenic polypeptide is a VZV gH polypeptide. In some embodiments, the antigenic polypeptide is a VZV gK polypeptide. In some embodiments, the antigenic polypeptide is a VZV gL polypeptide. In some embodiments, the antigenic polypeptide is a VZV gC polypeptide. In some embodiments, the antigenic polypeptide is a VZV gN polypeptide. In some embodiments, the antigenic polypeptide is a VZV gM polypeptide. In some embodiments, the VZV glycoprotein is encoded by a nucleic acid sequence of SEQ ID NO: 1 or SEQ ID NO: 2.

In some embodiments, the VZV glycoprotein is a variant gE polypeptide. In some embodiments, the variant VZV gE polypeptide is a truncated polypeptide lacking the anchor domain (ER retention domain). In some embodiments, the truncated VZV gE polypeptide comprises (or consists of, or consists essentially of) amino acids 1-561 of VZV gE polypeptide. In some embodiments, the truncated VZV gE polypeptide comprises (or consists of, or consists essentially of) amino acids 1-561 of SEQ ID NO: 10. In some embodiments, the truncated VZV gE polypeptide comprises (or consists of, or consists essentially of) amino acids 1-573 of SEQ ID NO: 18. In some embodiments, the truncated VZV gE polypeptide comprises (or consists of, or consists essentially of) amino acids 1-573 of SEQ ID NO: 10. In some embodiments, the variant VZV gE polypeptide is a truncated polypeptide lacking the carboxy terminal tail domain. In some embodiments, the truncated VZV gE polypeptide comprises (or consists of, or consists essentially of) amino acids 1-573 of VZV gE polypeptide. In some embodiments, the truncated VZV gE polypeptide comprises (or consists of, or consists essentially of) amino acids 1-573 of SEQ ID NO: 34.

In some embodiments, the variant VZV gE polypeptide has at least one mutation in one or more motif(s) associated with ER retention, wherein the mutation(s) in one or more motif(s) results in decreased retention of the VZV gE polypeptide in the ER and/or golgi. In some embodiments, the variant VZV gE polypeptide has at least one mutation in one or more motif(s) associated with targeting gE to the golgi or trans-golgi network (TGN), wherein the mutation(s) in one or more motif(s) results in decreased targeting or localization of the VZV gE polypeptide to the golgi or TGN. In some embodiments, the variant VZV gE polypeptide has at least one mutation in one or more motif(s) associated with the internalization of VZV gE or the endocytosis of gE, wherein the mutation(s) in one or more motif(s) results in decreased endocytosis of the VZV gE polypeptide. In some embodiments, the variant VZV gE polypeptide has at least one mutation in one or more phosphorylated acidic motif(s), such as SSTT (SEQ ID NO: 122). In some embodiments, the variant VZV gE polypeptide is a full-length VZV gE polypeptide having a Y582G mutation. In some embodiments, the variant VZV gE polypeptide is a full-length VZV gE polypeptide having a Y569A mutation. In some embodiments, the variant VZV gE polypeptide is a full-length VZV gE polypeptide having a Y582G mutation and a Y569A mutation. In some embodiments, the variant VZV gE polypeptide is an antigenic fragment comprising amino acids 1-573 of VZV gE and having a Y569A mutation. In some embodiments, the variant VZV gE polypeptide is an antigenic fragment comprising SEQ ID NO: 38.

In some embodiments, the variant VZV gE polypeptide is a full-length VZV gE polypeptide having an Igkappa sequence. In some embodiments, the variant VZV gE polypeptide is SEQ ID NO: 14. In some embodiments, the variant VZV gE polypeptide is a full-length VZV gE polypeptide having an A-E-A-A-D-A sequence (SEQ ID NO: 58) that replaces SESTDT (SEQ ID NO: 59). This is a replacement of the Ser/Thr-rich “SSTT” (SEQ ID NO: 122) acidic cluster with an Ala-rich sequence. In some embodiments, the variant VZV gE polypeptide is SEQ ID NO: 26. In some embodiments in which the VZV gE polypeptide has an A-E-A-A-D-A sequence (SEQ ID NO: 58), the variant VZV gE polypeptide also has at least one mutation in one or more motif(s) associated with ER/golgi retention, TGN localization, or endocytosis (e.g., has a Y582G mutation, a Y569A mutation, or both a Y582G mutation and a Y569A mutation) and/or has at least one mutation in one or more phosphorylated acidic motif(s), such as a SSTT (SEQ ID NO: 122) motif. In some embodiments, the variant VZV gE polypeptide is or comprises the amino acid sequence of SEQ ID NO: 30.

In some embodiments, the variant VZV gE polypeptide is a full-length VZV gE polypeptide having an additional sequence at the C-terminus that aids in secretion of the polypeptide or its localization to the cell membrane. In some embodiments, the variant VZV gE polypeptide is a full-length VZV gE polypeptide having an IgKappa sequence at the C-terminus. In some embodiments, the VZV gE polypeptide has additional sequence at the C-terminus that aids in secretion (e.g., has an IgKappa sequence at the C-terminus) and has at least one mutation in one or more motif(s) associated with ER retention, TGN localization, or endocytosis (e.g., has a Y582G mutation, a Y569A mutation, or both a Y582G mutation and a Y569A mutation) and/or has at least one mutation in one or more phosphorylated acidic motif(s), such as the SSTT (SEQ ID NO: 122) motif. In some embodiments, the variant VZV gE polypeptide is a truncated polypeptide lacking the anchor domain (ER retention domain) and having an additional sequence at the C-terminus that aids in secretion of the polypeptide (e.g., an IgKappa sequence at the C-terminus). In some embodiments, the truncated VZV gE polypeptide comprises amino acids 1-561 and has an IgKappa sequence at the C-terminus. In some embodiments, the variant polypeptide is SEQ ID NO: 22. In some embodiments, the variant VZV gE polypeptide is a truncated polypeptide lacking the carboxy terminal tail domain and having an additional sequence at the C-terminus that aids in secretion of the polypeptide, for example, having an IgKappa sequence at the C-terminus. In some embodiments, the truncated VZV gE polypeptide comprises amino acids 1-573 and has an IgKappa sequence at the C-terminus.

In some embodiments, the antigenic polypeptide comprises two or more glycoproteins. In some embodiments, the two or more glycoproteins are encoded by a single RNA polynucleotide. In some embodiments, the two or more glycoproteins are encoded by two or more RNA polynucleotides, for example, each glycoprotein is encoded by a separate RNA polynucleotide. In some embodiments, the two or more glycoproteins can be any combination of VZV gE, gI, gB, gH, gK, gL, gC, gN, and gM polypeptides or immunogenic fragments or epitopes thereof. In some embodiments, the two or more glycoproteins can be any combination of VZV gE and at least one of gI, gB, gH, gK, gL, gC, gN, and gM polypeptides or immunogenic fragments or epitopes thereof. In some embodiments, the two or more glycoproteins can be any combination of VZV gI and at least one of gE, gB, gH, gK, gL, gC, gN, and gM polypeptides or immunogenic fragments or epitopes thereof. In some embodiments, the two or more glycoproteins can be any combination of VZV gE, gI, and at least one of gB, gH, gK, gL, gC, gN, and gM polypeptides or immunogenic fragments or epitopes thereof.

In some embodiments, the two or more VZV glycoproteins are gE and gI. In some embodiments, the two or more VZV glycoproteins are gE and gB. In some embodiments, the two or more VZV glycoproteins are gI and gB. In some embodiments, the two or more VZV glycoproteins are gE, gI, and gB. In some embodiments, the two or more VZV glycoproteins are gE and gH. In some embodiments, the two or more VZV glycoproteins are gI and gH. In some embodiments, the two or more VZV glycoproteins are gE, gI, and gH. In some embodiments, the two or more VZV glycoproteins are gE and gK. In some embodiments, the two or more VZV glycoproteins are gI and gK. In some embodiments, the two or more VZV glycoproteins are gE, gI, and gK. In some embodiments, the two or more VZV glycoproteins are gE and gL. In some embodiments, the two or more VZV glycoproteins are gI and gL. In some embodiments, the two or more VZV glycoproteins are gE, gI, and gL. In some embodiments, the two or more VZV glycoproteins are gE and gC. In some embodiments, the two or more VZV glycoproteins are gI and gC. In some embodiments, the two or more VZV glycoproteins are gE, gI, and gC. In some embodiments, the two or more VZV glycoproteins are gE and gN. In some embodiments, the two or more VZV glycoproteins are gI and gN. In some embodiments, the two or more VZV glycoproteins are gE, gI, and gN. In some embodiments, the two or more VZV glycoproteins are gE and gM. In some embodiments, the two or more VZV glycoproteins are gI and gM. In some embodiments, the two or more VZV glycoproteins are gE, gI, and gM.

In some embodiments, the vaccine comprises any two or more VZV glycoproteins (e.g., any of the variant VZV gE disclosed in the preceding paragraphs and in the Examples and Figures), and the VZV gE is a variant gE, such as any of the variant VZV gE glycoproteins disclosed herein, for example, any of the variant VZV gE disclosed in the preceding paragraphs and in the Examples and Figures.

In some embodiments, the VZV vaccine includes two or more RNA polynucleotides having an open reading frame encoding two or more VZV antigenic polypeptides or an immunogenic fragment or epitope thereof (either encoded by a single RNA polynucleotide or encoded by two or more RNA polynucleotides, for example, each glycoprotein encoded by a separate RNA polynucleotide), and the two or more VZV glycoproteins are a variant gE (e.g., any of the variant gE polypeptides disclosed herein in the preceding paragraphs) and a VZV glycoprotein selected from gI, gB, gH, gK, gL, gC, gN, and gM polypeptides or immunogenic fragments or epitopes thereof. In some embodiments, the two or more VZV glycoproteins are a variant gE (e.g., any of the variant gE polypeptides disclosed herein in the preceding paragraphs) and gI. In some embodiments, the glycoproteins are VZV gI and variant VZV gE, and the variant VZV gE polypeptide is a truncated polypeptide lacking the anchor domain (ER retention domain) (e.g., a truncated VZV gE polypeptide comprising amino acids 1-561 of SEQ ID NO: 10). In some embodiments, the glycoproteins are VZV gI and variant VZV gE, and the variant VZV gE polypeptide is a truncated polypeptide lacking the carboxy terminal tail domain (e.g., a truncated VZV gE polypeptide comprising amino acids 1-573 of SEQ ID NO: 18). In some embodiments, the glycoproteins are VZV gI and variant VZV gE, and the variant VZV gE polypeptide has at least one mutation in one or more motif(s) associated with ER retention, TGN localization, and/or endocytosis (e.g., the variant VZV gE has a Y582G mutation, a Y569A mutation, or both a Y582G mutation and a Y569A mutation) and/or has at least one mutation in one or more phosphorylated acidic motif(s), such as SSTT (SEQ ID NO: 122) motif. In some embodiments, the glycoproteins are VZV gI and variant VZV gE, and the variant VZV gE polypeptide is an antigenic fragment comprising amino acids 1-573 of VZV gE and having a Y569A mutation. In some embodiments, the glycoproteins are VZV gI and variant VZV gE, and the variant VZV gE polypeptide is a full-length VZV gE polypeptide having an A-E-A-A-D-A (SEQ ID NO: 58) sequence. In some embodiments, the glycoproteins are VZV gI and variant VZV gE, and the VZV gE polypeptide has an A-E-A-A-D-A (SEQ ID NO: 58) sequence and a Y582G mutation, a Y569A mutation, or both a Y582G mutation and a Y569A mutation. In some embodiments, the glycoproteins are VZV gI and variant VZV gE, and the VZV gE polypeptide is a full-length VZV gE polypeptide having an additional sequence at the C-terminus that aids in secretion of the polypeptide (e.g., an IgKappa sequence). In some embodiments, the glycoproteins are VZV gI and variant VZV gE, and the VZV gE polypeptide is a full-length VZV gE polypeptide having an IgKappa sequence and a Y582G mutation, a Y569A mutation, or both a Y582G mutation and a Y569A mutation. In some embodiments, the glycoproteins are VZV gI and variant VZV gE, and the VZV gE polypeptide is a truncated VZV gE polypeptide lacking the anchor domain (ER retention domain) and having an IgKappa sequence. In some embodiments, the variant VZV gE polypeptide is a truncated polypeptide comprising amino acids 1-561 or amino acids 1-573 and having an IgKappa sequence at the C-terminus.

In any of the above-described embodiments, the VZV vaccine may further comprise a live attenuated VZV, a whole inactivated VZV, or a VZV virus-like particle (VLP). In some embodiments, the live attenuated VZV, whole inactivated VZV, or VZV VLP is selected from or derived from the following strains and genotypes: VZV E1 strain, genotypes E1_32_5, E1_Kcl, E1_Dumas, E1_Russia 1999, E1_SD, E1_MSP, E1_36, E1_49, E1_BC, E1_NH29; VZV E2 strain, genotypes E2_03-500, E2_2, E2_11, E2_HJO; VZV J strain, genotype pOka; VZV M1 strain, genotype M1_CA123; VZV M2 strain, genotypes M2_8 and M2_DR; and VZV M4 strain, genotypes Spain 4242, France 4415, and Italy 4053.

Alternate RNA vaccines comprising RNA polynucleotides encoding other viral protein components of VZV, for example, tegument proteins are encompassed by the present disclosure. Thus, some embodiments of the present disclosure provide VZV vaccines that include at least one ribonucleic acid (RNA) polynucleotide having an open reading frame encoding at least one VZV tegument protein or an antigenic fragment or epitope thereof. Some embodiments of the present disclosure provide VZV vaccines that include at least one RNA polynucleotide having an open reading frame encoding at least one VZV tegument protein or an immunogenic fragment or epitope thereof and at least one VZV glycoprotein or an immunogenic fragment or epitope thereof. Some embodiments of the present disclosure provide VZV vaccines that include at least one RNA polynucleotide having an open reading frame encoding at least one VZV tegument protein or an immunogenic fragment or epitope thereof and at least one RNA polynucleotide having an open reading frame encoding at least one VZV glycoprotein or an immunogenic fragment or epitope thereof. In some embodiments, RNA vaccines comprise RNA (e.g., mRNA) polynucleotide(s) encoding one or more VZV tegument protein(s) and one or more VZV glycoprotein(s) selected from VZV gE, gI, gB, gH, gK, gL, gC, gN, and gM polypeptides or immunogenic fragments or epitopes thereof. In some embodiments, the VZV glycoprotein is a VZV gE polypeptide or an immunogenic fragment or epitope thereof. In some embodiments, the VZV glycoprotein is a VZV gI polypeptide or immunogenic fragment or epitope thereof. In some embodiments, the VZV glycoprotein is a variant VZV gE polypeptide, such as any of the variant VZV gE polypeptides disclosed herein. In some embodiments, the VZV glycoproteins are VZV gE glycoproteins and VZV gI glycoproteins or immunogenic fragments or epitopes thereof.

In some embodiments, at least one RNA polynucleotide is encoded by at least one nucleic acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 41 and homologs having at least 80% (e.g., 85%, 90%, 95%, 98%, 99%) identity with a nucleic acid sequence selected from SEQ ID NO: 1-8 and 41. In some embodiments, at least one RNA polynucleotide is encoded by at least one nucleic acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 41 and homologs having at least 90% (e.g., 90%, 91%, 92%, 93%, 94%, 95%, 96%, 97%, 98%, 99%, 99.8% or 99.9%) identity with a nucleic acid sequence selected from SEQ ID NO: 1-8 and 41. In some embodiments, at least one RNA polynucleotide is encoded by at least one fragment of a nucleic acid sequence (e.g., a fragment having an antigenic sequence or at least one epitope) selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 41 and homologs having at least 80% (e.g., 85%, 90%, 95%, 98%, 99%) identity with a nucleic acid sequence selected from SEQ ID NO: 1-8 and 41. In some embodiments, at least one RNA polynucleotide is encoded by at least one epitope of a nucleic acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 41.

In some embodiments, at least one RNA polynucleotide is a gE polypeptide encoded by SEQ ID NO: 1. In some embodiments, at least one RNA polynucleotide is a gI polypeptide encoded by SEQ ID NO: 2. In some embodiments, at least one RNA polynucleotide is a truncated gE polypeptide encoded by SEQ ID NO: 3. In some embodiments, at least one RNA polynucleotide is a truncated gE polypeptide encoded by SEQ ID NO: 5. In some embodiments, at least one RNA polynucleotide is a truncated gE polypeptide having Y569A mutation encoded by SEQ ID NO: 6. In some embodiments, at least one RNA polynucleotide is a gE polypeptide having an AEAADA sequence SEQ ID NO: 58 encoded by SEQ ID NO: 7. In some embodiments, at least one RNA polynucleotide is a gE polypeptide having a Y582G mutation and a AEAADA sequence (SEQ ID NO: 58) encoded by SEQ ID NO: 8. In some embodiments, at least one RNA polynucleotide is a gE polypeptide encoded by SEQ ID NO: 41.

In some embodiments, at least one RNA (e.g., mRNA) polynucleotide encodes an antigenic polypeptide having at least 90% identity to the amino acid sequence of any of SEQ ID NO: 10, 14, 18, 22, 26, 30, 34, 38, 42 and 45-55. In some embodiments, at least one RNA (e.g., mRNA) polynucleotide encodes an antigenic polypeptide having at least 95% identity to the amino acid sequence of any of SEQ ID NO: 10, 14, 18, 22, 26, 30, 34, 38, 42 and 45-55. In some embodiments, at least one RNA polynucleotide encodes an antigenic polypeptide having at least 96% identity to the amino acid sequence of any of SEQ ID NO: 10, 14, 18, 22, 26, 30, 34, 38, 42 and 45-55. In some embodiments, at least one RNA (e.g., mRNA) polynucleotide encodes an antigenic polypeptide having at least 97% identity to the amino acid sequence of SEQ ID NO: 10, 14, 18, 22, 26, 30, 34, 38, 42 and 45-55. In some embodiments, at least one RNA (e.g., mRNA) polynucleotide encodes an antigenic polypeptide having at least 98% identity to the amino acid sequence of any of SEQ ID NO: 10, 14, 18, 22, 26, 30, 34, 38, 42 and 45-55. In some embodiments, at least one RNA (e.g., mRNA) polynucleotide encodes an antigenic polypeptide having at least 99% identity to the amino acid sequence of any of SEQ ID NO: 10, 14, 18, 22, 26, 30, 34, 38, 42 and 45-55.

In some embodiments, the open reading from which the VZV polypeptide is encoded is codon-optimized. In some embodiments, the at least one RNA polynucleotide encodes an antigenic protein of any of SEQ ID NO: 10, 14, 18, 22, 26, 30, 34, 38, 42 and 45-55, and wherein the RNA polynucleotide is codon-optimized mRNA. In some embodiments, the at least one RNA polynucleotide comprises a mRNA sequence identified by any one of SEQ ID NO: 92-108. In some embodiments, the mRNA sequence identified by any one of SEQ ID NO: 92-108 is codon optimized to encode antigenic VZV polypeptides that are as immunogenic, or more immunogenic than, the antigenic VZV polypeptides encoded by any one of SEQ ID NO: 92-108.

In some embodiments, the at least one RNA (e.g., mRNA) polynucleotide encodes an antigenic protein of SEQ ID NO: 10, wherein the RNA (e.g., mRNA) polynucleotide has less than 80% identity to wild-type mRNA sequence. In some embodiments, the at least one RNA polynucleotide encodes an antigenic protein of SEQ ID NO: 10, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 42, wherein the RNA e.g., mRNA) polynucleotide has less than 80% identity to wild-type mRNA sequence. In some embodiments, the at least one RNA (e.g., mRNA) polynucleotide encodes an antigenic protein of SEQ ID NO: 42, wherein the RNA polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 14, wherein the RNA (e.g., mRNA) polynucleotide has less than 80% identity to wild-type mRNA sequence. In some embodiments, the at least one RNA (e.g., mRNA) polynucleotide encodes an antigenic protein of SEQ ID NO: 14, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 26, wherein the RNA polynucleotide has less than 80% identity to wild-type mRNA sequence. In some embodiments, the at least one RNA (e.g., mRNA) polynucleotide encodes an antigenic protein of SEQ ID NO: 26, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 30, wherein the RNA polynucleotide has less than 80% identity to wild-type mRNA sequence. In some embodiments, the at least one RNA polynucleotide encodes an antigenic protein of SEQ ID NO: 30, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide is encoded by a sequence selected from any of SEQ ID NO: 1-8 and SEQ ID NO 41 and includes at least one chemical modification.

In some embodiments, the VZV vaccine is multivalent. In some embodiments, the RNA polynucleotide comprises a polynucleotide sequence derived from VZV E1 strain, including, for example, any one or more of genotypes E1_32_5, E1_Kel, E1_Dumas, E1_Russia 1999, E1_SD, E1_MSP, E1_36, E1_49, E1_BC, and E1_NH29. In some embodiments, the RNA (e.g., mRNA) polynucleotide comprises a polynucleotide sequence derived from VZV E2 strain, including, for example, any one or more of genotypes E2_03-500, E2_2, E2_11, and E2_HJO. In some embodiments, the RNA (e.g., mRNA) polynucleotide comprises a polynucleotide sequence derived from VZV J strain, including, for example, genotype pOka. In some embodiments, the RNA (e.g., mRNA) polynucleotide comprises a polynucleotide sequence derived from VZV M1 strain, including, for example, genotype M1_CA123. In some embodiments, the RNA (e.g., mRNA) polynucleotide comprises a polynucleotide sequence derived from VZV M2 strain, including, for example, genotypes M2_8 and M2_DR. In some embodiments, the RNA (e.g., mRNA) polynucleotide comprises a polynucleotide sequence derived from VZV M4 strain, including, for example, any one or more of genotypes Spain 4242, France 4415, and Italy 4053.

Some embodiments of the present disclosure provide a VZV vaccine that includes at least one ribonucleic acid (RNA) polynucleotide having an open reading frame encoding at least one VZV antigenic polypeptide or an immunogenic fragment thereof and at least one 5′ terminal cap. In some embodiments, a 5′ terminal cap is 7 mG (5′) ppp (5′) NImpNp. Some embodiments of the present disclosure provide a VZV vaccine that includes at least one RNA (e.g., mRNA) polynucleotide having an open reading frame encoding at least one VZV antigenic polypeptide or an immunogenic fragment thereof, wherein the at least one RNA (e.g., mRNA) polynucleotide has at least one chemical modification. In some embodiments, the at least one RNA (e.g., mRNA) polynucleotide further comprises a second chemical modification. In some embodiments, the at least one RNA (e.g., mRNA) polynucleotide having at least one chemical modification has a 5′ terminal cap. In some embodiments, the at least one chemical modification is selected from pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridinc, 2-thio-5-aza-uridine, 2-thio-dihydropseudouridine, 2-thio-dihydrouridine, 2-thio-pseudouridine, 4-methoxy-2-thio-pscudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridinc. In some embodiments, every (100%) of the uridines of the at least one RNA polynucleotide comprises a chemical modification, such as a N1-methylpseudouridine modification or a N1-ethylpseudouridinc modification.

Some embodiments of the present disclosure provide a VZV vaccine that includes at least one ribonucleic acid (RNA) polynucleotide having an open reading frame encoding at least one VZV antigenic polypeptide or an immunogenic fragment thereof, wherein at least 80% (e.g., 85%, 90%, 95%, 98%, 99%, 100%) of the uracil in the open reading frame have a chemical modification, optionally wherein the vaccine is formulated in a lipid nanoparticle. In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame are modified to include N1-methyl pseudouridine.

Some embodiments of the present disclosure provide a VZV vaccine that is formulated within a cationic lipid nanoparticle. In some embodiments, the cationic lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino) butanoyl)oxy) heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530).

In some embodiments, the lipid is

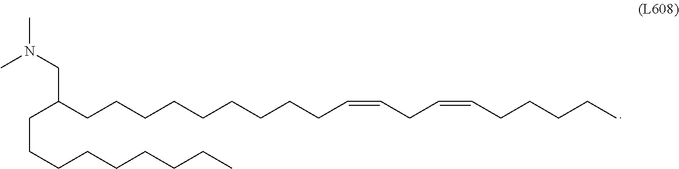 [SMILES: CCCCC/C=C\C/C=C\CCCCCCCCCC(CCCCCCCCC)CN(C)C]

In some embodiments, the lipid is

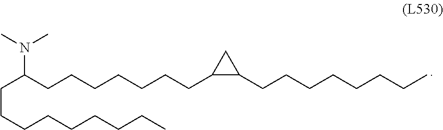 [SMILES: CCCCCCCCCC(CCCCCCCC1CC1CCCCCCCC)N(C)C]

In some embodiments, the cationic lipid nanoparticle has a molar ratio of about 20-60% cationic lipid, about 5-25% non-cationic lipid, about 25-55% sterol, and about 0.5-15% PEG-modified lipid. In some embodiments, the nanoparticle has a polydispersity value of less than 0.4. In some embodiments, the nanoparticle has a net neutral charge at a neutral pH value. In some embodiments, the nanoparticle has a mean diameter of 50-200 nm.

Some embodiments of the present disclosure provide methods of inducing an antigen specific immune response in a subject, comprising administering to the subject a VZV RNA (e.g., mRNA) vaccine in an amount effective to produce an antigen specific immune response. In some embodiments, an antigen specific immune response comprises a T cell response or a B cell response. In some embodiments, an antigen specific immune response comprises a T cell response and a B cell response. In some embodiments, a method of producing an antigen specific immune response involves a single administration of the vaccine. In some embodiments, a method further includes administering to the subject a booster dose of the vaccine. In some embodiments, a vaccine is administered to the subject by intradermal or intramuscular injection.

Also provided herein are VZV RNA (e.g., mRNA) vaccines for use in a method of inducing an antigen specific immune response in a subject, the method comprising administering the vaccine to the subject in an amount effective to produce an antigen specific immune response.

Further provided herein are uses of VZV RNA (e.g., mRNA) vaccines in the manufacture of a medicament for use in a method of inducing an antigen specific immune response in a subject, the method comprising administering the vaccine to the subject in an amount effective to produce an antigen specific immune response.

Some aspects of the present disclosure provide methods of preventing or treating VZV infection comprising administering to a subject the VZV RNA (e.g., mRNA) vaccine of the present disclosure. In some embodiments, the VZV RNA (e.g., mRNA) vaccine is formulated in an effective amount to produce an antigen specific immune response in a subject.

Some embodiments of the present disclosure provide methods of inducing an antigen specific immune response in a subject, the methods comprising administering to a subject a VZV RNA (e.g., mRNA) vaccine as provided herein in an effective amount to produce an antigen specific immune response in a subject.

In some embodiments, an anti-VZV antigenic polypeptide antibody titer produced in the subject is increased by at least 1 log relative to a control. In some embodiments, the anti-VZV antigenic polypeptide antibody titer produced in the subject is increased by 1-3 log relative to a control.

In some embodiments, the anti-VZV antigenic polypeptide antibody titer produced in the subject is increased at least 2 times relative to a control. In some embodiments, the anti-VZV antigenic polypeptide antibody titer produced in the subject is increased at least 5 times relative to a control. In some embodiments, the anti-VZV antigenic polypeptide antibody titer produced in the subject is increased at least 10 times relative to a control. In some embodiments, the anti-VZV antigenic polypeptide antibody titer produced in the subject is increased 2-10 times relative to a control.

In some embodiments, the control is an anti-VZV antigenic polypeptide antibody titer produced in a subject who has not been administered VZV vaccine. In some embodiments, the control is an anti-VZV antigenic polypeptide antibody titer produced in a subject who has been administered a live attenuated or inactivated VZV vaccine. In some embodiments, the control is an anti-VZV antigenic polypeptide antibody titer produced in a subject who has been administered a recombinant or purified VZV protein vaccine. In some embodiments, the control is an anti-VZV antigenic polypeptide antibody titer produced in a subject who has been administered an VZV virus-like particle (VLP) vaccine.

In some embodiments, the effective amount is a dose equivalent to at least a 2-fold reduction in the standard of care dose of a recombinant VZV protein vaccine, wherein an anti-VZV antigenic polypeptide antibody titer produced in the subject is equivalent to an anti-VZV antigenic polypeptide antibody titer produced in a control subject administered the standard of care dose of a recombinant or purified VZV protein vaccine, a live attenuated or inactivated VZV vaccine, or a VZV VLP vaccine.

In some embodiments, the effective amount is a dose equivalent to at least a 4-fold reduction in the standard of care dose of a recombinant VZV protein vaccine, wherein an anti-VZV antigenic polypeptide antibody titer produced in the subject is equivalent to an anti-VZV antigenic polypeptide antibody titer produced in a control subject administered the standard of care dose of a recombinant or purified VZV protein vaccine, a live attenuated or inactivated VZV vaccine, or a VZV VLP vaccine.

In some embodiments, the effective amount is a dose equivalent to at least a 10-fold reduction in the standard of care dose of a recombinant VZV protein vaccine, wherein an anti-VZV antigenic polypeptide antibody titer produced in the subject is equivalent to an anti-VZV antigenic polypeptide antibody titer produced in a control subject administered the standard of care dose of a recombinant or purified VZV protein vaccine, a live attenuated or inactivated VZV vaccine, or a VZV VLP vaccine.

In some embodiments, the effective amount is a dose equivalent to at least a 100-fold reduction in the standard of care dose of a recombinant VZV protein vaccine, wherein an anti-VZV antigenic polypeptide antibody titer produced in the subject is equivalent to an anti-VZV antigenic polypeptide antibody titer produced in a control subject administered the standard of care dose of a recombinant or purified VZV protein vaccine, a live attenuated or inactivated VZV vaccine, or a VZV VLP vaccine.

In some embodiments, the effective amount is a dose equivalent to at least a 1000-fold reduction in the standard of care dose of a recombinant VZV protein vaccine, wherein an anti-VZV antigenic polypeptide antibody titer produced in the subject is equivalent to an anti-VZV antigenic polypeptide antibody titer produced in a control subject administered the standard of care dose of a recombinant or purified VZV protein vaccine, a live attenuated or inactivated VZV vaccine, or a VZV VLP vaccine.

In some embodiments, the effective amount is a dose equivalent to a 2-fold to 1000-fold reduction in the standard of care dose of a recombinant VZV protein vaccine, wherein an anti-VZV antigenic polypeptide antibody titer produced in the subject is equivalent to an anti-VZV antigenic polypeptide antibody titer produced in a control subject administered the standard of care dose of a recombinant or purified VZV protein vaccine, a live attenuated or inactivated VZV vaccine, or a VZV VLP vaccine.

In some embodiments, the effective amount is a total dose of 25 μg to 1000 μg, or 50 μg to 1000 μg or 25 to 200 μg. In some embodiments, the effective amount is a total dose of 50 μg, 100 μg, 200 μg, 400 μg, 800 μg, or 1000 μg. In some embodiments, the effective amount is a total dose of 200 μg. In some embodiments, the effective amount is a total dose of 50 μg to 400 μg. In some embodiments, the effective amount is a total dose of 50 μg, 100 μg, 150 μg, 200 μg, 250 μg, 300 μg, 350 μg or 400 μg. In some embodiments, the effective amount is a dose of 25 μg administered to the subject a total of two times. In some embodiments, the effective amount is a dose of 50 μg administered to the subject a total of two times. In some embodiments, the effective amount is a dose of 100 μg administered to the subject a total of two times. In some embodiments, the effective amount is a dose of 200 μg administered to the subject a total of two times. In some embodiments, the effective amount is a dose of 400 μg administered to the subject a total of two times. In some embodiments, the effective amount is a dose of 500 μg administered to the subject a total of two times.

In some embodiments, the efficacy (or effectiveness) of the VZV RNA (e.g., mRNA) vaccine against VZV is greater than 60%.

Vaccine efficacy may be assessed using standard analyses (see, e.g., Weinberg et al., J Infect Dis. 2010 Jun. 1;201 (11): 1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( ARU - ARV ) / ARU \times 100 ; and$$

$$Efficacy = ( 1 - RR ) \times 100.$$

Likewise, vaccine effectiveness may be assessed using standard analyses (see, e.g., Weinberg et al., J Infect Dis. 2010 Jun. 1;201 (11): 1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In some embodiments, the efficacy (or effectiveness) of the VZV RNA (e.g., mRNA) vaccine against VZV is greater than 65%. In some embodiments, the efficacy (or effectiveness) of the vaccine against VZV is greater than 70%. In some embodiments, the efficacy (or effectiveness) of the vaccine against VZV is greater than 75%. In some embodiments, the efficacy (or effectiveness) of the vaccine against VZV is greater than 80%. In some embodiments, the efficacy (or effectiveness) of the vaccine against VZV is greater than 85%. In some embodiments, the efficacy (or effectiveness) of the vaccine against VZV is greater than 90%.

In some embodiments, the vaccine immunizes the subject against VZV up to 1 year (e.g. for a single VZV season). In some embodiments, the vaccine immunizes the subject against VZV for up to 2 years. In some embodiments, the vaccine immunizes the subject against VZV for more than 2 years. In some embodiments, the vaccine immunizes the subject against VZV for more than 3 years. In some embodiments, the vaccine immunizes the subject against VZV for more than 4 years. In some embodiments, the vaccine immunizes the subject against VZV for 5-10 years.

In some embodiments, the subject administered an VZV RNA (e.g., mRNA) vaccine is between the ages of about 12 months old and about 10 years old (e.g., about 1, 2, 3, 4, 5, 6, 7, 8, 9 or 10 years old). In some embodiments, the subject administered an VZV RNA (e.g., mRNA) vaccine is between the ages of about 12 months old and about 15 months old (e.g., about 12, 12.5, 13, 13.5, 14, 14.5 or 15 months old). In some embodiments, the subject administered an VZV RNA (e.g., mRNA) vaccine is between the ages of about 4 years old and about 6 years old (e.g., about 4, 4.5, 5, 5.6, or 6 years old).

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject has been exposed to VZV, is infected with (has) VZV, or is at risk of infection by VZV.

In some embodiments, the subject is immunocompromised (has an impaired immune system, e.g., has an immune disorder or autoimmune disorder).

Some aspects of the present disclosure provide varicella zoster virus (VZV) RNA (e.g., mRNA) vaccines containing a signal peptide linked to a VZV antigenic polypeptide. Thus, in some embodiments, the VZV RNA (e.g., mRNA) vaccines contain at least one ribonucleic acid (RNA) polynucleotide having an open reading frame encoding a signal peptide linked to a VZV antigenic peptide. Also provided herein are nucleic acids encoding the VZV RNA (e.g., mRNA) vaccines disclosed herein.

Other aspects of the present disclosure provide varicella zoster virus (VZV) vaccines containing a signal peptide linked to a VZV antigenic polypeptide. In some embodiments, the VZV antigenic polypeptide is a VZV glycoprotein. In some embodiments, the VZV glycoprotein is selected from VZV gE, gI, gB, gH, gK, gL, gC, gN, and gM. In some embodiments, the VZV glycoprotein is VZV gE or a variant VZV gE polypeptide.

In some embodiments, the signal peptide is a IgE signal peptide. In some embodiments, the signal peptide is an IgE HC (Ig heavy chain epsilon-1) signal peptide. In some embodiments, the signal peptide has the sequence MDWTWILFLVAAATRVHS (SEQ ID NO: 56). In some embodiments, the signal peptide is an IgGK signal peptide. In some embodiments, the signal peptide has the sequence METPAQLLFLLLLWLPDTTG (SEQ ID NO: 57). In some embodiments, the signal peptide is selected from: a Japanese encephalitis PRM signal sequence (MLGSNSGQRVVFTILLLLVAPAYS; SEQ ID NO: 109), VSVg protein signal sequence (MKCLLYLAFLFIGVNCA; SEQ ID NO: 110) and Japanese encephalitis JEV signal sequence (MWLVSLAIVTACAGA; SEQ ID NO: 111).

Further provided herein are nucleic acids encoding VZV vaccines disclosed herein. Such VZV vaccines include at least one ribonucleic acid (RNA) (e.g., mRNA) polynucleotide having an open reading frame encoding a signal peptide linked to a VZV antigenic polypeptide. In some embodiments, the VZV antigenic peptide is a VZV glycoprotein. In some embodiments, the VZV glycoprotein is selected from VZV gE, gI, gB, gH, gK, gL, gC, gN, and gM. In some embodiments, the VZV antigenic peptide is a VZV gE or a variant of the gE polypeptide.

In some embodiments, an effective amount of an VZV RNA (e.g., mRNA) vaccine (e.g., a single dose of the VZV vaccine) results in a 2 fold to 200 fold (e.g., about 2, 3, 4, 5, 6, 7, 8, 9, 10, 20, 30, 40, 50, 60, 70, 80, 90, 100, 110, 120, 130, 140, 150, 160, 170, 180, 190 or 200 fold) increase in serum neutralizing antibodies against VZV, relative to a control. In some embodiments, a single dose of the VZV RNA (e.g., mRNA) vaccine results in an about 5 fold, 50 fold, or 150 fold increase in serum neutralizing antibodies against VZV, relative to a control. In some embodiments, a single dose of the VZV RNA (e.g., mRNA) vaccine results in an about 2 fold to 10 fold, or an about 40 to 60 fold increase in serum neutralizing antibodies against VZV, relative to a control.

In some embodiments, efficacy of RNA vaccines RNA (e.g., mRNA) can be significantly enhanced when combined with a flagellin adjuvant, in particular, when one or more antigen-encoding mRNAs is combined with an mRNA encoding flagellin.

RNA (e.g., mRNA) vaccines combined with the flagellin adjuvant (e.g., mRNA-encoded flagellin adjuvant) have superior properties in that they may produce much larger antibody titers and produce responses earlier than commercially available vaccine formulations. While not wishing to be bound by theory, it is believed that the RNA vaccines, for example, as mRNA polynucleotides, are better designed to produce the appropriate protein conformation upon translation, for both the antigen and the adjuvant, as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Some embodiments of the present disclosure provide RNA (e.g., mRNA) vaccines that include at least one RNA (e.g., mRNA) polynucleotide having an open reading frame encoding at least one antigenic polypeptide or an immunogenic fragment thereof (e.g., an immunogenic fragment capable of inducing an immune response to the antigenic polypeptide) and at least one RNA (e.g., mRNA polynucleotide) having an open reading frame encoding a flagellin adjuvant.

In some embodiments, at least one flagellin polypeptide (e.g., encoded flagellin polypeptide) is a flagellin protein. In some embodiments, at least one flagellin polypeptide (e.g., encoded flagellin polypeptide) is an immunogenic flagellin fragment. In some embodiments, at least one flagellin polypeptide and at least one antigenic polypeptide are encoded by a single RNA (e.g., mRNA) polynucleotide. In other embodiments, at least one flagellin polypeptide and at least one antigenic polypeptide are each encoded by a different RNA polynucleotide.

In some embodiments at least one flagellin polypeptide has at least 80%, at least 85%, at least 90%, or at least 95% identity to a flagellin polypeptide having a sequence of any of SEQ ID NO: 115-117.

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first antigenic VZV polypeptide, wherein the RNA polynucleotide does not include a stabilization element, and wherein an adjuvant is not coformulated or co-administered with the vaccine.

In other aspects the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection.

In some embodiments, the nucleic acid vaccine is administered to the subject on day zero. In some embodiments, a second dose of the nucleic acid vaccine is administered to the subject on day twenty one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100 fold higher level in the local lymph node in comparison with the distal lymph node. In other embodiments the nucleic acid vaccine is chemically modified and in other embodiments the nucleic acid vaccine is not chemically modified.

Aspects of the invention provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no chemical modification, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no chemical modification, the open reading frame encoding a first antigenic polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 μg and 400 μg of one or more RNA polynucleotides having an open reading frame comprising at least one chemical modification or optionally no chemical modification, the open reading frame encoding a first antigenic polypeptide, and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a VZV strain in an individual or population of individuals comprising administering to said individual or population an antigenic memory booster nucleic acid vaccine comprising (a) at least one RNA polynucleotide, said polynucleotide comprising at least one chemical modification or optionally no chemical modification and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 μg/kg and 400 μg/kg of a nucleic acid vaccine comprising one or more RNA polynucleotides having an open reading frame encoding a first antigenic polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Other aspects provide nucleic acid vaccines comprising an LNP formulated RNA polynucleotide having an open reading frame comprising no modified nucleotides (unmodified), the open reading frame encoding a first antigenic polypeptide, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

The data presented in the Examples demonstrate significant enhanced immune responses using the formulations of the invention. The data demonstrated the effectiveness of both chemically modified and unmodified RNA vaccines of the invention. Surprisingly, in contrast to prior art reports that it was preferable to use chemically unmodified mRNA formulated in a carrier for the production of vaccines, it was discovered herein that chemically modified mRNA-LNP vaccines required a much lower effective mRNA dose than unmodified mRNA, i.e., tenfold less than unmodified mRNA when formulated in carriers other than LNP. Both the chemically modified and unmodified RNA vaccines of the invention produce better immune responses than mRNA vaccines formulated in a different lipid carrier.

In other aspects the invention encompasses a method of treating an elderly subject age 60 years or older comprising administering to the subject a nucleic acid vaccine comprising one or more RNA polynucleotides having an open reading frame encoding a VZV antigenic polypeptide in an effective amount to vaccinate the subject.

In other aspects the invention encompasses a method of treating a young subject age 17 years or younger comprising administering to the subject a nucleic acid vaccine comprising one or more RNA polynucleotides having an open reading frame encoding a VZV antigenic polypeptide in an effective amount to vaccinate the subject.

In other aspects the invention encompasses a method of treating an adult subject comprising administering to the subject a nucleic acid vaccine comprising one or more RNA polynucleotides having an open reading frame encoding a VZV antigenic polypeptide in an effective amount to vaccinate the subject.

The RNA polynucleotide is one of SEQ ID NO: 1-8 and 41 and includes at least one chemical modification. In other embodiments the RNA polynucleotide is one of SEQ ID NO: 1-8 and 41 and does not include any nucleotide modifications, or is unmodified. In yet other embodiments the at least one RNA polynucleotide encodes an antigenic protein of any of SEQ ID NO: 10, 14, 18, 22, 26, 30, 34, 38, 42 and 45-55 and includes at least one chemical modification. In other embodiments the RNA polynucleotide encodes an antigenic protein of any of SEQ ID NO: 10, 14, 18, 22, 26, 30, 34, 38, 42 and 45-55 and does not include any nucleotide modifications, or is unmodified.

In preferred aspects, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In exemplary aspects of the invention, antigen-specific antibodies are measured in units of μg/ml or are measured in units of IU/L (International Units per liter) or mIU/ml (milli International Units per ml). In exemplary embodiments of the invention, an efficacious vaccine produces>0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces>10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic depicting a proposed Varicella zoster virus pathway.

FIG. 2 is a schematic of the constructs encoding VZV gE (strain Oka).

FIG. 3 depicts the study design and injection schedule for the immunization of BALB/C mice with MC3 formulated mRNA encoded VZV gE antigens.

FIG. 4 is a schematic showing various variant VZV gE antigens. This figure depicts SEQ ID NOs: 120, 132, and 58 from top to bottom, respectively.

FIG. 5 is a graph showing the results of an ELISA assay that shows the levels of anti-VZV gE IgG in the serum of mice vaccinated with various VZV gE mRNAs in comparison with VARIVAX® vaccine.

FIG. 6 is a graph showing the results of an ELISA assay indicating the levels of anti-VZV gE IgG in the serum of mice vaccinated with various VZV gE mRNAs in comparison with VARIVAX® vaccine.

FIG. 7 is a graph showing the results of an ELISA assay indicating the levels of anti-VZV gE IgG in the serum of mice vaccinated with various VZV gE mRNAs in comparison with VARIVAX® vaccine.

FIGS. 8A and 8B show confocal microscopy of human melanoma (MeWo) cells stained with an antibodies to show the golgi apparatus.

FIGS. 9A-9C show confocal microscopy of MeWo cells stained with antibodies against VZV gE to show VZV gE expression and trafficking. The sequence AEAADA depicted in FIG. 9C is SEQ ID NO: 58

FIGS. 10A and 10B are schematics depicting various VZV wildtype genotypes.

FIGS. 11A and 11B are graphs showing the results of an ELISA assay, which shows the levels of anti-VZV gE IgG in the serum of mice vaccinated with VZV gE variant mRNAs in comparison with ZOSTAVAX® vaccine. The sequence AEAADA depicted throughout FIGS. 11A and 11B is SEQ ID NO: 58.

FIGS. 12A and 12B are graphs showing the results of an ELISA assay, which shows the levels of anti-VZV gE IgG in the serum of mice vaccinated with VZV gE variant mRNAs in comparison with ZOSTAVAX® vaccine. The sequence AEAADA depicted throughout

FIGS. 12A and 12B is SEQ ID NO: 58.

FIG. 13 is a graph showing the results of an ELISA assay measuring the antibody titer in the sera of mice immunized with VZV gE variant mRNA vaccines. Anti-VZV gE response induced by VZV gE variants mRNAs in mice are greater than that of ZOSTAVAX®. The gE variant mRNA for GE-delete_from_574-Y569A induced an immune response that is1 log greater than ZOSTAVAX®. The sequence AEAADA depicted throughout FIG. 13 is SEQ ID NO: 58.

FIG. 14A is a graph showing the results of an ELISA assay, which shows the levels of anti-VZV gE IgG in the serum of mice vaccinated with VZV gE variant mRNAs after primary exposure with ZOSTAVAX® vaccine (groups 1-5) or VZV-gE-del_574_Y569A (group 6). The sequence AEAADA depicted throughout FIG. 14A is SEQ ID NO: 58. FIG. 14B is a graph showing the determination of EC50.

FIGS. 15A and 15B are graphs showing the results of the T cell analysis of mice vaccinated with VZV gE variant mRNAs after primary exposure with ZOSTAVAX® vaccine (groups 1-5) or VZV-gE-del_574_Y569A (group 6). The sequence AEAADA depicted throughout FIGS. 15A and 15B is SEQ ID NO: 58.

FIGS. 16A and 16B are graphs showing the results of an ELISA assay, which shows the levels of anti-VZV gE IgG in serum of rhesus monkeys vaccinated with either VZV-gE-del_574_Y569A or ZOSTAVAX® after primary exposure with VZV-gE-del_574_Y569A or ZOSTAVAX. FIG. 16C is a graph showing the determination of EC50 and EC10. FIGS. 16D, 16E, and 16F are graphs showing the results of the T cell analysis of rhesus monkeys vaccinated with either VZV-gE-del_574_Y569A or ZOSTAVAX® after primary exposure with VZV-gE-del_574_Y569A or ZOSTAVAX®.

FIG. 17 shows a schematic of the immogenicity study described in Example 18.

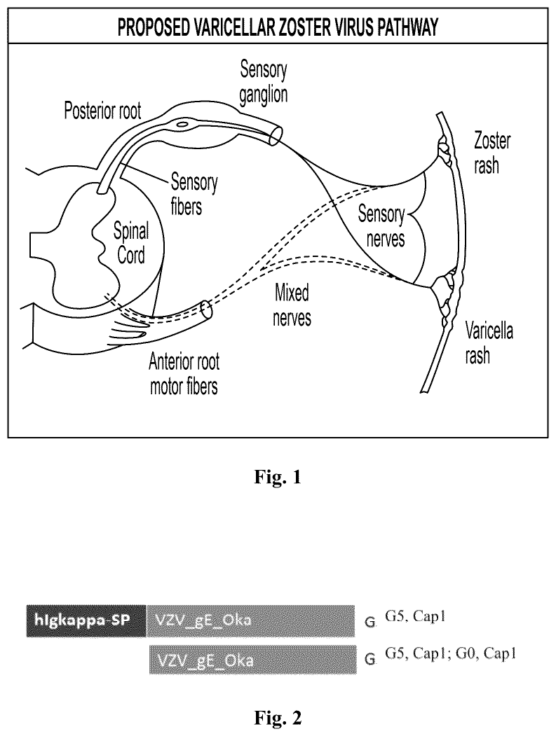

### DETAILED DESCRIPTION

Embodiments of the present disclosure provide RNA (e.g., mRNA) vaccines that include at least one RNA (e.g., mRNA) polynucleotide encoding a varicella zoster virus (VZV) antigen. There are at least five clades of varicella zoster virus (VZV). Clades 1 and 3 include European/North American strains; clade 2 includes Asian strains, especially from Japan; and clade 5 appears to be based in India. Clade 4 includes some strains from Europe, but its geographic origins need further clarification. Phylogenetic analysis of VZV genomic sequences resolves wild-type strains into 9 genotypes (E1, E2, J, M1, M2, M3, M4, VIII and IX). Sequence analysis of 342 clinical varicella and zoster specimens from 18 European countries identified the following distribution of VZV genotypes: E1, 221 (65%); E2, 87 (25%); M1, 20 (6%); M2, 3 (1%); M4, and 11 (3%). No M3 or J strains were observed. Of 165 clinical varicella and zoster isolates from Australia and New Zealand, 67 of 127 castern Australian isolates were E1, 30 were E2, 16 were J, 10 were M1, and 4 were M2; 25 of 38 New Zealand isolates were E1, 8 were E2, and 5 were M1.

VZV is an alphaherpesvirus that exists as a spherical multilayered structure approximately 200 nm in diameter. The viral genome is surrounded by a protein capsid structure that is covered by an amorphous layer of tegument proteins. These two structures are surrounded by a lipid envelope that is studded with viral glycoproteins, each about 8 nm in length, that are displayed on the exterior of the virion, and encloses the 100 nm nucleocapsid which is comprised of 162 hexameric and pentameric capsomeres arranged in an icosahedral form. The tegument, which is comprised of virally-encoded proteins and enzymes, is located in the space between the nucleocapsid and the viral envelope. The viral envelope is acquired from host cell membranes and contains viral-encoded glycoproteins.

The VZV genome is a single, linear, duplex DNA molecule of 124,884 base pairs having at least 70 open reading frames. The genome has 2 predominant isomers, depending on the orientation of the S segment, P (prototype) and IS (inverted S), which are present with equal frequency for a total frequency of 90-95%. The L segment can also be inverted resulting in a total of four linear isomers (IL and ILS).

VZV is closely related to the herpes simplex viruses (HSV), sharing much genome homology. The VZV genome is the smallest of the human herpesviruses and encodes at least 71 unique proteins (ORFO—ORF68) with three more opening reading frames (ORF69-ORF71) that duplicate earlier open reading frames (ORF64-62, respectively). Only a fraction of the encoded proteins form the structure of the virus particle. Among those proteins are nine glycoproteins: ORF5 (gK), ORF9A (gN), ORF14 (gC), ORF31 (gB), ORF37 (gH), ORF50 (gM), ORF60 (gL), ORF67 (gI), and ORF68 (gE). The known envelope glycoproteins (gB, gC, gE, gH, gI, gK, gL, gN, and gM) correspond with those in HSV; however, there is no equivalent of HSV gD. VZV also fails to produce the LAT (latency-associated transcripts) that play an important role in establishing HSV latency (herpes simplex virus). The encoded glycoproteins gE, gI, gB, gH, gK, gL, gC, gN, and gM function in different steps of the viral replication cycle. The most abundant glycoprotein found in infected cells, as well as in the mature virion, is glycoprotein E (gE, ORF 68), which is a major component of the virion envelope and is essential for viral replication. Glycoprotein I (gI, ORG 67) forms a complex with gE in infected cells, which facilitates the endocytosis of both glycoproteins and directs them to the trans-Golgi network (TGN) where the final viral envelope is acquired. Glycoprotein I (gI) is required within the TGN for VZV envelopment and for efficient membrane fusion during VZV replication. VZV gE and gI arc found complexed together on the infected host cell surface. Glycoprotein B (ORF 31), which is the second most prevalent glycoprotein and thought to play a role in virus entry, binds to neutralizing antibodies. Glycoprotein H is thought to have a fusion function facilitating cell to cell spread of the virus. Antibodies to gE, gB, and gH are prevalent after natural infection and following vaccination and have been shown to neutralize viral activity in vitro.

Embodiments of the present disclosure provide RNA (e.g., mRNA) vaccines that include at least one polynucleotide encoding at least one VZV antigenic polypeptide. The VZV RNA vaccines provided herein may be used to induce a balanced immune response, comprising both cellular and humoral immunity, without many of the risks associated with DNA vaccines and live attenuated vaccines. The various RNA (e.g., mRNA) vaccines disclosed herein produced an immune response in BALB/C mice, the results of which are discussed in detail in the Examples section. Specifically, RNA (e.g., mRNA) polynucleotide vaccines having an open reading frame encoding one or more of a variety of VZV antigens produced significant immune response, relative to a traditional VZV vaccine (e.g. attenuated VZV virus). The VZV RNA (e.g., mRNA) polynucleotide vaccines disclosed herein encoding either VZV gE or variant VZV gE demonstrated significant immune response after two administrations when administered intramuscularly (IM) or intradermally (ID). The VZV glycoproteins and tegument proteins have been shown to be antigenic. VZV glycoproteins, fragments thereof, and epitopes thereof are encompassed within the present disclosure.

The entire contents of International Application No. PCT/US2015/02740 is incorporated herein by reference.

It has been discovered that the mRNA vaccines described herein are superior to current vaccines in several ways. First, the lipid nanoparticle (LNP) delivery is superior to other formulations including a protamine base approach described in the literature and no additional adjuvants are to be necessary. The use of LNPs enables the effective delivery of chemically modified or unmodified mRNA vaccines. Additionally it has been demonstrated herein that both modified and unmodified LNP formulated mRNA vaccines were superior to conventional vaccines by a significant degree. In some embodiments the mRNA vaccines of the invention are superior to conventional vaccines by a factor of at least 10 fold, 20 fold, 40 fold, 50 fold, 100 fold, 500 fold or 1,000 fold.

Although attempts have been made to produce functional RNA vaccines, including mRNA vaccines and self-replicating RNA vaccines, the therapeutic efficacy of these RNA vaccines have not yet been fully established. Quite surprisingly, the inventors have discovered, according to aspects of the invention a class of formulations for delivering mRNA vaccines in vivo that results in significantly enhanced, and in many respects synergistic, immune responses including enhanced antigen generation and functional antibody production with neutralization capability. These results can be achieved even when significantly lower doses of the mRNA are administered in comparison with mRNA doses used in other classes of lipid based formulations. The formulations of the invention have demonstrated significant unexpected in vivo immune responses sufficient to establish the efficacy of functional mRNA vaccines as prophylactic and therapeutic agents. Additionally, self-replicating RNA vaccines rely on viral replication pathways to deliver enough RNA to a cell to produce an immunogenic response. The formulations of the invention do not require viral replication to produce enough protein to result in a strong immune response. Thus, the mRNA of the invention are not self-replicating RNA and do not include components necessary for viral replication.

The invention involves, in some aspects, the surprising finding that lipid nanoparticle (LNP) formulations significantly enhance the effectiveness of mRNA vaccines, including chemically modified and unmodified mRNA vaccines. The efficacy of mRNA vaccines formulated in LNP was examined in vivo using several distinct antigens. The results presented herein demonstrate the unexpected superior efficacy of the mRNA vaccines formulated in LNP over other commercially available vaccines.

In addition to providing an enhanced immune response, the formulations of the invention generate a more rapid immune response with fewer doses of antigen than other vaccines tested. The mRNA-LNP formulations of the invention also produce quantitatively and qualitatively better immune responses than vaccines formulated in a different carriers.

The data described herein demonstrate that the formulations of the invention produced significant unexpected improvements over existing VZV antigen vaccines, including significantly higher levels of IgG production by mRNA chemically modified and unmodified VZV vaccines formulated in LNP compared to VARIVAX and ZOSTAVAX. The onset of IgG production was significantly more rapid for the chemically modified LNP mRNA vaccines than the unmodified or commercially available vaccines tested.

Additionally, the mRNA-LNP formulations of the invention are superior to other vaccines even when the dose of mRNA is lower than other vaccines. The data demonstrate that all gE variants LNP mRNA vaccines induced much stronger immune response than ZOSTAVAX® after the two 10 μg doses as well as after the two 2 μg doses. When the sera were diluted more than 100 fold, the antibody titer is higher in VZV gE LNP mRNA vaccinated mice sera than in ZOSTAVAX® vaccinated mice sera, suggesting that the VZV gE LNP mRNA vaccines induced much stronger immune response than ZOSTAVAX® in mice.

The results in mice were consistent with the immunogenicity observed in non-human primates. Rhesus monkeys were primed with chemically modified VZV LNP mRNA vaccines or ZOSTAVAX®. The mRNA vaccines provided higher anti-gE titers than ZOSTAVAX® and produced reasonable frequency of CD4 T-cells producing IFNγ, IL-2 or TNFα cells, unlike the ZOSTAVAX® group. The data also demonstrated that a single dose of mRNA vaccination after Zostavax exposure was equivalent to two doses of mRNA vaccination in inducing comparable T-cell responses.

Some of the LNP used in the studies described herein has been used previously to deliver siRNA in various animal models as well as in humans. In view of the observations made in association with the siRNA delivery of LNP formulations, the fact that LNP is useful in vaccines is quite surprising. It has been observed that therapeutic delivery of siRNA formulated in LNP causes an undesirable inflammatory response associated with a transient IgM response, typically leading to a reduction in antigen production and a compromised immune response. In contrast to the findings observed with siRNA, the LNP-mRNA formulations of the invention are demonstrated herein to generate enhanced IgG levels, sufficient for prophylactic and therapeutic methods rather than transient IgM responses.

### Nucleic Acids/Polynucleotides

Varicella zoster virus (VZV) vaccines, as provided herein, comprise at least one (one or more) ribonucleic acid (RNA, e.g., mRNA) polynucleotide having an open reading frame encoding at least one VZV antigenic polypeptide. The term “nucleic acid,” in its broadest sense, includes any compound and/or substance that comprises a polymer of nucleotides. These polymers are referred to as polynucleotides.

In some embodiments, at least one RNA polynucleotide of a VZV vaccine is encoded by at least one nucleic acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 41.

In some embodiments, at least one RNA (e.g., mRNA) polynucleotide of a VZV vaccine is encoded by at least one fragment of a nucleic acid sequence (e.g., a fragment having an antigenic sequence or at least one epitope) selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 41.

Nucleic acids (also referred to as polynucleotides) may be or may include, for example, ribonucleic acids (RNAs), deoxyribonucleic acids (DNAs), threose nucleic acids

(TNAs), glycol nucleic acids (GNAs), peptide nucleic acids (PNAs), locked nucleic acids (LNAs, including LNA having a β-D-ribo configuration, β-LNA having an β-L-ribo configuration (a diastereomer of LNA), 2′-amino-LNA having a 2′-amino functionalization, and 2′-amino-β-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence, but where the sequence represents RNA (e.g., mRNA), the “T”'s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

In some embodiments, a RNA polynucleotide (e.g., mRNA) of a VZV vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 antigenic polypeptides. In some embodiments, a RNA polynucleotide (e.g., mRNA) of a VZV RNA (e.g., mRNA) vaccine encodes at least 10, 20, 30, 40, 50, 60, 70, 80, 90 or 100 antigenic polypeptides. In some embodiments, a RNA polynucleotide (e.g., mRNA) of a VZV vaccine encodes at least 100 antigenic polypeptides, or at least 200 antigenic polypeptides. In some embodiments, a RNA polynucleotide (e.g., mRNA) of a VZV vaccine encodes 1-10, 5-15, 10-20, 15-25, 20-30, 25-35, 30-40, 35-45, 40-50, 1-50, 1-100, 2-50 or 2-100 antigenic polypeptides.

Polynucleotides (e.g., mRNAs) of the present disclosure, in some embodiments, are codon optimized. Codon optimization methods are known in the art and may be used as provided herein. For example, any one or more of the sequences SEQ ID NO: 11, 15, 19, 23, 27, 31, 35, 39, 62, 66, 70, 74, 78, 82, 86, 90 or any one or more of the sequences of SEQ ID NO: 92-108 may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence (e.g., a codon-optimized sequence of SEQ ID NO: 92-108) shares less than 95% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 90% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 85% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 80% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 75% sequence identity to a naturally-occurring or wild-type sequence (e.g., a naturally-occurring or wild-type mRNA sequence encoding a polypeptide or protein of interest (e.g., an antigenic protein or polypeptide)).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares between 65% and 75% or about 80% sequence identity to a naturally-occurring or wild-type sequence (e.g., a naturally-occurring or wild-type mRNA sequence encoding a polypeptide or protein of interest (e.g., an antigenic protein or polypeptide)).

In some embodiments, a codon-optimized sequence (e.g., a codon-optimized sequence of SEQ ID NO: 92-108) encodes an antigenic polypeptide that is as immunogenic as, or more immunogenic than (e.g., at least 10%, at least 20%, at least 30%, at least 40%, at least 50%, at least 100%, or at least 200% more), than an antigenic polypeptide encoded by a (non-codon-optimized) sequence of SEQ ID NO: 92-108.

In some embodiments, the VZV vaccine includes at least one RNA polynucleotide having an open reading frame encoding at least one VZV antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G (5′) ppp (5′) G [the ARCA cap];G (5′) ppp (5′) A; G (5′) ppp (5′) G; m7G (5′) ppp (5′) A; m7G (5′) ppp (5′) G (New England BioLabs, Ipswich, MA). 5′-capping of modified RNA may be completed post-transcriptionally using a Vaccinia Virus Capping Enzyme to generate the “Cap 0” structure: m7G (5′) ppp (5′) G (New England BioLabs, Ipswich, MA). Cap 1 structure may be generated using both Vaccinia Virus Capping Enzyme and a 2′-O methyl-transferase to generate: m7G (5′) ppp (5′) G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

When transfected into mammalian cells, the modified mRNAs have a stability of between 12-18 hours, or greater than 18 hours, e.g., 24, 36, 48, 60, 72, or greater than 72 hours.

In some embodiments a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Antigens/Antigenic Polypeptides

In some embodiments, an antigenic polypeptide is a VZV glycoprotein. For example, a VZV glycoprotein may be VZV gE, gI, gB, gH, gK, gL, gC, gN, or gM or an immunogenic fragment or epitope thereof. In some embodiments, the antigenic polypeptide is a VZV gE polypeptide. In some embodiments, the antigenic polypeptide is a VZV gI polypeptide. In some embodiments, the antigenic polypeptide is a VZV gB polypeptide. In some embodiments, the antigenic polypeptide is a VZV gH polypeptide. In some embodiments, the antigenic polypeptide is a VZV gK polypeptide. In some embodiments, the antigenic polypeptide is a VZV gL polypeptide. In some embodiments, the antigenic polypeptide is a VZV gC polypeptide. In some embodiments, the antigenic polypeptide is a VZV gN polypeptide. In some embodiments, the antigenic polypeptide is a VZV gM polypeptide.

In some embodiments, the antigenic polypeptide comprises two or more glycoproteins. The two or more glycoproteins can be encoded by a single RNA polynucleotide or can be encoded by two or more RNA polynucleotides, for example, each glycoprotein encoded by a separate RNA polynucleotide. In some embodiments, the two or more glycoproteins can be any combination of VZV gE, gI, gB, gH, gK, gL, gC, gN, and gM polypeptides or immunogenic fragments or epitopes thereof. In some embodiments, the two or more glycoproteins can be any combination of VZV gE and a glycoprotein selected from gI, gB, gH, gK, gL, gC, gN, and gM polypeptides or immunogenic fragments or epitopes thereof. In some embodiments, the two or more glycoproteins can be any combination of VZV gI and a glycoprotein selected from gE, gB, gH, gK, gL, gC, gN, and gM polypeptides or immunogenic fragments or epitopes thereof. In some embodiments, the two or more glycoproteins can be any combination of VZV gE, gI, and a glycoprotein selected from gB, gH, gK, gL, gC, gN, and gM polypeptides or immunogenic fragments or epitopes thereof. In some embodiments, the two or more VZV glycoproteins are gE and gI. Alternate RNA vaccines comprising RNA polynucleotides encoding other viral protein components of VZV, for example, tegument proteins are encompassed by the present disclosure. Thus, some embodiments of the present disclosure provide VZV vaccines that include at least one ribonucleic acid (RNA) polynucleotide having an open reading frame encoding at least one VZV tegument protein or an antigenic fragment or epitope thereof. In some embodiments, the antigenic polypeptide is a VZV tegument protein or an antigenic fragment or epitope thereof. In other embodiments, the antigenic fragment(s) of the VZV vaccine may be at least one VZV tegument polypeptide and at least one VZV glycoprotein polypeptide, for example any VZV glycoprotein selected from gE, gI, gB, gH, gK, gL, gC, gN, and gM.

The present disclosure includes variant VZV antigenic polypeptides. In some embodiments, the variant VZV antigenic polypeptide is a variant VZV gE polypeptide. The variant VZV gE polypeptides are designed to avoid ER/golgi retention of polypeptides, leading to increased surface expression of the antigen. In some embodiments, the variant gE polypeptides are truncated to remove the ER retention portion or the cytoplasmic tail portion of the polypeptide. In some embodiments, the variant VZV gE polypeptides are mutated to reduce VZV polypeptide localization to the ER/golgi/TGN. Such modifications inhibit ER trapping and, as such, expedite trafficking to the cell membrane.

Thus, in some embodiments, the VZV glycoprotein is a variant gE polypeptide. VZV gE has targeting sequences for the TGN in its C-terminus and is transported from the ER to the TGN in infected and gE-transfected cells. Most gE in the TGN appears to be retrieved by endocytosis from the plasma membrane and delivered to the TGN by endosomes, which is followed by recycling to the plasma membranes. gE is accumulated in TGN, along with other VZV proteins (e.g., tegument proteins) associated with the production of fully enveloped VZV virions. Thus, mutations to reduce TGN localization and endocytosis aids in the trafficking of gE to the cell membrane.

The variant VZV gE polypeptide can be any truncated polypeptide lacking the anchor domain (ER retention domain). For example, the variant VZV gE polypeptide can be a truncated VZV gE polypeptide comprising at least amino acids 1-124, including, for example, amino acids 1-124, 1-140, 1-160, 1-200, 1-250, 1-300, 1-350, 1-360, 1-400, 1-450, 1-500, 1-511, 1-550, and 1-561, as well as polypeptide fragments having fragment sizes within the recited size ranges. In one embodiment, the truncated VZV gE polypeptide comprises amino acids 1-561 of SEQ ID NO: 10. In some embodiments, the variant VZV gE polypeptide is a truncated polypeptide lacking the carboxy terminal tail domain. Thus in some embodiments, the truncated VZV gE polypeptide comprises amino acids 1-573 of SEQ ID NO: 10.

In some embodiments, the variant VZV gE polypeptide has at least one mutation in one or more motif(s) associated with ER retention, wherein the mutation(s) in one or more motif(s) results in decreased retention of the VZV gE polypeptide in the ER and/or golgi. In some embodiments, the variant VZV gE polypeptide has at least one mutation in one or more phosphorylated acidic motif(s). For example, the variant VZV gE polypeptide can be a full-length VZV gE polypeptide having a Y582G mutation, a Y569A mutation, or both a Y582G mutation and a Y569A mutation. Alternatively, the variant VZV gE polypeptide can be an antigenic fragment comprising, for example, amino acids 1-573 of VZV gE and having a Y569A mutation. Alternatively, the variant VZV gE polypeptide can be an antigenic fragment having mutation in an acidic phosphorylation motif, such as an SST motif. For example, the variant VZV gE polypeptide can be an antigenic fragment having AEAADA sequence (SEQ ID NO: 58).

In some embodiments, the variant VZV gE polypeptide is a full-length VZV gE polypeptide having additional sequence at the C-terminus which aids in secretion of the polypeptide. For example, the variant VZV gE polypeptide can be a full-length VZV gE polypeptide having an IgKappa sequence at the C-terminus. In some embodiments, the VZV gE polypeptide has additional sequence at the C-terminus that aids in secretion (I., an IgKappa sequence at the C-terminus) and the variant VZV gE polypeptide has at least one mutation in one or more motif(s) associated with ER retention, TGN localization, and/or endocytosis (e.g., a Y582G mutation, a Y569A mutation, or both a Y582G mutation and a Y569A mutation) and/or at least one mutation in one or more phosphorylated acidic motif(s). In some embodiments, the variant VZV gE polypeptide is a truncated polypeptide lacking the anchor domain (ER retention domain) and having an additional sequence at the C-terminus which aids in secretion of the polypeptide, for example, an IgKappa sequence at the C-terminus. In some embodiments, the truncated VZV gE polypeptide comprises amino acids 1-561 of SEQ ID NO: 10 and has an IgKappa sequence at the C-terminus. In some embodiments, the variant VZV gE polypeptide is a truncated polypeptide lacking the carboxy terminal tail domain and having an additional sequence at the C-terminus that aids in secretion of the polypeptide (e.g., having an IgKappa sequence at the C-terminus). In some embodiments, the truncated VZV gE polypeptide comprises amino acids 1-573 of SEQ ID NO: 10 and has an IgKappa sequence at the C-terminus.

In some embodiments, a VZV antigenic polypeptide is longer than 25 amino acids and shorter than 50 amino acids. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain or multichain polypeptides such as antibodies or insulin and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90% identity with a native or reference sequence.

In some embodiments “variant mimics” are provided. As used herein, a “variant mimic” contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic. For example, phenylalanine may act as an inactivating substitution for tyrosine, or alanine may act as an inactivating substitution for serine.

“Orthologs” refers to genes in different species that evolved from a common ancestral gene by speciation. Normally, orthologs retain the same function in the course of evolution. Identification of orthologs is critical for reliable prediction of gene function in newly sequenced genomes.

“Analogs” is meant to include polypeptide variants that differ by one or more amino acid alterations, for example, substitutions, additions or deletions of amino acid residues that still maintain one or more of the properties of the parent or starting polypeptide.

“Paralogs” are genes (or proteins) related by duplication within a genome. Orthologs retain the same function in the course of evolution, whereas paralogs evolve new functions, even if these are related to the original one.

The present disclosure provides several types of compositions that are polynucleotide or polypeptide based, including variants and derivatives. These include, for example, substitutional, insertional, deletion and covalent variants and derivatives. The term “derivative” is used synonymously with the term “variant,” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In alternative embodiments, sequences for (or encoding) signal sequences, termination sequences, transmembrane domains, linkers, multimerization domains (such as, e.g., foldon regions) and the like may be substituted with alternative sequences that achieve the same or a similar function. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue, such as lysine, arginine or histidine for another, or the substitution of one acidic residue, such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini or any combination thereof.

As used herein when referring to polypeptides the term “domain” refers to a motif of a polypeptide having one or more identifiable structural or functional characteristics or properties (e.g., binding capacity, serving as a site for protein-protein interactions).

As used herein, when referring to polypeptides the terms “site” as it pertains to amino acid based embodiments, is used synonymously with “amino acid residue” and “amino acid side chain.” As used herein, when referring to polynucleotides the terms “site” as it pertains to nucleotide based embodiments, is used synonymously with “nucleotide.” A site represents a position within a peptide or polypeptide or polynucleotide that may be modified, manipulated, altered, derivatized or varied within the polypeptide or polynucleotide based molecules.

As used herein, the terms “termini” or “terminus,” when referring to polypeptides or polynucleotides, refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N-termini and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids that are 40%, 50%, 60%, 70%, 80%, 90%, 95%, or 100% identical to any of the sequences described herein can be utilized in accordance with the present disclosure. In some embodiments, a polypeptide includes 2, 3, 4, 5, 6, 7, 8, 9, 10, or more mutations, as shown in any of the sequences provided or referenced herein. In some embodiments, a protein fragment is longer than 25 amino acids and shorter than 50 amino acids.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication.

### Multiprotein and Multicomponent Vaccines

The present disclosure encompasses VZV vaccines comprising multiple RNA (e.g., mRNA) polynucleotides, each encoding a single antigenic polypeptide, as well as VZV vaccines comprising a single RNA polynucleotide encoding more than one antigenic polypeptide (e.g., as a fusion polypeptide). Thus, it should be understood that a vaccine composition comprising a RNA (e.g., mRNA) polynucleotide having an open reading frame encoding a first VZV antigenic polypeptide and a RNA polynucleotide (e.g., mRNA) having an open reading frame encoding a second VZV antigenic polypeptide encompasses (a) vaccines that comprise a first RNA polynucleotide encoding a first VZV antigenic polypeptide and a second RNA polynucleotide encoding a second VZV antigenic polypeptide, and (b) vaccines that comprise a single RNA polynucleotide encoding a first and second VZV antigenic polypeptide (e.g., as a fusion polypeptide). VZV RNA vaccines of the present disclosure, in some embodiments, comprise 2-10 (e.g., 2, 3, 4, 5, 6, 7, 8, 9 or 10), or more, RNA polynucleotides having an open reading frame, each of which encodes a different VZV antigenic polypeptide (or a single RNA polynucleotide encoding 2-10, or more, different VZV antigenic polypeptides). In some embodiments, a VZV RNA vaccine comprises a RNA polynucleotide having an open reading frame encoding a VZV gE protein, a RNA polynucleotide having an open reading frame encoding a VZV gI protein, a RNA polynucleotide having an open reading frame encoding a VZV gB protein, a RNA polynucleotide having an open reading frame encoding a VZV gH protein, a RNA polynucleotide having an open reading frame encoding a VZV gK protein, a RNA polynucleotide having an open reading frame encoding a VZV gL protein, a RNA polynucleotide having an open reading frame encoding a VZV gC protein, a RNA polynucleotide having an open reading frame encoding a VZV gN protein, and a RNA polynucleotide having an open reading frame encoding a VZV gM protein. In some embodiments, a VZV RNA vaccine comprises a RNA polynucleotide having an open reading frame encoding a VZV gE and a RNA polynucleotide having an open reading frame encoding a VZV gI protein. In some embodiments, a VZV RNA vaccine comprises a RNA polynucleotide having an open reading frame encoding a VZV gE protein or a gE variant.

In some embodiments, a RNA polynucleotide encodes a VZV antigenic polypeptide fused to a signal peptide (e.g., SEQ ID NO: 56, 57, 109, 110, or 111). The signal peptide may be fused at the N-terminus or the C-terminus of the antigenic polypeptide.

### Signal Peptides

In some embodiments, antigenic polypeptides encoded by VZV polynucleotides comprise a signal peptide. Signal peptides, comprising the N-terminal 15-60 amino acids of proteins, are typically needed for the translocation across the membrane on the secretory pathway and thus universally control the entry of most proteins both in eukaryotes and prokaryotes to the secretory pathway. Signal peptides generally include of three regions: an N-terminal region of differing length, which usually comprises positively charged amino acids; a hydrophobic region; and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it. The signal peptide is not responsible for the final destination of the mature protein, however. Secretory proteins devoid of further address tags in their sequence are by default secreted to the external environment. Signal peptides are cleaved from precursor proteins by an endoplasmic reticulum (ER)-resident signal peptidase or they remain uncleaved and function as a membrane anchor. During recent years, a more advanced view of signal peptides has evolved, showing that the functions and immunodominance of certain signal peptides are much more versatile than previously anticipated.

Signal peptides typically function to facilitate the targeting of newly synthesized protein to the endoplasmic reticulum (ER) for processing. ER processing produces a mature Envelope protein, wherein the signal peptide is cleaved, typically by a signal peptidase of the host cell. A signal peptide may also facilitate the targeting of the protein to the cell membrane. VZV vaccines of the present disclosure may comprise, for example, RNA polynucleotides encoding an artificial signal peptide, wherein the signal peptide coding sequence is operably linked to and is in frame with the coding sequence of the VZV antigenic polypeptide. Thus, VZV vaccines of the present disclosure, in some embodiments, produce an antigenic polypeptide comprising a VZV antigenic polypeptide fused to a signal peptide. In some embodiments, a signal peptide is fused to the N-terminus of the VZV antigenic polypeptide. In some embodiments, a signal peptide is fused to the C-terminus of the VZV antigenic polypeptide.

In some embodiments, the signal peptide fused to the VZV antigenic polypeptide is an artificial signal peptide. In some embodiments, an artificial signal peptide fused to the VZV antigenic polypeptide encoded by the VZV RNA (e.g., mRNA) vaccine is obtained from an immunoglobulin protein, e.g., an IgE signal peptide or an IgG signal peptide. In some embodiments, a signal peptide fused to the VZV antigenic polypeptide encoded by a VZV RNA (e.g., mRNA) vaccine is an Ig heavy chain epsilon-1 signal peptide (IgE HC SP) having the sequence of: MDWTWILFLVAAATRVHS (SEQ ID NO: 56). In some embodiments, a signal peptide fused to a VZV antigenic polypeptide encoded by the VZV RNA (e.g., mRNA) vaccine is an IgGk chain V-III region HAH signal peptide (IgGk SP) having the sequence of METPAQLLFLLLLWLPDTTG (SEQ ID NO: 57). In some embodiments, the VZV antigenic polypeptide encoded by a VZV RNA (e.g., mRNA) vaccine has an amino acid sequence set forth in one of 10, 14, 18, 22, 26, 30, 34, 38, 42 and 45-55 fused to a signal peptide of any of SEQ ID NO: 56, 57, 109, 110 and 111. The examples disclosed herein are not meant to be limiting and any signal peptide that is known in the art to facilitate targeting of a protein to ER for processing and/or targeting of a protein to the cell membrane may be used in accordance with the present disclosure.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide may have a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

A signal peptide is typically cleaved from the nascent polypeptide at the cleavage junction during ER processing. The mature VZV antigenic polypeptide produce by VZV RNA vaccine of the present disclosure typically does not comprise a signal peptide.

### Chemical Modifications

RNA (e.g., mRNA) vaccines of the present disclosure comprise, in some embodiments, at least one ribonucleic acid (RNA) polynucleotide having an open reading frame encoding at least one respiratory syncytial virus (VZV) antigenic polypeptide, wherein said RNA comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties.

Modifications of polynucleotides include, without limitation, those described herein, and include, but are expressly not limited to, those modifications that comprise chemical modifications. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

With respect to a polypeptide, the term “modification” refers to a modification relative to the canonical set of 20 amino acids. Polypeptides, as provided herein, are also considered “modified” if they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those polynucleotides having at least one chemical modification. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides), including but not limited to chemical modification, that are useful in the compositions, vaccines, methods and synthetic processes of the present disclosure include, but are not limited to the following: 2-methylthio-N6-(cis-hydroxyisopentenyl) adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl) adenosine; N6,2′-O-dimethyladenosine; N6,2′-O-dimethyladenosine; N6,N6,2′-O-trimethyladenosine; N6, N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl) adenine; N6-cis-hydroxy-isopentenyl-adenosine; β-thio-adenosine; 2 (amino) adenine; 2 (aminopropyl) adenine; 2 (methylthio) N6 (isopentenyl) adenine; 2-(alkyl) adenine; 2-(aminoalkyl) adenine; 2-(aminopropyl) adenine; 2-(halo) adenine; 2-(halo) adenine; 2-(propyl) adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl) adenine; 6 (methyl) adenine; 6-(alkyl) adenine; 6-(methyl) adenine; 7 (deaza) adenine; 8 (alkenyl) adenine; 8 (alkynyl) adenine; 8 (amino) adenine; 8 (thioalkyl) adenine; 8-(alkenyl) adenine; 8-(alkyl) adenine; 8-(alkynyl) adenine; 8-(amino) adenine; 8-(halo) adenine; 8-(hydroxyl) adenine; 8-(thioalkyl) adenine; 8-(thiol) adenine; 8-azido-adenosine; aza adenine; deaza adenine; N6 (methyl) adenine; N6-(isopentyl) adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; a-thio-cytidine; 2-(thio) cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza) cytosine; 3 (methyl) cytosine; 3-(alkyl) cytosine; 3-(deaza) 5 (aza) cytosine; 3-(methyl) cytidine; 4,2′-O-dimethylcytidine; 5 (halo) cytosine; 5 (methyl) cytosine; 5 (propynyl) cytosine; 5 (trifluoromethyl) cytosine; 5-(alkyl) cytosine; 5-(alkynyl) cytosine; 5-(halo) cytosine; 5-(propynyl) cytosine; 5-(trifluoromethyl) cytosine; 5-bromo-cytidine; 5-iodo-cytidine; 5-propynyl cytosine; 6-(azo) cytosine; 6-aza-cytidine; aza cytosine; deaza cytosine; N4 (acetyl) cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl) cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl) cytidine TP; 3′-Ethynylcytidine TP; 4′-Azidocytidine TP; 4′-Carbocyclic cytidine TP; 4′-Ethynylcytidine TP; 5-(1-Propynyl) ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archacosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; a-thio-guanosine; 2 (propyl) guanine; 2-(alkyl) guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl) guanine; 6-(alkyl) guanine; 6-(methyl) guanine; 6-methyl-guanosine; 7 (alkyl) guanine; 7 (deaza) guanine; 7 (methyl) guanine; 7-(alkyl) guanine; 7-(deaza) guanine; 7-(methyl) guanine; 8 (alkyl) guanine; 8 (alkynyl) guanine; 8 (halo) guanine; 8 (thioalkyl) guanine; 8-(alkenyl) guanine; 8-(alkyl) guanine; 8-(alkynyl) guanine; 8-(amino) guanine; 8-(halo) guanine; 8-(hydroxyl) guanine; 8-(thioalkyl) guanine; 8-(thiol) guanine; aza guanine; deaza guanine; N(methyl) guanine; N-(methyl) guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosinc TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridinc; 5-hydroxyuridinc; 5-methyluridine; 5-taurinomethyl-2-thiouridine; 5-taurinomethyluridine; Dihydrouridinc; Pseudouridinc; (3-(3-amino-3-carboxypropyl) uridinc; 1-methyl-3-(3-amino-5-carboxypropyl) pseudouridinc; 1-methylpscduouridine; 1-ethyl-pseudouridine; 2′-O-methyluridine; 2′-O-methylpseudouridinc; 2′-O-methyluridine; 2-thio-2′-O-methyluridinc; 3-(3-amino-3-carboxypropyl) uridine; 3,2′-O-dimethyluridine; 3-Methyl-pseudo-Uridine TP; 4-thiouridine; 5-(carboxyhydroxymethyl) uridine; 5-(carboxyhydroxymethyl) uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridinc; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridinc; 5-carboxymethylaminomethyluridinc; 5-Carbamoylmethyluridine TP; 5-methoxycarbonylmethyl-2′-O-methyluridine; 5-methoxycarbonylmethyl-2-thiouridinc; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl) uridinc TP; 5-propynyl uracil; a-thio-uridine; 1 (aminoalkylamino-carbonylethylenyl)-2 (thio)-pseudouracil; 1 (aminoalkylaminocarbonylethylenyl)-2,4-(dithio) pscudouracil; 1 (aminoalkylaminocarbonylethylenyl)-4 (thio) pseudouracil; 1 (aminoalkylaminocarbonylethylenyl)-pseudouracil; 1 (aminocarbonylethylenyl)-2 (thio)-pseudouracil; 1 (aminocarbonylethylenyl)-2,4-(dithio) pseudouracil; 1 (aminocarbonylethylenyl)-4 (thio) pseudouracil; 1 (aminocarbonylethylenyl)-pseudouracil; 1 substituted 2 (thio)-pscudouracil; 1 substituted 2,4-(dithio) pseudouracil; 1 substituted 4 (thio) pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl) pseudo-UTP; 1-Methyl-pseudo-UTP; 1-Ethyl-pseudo-UTP; 2 (thio) pscudouracil; 2′ deoxy uridine; 2′ fluorouridine; 2-(thio) uracil; 2,4-(dithio) psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridinc; 2′-Deoxy-2′-a-aminouridine TP; 2′-Deoxy-2′-a-azidouridine TP; 2-mcthylpscudouridinc; 3 (3 amino-3 carboxypropyl) uracil; 4 (thio) pseudouracil; 4-(thio) pscudouracil; 4-(thio) uracil; 4-thiouracil; 5 (1,3-diazole-1-alkyl) uracil; 5 (2-aminopropyl) uracil; 5 (aminoalkyl) uracil; 5 (dimethylaminoalkyl) uracil; 5 (guanidiniumalkyl) uracil; 5 (methoxycarbonylmethyl)-2-(thio) uracil; 5 (methoxycarbonyl-methyl) uracil; 5 (methyl) 2 (thio) uracil; 5 (methyl) 2,4 (dithio) uracil; 5 (methyl) 4 (thio) uracil; 5 (methylaminomethyl)-2 (thio) uracil; 5 (methylaminomethyl)-2,4 (dithio) uracil; 5 (methylaminomethyl)-4 (thio) uracil; 5 (propynyl) uracil; 5 (trifluoromethyl) uracil; 5-(2-aminopropyl) uracil; 5-(alkyl)-2-(thio) pseudouracil; 5-(alkyl)-2,4 (dithio) pseudouracil; 5-(alkyl)-4 (thio) pseudouracil; 5-(alkyl) pscudouracil; 5-(alkyl) uracil; 5-(alkynyl) uracil; 5-(allylamino) uracil; 5-(cyanoalkyl) uracil; 5-(dialkylaminoalkyl) uracil; 5-(dimethylaminoalkyl) uracil; 5-(guanidiniumalkyl) uracil; 5-(halo) uracil; 5-(1,3-diazole-1-alkyl) uracil; 5-(methoxy) uracil; 5-(methoxycarbonylmethyl)-2-(thio) uracil; 5-(methoxycarbonyl-methyl) uracil; 5-(methyl) 2 (thio) uracil; 5-(methyl) 2,4 (dithio) uracil; 5-(methyl) 4 (thio) uracil; 5-(methyl)-2-(thio) pseudouracil; 5-(methyl)-2,4 (dithio) pseudouracil; 5-(methyl)-4 (thio) pseudouracil; 5-(methyl) pseudouracil; 5-(methylaminomethyl)-2 (thio) uracil; 5-(methylaminomethyl)-2,4 (dithio) uracil; 5-(methylaminomethyl)-4-(thio) uracil; 5-(propynyl) uracil; 5-(trifluoromethyl) uracil; 5-aminoallyl-uridine; 5-bromo-uridine; 5-iodo-uridine; 5-uracil; 6 (azo) uracil; 6-(azo) uracil; 6-aza-uridine; allyamino-uracil; aza uracil; deaza uracil; N3 (methyl) uracil; P scudo-UTP-1-2-cthanoic acid; Pscudouracil; 4-Thio-pseudo-UTP; 1-carboxymethyl-pseudouridinc; 1-methyl-1-deaza-pscudouridine; 1-propynyl-uridinc; 1-taurinomethyl-1-methyl-uridine; 1-taurinomethyl-4-thio-uridine; 1-taurinomethyl-pseudouridinc; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±) 1-(2-Hydroxypropyl) pscudouridine TP; (2R)-1-(2-Hydroxypropyl) pseudouridine TP; (2S)-1-(2-Hydroxypropyl) pseudouridine TP; (E)-5-(2-Bromo-vinyl) ara-uridine TP; (E)-5-(2-Bromo-vinyl) uridine TP; (Z)-5-(2-Bromo-vinyl) ara-uridine TP; (Z)-5-(2-Bromo-vinyl) uridine TP; 1-(2,2,2-Trifluoroethyl)-pseudo-UTP; 1-(2,2,3,3,3-Pentafluoropropyl) pseudouridine TP; 1-(2,2-Dicthoxyethyl) pseudouridine TP; 1-(2,4,6-Trimethylbenzyl) pseudouridine TP; 1-(2,4,6-Trimethyl-benzyl) pseudo-UTP; 1-(2,4,6-Trimethyl-phenyl) pseudo-UTP; 1-(2-Amino-2-carboxyethyl) pseudo-UTP; 1-(2-Amino-ethyl) pseudo-UTP; 1-(2-Hydroxyethyl) pseudouridine TP; 1-(2-Methoxyethyl) pseudouridine TP; 1-(3,4-Bis-trifluoromethoxybenzyl) pseudouridine TP; 1-(3,4-Dimethoxybenzyl) pseudouridine TP; 1-(3-Amino-3-carboxypropyl) pseudo-UTP; 1-(3-Amino-propyl) pseudo-UTP; 1-(3-Cyclopropyl-prop-2-ynyl) pseudouridine TP; 1-(4-Amino-4-carboxybutyl) pseudo-UTP; 1-(4-Amino-benzyl) pseudo-UTP; 1-(4-Amino-butyl) pseudo-UTP; 1-(4-Amino-phenyl) pseudo-UTP; 1-(4-Azidobenzyl) pseudouridine TP; 1-(4-Bromobenzyl) pseudouridine TP; 1-(4-Chlorobenzyl) pseudouridine TP; 1-(4-Fluorobenzyl) pseudouridine TP; 1-(4-lodobenzyl) pseudouridine TP; 1-(4-Methanesulfonylbenzyl) pseudouridine TP; 1-(4-Methoxybenzyl) pseudouridine TP; 1-(4-Methoxy-benzyl) pseudo-UTP; 1-(4-Methoxy-phenyl) pseudo-UTP; 1-(4-Methylbenzyl) pseudouridine TP; 1-(4-Methyl-benzyl) pseudo-UTP; 1-(4-Nitrobenzyl) pseudouridine TP; 1-(4-Nitro-benzyl) pseudo-UTP; 1 (4-Nitro-phenyl) pseudo-UTP; 1-(4-Thiomethoxybenzyl) pseudouridine TP; 1-(4-Trifluoromethoxybenzyl) pseudouridine TP; 1-(4-Trifluoromethylbenzyl) pseudouridine TP; 1-(5-Amino-pentyl) pseudo-UTP; 1-(6-Amino-hexyl) pseudo-UTP; 1,6-Dimethyl-pseudo-UTP; 1-[3-(2-{2-[2-(2-Aminocthoxy)-ethoxy]-ethoxy}-ethoxy)-propionyl|pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pscudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl) pseudo-UTP; 1-Methyl-6-(4-morpholino)-pseudo-UTP; 1-Methyl-6-(4-thiomorpholino)-pseudo-UTP; 1-Methyl-6-(substituted phenyl) pseudo-UTP; 1-Methyl-6-amino-pseudo-UTP; 1-Methyl-6-azido-pscudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pscudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pscudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMc-5-Me-UTP; 2′-OMe-pscudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl) uridine TP; 3-Alkyl-pseudo-UTP; 4′-Azidouridine TP; 4′-Carbocyclic uridinc TP; 4′-Ethynyluridine TP; 5-(1-Propynyl) ara-uridine TP; 5-(2-Furanyl) uridinc TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridinc TP; 5-Trifluoromethyl-Uridin TP; 5-Vinylarauridinc TP; 6-(2,2,2-Trifluorocthyl)-pseudo-UTP; 6-(4-Morpholino)-pseudo-UTP; 6-(4-Thiomorpholino)-pseudo-UTP; 6-(Substituted-Phenyl)-pscudo-UTP; 6-Amino-pseudo-UTP; 6-Azido-pseudo-UTP; 6-Bromo-pseudo-UTP; 6-Butyl-pscudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pscudo-UTP; 6-Hydroxyamino-pscudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pscudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2 (2-ethoxy)-ethoxy}-ethoxy]-ethoxy)-ethoxy}]propionic acid; Pseudouridine TP 1-[3-{2-(2-[2-ethoxy]-ethoxy)-ethoxy}|propionic acid; Pseudouridine TP 1-[3-{2-(2-ethoxy)-ethoxy}| propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosinc; 2,6-(diamino) purinc; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl;1,3,5-(triaza)-2,6-(dioxa)-naphthalenc;2 (amino) purine;2,4,5-(trimethyl)phenyl;2′ methyl, 2′amino, 2′azido, 2′fluro-cytidine;2′ methyl, 2′amino, 2′azido, 2′fluro-adenine;2′methyl, 2′amino, 2′azido, 2′fluro-uridinc;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl; 4,6-(dimethyl) indolyl; 5 nitroindole; 5 substituted pyrimidines; 5-(methyl) isocarbostyrilyl; 5-nitroindole; 6-(aza)pyrimidinc; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza) indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl) uridine TP; and N6-(19-Amino-pentaoxanonadecyl) adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 1-ethyl-pseudouridine (e1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (prcQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl) pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQObase, preQlbase, and combinations of two or more thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polyribonucleotide (e.g., RNA polyribonucleotide, such as mRNA polyribonucleotide) includes a combination of at least two (e.g., 2, 3, 4 or more) of the aforementioned modified nucleobases. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m1ψ), 1-ethyl-pseudouridine (e1ψ), 5-methoxy-uridine (mo5U), 5-methyl-cytidine (m5C), pseudouridine (ψ), α-thio-guanosine and α-thio-adenosine. In some embodiments, the polyribonucleotide includes a combination of at least two (e.g., 2, 3, 4 or more) of the aforementioned modified nucleobases, including but not limited to chemical modifications.

In some embodiments, polynucleotides (e.g., RNA polynucleotides, such as mRNA polynucleotides) comprise pseudouridine (ψ) and 5-methyl-cytidine (m5C). In some embodiments, the polyribonucleotides (e.g., RNA, such as mRNA) comprise 1-methyl-pseudouridine (m1ψ). In some embodiments, the polyribonucleotides (e.g., RNA, such as mRNA) comprise 1-ethyl-pseudouridine (e1ψ). In some embodiments, the polyribonucleotides (e.g., RNA, such as mRNA) comprise 1-methyl-pseudouridine (m1ψ) and 5-methyl-cytidine (m5C). In some embodiments, the polyribonucleotides (e.g., RNA, such as mRNA) comprise 1-ethyl-pseudouridine (e1ψ) and 5-methyl-cytidine (m5C). In some embodiments, the polyribonucleotides (e.g., RNA, such as mRNA) comprise 2-thiouridine (s2U). In some embodiments, the polyribonucleotides (e.g., RNA, such as mRNA) comprise 2-thiouridine and 5-methyl-cytidine (m5C). In some embodiments, the polyribonucleotides (e.g., RNA, such as mRNA) comprise methoxy-uridine (mo5U). In some embodiments, the polyribonucleotides (e.g., RNA, such as mRNA) comprise 5-methoxy-uridine (mo5U) and 5-methyl-cytidine (m5C). In some embodiments, the polyribonucleotides (e.g., RNA, such as mRNA) comprise 2′-O-methyl uridine. In some embodiments, the polyribonucleotides (e.g., RNA, such as mRNA) comprise 2′-O-methyl uridine and 5-methyl-cytidine (m5C). In some embodiments, the polyribonucleotides (e.g., RNA, such as mRNA) comprise N6-methyl-adenosine (m6A). In some embodiments, the polyribonucleotides (e.g., RNA, such as mRNA) comprise N6-methyl-adenosine (m6A) and 5-methyl-cytidine (m5C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 1-methyl-pseudouridine, meaning that all uridine residues in the mRNA sequence are replaced with 1-methyl-pseudouridine. Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and nucleosides having a modified uridine include 1-methyl-pseudouridine (m1ψ), 1-ethyl-pseudouridine (e1ψ), 5-methoxy uridine, 2-thio uridine, 5-cyano uridine, 2′-O-methyl uridine and 4′-thio uridinc.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosinc (preQ0), 7-aminomethyl-7-deaza-guanosine (preQ1), 7-methyl-guanosine (m7G), 1-methyl-guanosine (m1G), 8-oxo-guanosine, and 7-methyl-8-oxo-guanosinc.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA vaccines comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridincor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵sc²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (tm⁵U), 1-taurinomethyl-pseudouridine, 5-taurinomethyl-2-thio-uridine (m⁵s²U), 1-taurinomethyl-4-thio-pseudouridine, 5-methyl-uridine (m⁵U, i.e., having the nucleobase deoxythymine), 1-methyl-pseudouridine (m¹ψ), 1-ethyl-pseudouridine (e1ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (vm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) Juridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac+C), 5-formyl-cytidine (f5° C.), N4-methyl-cytidine (m⁴° C.), 5-methyl-cytidine (m⁵° C.), 5-halo-cytidine (e.g., 5-iodo-cytidine), 5-hydroxymethyl-cytidine (hm5° C.), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularinc, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m+2 Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl) adenosine (io⁶A), 2-methylthio-N6-(cis-hydroxyisopentenyl) adenosine (ms²io⁶A), N6-glycinylcarbamoyl-adenosine (g⁶A), N6-threonylcarbamoyl-adenosine (t6A), N6-methyl-N6-threonylcarbamoyl-adenosine (m⁶t⁶A), 2-methylthio-N6-thrconylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac6A), 7-methyl-adenine, 2-methylthio-adenine, 2-methoxy-adenine, a-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m′I), wyosine (imG), methylwyosine (mimG), 4-demethyl-wyosine (imG-14), isowyosine (imG2), wybutosine (yW), peroxywybutosine (02yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQ1), archacosine (G+), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²²G), N2,7-dimethyl-guanosine (m^{2.7}G), N2, N2,7-dimethyl-guanosine (m^{2.2.7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosinc (Gm), N2-methyl-2′-O-methyl-guanosine (m²Gm), N2,N2-dimethyl-2′-O-methyl-guanosine (m²²Gm), 1-methyl-2′-O-methyl-guanosine (m¹Gm), N2,7-dimethyl-2′-O-methyl-guanosine (m^{2.7}Gm), 2′-O-methyl-inosine (Im), 1,2′-O-dimethyl-inosine (m′Im), 2′-O-ribosylguanosine (phosphate) (Gr (p)), 1-thio-guanosine, 06-methyl-guanosine, 2′-F-ara-guanosine, and 2′-F-guanosine.

In Vitro Transcription of RNA (e.g., mRNA)

VZV vaccines of the present disclosure comprise at least one RNA polynucleotide, such as a mRNA (e.g., modified mRNA). mRNA, for example, is transcribed in vitro from template DNA, referred to as an “in vitro transcription template.” In some embodiments, the at least one RNA polynucleotide has at least one chemical modification. The at least one chemical modification may include, but is expressly not limited to, any modification described herein.

In vitro transcription of RNA is known in the art and is described in International Publication WO/2014/152027, which is incorporated by reference herein in its entirety. For example, in some embodiments, the RNA transcript is generated using a non-amplified, linearized DNA template in an in vitro transcription reaction to generate the RNA transcript. In some embodiments the RNA transcript is capped via enzymatic capping. In some embodiments the RNA transcript is purified via chromatographic methods, e.g., use of an oligo dT substrate. Some embodiments exclude the use of DNase. In some embodiments the RNA transcript is synthesized from a non-amplified, linear DNA template coding for the gene of interest via an enzymatic in vitro transcription reaction utilizing a T7 phage RNA polymerase and nucleotide triphosphates of the desired chemistry. Any number of RNA polymerases or variants may be used in the method of the present invention. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides.

In some embodiments a non-amplified, linearized plasmid DNA is utilized as the template DNA for in vitro transcription. In some embodiments, the template DNA is isolated DNA. In some embodiments, the template DNA is cDNA. In some embodiments, the cDNA is formed by reverse transcription of a RNA polynucleotide, for example, but not limited to VZV RNA, e.g. VZV mRNA. In some embodiments, Cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5 ‘to and operably linked to the gene of interest.

In some embodiments, an in vitro transcription template encodes a 5’ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a poly A tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

### Methods of Treatment

Provided herein are compositions (e.g., pharmaceutical compositions), methods, kits and reagents for prevention and/or treatment of VZV in humans and other mammals. VZV RNA vaccines can be used as therapeutic or prophylactic agents. They may be used in medicine to prevent and/or treat infectious disease. In exemplary aspects, the VZV RNA vaccines of the invention are used to provide prophylactic protection from varicella and herpes zoster. Varicella is an acute infectious disease caused by VZV. The primary varicella zoster virus infection that results in chickenpox (varicella) may result in complications, including viral or secondary bacterial pneumonia. Even when the clinical symptoms of chickenpox have resolved, VZV remains dormant in the nervous system of the infected person in the trigeminal and dorsal root ganglia and may reactivate later in life, travelling from the sensory ganglia back to the skin where it produces a disease (rash) known as shingles or herpes zoster, and can also cause a number of neurologic conditions ranging from aseptic meningitis to encephalitis. The VZV vaccines of the present disclosure can be used to prevent and/or treat both the primary infection (Chicken pox) and also the re-activated viral infection (shingles or herpes zoster) and may be particularly useful for prevention and/or treatment of immunocompromised and elderly patients to prevent or to reduce the severity and/or duration of herpes zoster.

Prophylactic protection from VZV can be achieved following administration of a VZV RNA vaccine of the present disclosure. Vaccines can be administered once, twice, three times, four times or more but it is likely sufficient to administer the vaccine once (optionally followed by a single booster). It is possible, although less desirable, to administer the vaccine to an infected individual to achieve a therapeutic response. Dosing may need to be adjusted accordingly.

A method of eliciting an immune response in a subject against a VZV is provided in aspects of the present disclosure. The method involves administering to the subject a VZV RNA vaccine comprising at least one RNA (e.g., mRNA) polynucleotide having an open reading frame encoding at least one VZV antigenic polypeptide or an immunogenic fragment thereof, thereby inducing in the subject an immune response specific to VZV antigenic polypeptide or an immunogenic fragment thereof, wherein anti-antigenic polypeptide antibody titer in the subject is increased following vaccination relative to anti-antigenic polypeptide antibody titer in a subject vaccinated with a prophylactically effective dose of a traditional vaccine against the VZV. An “anti-antigenic polypeptide antibody” is a serum antibody the binds specifically to the antigenic polypeptide.

A prophylactically effective dose is a therapeutically effective dose that prevents infection with the virus at a clinically acceptable level. In some embodiments, the therapeutically effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, VLP vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

A method of eliciting an immune response in a subject against a VZV is provided in aspects of the invention. The method involves administering to the subject a VZV RNA vaccine comprising at least one RNA polynucleotide having an open reading frame encoding at least one VZV antigenic polypeptide or an immunogenic fragment thereof, thereby inducing in the subject an immune response specific to VZV antigenic polypeptide or an immunogenic fragment thereof, wherein anti-antigenic polypeptide antibody titer in the subject is increased following vaccination relative to anti-antigenic polypeptide antibody titer in a subject vaccinated with a prophylactically effective dose of a traditional vaccine against the VZV. An “anti-antigenic polypeptide antibody” is a serum antibody the binds specifically to the antigenic polypeptide.

A prophylactically effective dose is a therapeutically effective dose that prevents infection with the virus at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, etc.

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the VZV.

In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against the VZV.

In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against the VZV.

In some embodiments the anti-antigenic polypeptide antibody titer in the subject is increased 3 log following vaccination relative to anti-antigenic polypeptide antibody titer in a subject vaccinated with a prophylactically effective dose of a traditional vaccine against the VZV.

In some embodiments the anti-antigenic polypeptide antibody titer in the subject is increased 5 log following vaccination relative to anti-antigenic polypeptide antibody titer in a subject vaccinated with a prophylactically effective dose of a traditional vaccine against the VZV.

In some embodiments the anti-antigenic polypeptide antibody titer in the subject is increased 10 log following vaccination relative to anti-antigenic polypeptide antibody titer in a subject vaccinated with a prophylactically effective dose of a traditional vaccine against the VZV.

A method of eliciting an immune response in a subject against a VZV is provided in other aspects of the invention. The method involves administering to the subject a VZV RNA vaccine comprising at least one RNA polynucleotide having an open reading frame encoding at least one VZV antigenic polypeptide or an immunogenic fragment thereof, thereby inducing in the subject an immune response specific to VZV antigenic polypeptide or an immunogenic fragment thereof, wherein the immune response in the subject is equivalent to an immune response in a subject vaccinated with a traditional vaccine against the VZV at 2 times to 100 times the dosage level relative to the RNA vaccine.

In some embodiments the immune response in the subject is equivalent to an immune response in a subject vaccinated with a traditional vaccine at twice the dosage level relative to the VZV RNA vaccine.

In some embodiments the immune response in the subject is equivalent to an immune response in a subject vaccinated with a traditional vaccine at three times the dosage level relative to the VZV RNA vaccine.

In some embodiments the immune response in the subject is equivalent to an immune response in a subject vaccinated with a traditional vaccine at 4 times the dosage level relative to the VZV RNA vaccine.

In some embodiments the immune response in the subject is equivalent to an immune response in a subject vaccinated with a traditional vaccine at 5 times the dosage level relative to the VZV RNA vaccine.

In some embodiments the immune response in the subject is equivalent to an immune response in a subject vaccinated with a traditional vaccine at 10 times the dosage level relative to the VZV RNA vaccine.

In some embodiments the immune response in the subject is equivalent to an immune response in a subject vaccinated with a traditional vaccine at 50 times the dosage level relative to the VZV RNA vaccine.

In some embodiments the immune response in the subject is equivalent to an immune response in a subject vaccinated with a traditional vaccine at 100 times the dosage level relative to the VZV RNA vaccine.

In some embodiments the immune response in the subject is equivalent to an immune response in a subject vaccinated with a traditional vaccine at 10 times to 1000 times the dosage level relative to the VZV RNA vaccine.

In some embodiments the immune response in the subject is equivalent to an immune response in a subject vaccinated with a traditional vaccine at 100 times to 1000 times the dosage level relative to the VZV RNA vaccine.

In other embodiments the immune response is assessed by determining [protein]antibody titer in the subject.

In other aspects the invention is a method of eliciting an immune response in a subject against a VZV by administering to the subject a VZV RNA vaccine comprising at least one RNA polynucleotide having an open reading frame encoding at least one VZV antigenic polypeptide or an immunogenic fragment thereof, thereby inducing in the subject an immune response specific to VZV antigenic polypeptide or an immunogenic fragment thereof, wherein the immune response in the subject is induced 2 days to 10 weeks earlier relative to an immune response induced in a subject vaccinated with a prophylactically effective dose of a traditional vaccine against the VZV. In some embodiments the immune response in the subject is induced in a subject vaccinated with a prophylactically effective dose of a traditional vaccine at 2 times to 100 times the dosage level relative to the RNA vaccine.

In some embodiments the immune response in the subject is induced 2 days earlier relative to an immune response induced in a subject vaccinated with a prophylactically effective dose of a traditional vaccine.

In some embodiments the immune response in the subject is induced 3 days earlier relative to an immune response induced in a subject vaccinated a prophylactically effective dose of a traditional vaccine.

In some embodiments the immune response in the subject is induced 1 week earlier relative to an immune response induced in a subject vaccinated with a prophylactically effective dose of a traditional vaccine.

In some embodiments the immune response in the subject is induced 2 weeks earlier relative to an immune response induced in a subject vaccinated with a prophylactically effective dose of a traditional vaccine.

In some embodiments the immune response in the subject is induced 3 weeks earlier relative to an immune response induced in a subject vaccinated with a prophylactically effective dose of a traditional vaccine.

In some embodiments the immune response in the subject is induced 5 weeks earlier relative to an immune response induced in a subject vaccinated with a prophylactically effective dose of a traditional vaccine.

In some embodiments the immune response in the subject is induced 10 weeks earlier relative to an immune response induced in a subject vaccinated with a prophylactically effective dose of a traditional vaccine.

A method of v eliciting an immune response in a subject against a VZV by administering to the subject a VZV RNA vaccine having an open reading frame encoding a first antigenic polypeptide, wherein the RNA polynucleotide does not include a stabilization element, and wherein an adjuvant is not coformulated or co-administered with the vaccine.

### Broad Spectrum VZV Vaccines

It is envisioned that there may be situations where persons are at risk for infection with more than one strain of VZV. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one strain of VZV, a combination vaccine can be administered that includes RNA encoding at least one antigenic polypeptide protein (or antigenic portion thereof) of a first VZV and further includes RNA encoding at least one antigenic polypeptide protein (or antigenic portion thereof) of a second VZV. RNAs (mRNAs) can be co-formulated, for example, in a single lipid nanoparticle (LNP) or can be formulated in separate LNPs destined for co-administration.

### Flagellin Adjuvants

Flagellin is an approximately 500 amino acid monomeric protein that polymerizes to form the flagella associated with bacterial motion. Flagellin is expressed by a variety of flagellated bacteria (Salmonella typhimurium for example) as well as non-flagellated bacteria (such as Escherichia coli). Sensing of flagellin by cells of the innate immune system (dendritic cells, macrophages, etc.) is mediated by the Toll-like receptor 5 (TLR5) as well as by Nod-like receptors (NLRs) Ipaf and Naip5. TLRs and NLRs have been identified as playing a role in the activation of innate immune response and adaptive immune response. As such, flagellin provides an adjuvant effect in a vaccine.

The nucleotide and amino acid sequences encoding known flagellin polypeptides are publicly available in the NCBI GenBank database. The flagellin sequences from S. Typhimurium, H. Pylori, V. Cholera, S. marcesens, S. flexneri, T. Pallidum, L. pneumophila, B. burgdorferei, C. difficile, R. meliloti, A. tumefaciens, R. lupini, B. clarridgeiae, P. Mirabilis, B. subtilus, L. monocytogenes, P. aeruginosa, and E. coli, among others are known.

A flagellin polypeptide, as used herein, refers to a full length flagellin protein, immunogenic fragments thereof, and peptides having at least 50% sequence identify to a flagellin protein or immunogenic fragments thereof. Exemplary flagellin proteins include flagellin from Salmonella typhi (UniPro Entry number: Q56086), Salmonella typhimurium (AOAOC9DG09), Salmonella enteritidis (AOAOC9BAB7), and Salmonella choleraesuis (Q6V2X8), and SEQ ID NO: 115-117. In some embodiments, the flagellin polypeptide has at least 60%, 70%, 75%, 80%, 90%, 95%, 97%, 98%, or 99% sequence identify to a flagellin protein or immunogenic fragments thereof.

In some embodiments, the flagellin polypeptide is an immunogenic fragment. An immunogenic fragment is a portion of a flagellin protein that provokes an immune response.

In some embodiments, the immune response is a TLR5 immune response. An example of an immunogenic fragment is a flagellin protein in which all or a portion of a hinge region has been deleted or replaced with other amino acids. For example, an antigenic polypeptide may be inserted in the hinge region. Hinge regions are the hypervariable regions of a flagellin. Hinge regions of a flagellin are also referred to as “D3 domain or region, “propeller domain or region,” “hypervariable domain or region” and “variable domain or region.” “At least a portion of a hinge region,” as used herein, refers to any part of the hinge region of the flagellin, or the entirety of the hinge region. In other embodiments an immunogenic fragment of flagellin is a 20, 25, 30, 35, or 40 amino acid C-terminal fragment of flagellin.

The flagellin monomer is formed by domains DO through D3. DO and D1, which form the stem, are composed of tandem long alpha helices and are highly conserved among different bacteria. The DI domain includes several stretches of amino acids that are useful for TLR5 activation. The entire DI domain or one or more of the active regions within the domain are immunogenic fragments of flagellin. Examples of immunogenic regions within the DI domain include residues 88-114 and residues 411-431 (in Salmonella typhimurium FliC flagellin. Within the 13 amino acids in the 88-100 region, at least 6 substitutions are permitted between Salmonella flagellin and other flagellin proteins that still preserve TLR5 activation. Thus, immunogenic fragments of flagellin include flagellin like sequences that activate TLR5 and contain a 13 amino acid motif that is 53% or more identical to the Salmonella sequence in 88-100 of FliC (LQRVRELAVQSAN; SEQ ID NO: 118).

In some embodiments, the RNA (e.g., mRNA) vaccine includes an RNA that encodes a fusion protein of flagellin and one or more antigenic polypeptides. A “fusion protein” as used herein, refers to a linking of two components of the construct. In some embodiments, a carboxy-terminus of the antigenic polypeptide is fused or linked to an amino terminus of the flagellin polypeptide. In other embodiments, an amino-terminus of the antigenic polypeptide is fused or linked to a carboxy-terminus of the flagellin polypeptide. The fusion protein may include, for example, one, two, three, four, five, six or more flagellin polypeptides linked to one, two, three, four, five, six or more antigenic polypeptides. When two or more flagellin polypeptides and/or two or more antigenic polypeptides are linked such a construct may be referred to as a “multimer.”

Each of the components of a fusion protein may be directly linked to one another or they may be connected through a linker. For instance, the linker may be an amino acid linker. The amino acid linker encoded for by the RNA (e.g., mRNA) vaccine to link the components of the fusion protein may include, for instance, at least one member selected from the group consisting of a lysine residue, a glutamic acid residue, a serine residue and an arginine residue. In some embodiments the linker is 1-30, 1-25, 1-25, 5-10, 5, 15, or 5-20 amino acids in length.

In other embodiments the RNA (e.g., mRNA) vaccine includes at least two separate RNA polynucleotides, one encoding one or more antigenic polypeptides and the other encoding the flagellin polypeptide. The at least two RNA polynucleotides may be co-formulated in a carrier such as a lipid nanoparticle.

### Therapeutic and Prophylactic Compositions

Provided herein are compositions (e.g., pharmaceutical compositions), methods, kits and reagents for prevention, treatment or diagnosis of VZV in humans and other mammals, for example. VZV RNA (e.g., mRNA) vaccines can be used as therapeutic or prophylactic agents. They may be used in medicine to prevent and/or treat infectious disease. In some embodiments, the VZV vaccines of the invention can be envisioned for use in the priming of immune effector cells, for example, to activate peripheral blood mononuclear cells (PBMCs) ex vivo, which are then infused (re-infused) into a subject.

In exemplary embodiments, a VZV vaccine containing RNA polynucleotides as described herein can be administered to a subject (e.g., a mammalian subject, such as a human subject), and the RNA polynucleotides are translated in vivo to produce an antigenic polypeptide.

The VZV RNA vaccines may be induced for translation of a polypeptide (e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing a VZV RNA vaccine that contains a polynucleotide that has at least one a translatable region encoding an antigenic polypeptide.

An “effective amount” of the VZV RNA vaccine is provided based, at least in part, on the target tissue, target cell type, means of administration, physical characteristics of the polynucleotide (e.g., size, and extent of modified nucleosides) and other components of the VZV RNA vaccine, and other determinants. In general, an effective amount of the VZV RNA vaccine composition provides an induced or boosted immune response as a function of antigen production in the cell. In general, an effective amount of the VZV RNA vaccine containing RNA polynucleotides having at least one chemical modifications are preferably more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, RNA vaccines (including polynucleotides and their encoded polypeptides) in accordance with the present disclosure may be used for treatment or prevention of VZV.

VZV RNA vaccines may be administered prophylactically or therapeutically as part of an active immunization scheme to healthy individuals or early in infection during the incubation phase or during active infection after onset of symptoms. In some embodiments, the amount of RNA vaccines of the present disclosure provided to a cell, a tissue or a subject may be an amount effective for immune prophylaxis.

VZV RNA (e.g., mRNA) vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, VZV RNA vaccines may be administered intramuscularly, intranasally or intradermally, similarly to the administration of inactivated vaccines known in the art.

The VZV RNA vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers and produce responses early than commercially available anti-virals.

Provided herein are pharmaceutical compositions including VZV RNA vaccines and RNA vaccine compositions and/or complexes optionally in combination with one or more pharmaceutically acceptable excipients.

VZV RNA (e.g., mRNA) vaccines may be formulated or administered alone or in conjunction with one or more other components. For instance, VZV RNA vaccines (vaccine compositions) may comprise other components including, but not limited to, adjuvants.

In some embodiments, VZV RNA vaccines do not include an adjuvant (they are adjuvant free).

VZV RNA (e.g., mRNA) vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, VZV RNA vaccines are administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigenic polypeptides.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

VZV RNA vaccines can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with VZV RNA vaccines (e.g., for transplantation into a subject), hyaluronidase, nanoparticle mimics and combinations thereof.

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the RNA vaccine may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, B-Galactosidase, EGFP), or a marker or selection protein (e.g. alpha-Globin, Galactokinase and Xanthine: guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA vaccine does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

In some embodiments, the RNA vaccine may or may not contain a enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA vaccine may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Nanoparticle Formulations

In some embodiments, VZV RNA (e.g., mRNA) vaccines are formulated in a nanoparticle. In some embodiments, VZV RNA vaccines are formulated in a lipid nanoparticle. In some embodiments, VZV RNA vaccines are formulated in a lipid-polycation complex, referred to as a cationic lipid nanoparticle. The formation of the lipid nanoparticle may be accomplished by methods known in the art and/or as described in U.S. Publication No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Publication No. WO2012013326 or U.S. Publication No. US20130142818; each of which is herein incorporated by reference in its entirety. In some embodiments, VZV RNA vaccines are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176; herein incorporated by reference in its entirety), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid was shown to more effectively deliver siRNA to various antigen presenting cells (Basha et al. Mol Ther. 2011 19:2186-2200; herein incorporated by reference in its entirety).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(ω-methoxy-poly(ethyleneglycol) 2000) carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, a VZV RNA (e.g., mRNA) vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, (12Z,15Z)—N,N-dimethyl-2-nonylhenicosa-12,15-dien-1-amine (L608), N,N-dimethyl-1-[(1S,2R)-2-octylcyclopropyl]heptadecan-8-amine (L530), PEGylated lipids and amino alcohol lipids.

In some embodiments, the lipid is

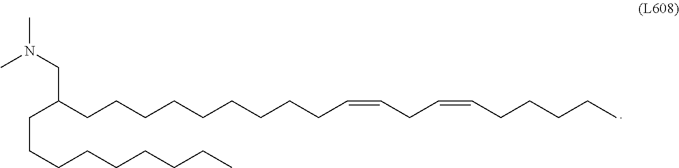 [SMILES: CCCCC/C=C\C/C=C\CCCCCCCCCC(CCCCCCCCC)CN(C)C]

In some embodiments, the lipid is

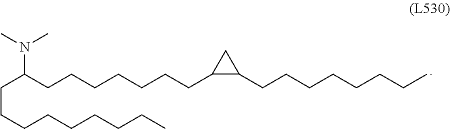 [SMILES: CCCCCCCCCC(CCCCCCCC1CC1CCCCCCCC)N(C)C]

In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Publication No. US20130150625, herein incorporated by reference in its entirety. As a non-limiting example, the cationic lipid may be 2-amino-3-[(9Z, 12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z, 12Z)-octadeca-9,12-dien-1-yloxy]-2-{[(9Z,12Z)-octadeca-9, 12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino) butanoyl)oxy) heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-CDMA, in a molar ratio of 20-60% cationic lipid: 5-25% neutral lipid: 25-55% sterol; 0.5-15% PEG-lipid.

In some embodiments, a lipid nanoparticle formulation includes 25% to 75% on a molar basis of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), e.g., 35 to 65%, 45 to 65%, 60%, 57.5%, 50% or 40% on a molar basis.

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-CDMA (further discussed in Reyes et al. J. Controlled Release, 107, 276-287 (2005) the content of which is herein incorporated by reference in its entirety).

In some embodiments, lipid nanoparticle formulations include 25-75% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 3-12% of the neutral lipid, 15-45% of the sterol, and 0.5-10% of the PEG or PEG-modified lipid on a molar basis.

In some embodiments, lipid nanoparticle formulations include 45-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 5-10% of the neutral lipid, 25-40% of the sterol, and 0.5-10% of the PEG or PEG-modified lipid on a molar basis.

In some embodiments, lipid nanoparticle formulations include 60% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 7.5% of the neutral lipid, 31% of the sterol, and 1.5% of the PEG or PEG-modified lipid on a molar basis.

In some embodiments, lipid nanoparticle formulations include 50% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 10% of the neutral lipid, 38.5% of the sterol, and 1.5% of the PEG or PEG-modified lipid on a molar basis.

In some embodiments, lipid nanoparticle formulations include 50% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 10% of the neutral lipid, 35% of the sterol, 4.5% or 5% of the PEG or PEG-modified lipid, and 0.5% of the targeting lipid on a molar basis.

In some embodiments, lipid nanoparticle formulations include 40% of a cationic lipid selected from the group consisting of 2,2-dilinoleyl-4-dimethylaminocthyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 15% of the neutral lipid, 40% of the sterol, and 5% of the PEG or PEG-modified lipid on a molar basis.

In some embodiments, lipid nanoparticle formulations include 57.2% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), 7.1% of the neutral lipid, 34.3% of the sterol, and 1.4% of the PEG or PEG-modified lipid on a molar basis.

In some embodiments, lipid nanoparticle formulations include 57.5% of a cationic lipid selected from the PEG lipid is PEG-CDMA (PEG-CDMA is further discussed in Reyes et al. (J. Controlled Release, 107, 276-287 (2005), the content of which is herein incorporated by reference in its entirety), 7.5% of the neutral lipid, 31.5% of the sterol, and 3.5% of the PEG or PEG-modified lipid on a molar basis.

In some embodiments, lipid nanoparticle formulations consists essentially of a lipid mixture in molar ratios of 20-70% cationic lipid: 5-45% neutral lipid: 20-55% cholesterol: 0.5-15% PEG-modified lipid. In some embodiments, lipid nanoparticle formulations consists essentially of a lipid mixture in a molar ratio of 20-60% cationic lipid: 5-25% neutral lipid: 25-55% cholesterol: 0.5-15% PEG-modified lipid.

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprise 55% of the cationic lipid L319, 10% of the non-cationic lipid DSPC, 2.5% of the PEG lipid PEG-DMG and 32.5% of the structural lipid cholesterol.

Relative amounts of the active ingredient, the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the RNA vaccine composition may comprise the polynucleotide described herein, formulated in a lipid nanoparticle comprising MC3, Cholesterol, DSPC and PEG2000-DMG, the buffer trisodium citrate, sucrose and water for injection. As a non-limiting example, the composition comprises: 2.0 mg/mL of drug substance (e.g., polynucleotides encoding VZV), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

### Liposomes, Lipoplexes, and Lipid Nanoparticles

In some embodiments, the RNA vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5 (12) 1708-1713); herein incorporated by reference in its entirety) and hyaluronan-coated liposomes (Quiet Therapeutics, Israel).

In some embodiments, the RNA vaccines may be formulated in a lyophilized gel-phase liposomal composition as described in U.S. Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Publication No. WO2013033438 or U.S. Publication No. US20130196948, the content of each of which is herein incorporated by reference in its entirety. As a non-limiting example, the phosphate conjugates may include a compound of any one of the formulas described in International Publication No. WO2013033438, herein incorporated by reference in its entirety.

The nanoparticle formulation may comprise a polymer conjugate. The polymer conjugate may be a water soluble conjugate. The polymer conjugate may have a structure as described in U.S. Publication No. 20130059360, the content of which is herein incorporated by reference in its entirety. In some aspects, polymer conjugates with the polynucleotides of the described in U.S. Publication No. 20130072709, herein incorporated by reference in its entirety. In other aspects, the polymer conjugate may have pendant side groups comprising ring moieties such as, but not limited to, the polymer conjugates described in U.S. Publication No. US20130196948, the contents of which is herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In some aspects, the conjugate may be a “self” peptide designed from the human membrane protein CD47 (e.g., the “self” particles described by Rodriguez et al (Science 2013, 339, 971-975), herein incorporated by reference in its entirety). As shown by Rodriguez et al. the self peptides delayed macrophage-mediated clearance of nanoparticles which enhanced delivery of the nanoparticles. In other aspects, the conjugate may be the membrane protein CD47 (e.g., see Rodriguez et al. Science 2013, 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, the RNA vaccines of the present invention are formulated in nanoparticles which comprise a conjugate to enhance the delivery of the nanoparticles of the present invention in a subject. The conjugate may be the CD47 membrane or the conjugate may be derived from the CD47 membrane protein, such as the “self” peptide described previously.

In other embodiments, the nanoparticle may comprise PEG and a conjugate of CD47 or a derivative thereof. In yet other embodiments, the nanoparticle may comprise both the “self” peptide described above and the membrane protein CD47.

In some embodiments, a “self” peptide and/or CD47 protein may be conjugated to a virus-like particle or pseudovirion, as described herein for delivery of the RNA vaccines of the present invention.

In other embodiments, RNA vaccine pharmaceutical compositions comprising the polynucleotides of the present invention and a conjugate, which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in U.S. Publication No. US20130184443, the content of which is herein incorporated by reference in its entirety.

The nanoparticle formulations may be a carbohydrate nanoparticle comprising a carbohydrate carrier and a RNA vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121, the content of which is herein incorporated by reference in its entirety).

Nanoparticle formulations of the present invention may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA vaccines within the central nervous system. As a non-limiting example nanoparticles comprising a hydrophilic coating and methods of making such nanoparticles are described in U.S. Publication No. US20130183244, the content of which is herein incorporated by reference in its entirety.

In some embodiments, the lipid nanoparticles of the present invention may be hydrophilic polymer particles. Non-limiting examples of hydrophilic polymer particles and methods of making hydrophilic polymer particles are described in U.S. Publication No. US20130210991, the content of which is herein incorporated by reference in its entirety.

In other embodiments, the lipid nanoparticles of the present invention may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the internal ester linkage may be located on either side of the saturated carbo.

In some embodiments, an immune response may be elicited by delivering a lipid nanoparticle which may include a nanospecies, a polymer and an immunogen. (U.S. Publication No. 20120189700 and International Publication No. WO2012099805, each of which is herein incorporated by reference in its entirety).

The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers.

Mucus is continuously secreted, shed, discarded or digested and recycled so most of the trapped particles may be removed from the mucosal tissue within seconds or within a few hours. Large polymeric nanoparticles (200 nm to 500 nm in diameter) which have been coated densely with a low molecular weight polyethylene glycol (PEG) diffused through mucus only 4 to 6-fold lower than the same particles diffusing in water (Lai et al. PNAS 2007 104 (5): 1482-487; Lai et al. Adv Drug Deliv Rev. 2009 61 (2): 158-171; each of which is herein incorporated by reference in its entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Publication No. WO2013110028, the content of each of which is herein incorporated by reference in its entirety.

The lipid nanoparticle engineered to penetrate mucus may comprise a polymeric material (e.g., a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyencimines, polyisocyanates, polyacrylates, polymethacrylates, polyacrylonitriles, and polyarylates. The polymeric material may be biodegradable and/or biocompatible. Non-limiting examples of biocompatible polymers are described in International Publication No. WO2013116804, the content of which is herein incorporated by reference in its entirety. The polymeric material may additionally be irradiated. As a non-limiting example, the polymeric material may be gamma irradiated (see e.g., International Publication No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactidc) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethanc, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl cthers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho) esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a copolymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol))triblock copolymer (see e.g., U.S. Publication 20120121718, U.S. Publication 20100003337 and U.S. Pat. No. 8,263,665, each of which is herein incorporated by reference in its entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:25972600, the content of which is herein incorporated by reference in its entirety). A non-limiting scalable method to produce nanoparticles which can penetrate human mucus is described by Xu et al. (see e.g., J Control Release 2013, 170 (2): 279-86, the content of which is herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

In some embodiments, the RNA (e.g., mRNA) vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5 (12) 1708-1713, herein incorporated by reference in its entirety)) and hyaluronan-coated liposomes (Quiet Therapeutics, Israel).

In some embodiments, the RNA vaccines may be formulated in a lyophilized gel-phase liposomal composition as described in U.S. Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Publication No. WO2013033438 or U.S. Publication No. 20130196948, the content of each of which is herein incorporated by reference in its entirety. As a non-limiting example, the phosphate conjugates may include a compound of any one of the formulas described in International Publication No. WO2013033438, herein incorporated by reference in its entirety.

The nanoparticle formulation may comprise a polymer conjugate. The polymer conjugate may be a water soluble conjugate. The polymer conjugate may have a structure as described in U.S. application No. 20130059360, the content of which is herein incorporated by reference in its entirety. In some aspects, polymer conjugates with the polynucleotides of the described in U.S. patent application No. 20130072709, herein incorporated by reference in its entirety. In other aspects, the polymer conjugate may have pendant side groups comprising ring moieties such as, but not limited to, the polymer conjugates described in U.S. Publication No. US20130196948, the content of which is herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In some aspects, the conjugate may be a “self” peptide designed from the human membrane protein CD47 (e.g., the “self” particles described by Rodriguez et al. (Science 2013, 339, 971-975), herein incorporated by reference in its entirety). As shown by Rodriguez et al. the self peptides delayed macrophage-mediated clearance of nanoparticles which enhanced delivery of the nanoparticles. In other aspects, the conjugate may be the membrane protein CD47 (e.g., see Rodriguez et al. Science 2013, 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, the RNA vaccines of the present invention are formulated in nanoparticles that comprise a conjugate to enhance the delivery of the nanoparticles of the present disclosure in a subject. The conjugate may be the CD47 membrane or the conjugate may be derived from the CD47 membrane protein, such as the “self” peptide described previously. In other aspects the nanoparticle may comprise PEG and a conjugate of CD47 or a derivative thereof. In yet other aspects, the nanoparticle may comprise both the “self” peptide described above and the membrane protein CD47.

In other aspects, a “self” peptide and/or CD47 protein may be conjugated to a virus-like particle or pseudovirion, as described herein for delivery of the RNA vaccines of the present invention.

In other embodiments, RNA vaccine pharmaceutical compositions comprising the polynucleotides of the present invention and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in U.S. Publication No. US20130184443, the content of which is herein incorporated by reference in its entirety.

The nanoparticle formulations may be a carbohydrate nanoparticle comprising a carbohydrate carrier and a RNA (e.g., mRNA) vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the content of which is herein incorporated by reference in its entirety).

Nanoparticle formulations of the present invention may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA vaccines within the central nervous system. As a non-limiting example nanoparticles comprising a hydrophilic coating and methods of making such nanoparticles are described in U.S. Publication No. US20130183244, the content of which is herein incorporated by reference in its entirety.

In some embodiments, the lipid nanoparticles of the present invention may be hydrophilic polymer particles. Non-limiting examples of hydrophilic polymer particles and methods of making hydrophilic polymer particles are described in U.S. Publication No. US20130210991, the content of which is herein incorporated by reference in its entirety.

In other embodiments, the lipid nanoparticles of the present invention may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the internal ester linkage may be located on either side of the saturated carbo.

In some embodiments, an immune response may be elicited by delivering a lipid nanoparticle which may include a nanospecies, a polymer and an immunogen (U.S. Publication No. 20120189700 and International Publication No. WO2012099805, each of which is herein incorporated by reference in its entirety).

The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosal tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104 (5): 1482-487; Lai et al. Adv Drug Deliv Rev. 2009 61 (2): 158-171; each of which is herein incorporated by reference in its entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Publication No. WO2013110028, the content of each of which is herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyencimines, polyisocyanates, polyacrylates, polymethacrylates, polyacrylonitriles, and polyarylates. The polymeric material may be biodegradable and/or biocompatible. Non-limiting examples of biocompatible polymers are described in International Publication No. WO2013116804, the content of which is herein incorporated by reference in its entirety. The polymeric material may additionally be irradiated. As a non-limiting example, the polymeric material may be gamma irradiated (see e.g., International Publication No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactidc) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethanc, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl cthers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(mcth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho) esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a copolymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol))triblock copolymer (scc e.g., U.S. Publication 20120121718 and U.S. Publication 20100003337 and U.S. Pat. No. 8,263,665; each of which is herein incorporated by reference in its entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:25972600; the content of which is herein incorporated by reference in its entirety). A non-limiting scalable method to produce nanoparticles which can penetrate human mucus is described by Xu et al. (see e.g., J Control Release 2013, 170 (2): 279-86, the content of which is herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin B4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle (see e.g., U.S. Publication 20100215580 and U.S. Publication 20080166414 and US20130164343 the content of each of which is herein incorporated by reference in its entirety).

In some embodiments, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In other embodiments, the mucus penetrating lipid nanoparticles may be a hypotonic formulation comprising a mucosal penetration enhancing coating. The formulation may be hypotonice for the epithelium to which it is being delivered.

Non-limiting examples of hypotonic formulations may be found in International Publication No. WO2013110028, the content of which is herein incorporated by reference in its entirety.

In some embodiments, in order to enhance the delivery through the mucosal barrier the RNA vaccine formulation may comprise or be a hypotonic solution. Hypotonic solutions were found to increase the rate at which mucoinert particles such as, but not limited to, mucus-penetrating particles, were able to reach the vaginal epithelial surface (see e.g., Ensign et al. Biomaterials 2013, 34 (28): 6922-9, the content of which is herein incorporated by reference in its entirety).

In some embodiments, the RNA vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293; Weide et al. J Immunother. 2009 32:498-507; Weide et al. J Immunother. 2008 31:180-188; Pascolo, Expert Opin. Biol. Ther. 4:1285-1294; Fotin-Mleczck et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; each of which is incorporated herein by reference in its entirety).

In some embodiments, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; each of which is incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; each of which is incorporated herein by reference in its entirety).

In some embodiments, the RNA (e.g., mRNA) vaccine is formulated as a solid lipid nanoparticle. A solid lipid nanoparticle (SLN) may be spherical with an average diameter between to 1000 nm. SLN possess a solid lipid core matrix that can solubilize lipophilic molecules and may be stabilized with surfactants and/or emulsifiers. In other embodiments, the lipid nanoparticle may be a self-assembly lipid-polymer nanoparticle (see Zhang et al., ACS Nano, 2008, 2 (8), pp 1696-1702; the content of which is herein incorporated by reference in its entirety). As a non-limiting example, the SLN may be the SLN described in International Publication No. WO2013105101, the content of which is herein incorporated by reference in its entirety. As another non-limiting example, the SLN may be made by the methods or processes described in International Publication No. WO2013105101, the content of which is herein incorporated by reference in its entirety.

Liposomes, lipoplexes, or lipid nanoparticles may be used to improve the efficacy of polynucleotides directed protein production as these formulations may be able to increase cell transfection by the RNA vaccine; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the polynucleotide.

In some embodiments, the RNA (e.g., mRNA) vaccines of the present invention can be formulated for controlled release and/or targeted delivery. As used herein, “controlled release” refers to a pharmaceutical composition or compound release profile that conforms to a particular pattern of release to effect a therapeutic outcome. In some embodiments, the RNA vaccines may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the present disclosure are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

In other embodiments, the RNA vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc Deerfield, IL), PEG-based sealants, and COSEAL® (Baxter International, Inc Deerfield, IL).

In other embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the RNA vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the RNA (e.g., mRNA) vaccine controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In other embodiments, the degradable polyesters may include a PEG conjugation to form a PEGylated polymer.

In some embodiments, the RNA vaccine controlled release and/or targeted delivery formulation comprising at least one polynucleotide may comprise at least one PEG and/or PEG related polymer derivatives as described in U.S. Pat. No. 8,404,222, herein incorporated by reference in its entirety.

In other embodiments, the RNA vaccine controlled release delivery formulation comprising at least one polynucleotide may be the controlled release polymer system described in U.S. Publication No. 20130130348, herein incorporated by reference in its entirety.

In some embodiments, the RNA (e.g., mRNA) vaccines of the present invention may be encapsulated in a therapeutic nanoparticle, referred to herein as “therapeutic nanoparticle RNA vaccines.” Therapeutic nanoparticles may be formulated by methods described herein and known in the art such as, but not limited to, International Publication Nos. WO2010005740, WO2010030763, WO2010005721, WO2010005723, WO2012054923, U.S. Publication Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211, the content of each of which is herein incorporated by reference in its entirety. In other embodiments, therapeutic polymer nanoparticles may be identified by the methods described in U.S. Publication No. US20120140790, the content of which is herein incorporated by reference in its entirety.

In some embodiments, the therapeutic nanoparticle RNA vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present invention (see International Publication No. 2010075072 and U.S. Publication Nos. US20100216804, US20110217377 and US20120201859, each of which is herein incorporated by reference in its entirety). In another non-limiting example, the sustained release formulation may comprise agents which permit persistent bioavailability such as, but not limited to, crystals, macromolecular gels and/or particulate suspensions (see U.S. Publication No. US20130150295, the content of which is herein incorporated by reference in its entirety).

In some embodiments, the therapeutic nanoparticle RNA vaccines may be formulated to be target specific. As a non-limiting example, the therapeutic nanoparticles may include a corticosteroid (see International Publication No. WO2011084518, herein incorporated by reference in its entirety). As a non-limiting example, the therapeutic nanoparticles may be formulated in nanoparticles described in International Publication Nos. WO2008121949, WO2010005726, WO2010005725, WO2011084521 and U.S. Publication Nos. US20100069426, US20120004293 and US20100104655, each of which is herein incorporated by reference in its entirety.

In some embodiments, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the therapeutic nanoparticle comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In yet other embodiments, the diblock copolymer may be a high-X diblock copolymer such as those described in International Publication No. WO2013120052, the content of which is herein incorporated by reference in its entirety.

As a non-limiting example, the therapeutic nanoparticle comprises a PLGA-PEG block copolymer (see U.S. Publication No. US20120004293 and U.S. Pat. No. 8,236,330, each of which is herein incorporated by reference in its entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the content of each of which is herein incorporated by reference in its entirety). In yet another non-limiting example, the therapeutic nanoparticle is a stealth nanoparticle or a target-specific stealth nanoparticle as described in U.S. Publication No. 20130172406, the content of which is herein incorporated by reference in its entirety.

In some embodiments, the therapeutic nanoparticle may comprise a multiblock copolymer (see e.g., U.S. Pat. Nos. 8,263,665 and 8,287,910 and U.S. Publication No. 20130195987, the content of each of which is herein incorporated by reference in its entirety).

In yet another non-limiting example, the lipid nanoparticle comprises the block copolymer PEG-PLGA-PEG (see e.g., the thermosensitive hydrogel (PEG-PLGA-PEG) used as a TGF-beta1 gene delivery vehicle in Lee et al. “Thermosensitive Hydrogel as a TGF-β1 Gene Delivery Vehicle Enhances Diabetic Wound Healing.” Pharmaceutical Research, 2003 20 (12): 1995-2000; and used as a controlled gene delivery system in Li et al. “Controlled Gene Delivery System Based on Thermosensitive Biodegradable Hydrogel” Pharmaceutical Research 2003 20 (6): 884-888; and Chang et al., “Non-ionic amphiphilic biodegradable PEG-PLGA-PEG copolymer enhances gene delivery efficiency in rat skeletal muscle.” J Controlled Release. 2007 118:245-253; each of which is herein incorporated by reference in its entirety). The RNA (e.g., mRNA) vaccines of the present disclosure may be formulated in lipid nanoparticles comprising the PEG-PLGA-PEG block copolymer.

In some embodiments, the block copolymers described herein may be included in a polyion complex comprising a non-polymeric micelle and the block copolymer. (see e.g., U.S. Publication No. 20120076836, herein incorporated by reference in its entirety).

In some embodiments, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In some embodiments, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Publication No. WO2013032829 or U.S. Publication No. 20130121954, the content of which is herein incorporated by reference in its entirety. In some aspects, the poly(vinyl ester) polymers may be conjugated to the polynucleotides described herein. In other aspects, the poly(vinyl ester) polymer which may be used in the present invention may be those described in.

In some embodiments, the therapeutic nanoparticle may comprise at least one diblock copolymer. The diblock copolymer may be, but it not limited to, a poly(lactic) acid-poly(ethylene)glycol copolymer (see e.g., International Publication No. WO2013044219; herein incorporated by reference in its entirety). As a non-limiting example, the therapeutic nanoparticle may be used to treat cancer (see International publication No. WO2013044219, herein incorporated by reference in its entirety).

In some embodiments, the therapeutic nanoparticles may comprise at least one cationic polymer described herein and/or known in the art.

In some embodiments, the therapeutic nanoparticles may comprise at least one amine-containing polymer such as, but not limited to polylysine, polyethyleneimine, poly(amidoamine) dendrimers, poly(beta-amino esters) (see e.g., U.S. Pat. No. 8,287,849, herein incorporated by reference in its entirety) and combinations thereof. In other embodiments, the nanoparticles described herein may comprise an amine cationic lipid such as those described in International Publication No. WO2013059496, the content of which is herein incorporated by reference in its entirety. In some aspects the cationic lipids may have an amino-amine or an amino-amide moiety.

In some embodiments, the therapeutic nanoparticles may comprise at least one degradable polyester, which may contain polycationic side chains. Degradeable polyesters include, but are not limited to, poly(serine ester), poly(L-lactide-co-L-lysine), poly(4-hydroxy-L-proline ester), and combinations thereof. In other embodiments, the degradable polyesters may include a PEG conjugation to form a PEGylated polymer.

In other embodiments, the therapeutic nanoparticle may include a conjugation of at least one targeting ligand. The targeting ligand may be any ligand known in the art such as, but not limited to, a monoclonal antibody (Kirpotin et al, Cancer Res. 2006 66:6732-6740, herein incorporated by reference in its entirety).

In some embodiments, the therapeutic nanoparticle may be formulated in an aqueous solution, which may be used to target cancer (see International Publication No. WO2011084513 and U.S. Publication No. 20110294717, each of which is herein incorporated by reference in its entirety).

In some embodiments, the therapeutic nanoparticle RNA vaccines, e.g., therapeutic nanoparticles comprising at least one RNA vaccine may be formulated using the methods described by Podobinski et al in U.S. Pat. No. 8,404,799, the content of which is herein incorporated by reference in its entirety.

In some embodiments, the RNA (e.g., mRNA) vaccines may be encapsulated in, linked to and/or associated with synthetic nanocarriers. Synthetic nanocarriers include, but are not limited to, those described in International Publication Nos. WO2010005740, WO2012149454 and WO2013019669, and U.S. Publication Nos. US20110262491, US20100104645, US20100087337 and US20120244222, each of which is herein incorporated by reference in its entirety. The synthetic nanocarriers may be formulated using methods known in the art and/or described herein. As a non-limiting example, the synthetic nanocarriers may be formulated by the methods described in International Publication Nos. WO2010005740, WO2010030763 and WO201213501, and U.S. Publication Nos.

US20110262491, US20100104645, US20100087337 and US2012024422, each of which is herein incorporated by reference in its entirety. In other embodiments, the synthetic nanocarrier formulations may be lyophilized by methods described in International Publication No. WO2011072218 and U.S. Pat. No. 8,211,473, the content of each of which is herein incorporated by reference in its entirety. In yet other embodiments, formulations of the present invention, including, but not limited to, synthetic nanocarriers, may be lyophilized or reconstituted by the methods described in U.S. Publication No. 20130230568, the content of which is herein incorporated by reference in its entirety.

In some embodiments, the synthetic nanocarriers may contain reactive groups to release the polynucleotides described herein (see International Publication No. WO20120952552 and U.S. Publication No. US20120171229, each of which is herein incorporated by reference in its entirety).

In some embodiments, the synthetic nanocarriers may contain an immunostimulatory agent to enhance the immune response from delivery of the synthetic nanocarrier. As a non-limiting example, the synthetic nanocarrier may comprise a Th1 immunostimulatory agent which may enhance a Th1-based response of the immune system (see International Publication No. WO2010123569 and U.S. Publication No. 20110223201, each of which is herein incorporated by reference in its entirety).

In some embodiments, the synthetic nanocarriers may be formulated for targeted release. In some embodiments, the synthetic nanocarrier is formulated to release the polynucleotides at a specified pH and/or after a desired time interval. As a non-limiting example, the synthetic nanoparticle may be formulated to release the RNA vaccines after 24 hours and/or at a pH of 4.5 (see International Publication Nos. WO2010138193 and WO2010138194 and U.S. Publication Nos. US20110020388 and US20110027217, each of which is herein incorporated by reference in their entireties).

In some embodiments, the synthetic nanocarriers may be formulated for controlled and/or sustained release of the polynucleotides described herein. As a non-limiting example, the synthetic nanocarriers for sustained release may be formulated by methods known in the art, described herein and/or as described in International Publication No. WO2010138192 and U.S. Publication No. 20100303850, each of which is herein incorporated by reference in its entirety.

In some embodiments, the RNA vaccine may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In some embodiments, the synthetic nanocarrier may be formulated for use as a vaccine. In some embodiments, the synthetic nanocarrier may encapsulate at least one polynucleotide which encode at least one antigen. As a non-limiting example, the synthetic nanocarrier may include at least one antigen and an excipient for a vaccine dosage form (see International Publication No. WO2011150264 and U.S. Publication No. 20110293723, each of which is herein incorporated by reference in its entirety). As another non-limiting example, a vaccine dosage form may include at least two synthetic nanocarriers with the same or different antigens and an excipient (see International Publication No. WO2011150249 and U.S. Publication No. 20110293701, each of which is herein incorporated by reference in its entirety). The vaccine dosage form may be selected by methods described herein, known in the art and/or described in International Publication No. WO2011150258 and U.S. Publication No. US20120027806, each of which is herein incorporated by reference in its entirety).

In some embodiments, the synthetic nanocarrier may comprise at least one polynucleotide which encodes at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (see e.g., U.S. Pat. No. 8,241,610; herein incorporated by reference in its entirety). In other embodiments, the synthetic nanocarrier may comprise at least one polynucleotide and an adjuvant. As a non-limiting example, the synthetic nanocarrier comprising and adjuvant may be formulated by the methods described in International Publication No. WO2011150240 and U.S. Publication No. US20110293700, each of which is herein incorporated by reference in its entirety.

In some embodiments, the synthetic nanocarrier may encapsulate at least one polynucleotide which encodes a peptide, fragment or region from a virus. As a non-limiting example, the synthetic nanocarrier may include, but is not limited to, the nanocarriers described in International Publication Nos. WO2012024621, WO201202629, WO2012024632 and U.S. Publication No. US20120064110, US20120058153 and US20120058154, each of which is herein incorporated by reference in its entirety.

In some embodiments, the synthetic nanocarrier may be coupled to a polynucleotide which may be able to trigger a humoral and/or cytotoxic T lymphocyte (CTL) response (See e.g., International Publication No. WO2013019669, herein incorporated by reference in its entirety).

In some embodiments, the RNA vaccine may be encapsulated in, linked to and/or associated with zwitterionic lipids. Non-limiting examples of zwitterionic lipids and methods of using zwitterionic lipids are described in U.S. Publication No. 20130216607, the content of which is herein incorporated by reference in its entirety. In some aspects, the zwitterionic lipids may be used in the liposomes and lipid nanoparticles described herein.

In some embodiments, the RNA vaccine may be formulated in colloid nanocarriers as described in U.S. Publication No. 20130197100, the content of which is herein incorporated by reference in its entirety.

In some embodiments, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Publication No. 20120282343; herein incorporated by reference in its entirety.

In some embodiments, LNPs comprise the lipid KL52 (an amino-lipid disclosed in U.S. Application Publication No. 2012/0295832 expressly incorporated herein by reference in its entirety). Activity and/or safety (as measured by examining one or more of ALT/AST, white blood cell count and cytokine induction) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, RNA vaccine may be delivered using smaller LNPs. Such particles may comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, or less than 975 μm.

In other embodiments, RNA (e.g., mRNA) vaccines may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:37; Chen, D. et al., Rapid discovery of potent siRNA-containing lipid nanoparticles enabled by controlled microfluidic formulation. J Am Chem Soc. 2012. 134 (16): 6948-51; each of which is herein incorporated by reference in its entirety).

In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety.

In some embodiments, the RNA vaccine of the present invention may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In some embodiments, the RNA (e.g., mRNA) vaccines of the present disclosure may be formulated in lipid nanoparticles created using microfluidic technology (see Whitesides, George M. The Origins and the Future of Microfluidics. Nature, 2006 442:368-373; and Abraham et al. Chaotic Mixer for Microchannels. Science, 2002 295:647-651; each of which is herein incorporated by reference in its entirety). As a non-limiting example, controlled microfluidic formulation includes a passive method for mixing streams of steady pressure-driven flows in micro channels at a low Reynolds number (see e.g., Abraham et al. Chaotic Mixer for Microchannels. Science, 2002 295:647651; which is herein incorporated by reference in its entirety).

In some embodiments, the RNA (e.g., mRNA) vaccines of the present invention may be formulated in lipid nanoparticles created using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In some embodiments, the RNA (e.g., mRNA) vaccines of the invention may be formulated for delivery using the drug encapsulating microspheres described in International Publication No. WO2013063468 or U.S. Pat. No. 8,440,614, each of which is herein incorporated by reference in its entirety. The microspheres may comprise a compound of the formula (I), (II), (III), (IV), (V) or (VI) as described in International Publication No. WO2013063468, the content of which is herein incorporated by reference in its entirety. In other aspects, the amino acid, peptide, polypeptide, lipids (APPL) are useful in delivering the RNA vaccines of the invention to cells (see International Publication No. WO2013063468, the contents of which is herein incorporated by reference in its entirety).

In some embodiments, the RNA (e.g., mRNA) vaccines of the present disclosure may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm.

In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some aspects, the lipid nanoparticle may be a limit size lipid nanoparticle described in International Publication No. WO2013059922, the content of which is herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In other aspects the limit size lipid nanoparticle may comprise a polyethylene glycol-lipid such as, but not limited to, DLPE-PEG, DMPE-PEG, DPPC-PEG and DSPE-PEG.

In some embodiments, the RNA vaccines may be delivered, localized and/or concentrated in a specific location using the delivery methods described in International Publication No. WO2013063530, the content of which is herein incorporated by reference in its entirety. As a non-limiting example, a subject may be administered an empty polymeric particle prior to, simultaneously with or after delivering the RNA vaccines to the subject. The empty polymeric particle undergoes a change in volume once in contact with the subject and becomes lodged, embedded, immobilized or entrapped at a specific location in the subject.

In some embodiments, the RNA vaccines may be formulated in an active substance release system (see e.g., U.S. Publication No. US20130102545, the contents of which is herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In some embodiments, the RNA (e.g., mRNA) vaccines may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Publication No. WO2013052167, herein incorporated by reference in its entirety. As another non-limiting example, the nanoparticle described in International Publication No. WO2013052167, herein incorporated by reference in its entirety, may be used to deliver the RNA vaccines described herein.

In some embodiments, the RNA vaccines may be formulated in porous nanoparticle-supported lipid bilayers (protocells). Protocells are described in International Publication No. WO2013056132, the content of which is herein incorporated by reference in its entirety.

In some embodiments, the RNA vaccines described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the content of which is herein incorporated by reference in its entirety. As another non-limiting example, the polymer nanoparticle for oral and parenteral formulations may be made by the methods described in European Patent No. EP2073848B1, the content of which is herein incorporated by reference in its entirety.

In other embodiments, the RNA (e.g., mRNA) vaccines described herein may be formulated in nanoparticles used in imaging. The nanoparticles may be liposome nanoparticles such as those described in U.S. Publication No. 20130129636, herein incorporated by reference in its entirety. As a non-limiting example, the liposome may comprise gadolinium (III) 2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid
component (see e.g., U.S. Publication No US20130129636, the contents of which is herein incorporated by reference in its entirety).

In some embodiments, the nanoparticles which may be used in the present invention are formed by the methods described in U.S. patent application No. 20130130348, the contents of which is herein incorporated by reference in its entirety.

The nanoparticles of the present invention may further include nutrients such as, but not limited to, those which deficiencies can lead to health hazards from anemia to neural tube defects (see e.g., the nanoparticles described in International Patent Publication No. WO2013072929, the contents of which is herein incorporated by reference in its entirety). As a non-limiting example, the nutrient may be iron in the form of ferrous, ferric salts or elemental iron, iodine, folic acid, vitamins or micronutrients.

In some embodiments, the RNA (e.g., mRNA) vaccines of the present invention may be formulated in a swellable nanoparticle. The swellable nanoparticle may be, but is not limited to, those described in U.S. Pat. No. 8,440,231, the contents of which is herein incorporated by reference in its entirety. As a non-limiting embodiment, the swellable nanoparticle may be used for delivery of the RNA (e.g., mRNA) vaccines of the present invention to the pulmonary system (see e.g., U.S. Pat. No. 8,440,231, the contents of which is herein incorporated by reference in its entirety).

The RNA (e.g., mRNA) vaccines of the present invention may be formulated in polyanhydride nanoparticles such as, but not limited to, those described in U.S. Pat. No. 8,449,916, the contents of which is herein incorporated by reference in its entirety. The nanoparticles and microparticles of the present invention may be geometrically engineered to modulate macrophage and/or the immune response. In some aspects, the geometrically engineered particles may have varied shapes, sizes and/or surface charges in order to incorporated the polynucleotides of the present invention for targeted delivery such as, but not limited to, pulmonary delivery (see e.g., International Publication No. WO2013082111, the content of which is herein incorporated by reference in its entirety). Other physical features the geometrically engineering particles may have include, but are not limited to, fenestrations, angled arms, asymmetry and surface roughness, charge which can alter the interactions with cells and tissues. As a non-limiting example, nanoparticles of the present invention may be made by the methods described in International Publication No. WO2013082111, the contents of which is herein incorporated by reference in its entirety.

In some embodiments, the nanoparticles of the present invention may be water soluble nanoparticles such as, but not limited to, those described in International Publication No. WO2013090601, the content of which is herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments the nanoparticles of the present invention may be developed by the methods described in U.S. Publication No. US20130172406, the content of which is herein incorporated by reference in its entirety.

In some embodiments, the nanoparticles of the present invention are stealth nanoparticles or target-specific stealth nanoparticles such as, but not limited to, those described in U.S. Publication No. 20130172406, the content of which is herein incorporated by reference in its entirety. The nanoparticles of the present invention may be made by the methods described in U.S. Publication No. 20130172406, the content of which is herein incorporated by reference in its entirety.

In other embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In some embodiments, the nanoparticle may be a nanoparticle-nucleic acid hybrid structure having a high density nucleic acid layer. As a non-limiting example, the nanoparticle-nucleic acid hybrid structure may made by the methods described in U.S. Publication No. 20130171646, the content of which is herein incorporated by reference in its entirety. The nanoparticle may comprise a nucleic acid such as, but not limited to, polynucleotides described herein and/or known in the art.

At least one of the nanoparticles of the present invention may be embedded in in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Publication No. WO2013123523, the content of which is herein incorporated by reference in its entirety.

### Modes of Vaccine Administration

VZV RNA (e.g., mRNA) vaccines may be administered by any route which results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. VZV RNA (e.g., mRNA) vaccines compositions are typically formulated in dosage unit form for case of administration and uniformity of dosage. It will be understood, however, that the total daily usage of VZV RNA (e.g., mRNA) vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, VZV RNA (e.g., mRNA) vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, VZV RNA (e.g., mRNA) vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, VZV RNA (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, VZV RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a VZV RNA (e.g., mRNA) vaccine composition may be administered three or four times.

In some embodiments, VZV RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments the VZV RNA (e.g., mRNA) vaccine for use in a method of vaccinating a subject is administered the subject a single dosage of between 10 μg/kg and 400 μg/kg of the nucleic acid vaccine in an effective amount to vaccinate the subject. In some embodiments the RNA vaccine for use in a method of vaccinating a subject is administered the subject a single dosage of between 10 μg and 400 μg of the nucleic acid vaccine in an effective amount to vaccinate the subject. In some embodiments, a VZV RNA (e.g., mRNA) vaccine for use in a method of vaccinating a subject is administered to the subject as a single dosage of 25-1000 μg (e.g., a single dosage of mRNA encoding an VZV antigen). In some embodiments, a VZV RNA vaccine is administered to the subject as a single dosage of 25, 50, 100, 150, 200, 250, 300, 350, 400, 450, 500, 550, 600, 650, 700, 750, 800, 850, 900, 950 or 1000 μg. For example, a VZV RNA vaccine may be administered to a subject as a single dose of 25-100, 25-500, 50-100, 50-500, 50-1000, 100-500, 100-1000, 250-500, 250-1000, or 500-1000 μg. In some embodiments, a VZV RNA (e.g., mRNA) vaccine for use in a method of vaccinating a subject is administered to the subject as two dosages, the combination of which equals 25-1000 μg of the VZV RNA (e.g., mRNA) vaccine.

A VZV RNA (e.g., mRNA) vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### VZV RNA Vaccine Formulations and Methods of Use

Some aspects of the present disclosure provide formulations of the VZV RNA (e.g., mRNA) vaccine, wherein the VZV RNA vaccine is formulated in an effective amount to produce an antigen specific immune response in a subject (e.g., production of antibodies specific to an anti-VZV antigenic polypeptide). “An effective amount” is a dose of an VZV RNA (e.g., mRNA) vaccine effective to produce an antigen-specific immune response. Also provided herein are methods of inducing an antigen-specific immune response in a subject.

In some embodiments, the antigen-specific immune response is characterized by measuring an anti-VZV antigenic polypeptide antibody titer produced in a subject administered a VZV RNA (e.g., mRNA) vaccine as provided herein. An antibody titer is a measurement of the amount of antibodies within a subject, for example, antibodies that are specific to a particular antigen (e.g., an anti-VZV antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the VZV RNA (e.g., mRNA) vaccine.

In some embodiments, an anti-VZV antigenic polypeptide antibody titer produced in a subject is increased by at least 1 log relative to a control. For example, anti-VZV antigenic polypeptide antibody titer produced in a subject may be increased by at least 1.5, at least 2, at least 2.5, or at least 3 log relative to a control. In some embodiments, the anti-VZV antigenic polypeptide antibody titer produced in the subject is increased by 1, 1.5, 2, 2.5 or 3 log relative to a control. In some embodiments, the anti-VZV antigenic polypeptide antibody titer produced in the subject is increased by 1-3 log relative to a control. For example, the anti-VZV antigenic polypeptide antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-VZV antigenic polypeptide antibody titer produced in a subject is increased at least 2 times relative to a control. For example, the anti-VZV antigenic polypeptide antibody titer produced in a subject may be increased at least 3 times, at least 4 times, at least 5 times, at least 6 times, at least 7 times, at least 8 times, at least 9 times, or at least 10 times relative to a control. In some embodiments, the anti-VZV antigenic polypeptide antibody titer produced in the subject is increased 2, 3, 4, 5, 6, 7, 8, 9, or 10 times relative to a control. In some embodiments, the anti-VZV antigenic polypeptide antibody titer produced in a subject is increased 2-10 times relative to a control. For example, the anti-VZV antigenic polypeptide antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-VZV antigenic polypeptide antibody titer produced in a subject who has not been administered a VZV RNA (e.g., mRNA) vaccine. In some embodiments, a control is an anti-VZV antigenic polypeptide antibody titer produced in a subject who has been administered a live attenuated VZV vaccine. An attenuated vaccine is a vaccine produced by reducing the virulence of a viable (live). An attenuated virus is altered in a manner that renders it harmless or less virulent relative to live, unmodified virus. In some embodiments, a control is an anti-VZV antigenic polypeptide antibody titer produced in a subject administered inactivated VZV vaccine. In some embodiments, a control is an anti-VZV antigenic polypeptide antibody titer produced in a subject administered a recombinant or purified VZV protein vaccine. Recombinant protein vaccines typically include protein antigens that either have been produced in a heterologous expression system (e.g., bacteria or yeast) or purified from large amounts of the pathogenic organism. In some embodiments, a control is an anti-VZV antigenic polypeptide antibody titer produced in a subject who has been administered a VZV virus-like particle (VLP) vaccine (e.g., particles that contain viral capsid protein but lack a viral genome and, therefore, cannot replicate/produce progeny virus). In some embodiments, the control is a VLP VZV vaccine that comprises prefusion or postfusion F proteins, or that comprises a combination of the two.

In some embodiments, an effective amount of a VZV RNA (e.g., mRNA) vaccine is a dose that is reduced compared to the standard of care dose of a recombinant VZV protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified VZV protein vaccine, or a live attenuated or inactivated VZV vaccine, or a VZV VLP vaccine, that a physician/clinician or other medical professional would administer to a subject to treat or prevent VZV, or a VZV-related condition, while following the standard of care guideline for treating or preventing VZV, or a VZV-related condition.

In some embodiments, the anti-VZV antigenic polypeptide antibody titer produced in a subject administered an effective amount of a VZV RNA vaccine is equivalent to an anti-VZV antigenic polypeptide antibody titer produced in a control subject administered a standard of care dose of a recombinant or purified VZV protein vaccine, or a live attenuated or inactivated VZV vaccine, or a VZV VLP vaccine.

In some embodiments, an effective amount of a VZV RNA (e.g., mRNA) vaccine is a dose equivalent to an at least 2-fold reduction in a standard of care dose of a recombinant or purified VZV protein vaccine. For example, an effective amount of a VZV RNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified VZV protein vaccine. In some embodiments, an effective amount of a VZV RNA vaccine is a dose equivalent to an at least at least 100-fold, at least 500-fold, or at least 1000-fold reduction in a standard of care dose of a recombinant or purified VZV protein vaccine. In some embodiments, an effective amount of a VZV RNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified VZV protein vaccine. In some embodiments, the anti-VZV antigenic polypeptide antibody titer produced in a subject administered an effective amount of a VZV RNA vaccine is equivalent to an anti-VZV antigenic polypeptide antibody titer produced in a control subject administered the standard of care dose of a recombinant or protein VZV protein vaccine, or a live attenuated or inactivated VZV vaccine, or a VZV VLP vaccine. In some embodiments, an effective amount of a VZV RNA (e.g., mRNA) vaccine is a dose equivalent to a 2-fold to 1000-fold (e.g., 2-fold to 100-fold, 10-fold to 1000-fold) reduction in the standard of care dose of a recombinant or purified VZV protein vaccine, wherein the anti-VZV antigenic polypeptide antibody titer produced in the subject is equivalent to an anti-VZV antigenic polypeptide antibody titer produced in a control subject administered the standard of care dose of a recombinant or purified VZV protein vaccine, or a live attenuated or inactivated VZV vaccine, or a VZV VLP vaccine.

In some embodiments, the effective amount of a VZV RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-, 2 to 5-, 2 to 4-, 2 to 3—, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10-, 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 4 to 00-, 4 to 200-, 4 to 100-, 4 to 90-, 4 to 80-, 4 to 70-, 4 to 60-, 4 to 50-, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9- , 5 to 8-, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-, 7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-, 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-, 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-, 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant VZV protein vaccine. In some embodiments, such as the foregoing, the anti-VZV antigenic polypeptide antibody titer produced in the subject is equivalent to an anti-VZV antigenic polypeptide antibody titer produced in a control subject administered the standard of care dose of a recombinant or purified VZV protein vaccine, or a live attenuated or inactivated VZV vaccine, or a VZV VLP vaccine. In some embodiments, the effective amount is a dose equivalent to (or equivalent to an at least) 2-, 3-,4-,5-,6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 4360-, 470-, 480-, 490-, 500-, 510-, 520-, 530-, 540-, 550-, 560-, 5760-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820--, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant VZV protein vaccine. In some embodiments, such as the foregoing, an anti-VZV antigenic polypeptide antibody titer produced in the subject is equivalent to an anti-VZV antigenic polypeptide antibody titer produced in a control subject administered the standard of care dose of a recombinant or purified VZV protein vaccine, or a live attenuated or inactivated VZV vaccine, or a VZV VLP vaccine.

In some embodiments, the effective amount of a VZV RNA (e.g., mRNA) vaccine is a total dose of 50-1000 μg. In some embodiments, the effective amount of a VZV RNA (e.g., mRNA) vaccine is a total dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 μg. In some embodiments, the effective amount of a VZV RNA (e.g., mRNA) vaccine is a total dose of 50, 100, 150, 200, 250, 300, 350, 400, 450, 500, 550, 600, 650, 700, 750, 800, 850, 900, 950 or 1000 μg. In some embodiments, the effective amount is a dose of 25-500 μg administered to the subject a total of two times. In some embodiments, the effective amount of a VZV RNA (e.g., mRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 μg administered to the subject a total of two times. In some embodiments, the effective amount of a VZV RNA (e.g., mRNA) vaccine is a total dose of 25, 50, 100, 150, 200, 250, 300, 350, 400, 450, or 500 μg administered to the subject a total of two times.

### Additional Embodiments

1. A varicella zoster virus (VZV) vaccine, comprising:
at least one messenger ribonucleic acid (mRNA) polynucleotide having a 5′ terminal cap, an open reading frame encoding at least one VZV antigenic polypeptide, and a 3′ polyA tail.

2. The vaccine of paragraph 1, wherein the at least one mRNA polynucleotide is encoded by a sequence identified by SEQ ID NO: 11.

3. The vaccine of paragraph 1, wherein the at least one mRNA polynucleotide comprises a sequence identified by SEQ ID NO: 92.

4. The vaccine of paragraph 1, wherein the at least one antigenic polypeptide comprises a sequence identified by SEQ ID NO: 10.

5. The vaccine of paragraph 1, wherein the at least one mRNA polynucleotide is encoded by a sequence identified by SEQ ID NO: 15.

6. The vaccine of paragraph 1, wherein the at least one mRNA polynucleotide comprises a sequence identified by SEQ ID NO: 93.

7. The vaccine of paragraph 1, wherein the at least one antigenic polypeptide comprises a sequence identified by SEQ ID NO: 14.

8. The vaccine of paragraph 1, wherein the at least one mRNA polynucleotide is encoded by a sequence identified by SEQ ID NO: 19.

9. The vaccine of paragraph 1, wherein the at least one mRNA polynucleotide comprises a sequence identified by SEQ ID NO: 94.

10 The vaccine of paragraph 1, wherein the at least one antigenic polypeptide comprises a sequence identified by SEQ ID NO: 18.

11. The vaccine of paragraph 1, wherein the at least one mRNA polynucleotide is encoded by a sequence identified by SEQ ID NO: 23.

12. The vaccine of paragraph 1, wherein the at least one mRNA polynucleotide comprises a sequence identified by SEQ ID NO: 95.

13. The vaccine of paragraph 1, wherein the at least one antigenic polypeptide comprises a sequence identified by SEQ ID NO: 22.

14. The vaccine of paragraph 1, wherein the at least one mRNA polynucleotide is encoded by a sequence identified by SEQ ID NO: 27.

15. The vaccine of paragraph 1, wherein the at least one mRNA polynucleotide comprises a sequence identified by SEQ ID NO: 96.

16. The vaccine of paragraph 1, wherein the at least one antigenic polypeptide comprises a sequence identified by SEQ ID NO: 26.

17. The vaccine of paragraph 1, wherein the at least one mRNA polynucleotide is encoded by a sequence identified by SEQ ID NO: 31.

18. The vaccine of paragraph 1, wherein the at least one mRNA polynucleotide comprises a sequence identified by SEQ ID NO: 97.

19. The vaccine of paragraph 1, wherein the at least one antigenic polypeptide comprises a sequence identified by SEQ ID NO: 30.

20. The vaccine of paragraph 1, wherein the at least one mRNA polynucleotide is encoded by a sequence identified by SEQ ID NO: 35.

21. The vaccine of paragraph 1, wherein the at least one mRNA polynucleotide comprises a sequence identified by SEQ ID NO: 98.

22. The vaccine of paragraph 1, wherein the at least one antigenic polypeptide comprises a sequence identified by SEQ ID NO: 34.

23. The vaccine of paragraph 1, wherein the at least one mRNA polynucleotide is encoded by a sequence identified by SEQ ID NO: 39.

24. The vaccine of paragraph 1, wherein the at least one mRNA polynucleotide comprises a sequence identified by SEQ ID NO: 99.

25. The vaccine of paragraph 1, wherein the at least one antigenic polypeptide comprises a sequence identified by SEQ ID NO: 38.

26. The vaccine of paragraph 1, wherein the at least one mRNA polynucleotide is encoded by a sequence identified by SEQ ID NO: 62.

27.

The vaccine of paragraph 1, wherein the at least one mRNA polynucleotide comprises a sequence identified by SEQ ID NO: 101.

28. The vaccine of paragraph 26 or 27, wherein the at least one antigenic polypeptide comprises a sequence identified by SEQ ID NO: 38.

29. The vaccine of paragraph 1, wherein the at least one mRNA polynucleotide is encoded by a sequence identified by SEQ ID NO: 66.

30. The vaccine of paragraph 1, wherein the at least one mRNA polynucleotide comprises a sequence identified by SEQ ID NO: 102.

31. The vaccine of paragraph 30 or 31, wherein the at least one antigenic polypeptide comprises a sequence identified by SEQ ID NO: 38.

32. The vaccine of paragraph 1, wherein the at least one mRNA polynucleotide is encoded by a sequence identified by SEQ ID NO: 70.

33. The vaccine of paragraph 1, wherein the at least one mRNA polynucleotide comprises a sequence identified by SEQ ID NO: 103.

34. The vaccine of paragraph 32 or 33, wherein the at least one antigenic polypeptide comprises a sequence identified by SEQ ID NO: 38.

35. The vaccine of paragraph 1, wherein the at least one mRNA polynucleotide is encoded by a sequence identified by SEQ ID NO: 74.

36. The vaccine of paragraph 1, wherein the at least one mRNA polynucleotide comprises a sequence identified by SEQ ID NO: 104.

37. The vaccine of paragraph 35 or 36, wherein the at least one antigenic polypeptide comprises a sequence identified by SEQ ID NO: 38.

38. The vaccine of paragraph 1, wherein the at least one mRNA polynucleotide is encoded by a sequence identified by SEQ ID NO: 78.

39. The vaccine of paragraph 1, wherein the at least one mRNA polynucleotide comprises a sequence identified by SEQ ID NO: 105.

40. The vaccine of paragraph 38 or 39, wherein the at least one antigenic polypeptide comprises a sequence identified by SEQ ID NO: 38.

41. The vaccine of paragraph 1, wherein the at least one mRNA polynucleotide is encoded by a sequence identified by SEQ ID NO: 82.

42. The vaccine of paragraph 1, wherein the at least one mRNA polynucleotide comprises a sequence identified by SEQ ID NO: 106.

43. The vaccine of paragraph 41 or 42, wherein the at least one antigenic polypeptide comprises a sequence identified by SEQ ID NO: 38.

44. The vaccine of paragraph 1, wherein the at least one mRNA polynucleotide is encoded by a sequence identified by SEQ ID NO: 86.

45. The vaccine of paragraph 1, wherein the at least one mRNA polynucleotide comprises a sequence identified by SEQ ID NO: 107 or 134.

46. The vaccine of paragraph 44 or 45, wherein the at least one antigenic polypeptide comprises a sequence identified by SEQ ID NO: 38.

47. The vaccine of paragraph 1, wherein the at least one mRNA polynucleotide is encoded by a sequence identified by SEQ ID NO: 90.

48. The vaccine of paragraph 1, wherein the at least one mRNA polynucleotide comprises a sequence identified by SEQ ID NO: 108.

49. The vaccine of paragraph 47 or 48, wherein the at least one antigenic polypeptide comprises a sequence identified by SEQ ID NO: 38.

50. The vaccine of any one of paragraphs 1-49, wherein the 5′ terminal cap is or comprises 7 mG (5′) ppp (5′) NlmpNp.

51. The vaccine of any one of paragraphs 1-50, wherein 100% of the uracil in the open reading frame is modified to include N1-methyl pseudouridine at the 5-position of the uracil.

52. The vaccine of any one of paragraphs 1-51, wherein the vaccine is formulated in a lipid nanoparticle comprising: DLin-MC3-DMA; cholesterol; 1,2-Distearoyl-sn-glycero-3-phosphocholine (DSPC); and polyethylene glycol (PEG) 2000-DMG.

53. The vaccine of paragraph 52, wherein the lipid nanoparticle further comprises trisodium citrate buffer, sucrose and water.

54. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 92 and a 3′ polyA tail, wherein the uracil nucleotides of the sequence identified by SEQ ID NO: 92 are modified to include N1-methyl pseudouridine at the 5-position of the uracil nucleotide.

55. A varicella zoster virus (VZV) vaccine, comprising:
at least one messenger ribonucleic acid (mRNA) polynucleotide having a 5′ terminal cap 7 mG (5′) ppp (5′) NlmpNp, a sequence identified by SEQ ID NO: 93 and a 3′ polyA tail, wherein the uracil nucleotides of the sequence identified by SEQ ID NO: 93 are modified to include N1-methyl pseudouridine at the 5-position of the uracil nucleotide.

56. A varicella zoster virus (VZV) vaccine, comprising:
at least one messenger ribonucleic acid (mRNA) polynucleotide having a 5′ terminal cap 7 mG (5′) ppp (5′) NlmpNp, a sequence identified by SEQ ID NO: 94 and a 3′ polyA tail, wherein the uracil nucleotides of the sequence identified by SEQ ID NO: 94 are modified to include N1-methyl pseudouridine at the 5-position of the uracil nucleotide.

57. A varicella zoster virus (VZV) vaccine, comprising:
at least one messenger ribonucleic acid (mRNA) polynucleotide having a 5′ terminal cap 7 mG (5′) ppp (5′) NlmpNp, a sequence identified by SEQ ID NO: 95 and a 3′ polyA tail, wherein the uracil nucleotides of the sequence identified by SEQ ID NO: 95 are modified to include N1-methyl pseudouridine at the 5-position of the uracil nucleotide.

58. A varicella zoster virus (VZV) vaccine, comprising:
at least one messenger ribonucleic acid (mRNA) polynucleotide having a 5′ terminal cap 7 mG (5′) ppp (5′) NlmpNp, a sequence identified by SEQ ID NO: 96 and a 3′ polyA tail, wherein the uracil nucleotides of the sequence identified by SEQ ID NO: 96 are modified to include N1-methyl pseudouridine at the 5-position of the uracil nucleotide.

59. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 97 and a 3′ polyA tail, wherein the uracil nucleotides of the sequence identified by SEQ ID NO: 97 are modified to include N1-methyl pseudouridine at the 5-position of the uracil nucleotide.

60. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 98 and a 3′ polyA tail, wherein the uracil nucleotides of the sequence identified by SEQ ID NO: 98 are modified to include N1-methyl pseudouridine at the 5-position of the uracil nucleotide.

61. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 99 and a 3′ polyA tail, wherein the uracil nucleotides of the sequence identified by SEQ ID NO: 99 are modified to include N1-methyl pseudouridine at the 5-position of the uracil nucleotide.

62. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 101 and a 3′ polyA tail, wherein the uracil nucleotides of the sequence identified by SEQ ID NO: 101 are modified to include N1-methyl pseudouridine at the 5-position of the uracil nucleotide.

63. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 102 and a 3′ polyA tail, wherein the uracil nucleotides of the sequence identified by SEQ ID NO: 102 are modified to include N1-methyl pseudouridine at the 5-position of the uracil nucleotide.

64. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 103 and a 3′ polyA tail, wherein the uracil nucleotides of the sequence identified by SEQ ID NO: 103 are modified to include N1-methyl pseudouridine at the 5-position of the uracil nucleotide.

65. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 104 and a 3′ polyA tail, wherein the uracil nucleotides of the sequence identified by SEQ ID NO: 104 are modified to include N1-methyl pseudouridine at the 5-position of the uracil nucleotide.

66. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 105 and a 3′ polyA tail, wherein the uracil nucleotides of the sequence identified by SEQ ID NO: 105 are modified to include N1-methyl pseudouridine at the 5-position of the uracil nucleotide.

67. A varicella zoster virus (VZV) vaccine, comprising:
at least one messenger ribonucleic acid (mRNA) polynucleotide having a 5′ terminal cap 7 mG (5′) ppp (5′) NlmpNp, a sequence identified by SEQ ID NO: 106 and a 3′ polyA tail, wherein the uracil nucleotides of the sequence identified by SEQ ID NO: 106 are modified to include N1-methyl pseudouridine at the 5-position of the uracil nucleotide.

68. A varicella zoster virus (VZV) vaccine, comprising:

at least one messenger ribonucleic acid (mRNA) polynucleotide having a 5′ terminal cap 7 mG (5′) ppp (5′) NImpNp, a sequence identified by SEQ ID NO: 107 or 134 and a 3′ polyA tail, wherein the uracil nucleotides of the sequence identified by SEQ ID NO: 107 or 134 are modified to include N1-methyl pseudouridine at the 5-position of the uracil nucleotide.

69. A varicella zoster virus (VZV) vaccine, comprising:
at least one messenger ribonucleic acid (mRNA) polynucleotide having a 5′ terminal cap 7 mG (5′) ppp (5′) NImpNp, a sequence identified by SEQ ID NO: 108 and a 3′ polyA tail, wherein the uracil nucleotides of the sequence identified by SEQ ID NO: 108 are modified to include N1-methyl pseudouridine at the 5-position of the uracil nucleotide.

70. The vaccine of any one of claims 54-69, wherein the vaccine is formulated in a lipid nanoparticle comprising DLin-MC3-DMA, cholesterol, 1,2-Distearoyl-sn-glycero-3-phosphocholine (DSPC), and polyethylene glycol (PEG) 2000-DMG.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1: Manufacture of Polynucleotides

According to the present disclosure, the manufacture of polynucleotides and/or parts or regions thereof may be accomplished utilizing the methods taught in International Publication WO2014/152027, entitled “Manufacturing Methods for Production of RNA Transcripts,” the contents of which is incorporated herein by reference in its entirety.

Purification methods may include those taught in International Publication WO2014/152030 and International Publication WO2014/152031, each of which is incorporated herein by reference in its entirety.

Detection and characterization methods of the polynucleotides may be performed as taught in International Publication WO2014/144039, which is incorporated herein by reference in its entirety.

Characterization of the polynucleotides of the disclosure may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Publication WO2014/144711 and International Publication WO2014/144767, the content of each of which is incorporated herein by reference in its entirety.

### Example 2: Chimeric Polynucleotide Synthesis

According to the present disclosure, two regions or parts of a chimeric polynucleotide may be joined or ligated using triphosphate chemistry. A first region or part of 100 nucleotides or less is chemically synthesized with a 5′ monophosphate and terminal 3′desOH or blocked OH, for example. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

For ligation methods, ligation with DNA T4 ligase, followed by treatment with DNAse should readily avoid concatenation.

The entire chimeric polynucleotide need not be manufactured with a phosphate-sugar backbone. If one of the regions or parts encodes a polypeptide, then such region or part may comprise a phosphate-sugar backbone.

Ligation is then performed using any known click chemistry, orthoclick chemistry, solulink, or other bioconjugate chemistries known to those in the art.

### Synthetic Route

The chimeric polynucleotide may be made using a series of starting segments. Such segments include:
(a) a capped and protected 5′ segment comprising a normal 3′OH (SEG. 1)
(b) a 5′ triphosphate segment, which may include the coding region of a polypeptide and a normal 3′OH (SEG. 2)
(c) a 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) may be treated with cordycepin and then with pyrophosphatase to create the 5′ monophosphate.

Segment 2 (SEG. 2) may then be ligated to SEG. 3 using RNA ligase. The ligated polynucleotide is then purified and treated with pyrophosphatase to cleave the diphosphate. The treated SEG.2-SEG. 3 construct may then be purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF (SEG. 2) and 3′UTR+PolyA (SEG. 3).

The yields of each step may be as much as 90-95%.

### Example 3: PCR for cDNA Production

PCR procedures for the preparation of cDNA may be performed using 2×KAPA HIFI™ HotStart ReadyMix by Kapa Biosystems (Woburn, MA). This system includes 2×KAPA ReadyMix 12.5 μl; Forward Primer (10 μM) 0.75 μl; Reverse Primer (10 μM) 0.75 μl; Template cDNA 100 ng; and dH₂0 diluted to 25.0 μl. The reaction conditions may be at 95° C. for 5 min. The reaction may be performed for 25 cycles of 98° C. for 20 sec, then 58° C. for 15 sec, then 72° C. for 45 sec, then 72° C. for 5 min, then 4° C. to termination.

The reaction may be cleaned up using Invitrogen's PURELINK™ PCR Micro Kit (Carlsbad, CA) per manufacturer's instructions (up to 5 μg). Larger reactions may require a cleanup using a product with a larger capacity. Following the cleanup, the cDNA may be quantified using the NANODROP™ and analyzed by agarose gel electrophoresis to confirm that the cDNA is the expected size. The cDNA may then be submitted for sequencing analysis before proceeding to the in vitro transcription reaction.

### Example 4: In Vitro Transcription (IVT)

The in vitro transcription reaction generates RNA polynucleotides. Such polynucleotides may comprise a region or part of the polynucleotides of the disclosure, including chemically modified RNA (e.g., mRNA) polynucleotides. The chemically modified RNA polynucleotides can be uniformly modified polynucleotides. The in vitro transcription reaction utilizes a custom mix of nucleotide triphosphates (NTPs). The NTPs may comprise chemically modified NTPs, or a mix of natural and chemically modified NTPs, or natural NTPs.

A typical in vitro transcription reaction includes the following:

| | | | |
|---|---|---|---|
| 1) | Template cDNA | 1.0 | μg |
| 2) | 10 × transcription buffer | 2.0 | μl |
| | (400 mM Tris-HCl pH 8.0, 190 mM | | |
| | MgCl₂, 50 mM DTT, 10 mM | | |
| | Spermidine) | | |
| 3) | Custom NTPs (25 mM each) | 0.2 | μl |
| 4) | RNase Inhibitor | 20 | U |
| 5) | T7 RNA polymerase | 3000 | U |

| | | |
|---|---|---|
| 6) | dH₂0 | up to 20.0 μl. and |
| 7) | Incubation at 37° C. for 3 hr-5 hrs. | |

The crude IVT mix may be stored at 4° C. overnight for cleanup the next day. 1 U of RNase-free DNase may then be used to digest the original template. After 15 minutes of incubation at 37° C., the mRNA may be purified using Ambion's MEGACLEAR™ Kit (Austin, TX) following the manufacturer's instructions. This kit can purify up to 500 μg of RNA. Following the cleanup, the RNA polynucleotide may be quantified using the NANODROP™ and analyzed by agarose gel electrophoresis to confirm the RNA polynucleotide is the proper size and that no degradation of the RNA has occurred.

### Example 5: Enzymatic Capping

Capping of a RNA polynucleotide is performed as follows where the mixture includes: IVT RNA 60 μg-180 μg and dH₂0 up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl2) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂O (Up to 28 μl); and incubation at 37° C. for 30 minutes for 60 μg RNA or up to 2 hours for 180 μg of RNA.

The RNA polynucleotide may then be purified using Ambion's MEGACLEAR™ Kit

(Austin, TX) following the manufacturer's instructions. Following the cleanup, the RNA may be quantified using the NANODROP™ (ThermoFisher, Waltham, MA) and analyzed by agarose gel electrophoresis to confirm the RNA polynucleotide is the proper size and that no degradation of the RNA has occurred. The RNA polynucleotide product may also be sequenced by running a reverse-transcription-PCR to generate the cDNA for sequencing.

### Example 6: PolyA Tailing Reaction

Without a poly-T in the cDNA, a poly-A tailing reaction must be performed before cleaning the final product. This is done by mixing capped IVT RNA (100 μl); RNase Inhibitor (20 U); 10× Tailing Buffer (0.5 M Tris-HCl (pH 8.0), 2.5 M NaCl, 100 mM MgCl2) (12.0 μl); 20 mM ATP (6.0 μl); Poly-A Polymerase (20 U); dH₂O up to 123.5 μl and incubation at 37° C. for 30 min. If the poly-A tail is already in the transcript, then the tailing reaction may be skipped and proceed directly to cleanup with Ambion's MEGACLEAR™ kit (Austin, TX) (up to 500 μg). Poly-A Polymerase may be a recombinant enzyme expressed in yeast.

It should be understood that the processivity or integrity of the poly A tailing reaction may not always result in an exact size polyA tail. Hence, polyA tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the present disclosure.

### Example 7: Capping Assays Protein Expression Assay

Polynucleotides (e.g., mRNA) encoding a polypeptide, containing any of the caps taught herein, can be transfected into cells at equal concentrations. The amount of protein secreted into the culture medium can be assayed by ELISA at 6, 12, 24 and/or 36 hours post-transfection. Synthetic polynucleotides that secrete higher levels of protein into the medium correspond to a synthetic polynucleotide with a higher translationally-competent cap structure.

### Purity Analysis Synthesis

RNA (e.g., mRNA) polynucleotides encoding a polypeptide, containing any of the caps taught herein can be compared for purity using denaturing Agarose-Urea gel electrophoresis or HPLC analysis. RNA polynucleotides with a single, consolidated band by electrophoresis correspond to the higher purity product compared to polynucleotides with multiple bands or streaking bands. Chemically modified RNA polynucleotides with a single HPLC peak also correspond to a higher purity product. The capping reaction with a higher efficiency provides a more pure polynucleotide population.

### Cytokine Analysis

RNA (e.g., mRNA) polynucleotides encoding a polypeptide, containing any of the caps taught herein can be transfected into cells at multiple concentrations. The amount of pro-inflammatory cytokines, such as TNF-alpha and IFN-beta, secreted into the culture medium can be assayed by ELISA at 6, 12, 24 and/or 36 hours post-transfection. RNA polynucleotides resulting in the secretion of higher levels of pro-inflammatory cytokines into the medium correspond to a polynucleotides containing an immune-activating cap structure.

### Capping Reaction Efficiency

RNA (e.g., mRNA) polynucleotides encoding a polypeptide, containing any of the caps taught herein can be analyzed for capping reaction efficiency by LC-MS after nuclease treatment. Nuclease treatment of capped polynucleotides yield a mixture of free nucleotides and the capped 5′-5-triphosphate cap structure detectable by LC-MS. The amount of capped product on the LC-MS spectra can be expressed as a percent of total polynucleotide from the reaction and correspond to capping reaction efficiency. The cap structure with a higher capping reaction efficiency has a higher amount of capped product by LC-MS.

### Example 8: Agarose Gel Electrophoresis of Modified RNA or RT PCR Products

Individual RNA polynucleotides (200-400 ng in a 20 μl volume) or reverse transcribed PCR products (200-400 ng) may be loaded into a well on a non-denaturing 1.2% Agarose E-Gel (Invitrogen, Carlsbad, CA) and run for 12-15 minutes, according to the manufacturer protocol.

### Example 9: NANODROP™ Modified RNA Quantification and UV Spectral Data

Chemically modified RNA polynucleotides in TE buffer (1 μl) are used for NANODROP™ UV absorbance readings to quantitate the yield of each polynucleotide from an chemical synthesis or in vitro transcription reaction.

### Example 10: Formulation of Modified mRNA Using Lipidoids

RNA (e.g., mRNA) polynucleotides may be formulated for in vitro experiments by mixing the polynucleotides with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations may be used as a starting point. After formation of the particle, polynucleotide is added and allowed to integrate with the complex. The encapsulation efficiency is determined using a standard dye exclusion assays.

### Example 11: Exemplary Nucleic Acid Encoding gE RNA Polynucleotide for Use in a VZV Vaccine

The following sequence is an exemplary sequence that can be used to encode a VZV RNA polynucleotide gE for use in a VZV vaccine. A VZV vaccine may comprise, for example, at least one RNA polynucleotide encoded by at least one of the following sequence or by at least one fragment of the following sequence. In some embodiments, the mRNA further comprises a 5′ cap, for example, any of the caps disclosed herein, e.g., a cap having sequence m⁷G (5′) ppp (5′) G-2′-O-methyl. In some embodiments, the mRNA does not have a cap sequence. In some embodiments, the mRNA has at least one chemical modification, for example, any of the chemical modifications disclosed herein, e.g., N1-methylpseudouridine modification or N1-ethylpseudouridine modification. In other embodiments, the mRNA does not have chemical modification.

Each of the sequences described herein encompasses a chemically modified sequence or an unmodified sequence which includes no modified nucleotides.

VZV gE-full-length Oka strain:
(SEQ ID NO: 1)
TCAAGCTTTTGGACCCTCGTACAGAAGCTAATACGACTCACTATAGGGAAA
TAAGAGAGAAAAGAAGAGTAAGAAGAAATATAAGAGCCACCATGGGGACAG
TGAATAAGCCGGTTGTGGGCGTGCTTATGGGCTTTGGGATTATTACCGGTA
CATTACGAATTACCAATCCAGTGCGCGCCAGTGTGCTGCGTTACGACGACT
TTCACATTGACGAGGATAAGCTGGATACTAACAGCGTGTACGAACCTTATT
ACCACTCAGATCATGCCGAATCAAGCTGGGTTAATAGAGGAGAAAGCAGCC
GAAAAGCCTACGACCACAACTCACCTTATATTTGGCCCAGAAACGATTATG
ACGGTTTCCTGGAAAACGCACATGAACACCATGGAGTCTACAACCAAGGCA
GGGGAATCGACAGTGGCGAGCGTCTTATGCAGCCAACACAGATGTCGGCAC
AGGAGGATCTCGGTGATGACACCGGCATACACGTGATTCCCACATTAAACG
GCGACGACAGACATAAGATCGTCAATGTGGATCAGCGTCAGTATGGGGATG
TCTTTAAAGGCGATTTGAATCCAAAGCCCCAAGGACAGAGACTGATCGAGG
TCTCTGTAGAAGAAAATCACCCCTTCACTTTGCGCGCTCCAATCCAGAGGA
TTTACGGGGTGCGTTATACCGAAACTTGGAGTTTCTTGCCGTCACTGACGT
GTACGGGGGATGCCGCCCCCGCAATCCAGCACATCTGTCTGAAACACACCA
CATGCTTTCAGGACGTGGTTGTGGATGTGGATTGCGCGGAAAACACAAAAG
AAGACCAACTCGCCGAAATCAGCTATCGTTTTCAGGGTAAAAAAGAGGCCG
ACCAACCGTGGATTGTTGTGAATACGAGCACGCTCTTCGATGAGCTTGAAC
TCGATCCCCCGGAAATCGAGCCTGGGGTTCTAAAAGTGTTGAGGACCGAGA
AGCAGTACCTCGGGGTTTATATCTGGAATATGAGAGGCTCCGATGGCACCT
CTACCTACGCAACGTTTCTGGTTACCTGGAAGGGAGACGAGAAGACACGGA
ATCCAACGCCCGCTGTGACCCCTCAGCCTAGGGGAGCCGAATTCCACATGT
GGAACTATCACTCCCATGTATTCAGTGTGGGTGACACTTTCAGCCTGGCCA
TGCACCTGCAGTATAAGATTCACGAGGCACCCTTCGACCTCCTGCTGGAGT
GGTTGTACGTACCTATTGATCCCACTTGTCAGCCCATGCGCCTGTACTCCA
CTTGCTTGTACCACCCCAATGCACCACAGTGTCTATCACACATGAACTCCG
GGTGTACCTTTACTTCACCCCATCTTGCCCAGCGGGTCGCCAGCACAGTGT
ATCAGAACTGTGAGCATGCTGACAACTATACTGCTTATTGCCTCGGAATAT
CCCATATGGAGCCAAGCTTCGGGCTCATACTGCACGATGGTGGTACGACAC
TCAAGTTCGTGGACACCCCCGAAAGCCTTTCTGGCTTGTACGTGTTCGTGG
TCTACTTCAATGGACATGTGGAGGCAGTGGCTTACACAGTGGTTTCGACAG
TTGATCACTTTGTAAATGCCATTGAGGAACGCGGCTTCCCGCCTACAGCGG
GCCAGCCCCCTGCGACAACAAAACCAAAAGAGATTACGCCCGTTAATCCTG
GGACTAGTCCATTGCTGAGGTATGCCGCCTGGACTGGCGGTCTGGCGGCCG
TGGTACTTCTGTGTTTAGTCATATTTCTGATCTGTACCGCTAAACGTATGC
GGGTCAAGGCTTACCGTGTTGACAAGTCTCCTTACAATCAGTCAATGTACT
ATGCAGGACTCCCTGTTGACGATTTCGAAGACTCAGAGAGTACAGACACAG
AAGAAGAATTCGGAAACGCTATAGGTGGCTCTCACGGAGGTAGCTCGTATA
CAGTGTACATCGATAAAACCAGATGATAATAGGCTGGAGCCTCGGTGGCCA
TGCTTCTTGCCCCTTGGGCCTCCCCCCAGCCCCTCCTCCCCTTCCTGCACC
CGTACCCCCGTGGTCTTTGAATAAAGTCTGAGTGGGCGGC
VZV gE-full-length Oka strain (mRNA):
(SEQ ID NO: 123)
UCAAGCUUUUGGACCCUCGUACAGAAGCUAAUACGACUCACUAUAGGGAAA
UAAGAGAGAAAAGAAGAGUAAGAAGAAAUAUAAGAGCCACCAUGGGGACAG
UGAAUAAGCCGGUUGUGGGCGUGCUUAUGGGCUUUGGGAUUAUUACCGGUA
CAUUACGAAUUACCAAUCCAGUGCGCGCCAGUGUGCUGCGUUACGACGACU
UUCACAUUGACGAGGAUAAGCUGGAUACUAACAGCGUGUACGAACCUUAUU
ACCACUCAGAUCAUGCCGAAUCAAGCUGGGUUAAUAGAGGAGAAAGCAGCC
GAAAAGCCUACGACCACAACUCACCUUAUAUUUGGCCCAGAAACGAUUAUG
ACGGUUUCCUGGAAAACGCACAUGAACACCAUGGAGUCUACAACCAAGGCA
GGGGAAUCGACAGUGGCGAGCGUCUUAUGCAGCCAACACAGAUGUCGGCAC
AGGAGGAUCUCGGUGAUGACACCGGCAUACACGUGAUUCCCACAUUAAACG
GCGACGACAGACAUAAGAUCGUCAAUGUGGAUCAGCGUCAGUAUGGGGAUG
UCUUUAAAGGCGAUUUGAAUCCAAAGCCCCAAGGACAGAGACUGAUCGAGG
UCUCUGUAGAAGAAAAUCACCCCUUCACUUUGCGCGCUCCAAUCCAGAGGA
UUUACGGGGUGCGUUAUACCGAAACUUGGAGUUUCUUGCCGUCACUGACGU
GUACGGGGGAUGCCGCCCCCGCAAUCCAGCACAUCUGUCUGAAACACACCA
CAUGCUUUCAGGACGUGGUUGUGGAUGUGGAUUGCGCGGAAAACACAAAAG
AAGACCAACUCGCCGAAAUCAGCUAUCGUUUUCAGGGUAAAAAAGAGGCCG
ACCAACCGUGGAUUGUUGUGAAUACGAGCACGCUCUUCGAUGAGCUUGAAC
UCGAUCCCCCGGAAAUCGAGCCUGGGGUUCUAAAAGUGUUGAGGACCGAGA
AGCAGUACCUCGGGGUUUAUAUCUGGAAUAUGAGAGGCUCCGAUGGCACCU
CUACCUACGCAACGUUUCUGGUUACCUGGAAGGGAGACGAGAAGACACGGA
AUCCAACGCCCGCUGUGACCCCUCAGCCUAGGGGAGCCGAAUUCCACAUGU
GGAACUAUCACUCCCAUGUAUUCAGUGUGGGUGACACUUUCAGCCUGGCCA
UGCACCUGCAGUAUAAGAUUCACGAGGCACCCUUCGACCUCCUGCUGGAGU
GGUUGUACGUACCUAUUGAUCCCACUUGUCAGCCCAUGCGCCUGUACUCCA
CUUGCUUGUACCACCCCAAUGCACCACAGUGUCUAUCACACAUGAACUCCG
GGUGUACCUUUACUUCACCCCAUCUUGCCCAGCGGGUCGCCAGCACAGUGU
AUCAGAACUGUGAGCAUGCUGACAACUAUACUGCUUAUUGCCUCGGAAUAU
CCCAUAUGGAGCCAAGCUUCGGGCUCAUACUGCACGAUGGUGGUACGACAC
UCAAGUUCGUGGACACCCCCGAAAGCCUUUCUGGCUUGUACGUGUUCGUGG
UCUACUUCAAUGGACAUGUGGAGGCAGUGGCUUACACAGUGGUUUCGACAG
UUGAUCACUUUGUAAAUGCCAUUGAGGAACGCGGCUUCCCGCCUACAGCGG
GCCAGCCCCCUGCGACAACAAAACCAAAAGAGAUUACGCCCGUUAAUCCUG
GGACUAGUCCAUUGCUGAGGUAUGCCGCCUGGACUGGCGGUCUGGCGGCCG
UGGUACUUCUGUGUUUAGUCAUAUUUCUGAUCUGUACCGCUAAACGUAUGC
GGGUCAAGGCUUACCGUGUUGACAAGUCUCCUUACAAUCAGUCAAUGUACU
AUGCAGGACUCCCUGUUGACGAUUUCGAAGACUCAGAGAGUACAGACACAG
AAGAAGAAUUCGGAAACGCUAUAGGUGGCUCUCACGGAGGUAGCUCGUAUA
CAGUGUACAUCGAUAAAACCAGAUGAUAAUAGGCUGGAGCCUCGGUGGCCA
UGCUUCUUGCCCCUUGGGCCUCCCCCCAGCCCCUCCUCCCCUUCCUGCACC
CGUACCCCCGUGGUCUUUGAAUAAAGUCUGAGUGGGCGGC

### Example 12: Exemplary Nucleic Acid Encoding gI RNA Polynucleotide for Use in a VZV Vaccine

The following sequence is an exemplary sequence that can be used to encode a VZV

RNA polynucleotide gI for use in a VZV RNA (e.g., mRNA) vaccine. The gI polypeptide forms a complex with gE in infected cells which facilitates the endocytosis of both glycoproteins and directs them to the trans-Golgi network (TGN) where the final viral envelope is acquired. A VZV vaccine may comprise, for example, at least one RNA (e.g., mRNA) polynucleotide encoded by at least one of the following sequence or by at least one fragment of the following sequence. In some embodiments, the mRNA further comprises a 5′ cap, for example, any of the caps disclosed herein, e.g., a cap having sequence m⁷G (5′) ppp (5′) G-2′-O-methyl. In other embodiments, the mRNA does not have a cap sequence. In some embodiments, the mRNA has at least one chemical modification, for example, any of the chemical modifications disclosed herein, e.g., N1-methylpseudouridine modification or N1-ethylpseudouridine modification. In other embodiments, the mRNA does not have chemical modification.

VZV-GI-full length (Oka strain):
(SEQ ID NO: 2)
TCAAGCTTTTGGACCCTCGTACAGAAGCTAATACGACTCACTATAGGGAAA
TAAGAGAGAAAAGAAGAGTAAGAAGAAATATAAGAGCCACCATGTTTTTAA
TCCAATGTTTGATATCGGCCGTTATATTTTACATACAAGTGACCAACGCTT
TGATCTTCAAGGGCGACCACGTGAGCTTGCAAGTTAACAGCAGTCTCACGT
CTATCCTTATTCCCATGCAAAATGATAATTATACAGAGATAAAAGGACAGC
TTGTCTTTATTGGAGAGCAACTACCTACCGGGACAAACTATAGCGGAACAC
TGGAACTGTTATACGCGGATACGGTGGCGTTTTGTTTCCGGTCAGTACAAG
TAATAAGATACGACGGATGTCCCCGGATTAGAACGAGCGCTTTTATTTCGT
GTAGGTACAAACATTCGTGGCATTATGGTAACTCAACGGATCGGATATCAA
CAGAGCCGGATGCTGGTGTAATGTTGAAAATTACCAAACCGGGAATAAATG
ATGCTGGTGTGTATGTACTTCTTGTTCGGTTAGACCATAGCAGATCCACCG
ATGGTTTCATTCTTGGTGTAAATGTATATACAGCGGGCTCGCATCACAACA
TTCACGGGGTTATCTACACTTCTCCATCTCTACAGAATGGATATTCTACAA
GAGCCCTTTTTCAACAAGCTCGTTTGTGTGATTTACCCGCGACACCCAAAG
GGTCCGGTACCTCCCTGTTTCAACATATGCTTGATCTTCGTGCCGGTAAAT
CGTTAGAGGATAACCCTTGGTTACATGAGGACGTTGTTACGACAGAAACTA
AGTCCGTTGTTAAGGAGGGGATAGAAAATCACGTATATCCAACGGATATGT
CCACGTTACCCGAAAAGTCCCTTAATGATCCTCCAGAAAATCTACTTATAA
TTATTCCTATAGTAGCGTCTGTCATGATCCTCACCGCCATGGTTATTGTTA
TTGTAATAAGCGTTAAGCGACGTAGAATTAAAAAACATCCAATTTATCGCC
CAAATACAAAAACAAGAAGGGGCATACAAAATGCGACACCAGAATCCGATG
TGATGTTGGAGGCCGCCATTGCACAACTAGCAACGATTCGCGAAGAATCCC
CCCCACATTCCGTTGTAAACCCGTTTGTTAAATAGTGATAATAGGCTGGAG
CCTCGGTGGCCATGCTTCTTGCCCCTTGGGCCTCCCCCCAGCCCCTCCTCC
CCTTCCTGCACCCGTACCCCCGTGGTCTTTGAATAAAGTCTGAGTGGGCGG
C
VZV-GI-full length (Oka strain) (mRNA):
(SEQ ID NO: 124)
UCAAGCUUUUGGACCCUCGUACAGAAGCUAAUACGACUCACUAUAGGGAAA
UAAGAGAGAAAAGAAGAGUAAGAAGAAAUAUAAGAGCCACCAUGUUUUUAA
UCCAAUGUUUGAUAUCGGCCGUUAUAUUUUACAUACAAGUGACCAACGCUU
UGAUCUUCAAGGGCGACCACGUGAGCUUGCAAGUUAACAGCAGUCUCACGU
CUAUCCUUAUUCCCAUGCAAAAUGAUAAUUAUACAGAGAUAAAAGGACAGC
UUGUCUUUAUUGGAGAGCAACUACCUACCGGGACAAACUAUAGCGGAACAC
UGGAACUGUUAUACGCGGAUACGGUGGCGUUUUGUUUCCGGUCAGUACAAG
UAAUAAGAUACGACGGAUGUCCCCGGAUUAGAACGAGCGCUUUUAUUUCGU
GUAGGUACAAACAUUCGUGGCAUUAUGGUAACUCAACGGAUCGGAUAUCAA
CAGAGCCGGAUGCUGGUGUAAUGUUGAAAAUUACCAAACCGGGAAUAAAUG
AUGCUGGUGUGUAUGUACUUCUUGUUCGGUUAGACCAUAGCAGAUCCACCG
AUGGUUUCAUUCUUGGUGUAAAUGUAUAUACAGCGGGCUCGCAUCACAACA
UUCACGGGGUUAUCUACACUUCUCCAUCUCUACAGAAUGGAUAUUCUACAA
GAGCCCUUUUUCAACAAGCUCGUUUGUGUGAUUUACCCGCGACACCCAAAG
GGUCCGGUACCUCCCUGUUUCAACAUAUGCUUGAUCUUCGUGCCGGUAAAU
CGUUAGAGGAUAACCCUUGGUUACAUGAGGACGUUGUUACGACAGAAACUA
AGUCCGUUGUUAAGGAGGGGAUAGAAAAUCACGUAUAUCCAACGGAUAUGU
CCACGUUACCCGAAAAGUCCCUUAAUGAUCCUCCAGAAAAUCUACUUAUAA
UUAUUCCUAUAGUAGCGUCUGUCAUGAUCCUCACCGCCAUGGUUAUUGUUA
UUGUAAUAAGCGUUAAGCGACGUAGAAUUAAAAAACAUCCAAUUUAUCGCC
CAAAUACAAAAACAAGAAGGGGCAUACAAAAUGCGACACCAGAAUCCGAUG
UGAUGUUGGAGGCCGCCAUUGCACAACUAGCAACGAUUCGCGAAGAAUCCC
CCCCACAUUCCGUUGUAAACCCGUUUGUUAAAUAGUGAUAAUAGGCUGGAG
CCUCGGUGGCCAUGCUUCUUGCCCCUUGGGCCUCCCCCCAGCCCCUCCUCC
CCUUCCUGCACCCGUACCCCCGUGGUCUUUGAAUAAAGUCUGAGUGGGCGG
C

### Example 13: mRNAs Encoding Variant gE Antigens Having Different C-Terminal Sequence for Use in a VZV Vaccine

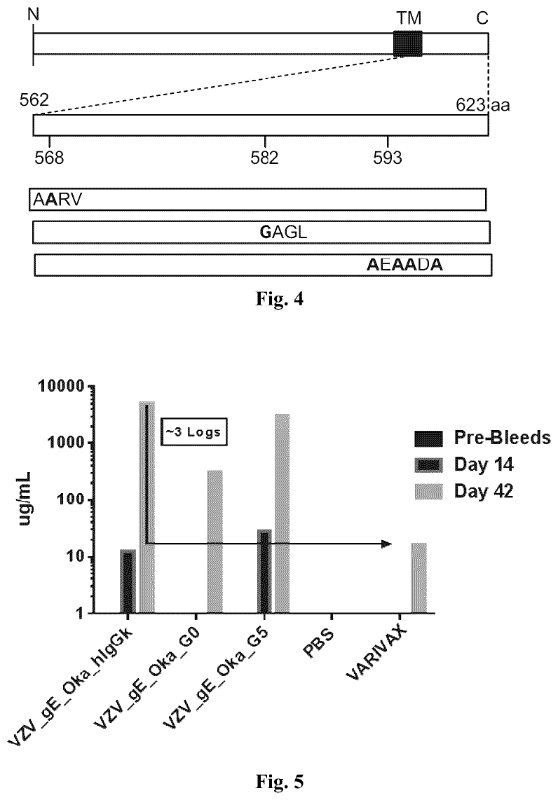

VZV is enveloped in the trans-golgi network. Glycoprotein I (gI) forms a complex with gE in infected cells which facilitates the endocytosis of both glycoproteins and directs them to the trans-Golgi network (TGN) where the final viral envelope is acquired. mRNAs encoding gE antigens having different C-terminal variant sequence were designed to avoid gE being trapped in the ER/golgi/TGN, leading to an increase in the localization of gE antigen to the plasma membrane and improved immune-stimulating capabilities. A schematic of the gE antigen is shown in FIG. 4.

Several different gE variant mRNA sequences (Oka strain) were engineered:

(1) gE variant mRNA encoding a truncated polypeptide having the terminal 62 amino acids of the C terminal region deleted (SEQ ID NO: 17-20). The resultant polypeptide has reduced localization to the trans-golgi network and reduced endocytosis.
(2) gE variant mRNA encoding a truncated polypeptide having the terminal 62 amino acids of the C terminal region deleted and also having the signal peptide replaced with IgKappa, which results in a secreted form of the truncated gE polypeptide (SEQ ID NO: 21-24). The resultant polypeptide has reduced localization to the trans-golgi network and reduced endocytosis.

(3) gE variant mRNA encoding a truncated polypeptide having the terminal 50 amino acids of the C terminal region deleted (SEQ ID NO: 33-36). The resultant polypeptide has reduced localization to the trans-golgi network and reduced endocytosis.

(4) gE variant mRNA encoding a truncated polypeptide having the terminal 50 amino acids of the C terminal region deleted and also having the point mutation Y569A (SEQ ID NO: 37-40). The “AYRV” motif (SEQ ID NO: 119) is a trafficking motif which targets the gE polypeptide to the trans-golgi network. Thus, mutating the AARV sequence SEQ ID NO: 120 to AYRV SEQ ID NO: 119 results in reduced localization of the gE polypeptide to the trans-golgi network.

(5) gE variant mRNA encoding full-length gE polypeptide with an AEAADA (SEQ ID NO: 58) sequence (SEQ ID NO: 25-28). The A-E-A-A-D-A (SEQ ID NO: 58) sequence replaces SESTDT (SEQ ID NO: 59). This is a replacement of the Ser/Thr-rich “SSTT” (SEQ ID NO: 122) acidic cluster with an Ala-rich sequence. This reduces CKII phosphorylation, which in turn results in reduced localization of the gE polypeptide to the trans-golgi network.

(6) gE variant mRNA encoding full-length gE polypeptide with an AEAADA (SEQ ID NO: 58) sequence and also having the point mutation Y582G (SEQ ID NO: 29-32). The “YAGL” (SEQ ID NO: 121) motif is an endocytosis motif which enhances localization of the gE polypeptide to the trans-golgi network. Thus, mutating the GAGL sequence (SEQ ID NO: 132) to YAGL (SEQ ID NO: 121) results in reduced endocytosis of the resultant polypeptide.

Each of these variants have modifications that reduce localization of the encoded gE protein to the trans-golgi network and enhance trafficking to the plasma membrane. Table 1 summarizes mRNAs encoding the variant gE antigens having different C-terminal sequence. In some embodiments, the variant mRNA further comprises a 5′ cap, for example, any of the caps disclosed herein, e.g., a cap having sequence m⁷G (5′) ppp (5′) G-2′-O-methyl. In some embodiments, the variant mRNA does not have a 5′ cap. In some embodiments, the variant mRNA has at least one chemical modification, for example, any of the chemical modifications disclosed herein, e.g., N1-methylpseudouridine modification or N1-ethylpseudouridine modification. In some embodiments, the mRNA does not have chemical modification. The sequences encoding the mRNA variants are provided beneath the table.

TABLE 1
mRNA Constructs

| SEQ | | | |
|---|---|---|---|
| ID | Name of | | |
| NO. | mRNA construct | Description | Function |
| 3 | VZV-GE-delete- | Truncated VZV gE | The C-terminal |
| | 562 | sequence—deletion from | sequence targets |
| | | aa 562 (62 aa deletion | gE to the trans- |
| | | from C terminal domain) | Golgi network |
| | | | (TGN); truncation |
| | | | assists in reducing |
| | | | gE localization to |
| | | | TGN |
| 4 | VZV-GE-delete- | Secreted form of truncated | The C-terminal |
| | 562-IgKappa | VZV gE sequence— | sequence targets |
| | | deletion from aa 562 (62 | gE to the trans- |
| | | aa deletion from C terminal | Golgi networks |
| | | domain) and signal peptide | (TGN); truncation |
| | | replaced with IgKappa | assists in reducing |
| | | | gE localization to |
| | | | TGN |
| 5 | VZV-GE-delete- | Truncated VZV gE | The C-terminal |
| | 574 | sequence—deletion from | sequence targets |
| | | aa 574 (50 aa deletion from | gE to the trans- |
| | | C terminal domain) | Golgi network |
| | | | (TGN); truncation |
| | | | assists in reducing |
| | | | gE localization to |
| | | | TGN |
| 6 | VZV-GE-delete- | Truncated VZV gE | The C-terminal |
| | 574-Y569A | sequence—deletion from | sequence targets |
| | | aa 574 (50 aa deletion | gE to the trans- |
| | | from C terminal domain) | Golgi network |
| | | and Y569A point mutation | (TGN); the AYRV |
| | | | (SEQ ID NO: 119) |
| | | | sequence is |
| | | | required for |
| | | | targeting gE to the |
| | | | TGN; |
| | | | truncation/mutation |
| | | | reduces |
| | | | localization to |
| | | | TGN) |
| 7 | VZV-GE-full- | VZV gE full length | AEAADA (SEQ |
| | length- | sequence with AEAADA | ID NO: 58) |
| | AEAADA (SEQ | (SEQ ID NO: 58) sequence | replaces SSTT |
| | ID NO: 58) | | (SEQ ID NO: 122) |
| | | | (acid cluster) |
| | | | comprising a |
| | | | phosphorylation |
| | | | motif, which |
| | | | phosphorylation |
| | | | assists in localizing |
| | | | gE to the TGN; |
| | | | mutation reduces |
| | | | localization of gE |
| | | | to TGN |
| 8 | VZV-GE-full- | VZV gE-full length | Mutations assist in |
| | length- | sequence with AEAADA | reducing |
| | AEAADA (SEQ | sequence (SEQ ID NO: | endocytosis and |
| | ID NO: 58)- | 58) and Y582G point | localization of gE |
| | Y582G | mutation | to the TGN |

VZV-GE-delete-562
(SEQ ID NO: 3)
TCAAGCTTTTGGACCCTCGTACAGAAGCTAATACGACTCACTATAGGGAAATAAGAGAGAAAAGA
AGAGTAAGAAGAAATATAAGAGCCACCATGGGGACAGTTAATAAACCTGTGGTGGGGGTATTGAT
GGGGTTCGGAATTATCACGGGAACGTTGCGTATAACGAATCCGGTCAGAGCATCCGTCTTGCGATA
CGATGATTTTCACATCGATGAAGACAAACTGGATACAAACTCCGTATATGAGCCTTACTACCATTC
AGATCATGCGGAGTCTTCATGGGTAAATCGGGGAGAGTCTTCGCGAAAAGCGTACGATCATAACT
CACCTTATATATGGCCACGTAATGATTATGATGGATTTTTAGAGAACGCACACGAACACCATGGGG
TGTATAATCAGGGCCGTGGTATCGATAGCGGGGAACGGTTAATGCAACCCACACAAATGTCTGCA
CAGGAGGATCTTGGGGACGATACGGGCATCCACGTTATCCCTACGTTAAACGGCGATGACAGACA
TAAAATTGTAAATGTGGACCAACGTCAATACGGTGACGTGTTTAAAGGAGATCTTAATCCAAAAC
CCCAAGGCCAAAGACTCATTGAGGTGTCAGTGGAAGAAAATCACCCGTTTACTTTACGCGCACCG
ATTCAGCGGATTTATGGAGTCCGGTACACCGAGACTTGGAGCTTTTTGCCGTCATTAACCTGTACG
GGAGACGCAGCGCCCGCCATCCAGCATATATGTTTAAAACATACAACATGCTTTCAAGACGTGGT
GGTGGATGTGGATTGCGCGGAAAATACTAAAGAGGATCAGTTGGCCGAAATCAGTTACCGTTTTC
AAGGTAAGAAGGAAGCGGACCAACCGTGGATTGTTGTAAACACGAGCACACTGTTTGATGAACTC
GAATTAGACCCCCCCGAGATTGAACCGGGTGTCTTGAAAGTACTTCGGACAGAAAAACAATACTT
GGGTGTGTACATTTGGAACATGCGCGGCTCCGATGGTACGTCTACCTACGCCACGTTTTTGGTCAC
CTGGAAAGGGGATGAAAAAACAAGAAACCCTACGCCCGCAGTAACTCCTCAACCAAGAGGGGCT
GAGTTTCATATGTGGAATTACCACTCGCATGTATTTTCAGTTGGTGATACGTTTAGCTTGGCAATGC
ATCTTCAGTATAAGATACATGAAGCGCCATTTGATTTGCTGTTAGAGTGGTTGTATGTCCCCATCG
ATCCTACATGTCAACCAATGCGGTTATATTCTACGTGTTTGTATCATCCCAACGCACCCCAATGCCT
CTCTCATATGAATTCCGGTTGTACATTTACCTCGCCACATTTAGCCCAGCGTGTTGCAAGCACAGTG
TATCAAAATTGTGAACATGCAGATAACTACACCGCATATTGTCTGGGAATATCTCATATGGAGCCT
AGCTTTGGTCTAATCTTACACGACGGGGGCACCACGTTAAAGTTTGTAGATACACCCGAGAGTTTG
TCGGGATTATACGTTTTTGTGGTGTATTTTAACGGGCATGTTGAAGCCGTAGCATACACTGTTGTAT
CCACAGTAGATCATTTTGTAAACGCAATTGAAGAGCGTGGATTTCCGCCAACGGCCGGTCAGCCAC
CGGCGACTACTAAACCCAAGGAAATTACCCCCGTAAACCCCGGAACGTCACCACTTCTACGATAT
GCCGCATGGACCGGAGGGCTTGCAGCAGTAGTACTTTTATGTCTCGTAATATTTTTAATCTGTACG
GCTTGATGATAATAGGCTGGAGCCTCGGTGGCCATGCTTCTTGCCCCTTGGGCCTCCCCCCAGCCC
CTCCTCCCCTTCCTGCACCCGTACCCCCGTGGTCTTTGAATAAAGTCTGAGTGGGCGGC
VZV-GE-delete-562 (mRNA)
(SEQ ID NO: 125)
UCAAGCUUUUGGACCCUCGUACAGAAGCUAAUACGACUCACUAUAGGGAAAUAAGAGAGAAAA
GAAGAGUAAGAAGAAAUAUAAGAGCCACCAUGGGGACAGUUAAUAAACCUGUGGUGGGGGUAU
UGAUGGGGUUCGGAAUUAUCACGGGAACGUUGCGUAUAACGAAUCCGGUCAGAGCAUCCGUCU
UGCGAUACGAUGAUUUUCACAUCGAUGAAGACAAACUGGAUACAAACUCCGUAUAUGAGCCUU
ACUACCAUUCAGAUCAUGCGGAGUCUUCAUGGGUAAAUCGGGGAGAGUCUUCGCGAAAAGCGU
ACGAUCAUAACUCACCUUAUAUAUGGCCACGUAAUGAUUAUGAUGGAUUUUUAGAGAACGCAC
ACGAACACCAUGGGGUGUAUAAUCAGGGCCGUGGUAUCGAUAGCGGGGAACGGUUAAUGCAAC
CCACACAAAUGUCUGCACAGGAGGAUCUUGGGGACGAUACGGGCAUCCACGUUAUCCCUACGU
UAAACGGCGAUGACAGACAUAAAAUUGUAAAUGUGGACCAACGUCAAUACGGUGACGUGUUUA
AAGGAGAUCUUAAUCCAAAACCCCAAGGCCAAAGACUCAUUGAGGUGUCAGUGGAAGAAAAUC
ACCCGUUUACUUUACGCGCACCGAUUCAGCGGAUUUAUGGAGUCCGGUACACCGAGACUUGGA
GCUUUUUGCCGUCAUUAACCUGUACGGGAGACGCAGCGCCCGCCAUCCAGCAUAUAUGUUUAA
AACAUACAACAUGCUUUCAAGACGUGGUGGUGGAUGUGGAUUGCGCGGAAAAUACUAAAGAGG
AUCAGUUGGCCGAAAUCAGUUACCGUUUUCAAGGUAAGAAGGAAGCGGACCAACCGUGGAUUG
UUGUAAACACGAGCACACUGUUUGAUGAACUCGAAUUAGACCCCCCCGAGAUUGAACCGGGUG
UCUUGAAAGUACUUCGGACAGAAAAACAAUACUUGGGUGUGUACAUUUGGAACAUGCGCGGCU
CCGAUGGUACGUCUACCUACGCCACGUUUUUGGUCACCUGGAAAGGGGAUGAAAAAACAAGAA
ACCCUACGCCCGCAGUAACUCCUCAACCAAGAGGGGCUGAGUUUCAUAUGUGGAAUUACCACUC
GCAUGUAUUUUCAGUUGGUGAUACGUUUAGCUUGGCAAUGCAUCUUCAGUAUAAGAUACAUGA
AGCGCCAUUUGAUUUGCUGUUAGAGUGGUUGUAUGUCCCCAUCGAUCCUACAUGUCAACCAAU
GCGGUUAUAUUCUACGUGUUUGUAUCAUCCCAACGCACCCCAAUGCCUCUCUCAUAUGAAUUCC
GGUUGUACAUUUACCUCGCCACAUUUAGCCCAGCGUGUUGCAAGCACAGUGUAUCAAAAUUGU
GAACAUGCAGAUAACUACACCGCAUAUUGUCUGGGAAUAUCUCAUAUGGAGCCUAGCUUUGGU
CUAAUCUUACACGACGGGGGCACCACGUUAAAGUUUGUAGAUACACCCGAGAGUUUGUCGGGA
UUAUACGUUUUUGUGGUGUAUUUUAACGGGCAUGUUGAAGCCGUAGCAUACACUGUUGUAUCC
ACAGUAGAUCAUUUUGUAAACGCAAUUGAAGAGCGUGGAUUUCCGCCAACGGCCGGUCAGCCA
CCGGCGACUACUAAACCCAAGGAAAUUACCCCCGUAAACCCCGGAACGUCACCACUUCUACGAU
AUGCCGCAUGGACCGGAGGGCUUGCAGCAGUAGUACUUUUAUGUCUCGUAAUAUUUUUAAUCU
GUACGGCUUGAUGAUAAUAGGCUGGAGCCUCGGUGGCCAUGCUUCUUGCCCCUUGGGCCUCCCC
CCAGCCCCUCCUCCCCUUCCUGCACCCGUACCCCCGUGGUCUUUGAAUAAAGUCUGAGUGGGCG
GC
VZV-GE-delete-562-IgKappa
(SEQ ID NO: 4)
TCAAGCTTTTGGACCCTCGTACAGAAGCTAATACGACTCACTATAGGGAAATAAGAGAGAAAAGA
AGAGTAAGAAGAAATATAAGAGCCACCATGGAAACCCCGGCGCAGCTGCTGTTTCTGCTGCTGCT
GTGGCTGCCGGATACCACCGGCTCCGTCTTGCGATACGATGATTTTCACATCGATGAAGACAAACT
GGATACAAACTCCGTATATGAGCCTTACTACCATTCAGATCATGCGGAGTCTTCATGGGTAAATCG
GGGAGAGTCTTCGCGAAAAGCGTACGATCATAACTCACCTTATATATGGCCACGTAATGATTATGA
TGGATTTTTAGAGAACGCACACGAACACCATGGGGTGTATAATCAGGGCCGTGGTATCGATAGCG
GGGAACGGTTAATGCAACCCACACAAATGTCTGCACAGGAGGATCTTGGGGACGATACGGGCATC
CACGTTATCCCTACGTTAAACGGCGATGACAGACATAAAATTGTAAATGTGGACCAACGTCAATA
CGGTGACGTGTTTAAAGGAGATCTTAATCCAAAACCCCAAGGCCAAAGACTCATTGAGGTGTCAG
TGGAAGAAAATCACCCGTTTACTTTACGCGCACCGATTCAGCGGATTTATGGAGTCCGGTACACCG
AGACTTGGAGCTTTTTGCCGTCATTAACCTGTACGGGAGACGCAGCGCCCGCCATCCAGCATATAT
GTTTAAAACATACAACATGCTTTCAAGACGTGGTGGTGGATGTGGATTGCGCGGAAAATACTAAA
GAGGATCAGTTGGCCGAAATCAGTTACCGTTTTCAAGGTAAGAAGGAAGCGGACCAACCGTGGAT
TGTTGTAAACACGAGCACACTGTTTGATGAACTCGAATTAGACCCCCCCGAGATTGAACCGGGTGT
CTTGAAAGTACTTCGGACAGAAAAACAATACTTGGGTGTGTACATTTGGAACATGCGCGGCTCCG
ATGGTACGTCTACCTACGCCACGTTTTTGGTCACCTGGAAAGGGGATGAAAAAACAAGAAACCCT
ACGCCCGCAGTAACTCCTCAACCAAGAGGGGCTGAGTTTCATATGTGGAATTACCACTCGCATGTA
TTTTCAGTTGGTGATACGTTTAGCTTGGCAATGCATCTTCAGTATAAGATACATGAAGCGCCATTTG
ATTTGCTGTTAGAGTGGTTGTATGTCCCCATCGATCCTACATGTCAACCAATGCGGTTATATTCTAC
GTGTTTGTATCATCCCAACGCACCCCAATGCCTCTCTCATATGAATTCCGGTTGTACATTTACCTCG
CCACATTTAGCCCAGCGTGTTGCAAGCACAGTGTATCAAAATTGTGAACATGCAGATAACTACACC
GCATATTGTCTGGGAATATCTCATATGGAGCCTAGCTTTGGTCTAATCTTACACGACGGGGGCACC
ACGTTAAAGTTTGTAGATACACCCGAGAGTTTGTCGGGATTATACGTTTTTGTGGTGTATTTTAACG
GGCATGTTGAAGCCGTAGCATACACTGTTGTATCCACAGTAGATCATTTTGTAAACGCAATTGAAG
AGCGTGGATTTCCGCCAACGGCCGGTCAGCCACCGGCGACTACTAAACCCAAGGAAATTACCCCC
GTAAACCCCGGAACGTCACCACTTCTACGATATGCCGCATGGACCGGAGGGCTTGCAGCAGTAGT
ACTTTTATGTCTCGTAATATTTTTAATCTGTACGGCTTGATGATAATAGGCTGGAGCCTCGGTGGCC
ATGCTTCTTGCCCCTTGGGCCTCCCCCCAGCCCCTCCTCCCCTTCCTGCACCCGTACCCCCGTGGTC
TTTGAATAAAGTCTGAGTGGGCGGC
VZV-GE-delete-562-IgKappa (mRNA)
(SEQ ID NO: 126)
UCAAGCUUUUGGACCCUCGUACAGAAGCUAAUACGACUCACUAUAGGGAAAUAAGAGAGAAAA
GAAGAGUAAGAAGAAAUAUAAGAGCCACCAUGGAAACCCCGGCGCAGCUGCUGUUUCUGCUGC
UGCUGUGGCUGCCGGAUACCACCGGCUCCGUCUUGCGAUACGAUGAUUUUCACAUCGAUGAAG
ACAAACUGGAUACAAACUCCGUAUAUGAGCCUUACUACCAUUCAGAUCAUGCGGAGUCUUCAU
GGGUAAAUCGGGGAGAGUCUUCGCGAAAAGCGUACGAUCAUAACUCACCUUAUAUAUGGCCAC
GUAAUGAUUAUGAUGGAUUUUUAGAGAACGCACACGAACACCAUGGGGUGUAUAAUCAGGGCC
GUGGUAUCGAUAGCGGGGAACGGUUAAUGCAACCCACACAAAUGUCUGCACAGGAGGAUCUUG
GGGACGAUACGGGCAUCCACGUUAUCCCUACGUUAAACGGCGAUGACAGACAUAAAAUUGUAA
AUGUGGACCAACGUCAAUACGGUGACGUGUUUAAAGGAGAUCUUAAUCCAAAACCCCAAGGCC
AAAGACUCAUUGAGGUGUCAGUGGAAGAAAAUCACCCGUUUACUUUACGCGCACCGAUUCAGC
GGAUUUAUGGAGUCCGGUACACCGAGACUUGGAGCUUUUUGCCGUCAUUAACCUGUACGGGAG
ACGCAGCGCCCGCCAUCCAGCAUAUAUGUUUAAAACAUACAACAUGCUUUCAAGACGUGGUGG
UGGAUGUGGAUUGCGCGGAAAAUACUAAAGAGGAUCAGUUGGCCGAAAUCAGUUACCGUUUUC
AAGGUAAGAAGGAAGCGGACCAACCGUGGAUUGUUGUAAACACGAGCACACUGUUUGAUGAAC
UCGAAUUAGACCCCCCCGAGAUUGAACCGGGUGUCUUGAAAGUACUUCGGACAGAAAAACAAU
ACUUGGGUGUGUACAUUUGGAACAUGCGCGGCUCCGAUGGUACGUCUACCUACGCCACGUUUU
UGGUCACCUGGAAAGGGGAUGAAAAAACAAGAAACCCUACGCCCGCAGUAACUCCUCAACCAA
GAGGGGCUGAGUUUCAUAUGUGGAAUUACCACUCGCAUGUAUUUUCAGUUGGUGAUACGUUUA
GCUUGGCAAUGCAUCUUCAGUAUAAGAUACAUGAAGCGCCAUUUGAUUUGCUGUUAGAGUGGU
UGUAUGUCCCCAUCGAUCCUACAUGUCAACCAAUGCGGUUAUAUUCUACGUGUUUGUAUCAUC
CCAACGCACCCCAAUGCCUCUCUCAUAUGAAUUCCGGUUGUACAUUUACCUCGCCACAUUUAGC
CCAGCGUGUUGCAAGCACAGUGUAUCAAAAUUGUGAACAUGCAGAUAACUACACCGCAUAUUG
UCUGGGAAUAUCUCAUAUGGAGCCUAGCUUUGGUCUAAUCUUACACGACGGGGGCACCACGUU
AAAGUUUGUAGAUACACCCGAGAGUUUGUCGGGAUUAUACGUUUUUGUGGUGUAUUUUAACGG
GCAUGUUGAAGCCGUAGCAUACACUGUUGUAUCCACAGUAGAUCAUUUUGUAAACGCAAUUGA
AGAGCGUGGAUUUCCGCCAACGGCCGGUCAGCCACCGGCGACUACUAAACCCAAGGAAAUUACC
CCCGUAAACCCCGGAACGUCACCACUUCUACGAUAUGCCGCAUGGACCGGAGGGCUUGCAGCAG
UAGUACUUUUAUGUCUCGUAAUAUUUUUAAUCUGUACGGCUUGAUGAUAAUAGGCUGGAGCCU
CGGUGGCCAUGCUUCUUGCCCCUUGGGCCUCCCCCCAGCCCCUCCUCCCCUUCCUGCACCCGUAC
CCCCGUGGUCUUUGAAUAAAGUCUGAGUGGGCGGC
VZV-GE-delete-574
(SEQ ID NO: 5)
TCAAGCTTTTGGACCCTCGTACAGAAGCTAATACGACTCACTATAGGGAAATAAGAGAGAAAAGA
AGAGTAAGAAGAAATATAAGAGCCACCATGGGGACAGTTAATAAACCTGTGGTGGGGGTATTGAT
GGGGTTCGGAATTATCACGGGAACGTTGCGTATAACGAATCCGGTCAGAGCATCCGTCTTGCGATA
CGATGATTTTCACATCGATGAAGACAAACTGGATACAAACTCCGTATATGAGCCTTACTACCATTC
AGATCATGCGGAGTCTTCATGGGTAAATCGGGGAGAGTCTTCGCGAAAAGCGTACGATCATAACT
CACCTTATATATGGCCACGTAATGATTATGATGGATTTTTAGAGAACGCACACGAACACCATGGGG
TGTATAATCAGGGCCGTGGTATCGATAGCGGGGAACGGTTAATGCAACCCACACAAATGTCTGCA
CAGGAGGATCTTGGGGACGATACGGGCATCCACGTTATCCCTACGTTAAACGGCGATGACAGACA
TAAAATTGTAAATGTGGACCAACGTCAATACGGTGACGTGTTTAAAGGAGATCTTAATCCAAAAC
CCCAAGGCCAAAGACTCATTGAGGTGTCAGTGGAAGAAAATCACCCGTTTACTTTACGCGCACCG
ATTCAGCGGATTTATGGAGTCCGGTACACCGAGACTTGGAGCTTTTTGCCGTCATTAACCTGTACG
GGAGACGCAGCGCCCGCCATCCAGCATATATGTTTAAAACATACAACATGCTTTCAAGACGTGGT
GGTGGATGTGGATTGCGCGGAAAATACTAAAGAGGATCAGTTGGCCGAAATCAGTTACCGTTTTC
AAGGTAAGAAGGAAGCGGACCAACCGTGGATTGTTGTAAACACGAGCACACTGTTTGATGAACTC
GAATTAGACCCCCCCGAGATTGAACCGGGTGTCTTGAAAGTACTTCGGACAGAAAAACAATACTT
GGGTGTGTACATTTGGAACATGCGCGGCTCCGATGGTACGTCTACCTACGCCACGTTTTTGGTCAC
CTGGAAAGGGGATGAAAAAACAAGAAACCCTACGCCCGCAGTAACTCCTCAACCAAGAGGGGCT
GAGTTTCATATGTGGAATTACCACTCGCATGTATTTTCAGTTGGTGATACGTTTAGCTTGGCAATGC
ATCTTCAGTATAAGATACATGAAGCGCCATTTGATTTGCTGTTAGAGTGGTTGTATGTCCCCATCG
ATCCTACATGTCAACCAATGCGGTTATATTCTACGTGTTTGTATCATCCCAACGCACCCCAATGCCT
CTCTCATATGAATTCCGGTTGTACATTTACCTCGCCACATTTAGCCCAGCGTGTTGCAAGCACAGTG
TATCAAAATTGTGAACATGCAGATAACTACACCGCATATTGTCTGGGAATATCTCATATGGAGCCT
AGCTTTGGTCTAATCTTACACGACGGGGGCACCACGTTAAAGTTTGTAGATACACCCGAGAGTTTG
TCGGGATTATACGTTTTTGTGGTGTATTTTAACGGGCATGTTGAAGCCGTAGCATACACTGTTGTAT
CCACAGTAGATCATTTTGTAAACGCAATTGAAGAGCGTGGATTTCCGCCAACGGCCGGTCAGCCAC
CGGCGACTACTAAACCCAAGGAAATTACCCCCGTAAACCCCGGAACGTCACCACTTCTACGATAT
GCCGCATGGACCGGAGGGCTTGCAGCAGTAGTACTTTTATGTCTCGTAATATTTTTAATCTGTACG
GCTAAACGAATGAGGGTTAAAGCCTATAGGGTAGACAAGTGATGATAATAGGCTGGAGCCTCGGT
GGCCATGCTTCTTGCCCCTTGGGCCTCCCCCCAGCCCCTCCTCCCCTTCCTGCACCCGTACCCCCGT
GGTCTTTGAATAAAGTCTGAGTGGGCGGC
VZV-GE- delete-574 (mRNA)
(SEQ ID NO: 127)
UCAAGCUUUUGGACCCUCGUACAGAAGCUAAUACGACUCACUAUAGGGAAAUAAGAGAGAAAA
GAAGAGUAAGAAGAAAUAUAAGAGCCACCAUGGGGACAGUUAAUAAACCUGUGGUGGGGGUAU
UGAUGGGGUUCGGAAUUAUCACGGGAACGUUGCGUAUAACGAAUCCGGUCAGAGCAUCCGUCU
UGCGAUACGAUGAUUUUCACAUCGAUGAAGACAAACUGGAUACAAACUCCGUAUAUGAGCCUU
ACUACCAUUCAGAUCAUGCGGAGUCUUCAUGGGUAAAUCGGGGAGAGUCUUCGCGAAAAGCGU
ACGAUCAUAACUCACCUUAUAUAUGGCCACGUAAUGAUUAUGAUGGAUUUUUAGAGAACGCAC
ACGAACACCAUGGGGUGUAUAAUCAGGGCCGUGGUAUCGAUAGCGGGGAACGGUUAAUGCAAC
CCACACAAAUGUCUGCACAGGAGGAUCUUGGGGACGAUACGGGCAUCCACGUUAUCCCUACGU
UAAACGGCGAUGACAGACAUAAAAUUGUAAAUGUGGACCAACGUCAAUACGGUGACGUGUUUA
AAGGAGAUCUUAAUCCAAAACCCCAAGGCCAAAGACUCAUUGAGGUGUCAGUGGAAGAAAAUC
ACCCGUUUACUUUACGCGCACCGAUUCAGCGGAUUUAUGGAGUCCGGUACACCGAGACUUGGA
GCUUUUUGCCGUCAUUAACCUGUACGGGAGACGCAGCGCCCGCCAUCCAGCAUAUAUGUUUAA
AACAUACAACAUGCUUUCAAGACGUGGUGGUGGAUGUGGAUUGCGCGGAAAAUACUAAAGAGG
AUCAGUUGGCCGAAAUCAGUUACCGUUUUCAAGGUAAGAAGGAAGCGGACCAACCGUGGAUUG
UUGUAAACACGAGCACACUGUUUGAUGAACUCGAAUUAGACCCCCCCGAGAUUGAACCGGGUG
UCUUGAAAGUACUUCGGACAGAAAAACAAUACUUGGGUGUGUACAUUUGGAACAUGCGCGGCU
CCGAUGGUACGUCUACCUACGCCACGUUUUUGGUCACCUGGAAAGGGGAUGAAAAAACAAGAA
ACCCUACGCCCGCAGUAACUCCUCAACCAAGAGGGGCUGAGUUUCAUAUGUGGAAUUACCACUC
GCAUGUAUUUUCAGUUGGUGAUACGUUUAGCUUGGCAAUGCAUCUUCAGUAUAAGAUACAUGA
AGCGCCAUUUGAUUUGCUGUUAGAGUGGUUGUAUGUCCCCAUCGAUCCUACAUGUCAACCAAU
GCGGUUAUAUUCUACGUGUUUGUAUCAUCCCAACGCACCCCAAUGCCUCUCUCAUAUGAAUUCC
GGUUGUACAUUUACCUCGCCACAUUUAGCCCAGCGUGUUGCAAGCACAGUGUAUCAAAAUUGU
GAACAUGCAGAUAACUACACCGCAUAUUGUCUGGGAAUAUCUCAUAUGGAGCCUAGCUUUGGU
CUAAUCUUACACGACGGGGGCACCACGUUAAAGUUUGUAGAUACACCCGAGAGUUUGUCGGGA
UUAUACGUUUUUGUGGUGUAUUUUAACGGGCAUGUUGAAGCCGUAGCAUACACUGUUGUAUCC
ACAGUAGAUCAUUUUGUAAACGCAAUUGAAGAGCGUGGAUUUCCGCCAACGGCCGGUCAGCCA
CCGGCGACUACUAAACCCAAGGAAAUUACCCCCGUAAACCCCGGAACGUCACCACUUCUACGAU
AUGCCGCAUGGACCGGAGGGCUUGCAGCAGUAGUACUUUUAUGUCUCGUAAUAUUUUUAAUCU
GUACGGCUAAACGAAUGAGGGUUAAAGCCUAUAGGGUAGACAAGUGAUGAUAAUAGGCUGGAG
CCUCGGUGGCCAUGCUUCUUGCCCCUUGGGCCUCCCCCCAGCCCCUCCUCCCCUUCCUGCACCCG
UACCCCCGUGGUCUUUGAAUAAAGUCUGAGUGGGCGGC
VZV-GE-delete-574-Y569A
(SEQ ID NO: 6)
TCAAGCTTTTGGACCCTCGTACAGAAGCTAATACGACTCACTATAGGGAAATAAGAGAGAAAAGA
AGAGTAAGAAGAAATATAAGAGCCACCATGGGGACAGTTAATAAACCTGTGGTGGGGGTATTGAT
GGGGTTCGGAATTATCACGGGAACGTTGCGTATAACGAATCCGGTCAGAGCATCCGTCTTGCGATA
CGATGATTTTCACATCGATGAAGACAAACTGGATACAAACTCCGTATATGAGCCTTACTACCATTC
AGATCATGCGGAGTCTTCATGGGTAAATCGGGGAGAGTCTTCGCGAAAAGCGTACGATCATAACT
CACCTTATATATGGCCACGTAATGATTATGATGGATTTTTAGAGAACGCACACGAACACCATGGGG
TGTATAATCAGGGCCGTGGTATCGATAGCGGGGAACGGTTAATGCAACCCACACAAATGTCTGCA
CAGGAGGATCTTGGGGACGATACGGGCATCCACGTTATCCCTACGTTAAACGGCGATGACAGACA
TAAAATTGTAAATGTGGACCAACGTCAATACGGTGACGTGTTTAAAGGAGATCTTAATCCAAAAC
CCCAAGGCCAAAGACTCATTGAGGTGTCAGTGGAAGAAAATCACCCGTTTACTTTACGCGCACCG
ATTCAGCGGATTTATGGAGTCCGGTACACCGAGACTTGGAGCTTTTTGCCGTCATTAACCTGTACG
GGAGACGCAGCGCCCGCCATCCAGCATATATGTTTAAAACATACAACATGCTTTCAAGACGTGGT
GGTGGATGTGGATTGCGCGGAAAATACTAAAGAGGATCAGTTGGCCGAAATCAGTTACCGTTTTC
AAGGTAAGAAGGAAGCGGACCAACCGTGGATTGTTGTAAACACGAGCACACTGTTTGATGAACTC
GAATTAGACCCCCCCGAGATTGAACCGGGTGTCTTGAAAGTACTTCGGACAGAAAAACAATACTT
GGGTGTGTACATTTGGAACATGCGCGGCTCCGATGGTACGTCTACCTACGCCACGTTTTTGGTCAC
CTGGAAAGGGGATGAAAAAACAAGAAACCCTACGCCCGCAGTAACTCCTCAACCAAGAGGGGCT
GAGTTTCATATGTGGAATTACCACTCGCATGTATTTTCAGTTGGTGATACGTTTAGCTTGGCAATGC
ATCTTCAGTATAAGATACATGAAGCGCCATTTGATTTGCTGTTAGAGTGGTTGTATGTCCCCATCG
ATCCTACATGTCAACCAATGCGGTTATATTCTACGTGTTTGTATCATCCCAACGCACCCCAATGCCT
CTCTCATATGAATTCCGGTTGTACATTTACCTCGCCACATTTAGCCCAGCGTGTTGCAAGCACAGTG
TATCAAAATTGTGAACATGCAGATAACTACACCGCATATTGTCTGGGAATATCTCATATGGAGCCT
AGCTTTGGTCTAATCTTACACGACGGGGGCACCACGTTAAAGTTTGTAGATACACCCGAGAGTTTG
TCGGGATTATACGTTTTTGTGGTGTATTTTAACGGGCATGTTGAAGCCGTAGCATACACTGTTGTAT
CCACAGTAGATCATTTTGTAAACGCAATTGAAGAGCGTGGATTTCCGCCAACGGCCGGTCAGCCAC
CGGCGACTACTAAACCCAAGGAAATTACCCCCGTAAACCCCGGAACGTCACCACTTCTACGATAT
GCCGCATGGACCGGAGGGCTTGCAGCAGTAGTACTTTTATGTCTCGTAATATTTTTAATCTGTACG
GCTAAACGAATGAGGGTTAAAGCCGCCAGGGTAGACAAGTGATGATAATAGGCTGGAGCCTCGGT
GGCCATGCTTCTTGCCCCTTGGGCCTCCCCCCAGCCCCTCCTCCCCTTCCTGCACCCGTACCCCCGT
GGTCTTTGAATAAAGTCTGAGTGGGCGGC
VZV-GE-delete-574-Y569A (mRNA)
(SEQ ID NO: 128)
UCAAGCUUUUGGACCCUCGUACAGAAGCUAAUACGACUCACUAUAGGGAAAUAAGAGAGAAAA
GAAGAGUAAGAAGAAAUAUAAGAGCCACCAUGGGGACAGUUAAUAAACCUGUGGUGGGGGUAU
UGAUGGGGUUCGGAAUUAUCACGGGAACGUUGCGUAUAACGAAUCCGGUCAGAGCAUCCGUCU
UGCGAUACGAUGAUUUUCACAUCGAUGAAGACAAACUGGAUACAAACUCCGUAUAUGAGCCUU
ACUACCAUUCAGAUCAUGCGGAGUCUUCAUGGGUAAAUCGGGGAGAGUCUUCGCGAAAAGCGU
ACGAUCAUAACUCACCUUAUAUAUGGCCACGUAAUGAUUAUGAUGGAUUUUUAGAGAACGCAC
ACGAACACCAUGGGGUGUAUAAUCAGGGCCGUGGUAUCGAUAGCGGGGAACGGUUAAUGCAAC
CCACACAAAUGUCUGCACAGGAGGAUCUUGGGGACGAUACGGGCAUCCACGUUAUCCCUACGU
UAAACGGCGAUGACAGACAUAAAAUUGUAAAUGUGGACCAACGUCAAUACGGUGACGUGUUUA
AAGGAGAUCUUAAUCCAAAACCCCAAGGCCAAAGACUCAUUGAGGUGUCAGUGGAAGAAAAUC
ACCCGUUUACUUUACGCGCACCGAUUCAGCGGAUUUAUGGAGUCCGGUACACCGAGACUUGGA
GCUUUUUGCCGUCAUUAACCUGUACGGGAGACGCAGCGCCCGCCAUCCAGCAUAUAUGUUUAA
AACAUACAACAUGCUUUCAAGACGUGGUGGUGGAUGUGGAUUGCGCGGAAAAUACUAAAGAGG
AUCAGUUGGCCGAAAUCAGUUACCGUUUUCAAGGUAAGAAGGAAGCGGACCAACCGUGGAUUG
UUGUAAACACGAGCACACUGUUUGAUGAACUCGAAUUAGACCCCCCCGAGAUUGAACCGGGUG
UCUUGAAAGUACUUCGGACAGAAAAACAAUACUUGGGUGUGUACAUUUGGAACAUGCGCGGCU
CCGAUGGUACGUCUACCUACGCCACGUUUUUGGUCACCUGGAAAGGGGAUGAAAAAACAAGAA
ACCCUACGCCCGCAGUAACUCCUCAACCAAGAGGGGCUGAGUUUCAUAUGUGGAAUUACCACUC
GCAUGUAUUUUCAGUUGGUGAUACGUUUAGCUUGGCAAUGCAUCUUCAGUAUAAGAUACAUGA
AGCGCCAUUUGAUUUGCUGUUAGAGUGGUUGUAUGUCCCCAUCGAUCCUACAUGUCAACCAAU
GCGGUUAUAUUCUACGUGUUUGUAUCAUCCCAACGCACCCCAAUGCCUCUCUCAUAUGAAUUCC
GGUUGUACAUUUACCUCGCCACAUUUAGCCCAGCGUGUUGCAAGCACAGUGUAUCAAAAUUGU
GAACAUGCAGAUAACUACACCGCAUAUUGUCUGGGAAUAUCUCAUAUGGAGCCUAGCUUUGGU
CUAAUCUUACACGACGGGGGCACCACGUUAAAGUUUGUAGAUACACCCGAGAGUUUGUCGGGA
UUAUACGUUUUUGUGGUGUAUUUUAACGGGCAUGUUGAAGCCGUAGCAUACACUGUUGUAUCC
ACAGUAGAUCAUUUUGUAAACGCAAUUGAAGAGCGUGGAUUUCCGCCAACGGCCGGUCAGCCA
CCGGCGACUACUAAACCCAAGGAAAUUACCCCCGUAAACCCCGGAACGUCACCACUUCUACGAU
AUGCCGCAUGGACCGGAGGGCUUGCAGCAGUAGUACUUUUAUGUCUCGUAAUAUUUUUAAUCU
GUACGGCUAAACGAAUGAGGGUUAAAGCCGCCAGGGUAGACAAGUGAUGAUAAUAGGCUGGAG
CCUCGGUGGCCAUGCUUCUUGCCCCUUGGGCCUCCCCCCAGCCCCUCCUCCCCUUCCUGCACCCG
UACCCCCGUGGUCUUUGAAUAAAGUCUGAGUGGGCGGC
VZV-gE-full length-AEAADA (SEQ ID NO: 58)
(SEQ ID NO: 7)
TCAAGCTTTTGGACCCTCGTACAGAAGCTAATACGACTCACTATAGGGAAATAAGAGAGAAAAGA
AGAGTAAGAAGAAATATAAGAGCCACCATGGGGACAGTTAATAAACCTGTGGTGGGGGTATTGAT
GGGGTTCGGAATTATCACGGGAACGTTGCGTATAACGAATCCGGTCAGAGCATCCGTCTTGCGATA
CGATGATTTTCACATCGATGAAGACAAACTGGATACAAACTCCGTATATGAGCCTTACTACCATTC
AGATCATGCGGAGTCTTCATGGGTAAATCGGGGAGAGTCTTCGCGAAAAGCGTACGATCATAACT
CACCTTATATATGGCCACGTAATGATTATGATGGATTTTTAGAGAACGCACACGAACACCATGGGG
TGTATAATCAGGGCCGTGGTATCGATAGCGGGGAACGGTTAATGCAACCCACACAAATGTCTGCA
CAGGAGGATCTTGGGGACGATACGGGCATCCACGTTATCCCTACGTTAAACGGCGATGACAGACA
TAAAATTGTAAATGTGGACCAACGTCAATACGGTGACGTGTTTAAAGGAGATCTTAATCCAAAAC
CCCAAGGCCAAAGACTCATTGAGGTGTCAGTGGAAGAAAATCACCCGTTTACTTTACGCGCACCG
ATTCAGCGGATTTATGGAGTCCGGTACACCGAGACTTGGAGCTTTTTGCCGTCATTAACCTGTACG
GGAGACGCAGCGCCCGCCATCCAGCATATATGTTTAAAACATACAACATGCTTTCAAGACGTGGT
GGTGGATGTGGATTGCGCGGAAAATACTAAAGAGGATCAGTTGGCCGAAATCAGTTACCGTTTTC
AAGGTAAGAAGGAAGCGGACCAACCGTGGATTGTTGTAAACACGAGCACACTGTTTGATGAACTC
GAATTAGACCCCCCCGAGATTGAACCGGGTGTCTTGAAAGTACTTCGGACAGAAAAACAATACTT
GGGTGTGTACATTTGGAACATGCGCGGCTCCGATGGTACGTCTACCTACGCCACGTTTTTGGTCAC
CTGGAAAGGGGATGAAAAAACAAGAAACCCTACGCCCGCAGTAACTCCTCAACCAAGAGGGGCT
GAGTTTCATATGTGGAATTACCACTCGCATGTATTTTCAGTTGGTGATACGTTTAGCTTGGCAATGC
ATCTTCAGTATAAGATACATGAAGCGCCATTTGATTTGCTGTTAGAGTGGTTGTATGTCCCCATCG
ATCCTACATGTCAACCAATGCGGTTATATTCTACGTGTTTGTATCATCCCAACGCACCCCAATGCCT
CTCTCATATGAATTCCGGTTGTACATTTACCTCGCCACATTTAGCCCAGCGTGTTGCAAGCACAGTG
TATCAAAATTGTGAACATGCAGATAACTACACCGCATATTGTCTGGGAATATCTCATATGGAGCCT
AGCTTTGGTCTAATCTTACACGACGGGGGCACCACGTTAAAGTTTGTAGATACACCCGAGAGTTTG
TCGGGATTATACGTTTTTGTGGTGTATTTTAACGGGCATGTTGAAGCCGTAGCATACACTGTTGTAT
CCACAGTAGATCATTTTGTAAACGCAATTGAAGAGCGTGGATTTCCGCCAACGGCCGGTCAGCCAC
CGGCGACTACTAAACCCAAGGAAATTACCCCCGTAAACCCCGGAACGTCACCACTTCTACGATAT
GCCGCATGGACCGGAGGGCTTGCAGCAGTAGTACTTTTATGTCTCGTAATATTTTTAATCTGTACG
GCTAAACGAATGAGGGTTAAAGCCTATAGGGTAGACAAGTCCCCGTATAACCAAAGCATGTATTA
CGCTGGCCTTCCAGTGGACGATTTCGAGGACGCCGAAGCCGCCGATGCCGAAGAAGAGTTTGGTA
ACGCGATTGGAGGGAGTCACGGGGGTTCGAGTTACACGGTGTATATAGATAAGACCCGGTGATGA
TAATAGGCTGGAGCCTCGGTGGCCATGCTTCTTGCCCCTTGGGCCTCCCCCCAGCCCCTCCTCCCCT
TCCTGCACCCGTACCCCCGTGGTCTTTGAATAAAGTCTGAGTGGGCGGC
VZV-gE-full length-AEAADA (SEQ ID NO: 58) (mRNA)
(SEQ ID NO: 129)
UCAAGCUUUUGGACCCUCGUACAGAAGCUAAUACGACUCACUAUAGGGAAAUAAGAGAGAAAA
GAAGAGUAAGAAGAAAUAUAAGAGCCACCAUGGGGACAGUUAAUAAACCUGUGGUGGGGGUAU
UGAUGGGGUUCGGAAUUAUCACGGGAACGUUGCGUAUAACGAAUCCGGUCAGAGCAUCCGUCU
UGCGAUACGAUGAUUUUCACAUCGAUGAAGACAAACUGGAUACAAACUCCGUAUAUGAGCCUU
ACUACCAUUCAGAUCAUGCGGAGUCUUCAUGGGUAAAUCGGGGAGAGUCUUCGCGAAAAGCGU
ACGAUCAUAACUCACCUUAUAUAUGGCCACGUAAUGAUUAUGAUGGAUUUUUAGAGAACGCAC
ACGAACACCAUGGGGUGUAUAAUCAGGGCCGUGGUAUCGAUAGCGGGGAACGGUUAAUGCAAC
CCACACAAAUGUCUGCACAGGAGGAUCUUGGGGACGAUACGGGCAUCCACGUUAUCCCUACGU
UAAACGGCGAUGACAGACAUAAAAUUGUAAAUGUGGACCAACGUCAAUACGGUGACGUGUUUA
AAGGAGAUCUUAAUCCAAAACCCCAAGGCCAAAGACUCAUUGAGGUGUCAGUGGAAGAAAAUC
ACCCGUUUACUUUACGCGCACCGAUUCAGCGGAUUUAUGGAGUCCGGUACACCGAGACUUGGA
GCUUUUUGCCGUCAUUAACCUGUACGGGAGACGCAGCGCCCGCCAUCCAGCAUAUAUGUUUAA
AACAUACAACAUGCUUUCAAGACGUGGUGGUGGAUGUGGAUUGCGCGGAAAAUACUAAAGAGG
AUCAGUUGGCCGAAAUCAGUUACCGUUUUCAAGGUAAGAAGGAAGCGGACCAACCGUGGAUUG
UUGUAAACACGAGCACACUGUUUGAUGAACUCGAAUUAGACCCCCCCGAGAUUGAACCGGGUG
UCUUGAAAGUACUUCGGACAGAAAAACAAUACUUGGGUGUGUACAUUUGGAACAUGCGCGGCU
CCGAUGGUACGUCUACCUACGCCACGUUUUUGGUCACCUGGAAAGGGGAUGAAAAAACAAGAA
ACCCUACGCCCGCAGUAACUCCUCAACCAAGAGGGGCUGAGUUUCAUAUGUGGAAUUACCACUC
GCAUGUAUUUUCAGUUGGUGAUACGUUUAGCUUGGCAAUGCAUCUUCAGUAUAAGAUACAUGA
AGCGCCAUUUGAUUUGCUGUUAGAGUGGUUGUAUGUCCCCAUCGAUCCUACAUGUCAACCAAU
GCGGUUAUAUUCUACGUGUUUGUAUCAUCCCAACGCACCCCAAUGCCUCUCUCAUAUGAAUUCC
GGUUGUACAUUUACCUCGCCACAUUUAGCCCAGCGUGUUGCAAGCACAGUGUAUCAAAAUUGU
GAACAUGCAGAUAACUACACCGCAUAUUGUCUGGGAAUAUCUCAUAUGGAGCCUAGCUUUGGU
CUAAUCUUACACGACGGGGGCACCACGUUAAAGUUUGUAGAUACACCCGAGAGUUUGUCGGGA
UUAUACGUUUUUGUGGUGUAUUUUAACGGGCAUGUUGAAGCCGUAGCAUACACUGUUGUAUCC
ACAGUAGAUCAUUUUGUAAACGCAAUUGAAGAGCGUGGAUUUCCGCCAACGGCCGGUCAGCCA
CCGGCGACUACUAAACCCAAGGAAAUUACCCCCGUAAACCCCGGAACGUCACCACUUCUACGAU
AUGCCGCAUGGACCGGAGGGCUUGCAGCAGUAGUACUUUUAUGUCUCGUAAUAUUUUUAAUCU
GUACGGCUAAACGAAUGAGGGUUAAAGCCUAUAGGGUAGACAAGUCCCCGUAUAACCAAAGCA
UGUAUUACGCUGGCCUUCCAGUGGACGAUUUCGAGGACGCCGAAGCCGCCGAUGCCGAAGAAG
AGUUUGGUAACGCGAUUGGAGGGAGUCACGGGGGUUCGAGUUACACGGUGUAUAUAGAUAAGA
CCCGGUGAUGAUAAUAGGCUGGAGCCUCGGUGGCCAUGCUUCUUGCCCCUUGGGCCUCCCCCCA
GCCCCUCCUCCCCUUCCUGCACCCGUACCCCCGUGGUCUUUGAAUAAAGUCUGAGUGGGCGGC
VZV-GE-full-AEAADA (SEQ ID NO: 58)-Y582G
(SEQ ID NO: 8)
TCAAGCTTTTGGACCCTCGTACAGAAGCTAATACGACTCACTATAGGGAAATAAGAGAGAAAAGA
AGAGTAAGAAGAAATATAAGAGCCACCATGGGGACAGTTAATAAACCTGTGGTGGGGGTATTGAT
GGGGTTCGGAATTATCACGGGAACGTTGCGTATAACGAATCCGGTCAGAGCATCCGTCTTGCGATA
CGATGATTTTCACATCGATGAAGACAAACTGGATACAAACTCCGTATATGAGCCTTACTACCATTC
AGATCATGCGGAGTCTTCATGGGTAAATCGGGGAGAGTCTTCGCGAAAAGCGTACGATCATAACT
CACCTTATATATGGCCACGTAATGATTATGATGGATTTTTAGAGAACGCACACGAACACCATGGGG
TGTATAATCAGGGCCGTGGTATCGATAGCGGGGAACGGTTAATGCAACCCACACAAATGTCTGCA
CAGGAGGATCTTGGGGACGATACGGGCATCCACGTTATCCCTACGTTAAACGGCGATGACAGACA
TAAAATTGTAAATGTGGACCAACGTCAATACGGTGACGTGTTTAAAGGAGATCTTAATCCAAAAC
CCCAAGGCCAAAGACTCATTGAGGTGTCAGTGGAAGAAAATCACCCGTTTACTTTACGCGCACCG
ATTCAGCGGATTTATGGAGTCCGGTACACCGAGACTTGGAGCTTTTTGCCGTCATTAACCTGTACG
GGAGACGCAGCGCCCGCCATCCAGCATATATGTTTAAAACATACAACATGCTTTCAAGACGTGGT
GGTGGATGTGGATTGCGCGGAAAATACTAAAGAGGATCAGTTGGCCGAAATCAGTTACCGTTTTC
AAGGTAAGAAGGAAGCGGACCAACCGTGGATTGTTGTAAACACGAGCACACTGTTTGATGAACTC
GAATTAGACCCCCCCGAGATTGAACCGGGTGTCTTGAAAGTACTTCGGACAGAAAAACAATACTT
GGGTGTGTACATTTGGAACATGCGCGGCTCCGATGGTACGTCTACCTACGCCACGTTTTTGGTCAC
CTGGAAAGGGGATGAAAAAACAAGAAACCCTACGCCCGCAGTAACTCCTCAACCAAGAGGGGCT
GAGTTTCATATGTGGAATTACCACTCGCATGTATTTTCAGTTGGTGATACGTTTAGCTTGGCAATGC
ATCTTCAGTATAAGATACATGAAGCGCCATTTGATTTGCTGTTAGAGTGGTTGTATGTCCCCATCG
ATCCTACATGTCAACCAATGCGGTTATATTCTACGTGTTTGTATCATCCCAACGCACCCCAATGCCT
CTCTCATATGAATTCCGGTTGTACATTTACCTCGCCACATTTAGCCCAGCGTGTTGCAAGCACAGTG
TATCAAAATTGTGAACATGCAGATAACTACACCGCATATTGTCTGGGAATATCTCATATGGAGCCT
AGCTTTGGTCTAATCTTACACGACGGGGGCACCACGTTAAAGTTTGTAGATACACCCGAGAGTTTG
TCGGGATTATACGTTTTTGTGGTGTATTTTAACGGGCATGTTGAAGCCGTAGCATACACTGTTGTAT
CCACAGTAGATCATTTTGTAAACGCAATTGAAGAGCGTGGATTTCCGCCAACGGCCGGTCAGCCAC
CGGCGACTACTAAACCCAAGGAAATTACCCCCGTAAACCCCGGAACGTCACCACTTCTACGATAT
GCCGCATGGACCGGAGGGCTTGCAGCAGTAGTACTTTTATGTCTCGTAATATTTTTAATCTGTACG
GCTAAACGAATGAGGGTTAAAGCCTATAGGGTAGACAAGTCCCCGTATAACCAAAGCATGTATGG
CGCTGGCCTTCCAGTGGACGATTTCGAGGACGCCGAAGCCGCCGATGCCGAAGAAGAGTTTGGTA
ACGCGATTGGAGGGAGTCACGGGGGTTCGAGTTACACGGTGTATATAGATAAGACCCGGTGATGA
TAATAGGCTGGAGCCTCGGTGGCCATGCTTCTTGCCCCTTGGGCCTCCCCCCAGCCCCTCCTCCCCT
TCCTGCACCCGTACCCCCGTGGTCTTTGAATAAAGTCTGAGTGGGCGGC
VZV-GE-full-AEAADA (SEQ ID NO: 58)-Y582G (mRNA)
(SEQ ID NO: 130)
UCAAGCUUUUGGACCCUCGUACAGAAGCUAAUACGACUCACUAUAGGGAAAUAAGAGAGAAAA
GAAGAGUAAGAAGAAAUAUAAGAGCCACCAUGGGGACAGUUAAUAAACCUGUGGUGGGGGUAU
UGAUGGGGUUCGGAAUUAUCACGGGAACGUUGCGUAUAACGAAUCCGGUCAGAGCAUCCGUCU
UGCGAUACGAUGAUUUUCACAUCGAUGAAGACAAACUGGAUACAAACUCCGUAUAUGAGCCUU
ACUACCAUUCAGAUCAUGCGGAGUCUUCAUGGGUAAAUCGGGGAGAGUCUUCGCGAAAAGCGU
ACGAUCAUAACUCACCUUAUAUAUGGCCACGUAAUGAUUAUGAUGGAUUUUUAGAGAACGCAC
ACGAACACCAUGGGGUGUAUAAUCAGGGCCGUGGUAUCGAUAGCGGGGAACGGUUAAUGCAAC
CCACACAAAUGUCUGCACAGGAGGAUCUUGGGGACGAUACGGGCAUCCACGUUAUCCCUACGU
UAAACGGCGAUGACAGACAUAAAAUUGUAAAUGUGGACCAACGUCAAUACGGUGACGUGUUUA
AAGGAGAUCUUAAUCCAAAACCCCAAGGCCAAAGACUCAUUGAGGUGUCAGUGGAAGAAAAUC
ACCCGUUUACUUUACGCGCACCGAUUCAGCGGAUUUAUGGAGUCCGGUACACCGAGACUUGGA
GCUUUUUGCCGUCAUUAACCUGUACGGGAGACGCAGCGCCCGCCAUCCAGCAUAUAUGUUUAA
AACAUACAACAUGCUUUCAAGACGUGGUGGUGGAUGUGGAUUGCGCGGAAAAUACUAAAGAGG
AUCAGUUGGCCGAAAUCAGUUACCGUUUUCAAGGUAAGAAGGAAGCGGACCAACCGUGGAUUG
UUGUAAACACGAGCACACUGUUUGAUGAACUCGAAUUAGACCCCCCCGAGAUUGAACCGGGUG
UCUUGAAAGUACUUCGGACAGAAAAACAAUACUUGGGUGUGUACAUUUGGAACAUGCGCGGCU
CCGAUGGUACGUCUACCUACGCCACGUUUUUGGUCACCUGGAAAGGGGAUGAAAAAACAAGAA
ACCCUACGCCCGCAGUAACUCCUCAACCAAGAGGGGCUGAGUUUCAUAUGUGGAAUUACCACUC
GCAUGUAUUUUCAGUUGGUGAUACGUUUAGCUUGGCAAUGCAUCUUCAGUAUAAGAUACAUGA
AGCGCCAUUUGAUUUGCUGUUAGAGUGGUUGUAUGUCCCCAUCGAUCCUACAUGUCAACCAAU
GCGGUUAUAUUCUACGUGUUUGUAUCAUCCCAACGCACCCCAAUGCCUCUCUCAUAUGAAUUCC
GGUUGUACAUUUACCUCGCCACAUUUAGCCCAGCGUGUUGCAAGCACAGUGUAUCAAAAUUGU
GAACAUGCAGAUAACUACACCGCAUAUUGUCUGGGAAUAUCUCAUAUGGAGCCUAGCUUUGGU
CUAAUCUUACACGACGGGGGCACCACGUUAAAGUUUGUAGAUACACCCGAGAGUUUGUCGGGA
UUAUACGUUUUUGUGGUGUAUUUUAACGGGCAUGUUGAAGCCGUAGCAUACACUGUUGUAUCC
ACAGUAGAUCAUUUUGUAAACGCAAUUGAAGAGCGUGGAUUUCCGCCAACGGCCGGUCAGCCA
CCGGCGACUACUAAACCCAAGGAAAUUACCCCCGUAAACCCCGGAACGUCACCACUUCUACGAU
AUGCCGCAUGGACCGGAGGGCUUGCAGCAGUAGUACUUUUAUGUCUCGUAAUAUUUUUAAUCU
GUACGGCUAAACGAAUGAGGGUUAAAGCCUAUAGGGUAGACAAGUCCCCGUAUAACCAAAGCA
UGUAUGGCGCUGGCCUUCCAGUGGACGAUUUCGAGGACGCCGAAGCCGCCGAUGCCGAAGAAG
AGUUUGGUAACGCGAUUGGAGGGAGUCACGGGGGUUCGAGUUACACGGUGUAUAUAGAUAAGA
CCCGGUGAUGAUAAUAGGCUGGAGCCUCGGUGGCCAUGCUUCUUGCCCCUUGGGCCUCCCCCCA
GCCCCUCCUCCCCUUCCUGCACCCGUACCCCCGUGGUCUUUGAAUAAAGUCUGAGUGGGCGGC
VZV_gE_Oka_hIgkappa
(SEQ ID NO: 41)
TCAAGCTTTTGGACCCTCGTACAGAAGCTAATACGACTCACTATAGGGAAATAAGAGAGAAAAGA
AGAGTAAGAAGAAATATAAGAGCCACCATGGAGACTCCCGCTCAGCTACTGTTCCTCCTGCTCCTT
TGGCTGCCTGATACTACAGGCTCTGTTTTGCGGTACGACGACTTTCACATCGATGAGGACAAGCTC
GACACTAATAGCGTGTATGAGCCCTACTACCATTCAGATCACGCCGAGTCCTCTTGGGTGAACAGG
GGTGAAAGTTCTAGGAAAGCCTATGATCACAACAGCCCTTATATTTGGCCACGGAATGATTACGAC
GGATTTCTCGAAAATGCCCACGAGCATCACGGAGTGTACAACCAGGGCCGTGGAATCGACTCTGG
GGAGAGATTGATGCAACCTACACAGATGAGCGCCCAGGAAGATCTCGGGGATGATACAGGAATTC
ACGTTATCCCTACATTAAACGGAGATGACCGCCACAAAATCGTCAATGTCGATCAAAGACAGTAT
GGAGATGTGTTCAAAGGCGATCTCAACCCTAAGCCGCAGGGCCAGAGACTCATTGAGGTGTCTGT
CGAAGAGAACCACCCTTTCACTCTGCGCGCTCCCATTCAGAGAATCTATGGAGTTCGCTATACGGA
GACTTGGTCATTCCTTCCTTCCCTGACATGCACCGGAGACGCCGCCCCTGCCATTCAGCACATATG
CCTGAAACATACCACCTGTTTCCAGGATGTGGTGGTTGATGTTGATTGTGCTGAAAATACCAAGGA
AGACCAACTGGCCGAGATTAGTTACCGGTTCCAAGGGAAAAAGGAAGCCGACCAGCCATGGATTG
TGGTTAATACAAGCACTCTGTTCGATGAGCTCGAGCTGGATCCCCCCGAGATAGAACCCGGAGTTC
TGAAAGTGCTCCGGACAGAAAAACAATATCTGGGAGTCTACATATGGAACATGCGCGGTTCCGAT
GGGACCTCCACTTATGCAACCTTTCTCGTCACGTGGAAGGGAGATGAGAAAACTAGGAATCCCAC
ACCCGCTGTCACACCACAGCCAAGAGGGGCTGAGTTCCATATGTGGAACTATCATAGTCACGTGTT
TAGTGTCGGAGATACGTTTTCATTGGCTATGCATCTCCAGTACAAGATTCATGAGGCTCCCTTCGAT
CTGTTGCTTGAGTGGTTGTACGTCCCGATTGACCCGACCTGCCAGCCCATGCGACTGTACAGCACC
TGTCTCTACCATCCAAACGCTCCGCAATGTCTGAGCCACATGAACTCTGGGTGTACTTTCACCAGT
CCCCACCTCGCCCAGCGGGTGGCCTCTACTGTTTACCAGAACTGTGAGCACGCCGACAACTACACC
GCATACTGCCTCGGTATTTCTCACATGGAACCCTCCTTCGGACTCATCCTGCACGATGGGGGCACT
ACCCTGAAGTTCGTTGATACGCCAGAATCTCTGTCTGGGCTCTATGTTTTCGTGGTCTACTTCAATG
GCCATGTCGAGGCCGTGGCCTATACTGTCGTTTCTACCGTGGATCATTTTGTGAACGCCATCGAAG
AACGGGGATTCCCCCCTACGGCAGGCCAGCCGCCTGCAACCACCAAGCCCAAGGAAATAACACCA
GTGAACCCTGGCACCTCACCTCTCCTAAGATATGCCGCGTGGACAGGGGGACTGGCGGCAGTGGT
GCTCCTCTGTCTCGTGATCTTTCTGATCTGTACAGCCAAGAGGATGAGGGTCAAGGCTTATAGAGT
GGACAAGTCCCCCTACAATCAGTCAATGTACTACGCCGGCCTTCCCGTTGATGATTTTGAGGATTC
CGAGTCCACAGATACTGAGGAAGAGTTCGGTAACGCTATAGGCGGCTCTCACGGGGGTTCAAGCT
ACACGGTTTACATTGACAAGACACGCTGATAATAGGCTGGAGCCTCGGTGGCCATGCTTCTTGCCC
CTTGGGCCTCCCCCCAGCCCCTCCTCCCCTTCCTGCACCCGTACCCCCGTGGTCTTTGAATAAAGTC
TGAGTGGGCGGC
VZV_gE_Oka_hIgkappa (mRNA)
(SEQ ID NO: 131)
UCAAGCUUUUGGACCCUCGUACAGAAGCUAAUACGACUCACUAUAGGGAAAUAAGAGAGAAAA
GAAGAGUAAGAAGAAAUAUAAGAGCCACCAUGGAGACUCCCGCUCAGCUACUGUUCCUCCUGC
UCCUUUGGCUGCCUGAUACUACAGGCUCUGUUUUGCGGUACGACGACUUUCACAUCGAUGAGG
ACAAGCUCGACACUAAUAGCGUGUAUGAGCCCUACUACCAUUCAGAUCACGCCGAGUCCUCUUG
GGUGAACAGGGGUGAAAGUUCUAGGAAAGCCUAUGAUCACAACAGCCCUUAUAUUUGGCCACG
GAAUGAUUACGACGGAUUUCUCGAAAAUGCCCACGAGCAUCACGGAGUGUACAACCAGGGCCG
UGGAAUCGACUCUGGGGAGAGAUUGAUGCAACCUACACAGAUGAGCGCCCAGGAAGAUCUCGG
GGAUGAUACAGGAAUUCACGUUAUCCCUACAUUAAACGGAGAUGACCGCCACAAAAUCGUCAA
UGUCGAUCAAAGACAGUAUGGAGAUGUGUUCAAAGGCGAUCUCAACCCUAAGCCGCAGGGCCA
GAGACUCAUUGAGGUGUCUGUCGAAGAGAACCACCCUUUCACUCUGCGCGCUCCCAUUCAGAG
AAUCUAUGGAGUUCGCUAUACGGAGACUUGGUCAUUCCUUCCUUCCCUGACAUGCACCGGAGA
CGCCGCCCCUGCCAUUCAGCACAUAUGCCUGAAACAUACCACCUGUUUCCAGGAUGUGGUGGUU
GAUGUUGAUUGUGCUGAAAAUACCAAGGAAGACCAACUGGCCGAGAUUAGUUACCGGUUCCAA
GGGAAAAAGGAAGCCGACCAGCCAUGGAUUGUGGUUAAUACAAGCACUCUGUUCGAUGAGCUC
GAGCUGGAUCCCCCCGAGAUAGAACCCGGAGUUCUGAAAGUGCUCCGGACAGAAAAACAAUAU
CUGGGAGUCUACAUAUGGAACAUGCGCGGUUCCGAUGGGACCUCCACUUAUGCAACCUUUCUC
GUCACGUGGAAGGGAGAUGAGAAAACUAGGAAUCCCACACCCGCUGUCACACCACAGCCAAGA
GGGGCUGAGUUCCAUAUGUGGAACUAUCAUAGUCACGUGUUUAGUGUCGGAGAUACGUUUUCA
UUGGCUAUGCAUCUCCAGUACAAGAUUCAUGAGGCUCCCUUCGAUCUGUUGCUUGAGUGGUUG
UACGUCCCGAUUGACCCGACCUGCCAGCCCAUGCGACUGUACAGCACCUGUCUCUACCAUCCAA
ACGCUCCGCAAUGUCUGAGCCACAUGAACUCUGGGUGUACUUUCACCAGUCCCCACCUCGCCCA
GCGGGUGGCCUCUACUGUUUACCAGAACUGUGAGCACGCCGACAACUACACCGCAUACUGCCUC
GGUAUUUCUCACAUGGAACCCUCCUUCGGACUCAUCCUGCACGAUGGGGGCACUACCCUGAAGU
UCGUUGAUACGCCAGAAUCUCUGUCUGGGCUCUAUGUUUUCGUGGUCUACUUCAAUGGCCAUG
UCGAGGCCGUGGCCUAUACUGUCGUUUCUACCGUGGAUCAUUUUGUGAACGCCAUCGAAGAAC
GGGGAUUCCCCCCUACGGCAGGCCAGCCGCCUGCAACCACCAAGCCCAAGGAAAUAACACCAGU
GAACCCUGGCACCUCACCUCUCCUAAGAUAUGCCGCGUGGACAGGGGGACUGGCGGCAGUGGU
GCUCCUCUGUCUCGUGAUCUUUCUGAUCUGUACAGCCAAGAGGAUGAGGGUCAAGGCUUAUAG
AGUGGACAAGUCCCCCUACAAUCAGUCAAUGUACUACGCCGGCCUUCCCGUUGAUGAUUUUGA
GGAUUCCGAGUCCACAGAUACUGAGGAAGAGUUCGGUAACGCUAUAGGCGGCUCUCACGGGGG
UUCAAGCUACACGGUUUACAUUGACAAGACACGCUGAUAAUAGGCUGGAGCCUCGGUGGCCAU
GCUUCUUGCCCCUUGGGCCUCCCCCCAGCCCCUCCUCCCCUUCCUGCACCCGUACCCCCGUGGUC
UUUGAAUAAAGUCUGAGUGGGCGGC

TABLE 2
Sequences of Variant VZV gE Constructs

| | Sequence, NT | | | mRNA Sequence |
|---|---|---|---|---|
| | (5′UTR, ORF, | | | (assumes T100 |
| mRNA | 3′UTR) | ORF Sequence, AA | ORF Sequence, NT | tail) |
| Name(s) | SEQ ID NO: 9 | SEQ ID NO: 10 | SEQ ID NO: 11 | SEQ ID NO: 12 |
| VZV_gE, | TCAAGCTTTTGG | MGTVNKPVVGVLMGFGI | ATGGGGACAGTGAATAA | G*GGGAAATAAG |
| Oka | ACCCTCGTACAG | ITGTLRITNPVRASVLR | GCCGGTTGTGGGCGTGC | AGAGAAAAGAAG |
| | AAGCTAATACGA | YDDFHIDEDKLDTNSVY | TTATGGGCTTTGGGATT | AGTAAGAAGAAA |
| | CTCACTATAGGG | EPYYHSDHAESSWVNRG | ATTACCGGTACATTACG | TATAAGAGCCAC |
| | AAATAAGAGAGA | ESSRKAYDHNSPYIWPR | AATTACCAATCCAGTGC | CATGGGGACAGT |
| | AAAGAAGAGTAA | NDYDGFLENAHEHHGVY | GCGCCAGTGTGCTGCGT | GAATAAGCCGGT |
| | GAAGAAATATAA | NQGRGIDSGERLMQPTQ | TACGACGACTTTCACAT | TGTGGGCGTGCT |
| | GAGCCACCATGG | MSAQEDLGDDTGIHVIP | TGACGAGGATAAGCTGG | TATGGGCTTTGG |
| | GGACAGTGAATA | TLNGDDRHKIVNVDQRQ | ATACTAACAGCGTGTAC | GATTATTACCGG |
| | AGCCGGTTGTGG | YGDVFKGDLNPKPQGQR | GAACCTTATTACCACTC | TACATTACGAAT |
| | GCGTGCTTATGG | LIEVSVEENHPFTLRAP | AGATCATGCCGAATCAA | TACCAATCCAGT |
| | GCTTTGGGATTA | IQRIYGVRYTETWSFLP | GCTGGGTTAATAGAGGA | GCGCGCCAGTGT |
| | TTACCGGTACAT | SLTCTGDAAPAIQHICL | GAAAGCAGCCGAAAAGC | GCTGCGTTACGA |
| | TACGAATTACCA | KHTTCFQDVVVDVDCAE | CTACGACCACAACTCAC | CGACTTTCACAT |
| | ATCCAGTGCGCG | NTKEDQLAEISYRFQGK | CTTATATTTGGCCCAGA | TGACGAGGATAA |
| | CCAGTGTGCTGC | KEADQPWIVVNTSTLFD | AACGATTATGACGGTTT | GCTGGATACTAA |
| | GTTACGACGACT | ELELDPPEIEPGVLKVL | CCTGGAAAACGCACATG | CAGCGTGTACGA |
| | TTCACATTGACG | RTEKQYLGVYIWNMRGS | AACACCATGGAGTCTAC | ACCTTATTACCA |
| | AGGATAAGCTGG | DGTSTYATFLVTWKGDE | AACCAAGGCAGGGGAAT | CTCAGATCATGC |
| | ATACTAACAGCG | KTRNPTPAVTPQPRGAE | CGACAGTGGCGAGCGTC | CGAATCAAGCTG |
| | TGTACGAACCTT | FHMWNYHSHVFSVGDTF | TTATGCAGCCAACACAG | GGTTAATAGAGG |
| | ATTACCACTCAG | SLAMHLQYKIHEAPFDL | ATGTCGGCACAGGAGGA | AGAAAGCAGCCG |
| | ATCATGCCGAAT | LLEWLYVPIDPTCQPMR | TCTCGGTGATGACACCG | AAAAGCCTACGA |
| | CAAGCTGGGTTA | LYSTCLYHPNAPQCLSH | GCATACACGTGATTCCC | CCACAACTCACC |
| | ATAGAGGAGAAA | MNSGCTFTSPHLAQRVA | ACATTAAACGGCGACGA | TTATATTTGGCC |
| | GCAGCCGAAAAG | STVYQNCEHADNYTAYC | CAGACATAAGATCGTCA | CAGAAACGATTA |
| | CCTACGACCACA | LGISHMEPSFGLILHDG | ATGTGGATCAGCGTCAG | TGACGGTTTCCT |
| | ACTCACCTTATA | GTTLKFVDTPESLSGLY | TATGGGGATGTCTTTAA | GGAAAACGCACA |
| | TTTGGCCCAGAA | VFVVYFNGHVEAVAYTV | AGGCGATTTGAATCCAA | TGAACACCATGG |
| | ACGATTATGACG | VSTVDHFVNAIEERGFP | AGCCCCAAGGACAGAGA | AGTCTACAACCA |
| | GTTTCCTGGAAA | PTAGQPPATTKPKEITP | CTGATCGAGGTCTCTGT | AGGCAGGGGAAT |
| | ACGCACATGAAC | VNPGTSPLLRYAAWTGG | AGAAGAAAATCACCCCT | CGACAGTGGCGA |
| | ACCATGGAGTCT | LAAVVLLCLVIFLICTA | TCACTTTGCGCGCTCCA | GCGTCTTATGCA |
| | ACAACCAAGGCA | KRMRVKAYRVDKSPYNQ | ATCCAGAGGATTTACGG | GCCAACACAGAT |
| | GGGGAATCGACA | SMYYAGLPVDDFEDSES | GGTGCGTTATACCGAAA | GTCGGCACAGGA |
| | GTGGCGAGCGTC | TDTEEEFGNAIGGSHGG | CTTGGAGTTTCTTGCCG | GGATCTCGGTGA |
| | TTATGCAGCCAA | SSYTVYIDKTR | TCACTGACGTGTACGGG | TGACACCGGCAT |
| | CACAGATGTCGG | | GGATGCCGCCCCCGCAA | ACACGTGATTCC |
| | CACAGGAGGATC | | TCCAGCACATCTGTCTG | CACATTAAACGG |
| | TCGGTGATGACA | | AAACACACCACATGCTT | CGACGACAGACA |
| | CCGGCATACACG | | TCAGGACGTGGTTGTGG | TAAGATCGTCAA |
| | TGATTCCCACAT | | ATGTGGATTGCGCGGAA | TGTGGATCAGCG |
| | TAAACGGCGACG | | AACACAAAAGAAGACCA | TCAGTATGGGGA |
| | ACAGACATAAGA | | ACTCGCCGAAATCAGCT | TGTCTTTAAAGG |
| | TCGTCAATGTGG | | ATCGTTTTCAGGGTAAA | CGATTTGAATCC |
| | ATCAGCGTCAGT | | AAAGAGGCCGACCAACC | AAAGCCCCAAGG |
| | ATGGGGATGTCT | | GTGGATTGTTGTGAATA | ACAGAGACTGAT |
| | TTAAAGGCGATT | | CGAGCACGCTCTTCGAT | CGAGGTCTCTGT |
| | TGAATCCAAAGC | | GAGCTTGAACTCGATCC | AGAAGAAAATCA |
| | CCCAAGGACAGA | | CCCGGAAATCGAGCCTG | CCCCTTCACTTT |
| | GACTGATCGAGG | | GGGTTCTAAAAGTGTTG | GCGCGCTCCAAT |
| | TCTCTGTAGAAG | | AGGACCGAGAAGCAGTA | CCAGAGGATTTA |
| | AAAATCACCCCT | | CCTCGGGGTTTATATCT | CGGGGTGCGTTA |
| | TCACTTTGCGCG | | GGAATATGAGAGGCTCC | TACCGAAACTTG |
| | CTCCAATCCAGA | | GATGGCACCTCTACCTA | GAGTTTCTTGCC |
| | GGATTTACGGGG | | CGCAACGTTTCTGGTTA | GTCACTGACGTG |
| | TGCGTTATACCG | | CCTGGAAGGGAGACGAG | TACGGGGGATGC |
| | AAACTTGGAGTT | | AAGACACGGAATCCAAC | CGCCCCCGCAAT |
| | TCTTGCCGTCAC | | GCCCGCTGTGACCCCTC | CCAGCACATCTG |
| | TGACGTGTACGG | | AGCCTAGGGGAGCCGAA | TCTGAAACACAC |
| | GGGATGCCGCCC | | TTCCACATGTGGAACTA | CACATGCTTTCA |
| | CCGCAATCCAGC | | TCACTCCCATGTATTCA | GGACGTGGTTGT |
| | ACATCTGTCTGA | | GTGTGGGTGACACTTTC | GGATGTGGATTG |
| | AACACACCACAT | | AGCCTGGCCATGCACCT | CGCGGAAAACAC |
| | GCTTTCAGGACG | | GCAGTATAAGATTCACG | AAAAGAAGACCA |
| | TGGTTGTGGATG | | AGGCACCCTTCGACCTC | ACTCGCCGAAAT |
| | TGGATTGCGCGG | | CTGCTGGAGTGGTTGTA | CAGCTATCGTTT |
| | AAAACACAAAAG | | CGTACCTATTGATCCCA | TCAGGGTAAAAA |
| | AAGACCAACTCG | | CTTGTCAGCCCATGCGC | AGAGGCCGACCA |
| | CCGAAATCAGCT | | CTGTACTCCACTTGCTT | ACCGTGGATTGT |
| | ATCGTTTTCAGG | | GTACCACCCCAATGCAC | TGTGAATACGAG |
| | GTAAAAAAGAGG | | CACAGTGTCTATCACAC | CACGCTCTTCGA |
| | CCGACCAACCGT | | ATGAACTCCGGGTGTAC | TGAGCTTGAACT |
| | GGATTGTTGTGA | | CTTTACTTCACCCCATC | CGATCCCCCGGA |
| | ATACGAGCACGC | | TTGCCCAGCGGGTCGCC | AATCGAGCCTGG |
| | TCTTCGATGAGC | | AGCACAGTGTATCAGAA | GGTTCTAAAAGT |
| | TTGAACTCGATC | | CTGTGAGCATGCTGACA | GTTGAGGACCGA |
| | CCCCGGAAATCG | | ACTATACTGCTTATTGC | GAAGCAGTACCT |
| | AGCCTGGGGTTC | | CTCGGAATATCCCATAT | CGGGGTTTATAT |
| | TAAAAGTGTTGA | | GGAGCCAAGCTTCGGGC | CTGGAATATGAG |
| | GGACCGAGAAGC | | TCATACTGCACGATGGT | AGGCTCCGATGG |
| | AGTACCTCGGGG | | GGTACGACACTCAAGTT | CACCTCTACCTA |
| | TTTATATCTGGA | | CGTGGACACCCCCGAAA | CGCAACGTTTCT |
| | ATATGAGAGGCT | | GCCTTTCTGGCTTGTAC | GGTTACCTGGAA |
| | CCGATGGCACCT | | GTGTTCGTGGTCTACTT | GGGAGACGAGAA |
| | CTACCTACGCAA | | CAATGGACATGTGGAGG | GACACGGAATCC |
| | CGTTTCTGGTTA | | CAGTGGCTTACACAGTG | AACGCCCGCTGT |
| | CCTGGAAGGGAG | | GTTTCGACAGTTGATCA | GACCCCTCAGCC |
| | ACGAGAAGACAC | | CTTTGTAAATGCCATTG | TAGGGGAGCCGA |
| | GGAATCCAACGC | | AGGAACGCGGCTTCCCG | ATTCCACATGTG |
| | CCGCTGTGACCC | | CCTACAGCGGGCCAGCC | GAACTATCACTC |
| | CTCAGCCTAGGG | | CCCTGCGACAACAAAAC | CCATGTATTCAG |
| | GAGCCGAATTCC | | CAAAAGAGATTACGCCC | TGTGGGTGACAC |
| | ACATGTGGAACT | | GTTAATCCTGGGACTAG | TTTCAGCCTGGC |
| | ATCACTCCCATG | | TCCATTGCTGAGGTATG | CATGCACCTGCA |
| | TATTCAGTGTGG | | CCGCCTGGACTGGCGGT | GTATAAGATTCA |
| | GTGACACTTTCA | | CTGGCGGCCGTGGTACT | CGAGGCACCCTT |
| | GCCTGGCCATGC | | TCTGTGTTTAGTCATAT | CGACCTCCTGCT |
| | ACCTGCAGTATA | | TTCTGATCTGTACCGCT | GGAGTGGTTGTA |
| | AGATTCACGAGG | | AAACGTATGCGGGTCAA | CGTACCTATTGA |
| | CACCCTTCGACC | | GGCTTACCGTGTTGACA | TCCCACTTGTCA |
| | TCCTGCTGGAGT | | AGTCTCCTTACAATCAG | GCCCATGCGCCT |
| | GGTTGTACGTAC | | TCAATGTACTATGCAGG | GTACTCCACTTG |
| | CTATTGATCCCA | | ACTCCCTGTTGACGATT | CTTGTACCACCC |
| | CTTGTCAGCCCA | | TCGAAGACTCAGAGAGT | CAATGCACCACA |
| | TGCGCCTGTACT | | ACAGACACAGAAGAAGA | GTGTCTATCACA |
| | CCACTTGCTTGT | | ATTCGGAAACGCTATAG | CATGAACTCCGG |
| | ACCACCCCAATG | | GTGGCTCTCACGGAGGT | GTGTACCTTTAC |
| | CACCACAGTGTC | | AGCTCGTATACAGTGTA | TTCACCCCATCT |
| | TATCACACATGA | | CATCGATAAAACCAGA | TGCCCAGCGGGT |
| | ACTCCGGGTGTA | | | CGCCAGCACAGT |
| | CCTTTACTTCAC | | | GTATCAGAACTG |
| | CCCATCTTGCCC | | | TGAGCATGCTGA |
| | AGCGGGTCGCCA | | | CAACTATACTGC |
| | GCACAGTGTATC | | | TTATTGCCTCGG |
| | AGAACTGTGAGC | | | AATATCCCATAT |
| | ATGCTGACAACT | | | GGAGCCAAGCTT |
| | ATACTGCTTATT | | | CGGGCTCATACT |
| | GCCTCGGAATAT | | | GCACGATGGTGG |
| | CCCATATGGAGC | | | TACGACACTCAA |
| | CAAGCTTCGGGC | | | GTTCGTGGACAC |
| | TCATACTGCACG | | | CCCCGAAAGCCT |
| | ATGGTGGTACGA | | | TTCTGGCTTGTA |
| | CACTCAAGTTCG | | | CGTGTTCGTGGT |
| | TGGACACCCCCG | | | CTACTTCAATGG |
| | AAAGCCTTTCTG | | | ACATGTGGAGGC |
| | GCTTGTACGTGT | | | AGTGGCTTACAC |
| | TCGTGGTCTACT | | | AGTGGTTTCGAC |
| | TCAATGGACATG | | | AGTTGATCACTT |
| | TGGAGGCAGTGG | | | TGTAAATGCCAT |
| | CTTACACAGTGG | | | TGAGGAACGCGG |
| | TTTCGACAGTTG | | | CTTCCCGCCTAC |
| | ATCACTTTGTAA | | | AGCGGGCCAGCC |
| | ATGCCATTGAGG | | | CCCTGCGACAAC |
| | AACGCGGCTTCC | | | AAAACCAAAAGA |
| | CGCCTACAGCGG | | | GATTACGCCCGT |
| | GCCAGCCCCCTG | | | TAATCCTGGGAC |
| | CGACAACAAAAC | | | TAGTCCATTGCT |
| | CAAAAGAGATTA | | | GAGGTATGCCGC |
| | CGCCCGTTAATC | | | CTGGACTGGCGG |
| | CTGGGACTAGTC | | | TCTGGCGGCCGT |
| | CATTGCTGAGGT | | | GGTACTTCTGTG |
| | ATGCCGCCTGGA | | | TTTAGTCATATT |
| | CTGGCGGTCTGG | | | TCTGATCTGTAC |
| | CGGCCGTGGTAC | | | CGCTAAACGTAT |
| | TTCTGTGTTTAG | | | GCGGGTCAAGGC |
| | TCATATTTCTGA | | | TTACCGTGTTGA |
| | TCTGTACCGCTA | | | CAAGTCTCCTTA |
| | AACGTATGCGGG | | | CAATCAGTCAAT |
| | TCAAGGCTTACC | | | GTACTATGCAGG |
| | GTGTTGACAAGT | | | ACTCCCTGTTGA |
| | CTCCTTACAATC | | | CGATTTCGAAGA |
| | AGTCAATGTACT | | | CTCAGAGAGTAC |
| | ATGCAGGACTCC | | | AGACACAGAAGA |
| | CTGTTGACGATT | | | AGAATTCGGAAA |
| | TCGAAGACTCAG | | | CGCTATAGGTGG |
| | AGAGTACAGACA | | | CTCTCACGGAGG |
| | CAGAAGAAGAAT | | | TAGCTCGTATAC |
| | TCGGAAACGCTA | | | AGTGTACATCGA |
| | TAGGTGGCTCTC | | | TAAAACCAGATG |
| | ACGGAGGTAGCT | | | ATAATAGGCTGG |
| | CGTATACAGTGT | | | AGCCTCGGTGGC |
| | ACATCGATAAAA | | | CATGCTTCTTGC |
| | CCAGATGATAAT | | | CCCTTGGGCCTC |
| | AGGCTGGAGCCT | | | CCCCCAGCCCCT |
| | CGGTGGCCATGC | | | CCTCCCCTTCCT |
| | TTCTTGCCCCTT | | | GCACCCGTACCC |
| | GGGCCTCCCCCC | | | CCGTGGTCTTTG |
| | AGCCCCTCCTCC | | | AATAAAGTCTGA |
| | CCTTCCTGCACC | | | GTGGGCGGCAAA |
| | CGTACCCCCGTG | | | AAAAAAAAAAAA |
| | GTCTTTGAATAA | | | AAAAAAAAAAAA |
| | AGTCTGAGTGGG | | | AAAAAAAAAAAA |
| | CGGC | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | ATCTAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 13 | SEQ ID NO: 14 | SEQ ID NO:15 | SEQ ID NO: 16 |
| VZV_gE_ | TCAAGCTTTTGG | METPAQLLFLLLLWLPD | ATGGAGACTCCCGCTCA | G*GGGAAATAAG |
| Oka_ | ACCCTCGTACAG | TTGSVLRYDDFHIDEDK | GCTACTGTTCCTCCTGC | AGAGAAAAGAAG |
| hIgkappa | AAGCTAATACGA | LDTNSVYEPYYHSDHAE | TCCTTTGGCTGCCTGAT | AGTAAGAAGAAA |
| | CTCACTATAGGG | SSWVNRGESSRKAYDHN | ACTACAGGCTCTGTTTT | TATAAGAGCCAC |
| | AAATAAGAGAGA | SPYIWPRNDYDGFLENA | GCGGTACGACGACTTTC | CATGGAGACTCC |
| | AAAGAAGAGTAA | HEHHGVYNQGRGIDSGE | ACATCGATGAGGACAAG | CGCTCAGCTACT |
| | GAAGAAATATAA | RLMQPTQMSAQEDLGDD | CTCGACACTAATAGCGT | GTTCCTCCTGCT |
| | GAGCCACCATGG | TGIHVIPTLNGDDRHKI | GTATGAGCCCTACTACC | CCTTTGGCTGCC |
| | AGACTCCCGCTC | VNVDQRQYGDVFKGDLN | ATTCAGATCACGCCGAG | TGATACTACAGG |
| | AGCTACTGTTCC | PKPQGQRLIEVSVEENH | TCCTCTTGGGTGAACAG | CTCTGTTTTGCG |
| | TCCTGCTCCTTT | PFTLRAPIQRIYGVRYT | GGGTGAAAGTTCTAGGA | GTACGACGACTT |
| | GGCTGCCTGATA | ETWSFLPSLICTGDAAP | AAGCCTATGATCACAAC | TCACATCGATGA |
| | CTACAGGCTCTG | AIQHICLKHTTCFQDVV | AGCCCTTATATTTGGCC | GGACAAGCTCGA |
| | TTTTGCGGTACG | VDVDCAENTKEDQLAEI | ACGGAATGATTACGACG | CACTAATAGCGT |
| | ACGACTTTCACA | SYRFQGKKEADQPWIVV | GATTTCTCGAAAATGCC | GTATGAGCCCTA |
| | TCGATGAGGACA | NTSTLFDELELDPPEIE | CACGAGCATCACGGAGT | CTACCATTCAGA |
| | AGCTCGACACTA | PGVLKVLRTEKQYLGVY | GTACAACCAGGGCCGTG | TCACGCCGAGTC |
| | ATAGCGTGTATG | IWNMRGSDGTSTYATFL | GAATCGACTCTGGGGAG | CTCTTGGGTGAA |
| | AGCCCTACTACC | VTWKGDEKTRNPTPAVT | AGATTGATGCAACCTAC | CAGGGGTGAAAG |
| | ATTCAGATCACG | PQPRGAEFHMWNYHSHV | ACAGATGAGCGCCCAGG | TTCTAGGAAAGC |
| | CCGAGTCCTCTT | FSVGDTFSLAMHLQYKI | AAGATCTCGGGGATGAT | CTATGATCACAA |
| | GGGTGAACAGGG | HEAPFDLLLEWLYVPID | ACAGGAATTCACGTTAT | CAGCCCTTATAT |
| | GTGAAAGTTCTA | PTCQPMRLYSTCLYHPN | CCCTACATTAAACGGAG | TTGGCCACGGAA |
| | GGAAAGCCTATG | APQCLSHMNSGCTFTSP | ATGACCGCCACAAAATC | TGATTACGACGG |
| | ATCACAACAGCC | HLAQRVASTVYQNCEHA | GTCAATGTCGATCAAAG | ATTTCTCGAAAA |
| | CTTATATTTGGC | DNYTAYCLGISHMEPSF | ACAGTATGGAGATGTGT | TGCCCACGAGCA |
| | CACGGAATGATT | GLILHDGGTTLKFVDTP | TCAAAGGCGATCTCAAC | TCACGGAGTGTA |
| | ACGACGGATTTC | ESLSGLYVFVVYFNGHV | CCTAAGCCGCAGGGCCA | CAACCAGGGCCG |
| | TCGAAAATGCCC | EAVAYTVVSTVDHFVNA | GAGACTCATTGAGGTGT | TGGAATCGACTC |
| | ACGAGCATCACG | IEERGFPPTAGQPPATT | CTGTCGAAGAGAACCAC | TGGGGAGAGATT |
| | GAGTGTACAACC | KPKEITPVNPGTSPLLR | CCTTTCACTCTGCGCGC | GATGCAACCTAC |
| | AGGGCCGTGGAA | YAAWTGGLAAVVLLCLV | TCCCATTCAGAGAATCT | ACAGATGAGCGC |
| | TCGACTCTGGGG | IFLICTAKRMRVKAYRV | ATGGAGTTCGCTATACG | CCAGGAAGATCT |
| | AGAGATTGATGC | DKSPYNQSMYYAGLPVD | GAGACTTGGTCATTCCT | CGGGGATGATAC |
| | AACCTACACAGA | DFEDSESTDTEEEFGNA | TCCTTCCCTGACATGCA | AGGAATTCACGT |
| | TGAGCGCCCAGG | IGGSHGGSSYTVYIDKT | CCGGAGACGCCGCCCCT | TATCCCTACATT |
| | AAGATCTCGGGG | R | GCCATTCAGCACATATG | AAACGGAGATGA |
| | ATGATACAGGAA | | CCTGAAACATACCACCT | CCGCCACAAAAT |
| | TTCACGTTATCC | | GTTTCCAGGATGTGGTG | CGTCAATGTCGA |
| | CTACATTAAACG | | GTTGATGTTGATTGTGC | TCAAAGACAGTA |
| | GAGATGACCGCC | | TGAAAATACCAAGGAAG | TGGAGATGTGTT |
| | ACAAAATCGTCA | | ACCAACTGGCCGAGATT | CAAAGGCGATCT |
| | ATGTCGATCAAA | | AGTTACCGGTTCCAAGG | CAACCCTAAGCC |
| | GACAGTATGGAG | | GAAAAAGGAAGCCGACC | GCAGGGCCAGAG |
| | ATGTGTTCAAAG | | AGCCATGGATTGTGGTT | ACTCATTGAGGT |
| | GCGATCTCAACC | | AATACAAGCACTCTGTT | GTCTGTCGAAGA |
| | CTAAGCCGCAGG | | CGATGAGCTCGAGCTGG | GAACCACCCTTT |
| | GCCAGAGACTCA | | ATCCCCCCGAGATAGAA | CACTCTGCGCGC |
| | TTGAGGTGTCTG | | CCCGGAGTTCTGAAAGT | TCCCATTCAGAG |
| | TCGAAGAGAACC | | GCTCCGGACAGAAAAAC | AATCTATGGAGT |
| | ACCCTTTCACTC | | AATATCTGGGAGTCTAC | TCGCTATACGGA |
| | TGCGCGCTCCCA | | ATATGGAACATGCGCGG | GACTTGGTCATT |
| | TTCAGAGAATCT | | TTCCGATGGGACCTCCA | CCTTCCTTCCCT |
| | ATGGAGTTCGCT | | CTTATGCAACCTTTCTC | GACATGCACCGG |
| | ATACGGAGACTT | | GTCACGTGGAAGGGAGA | AGACGCCGCCCC |
| | GGTCATTCCTTC | | TGAGAAAACTAGGAATC | TGCCATTCAGCA |
| | CTTCCCTGACAT | | CCACACCCGCTGTCACA | CATATGCCTGAA |
| | GCACCGGAGACG | | CCACAGCCAAGAGGGGC | ACATACCACCTG |
| | CCGCCCCTGCCA | | TGAGTTCCATATGTGGA | TTTCCAGGATGT |
| | TTCAGCACATAT | | ACTATCATAGTCACGTG | GGTGGTTGATGT |
| | GCCTGAAACATA | | TTTAGTGTCGGAGATAC | TGATTGTGCTGA |
| | CCACCTGTTTCC | | GTTTTCATTGGCTATGC | AAATACCAAGGA |
| | AGGATGTGGTGG | | ATCTCCAGTACAAGATT | AGACCAACTGGC |
| | TTGATGTTGATT | | CATGAGGCTCCCTTCGA | CGAGATTAGTTA |
| | GTGCTGAAAATA | | TCTGTTGCTTGAGTGGT | CCGGTTCCAAGG |
| | CCAAGGAAGACC | | TGTACGTCCCGATTGAC | GAAAAAGGAAGC |
| | AACTGGCCGAGA | | CCGACCTGCCAGCCCAT | CGACCAGCCATG |
| | TTAGTTACCGGT | | GCGACTGTACAGCACCT | GATTGTGGTTAA |
| | TCCAAGGGAAAA | | GTCTCTACCATCCAAAC | AGAGAAAAGAAG |
| | AGGAAGCCGACC | | GCTCCGCAATGTCTGAG | TACAAGCACTCT |
| | AGCCATGGATTG | | CCACATGAACTCTGGGT | GTTCGATGAGCT |
| | TGGTTAATACAA | | GTACTTTCACCAGTCCC | CGAGCTGGATCC |
| | GCACTCTGTTCG | | CACCTCGCCCAGCGGGT | CCCCGAGATAGA |
| | ATGAGCTCGAGC | | GGCCTCTACTGTTTACC | ACCCGGAGTTCT |
| | TGGATCCCCCCG | | AGAACTGTGAGCACGCC | GAAAGTGCTCCG |
| | AGATAGAACCCG | | GACAACTACACCGCATA | GACAGAAAAACA |
| | GAGTTCTGAAAG | | CTGCCTCGGTATTTCTC | ATATCTGGGAGT |
| | TGCTCCGGACAG | | ACATGGAACCCTCCTTC | CTACATATGGAA |
| | AAAAACAATATC | | GGACTCATCCTGCACGA | CATGCGCGGTTC |
| | TGGGAGTCTACA | | TGGGGGCACTACCCTGA | CGATGGGACCTC |
| | TATGGAACATGC | | AGTTCGTTGATACGCCA | CACTTATGCAAC |
| | GCGGTTCCGATG | | GAATCTCTGTCTGGGCT | CTTTCTCGTCAC |
| | GGACCTCCACTT | | CTATGTTTTCGTGGTCT | GTGGAAGGGAGA |
| | ATGCAACCTTTC | | ACTTCAATGGCCATGTC | TGAGAAAACTAG |
| | TCGTCACGTGGA | | GAGGCCGTGGCCTATAC | GAATCCCACACC |
| | AGGGAGATGAGA | | TGTCGTTTCTACCGTGG | CGCTGTCACACC |
| | AAACTAGGAATC | | ATCATTTTGTGAACGCC | ACAGCCAAGAGG |
| | CCACACCCGCTG | | ATCGAAGAACGGGGATT | GGCTGAGTTCCA |
| | TCACACCACAGC | | CCCCCCTACGGCAGGCC | TATGTGGAACTA |
| | CAAGAGGGGCTG | | AGCCGCCTGCAACCACC | TCATAGTCACGT |
| | AGTTCCATATGT | | AAGCCCAAGGAAATAAC | GTTTAGTGTCGG |
| | GGAACTATCATA | | ACCAGTGAACCCTGGCA | AGATACGTTTTC |
| | GTCACGTGTTTA | | CCTCACCTCTCCTAAGA | ATTGGCTATGCA |
| | GTGTCGGAGATA | | TATGCCGCGTGGACAGG | TCTCCAGTACAA |
| | CGTTTTCATTGG | | GGGACTGGCGGCAGTGG | GATTCATGAGGC |
| | CTATGCATCTCC | | TGCTCCTCTGTCTCGTG | TCCCTTCGATCT |
| | AGTACAAGATTC | | ATCTTTCTGATCTGTAC | GTTGCTTGAGTG |
| | ATGAGGCTCCCT | | AGCCAAGAGGATGAGGG | GTTGTACGTCCC |
| | TCGATCTGTTGC | | TCAAGGCTTATAGAGTG | GATTGACCCGAC |
| | TTGAGTGGTTGT | | GACAAGTCCCCCTACAA | CTGCCAGCCCAT |
| | ACGTCCCGATTG | | TCAGTCAATGTACTACG | GCGACTGTACAG |
| | ACCCGACCTGCC | | CCGGCCTTCCCGTTGAT | CACCTGTCTCTA |
| | AGCCCATGCGAC | | GATTTTGAGGATTCCGA | CCATCCAAACGC |
| | TGTACAGCACCT | | GTCCACAGATACTGAGG | TCCGCAATGTCT |
| | GTCTCTACCATC | | AAGAGTTCGGTAACGCT | GAGCCACATGAA |
| | CAAACGCTCCGC | | ATAGGCGGCTCTCACGG | CTCTGGGTGTAC |
| | AATGTCTGAGCC | | GGGTTCAAGCTACACGG | TTTCACCAGTCC |
| | ACATGAACTCTG | | TTTACATTGACAAGACA | CCACCTCGCCCA |
| | GGTGTACTTTCA | | CGC | GCGGGTGGCCTC |
| | CCAGTCCCCACC | | | TACTGTTTACCA |
| | TCGCCCAGCGGG | | | GAACTGTGAGCA |
| | TGGCCTCTACTG | | | CGCCGACAACTA |
| | TTTACCAGAACT | | | CACCGCATACTG |
| | GTGAGCACGCCG | | | CCTCGGTATTTC |
| | ACAACTACACCG | | | TCACATGGAACC |
| | CATACTGCCTCG | | | CTCCTTCGGACT |
| | GTATTTCTCACA | | | CATCCTGCACGA |
| | TGGAACCCTCCT | | | TGGGGGCACTAC |
| | TCGGACTCATCC | | | CCTGAAGTTCGT |
| | TGCACGATGGGG | | | TGATACGCCAGA |
| | GCACTACCCTGA | | | ATCTCTGTCTGG |
| | AGTTCGTTGATA | | | GCTCTATGTTTT |
| | CGCCAGAATCTC | | | CGTGGTCTACTT |
| | TGTCTGGGCTCT | | | CAATGGCCATGT |
| | ATGTTTTCGTGG | | | CGAGGCCGTGGC |
| | TCTACTTCAATG | | | CTATACTGTCGT |
| | GCCATGTCGAGG | | | TTCTACCGTGGA |
| | CCGTGGCCTATA | | | TCATTTTGTGAA |
| | CTGTCGTTTCTA | | | CGCCATCGAAGA |
| | CCGTGGATCATT | | | ACGGGGATTCCC |
| | TTGTGAACGCCA | | | CCCTACGGCAGG |
| | TCGAAGAACGGG | | | CCAGCCGCCTGC |
| | GATTCCCCCCTA | | | AACCACCAAGCC |
| | CGGCAGGCCAGC | | | CAAGGAAATAAC |
| | CGCCTGCAACCA | | | ACCAGTGAACCC |
| | CCAAGCCCAAGG | | | TGGCACCTCACC |
| | AAATAACACCAG | | | TCTCCTAAGATA |
| | TGAACCCTGGCA | | | TGCCGCGTGGAC |
| | CCTCACCTCTCC | | | AGGGGGACTGGC |
| | TAAGATATGCCG | | | GGCAGTGGTGCT |
| | CGTGGACAGGGG | | | CCTCTGTCTCGT |
| | GACTGGCGGCAG | | | GATCTTTCTGAT |
| | TGGTGCTCCTCT | | | CTGTACAGCCAA |
| | GTCTCGTGATCT | | | GAGGATGAGGGT |
| | TTCTGATCTGTA | | | CAAGGCTTATAG |
| | CAGCCAAGAGGA | | | AGTGGACAAGTC |
| | TGAGGGTCAAGG | | | CCCCTACAATCA |
| | CTTATAGAGTGG | | | GTCAATGTACTA |
| | ACAAGTCCCCCT | | | CGCCGGCCTTCC |
| | ACAATCAGTCAA | | | CGTTGATGATTT |
| | TGTACTACGCCG | | | TGAGGATTCCGA |
| | GCCTTCCCGTTG | | | GTCCACAGATAC |
| | ATGATTTTGAGG | | | TGAGGAAGAGTT |
| | ATTCCGAGTCCA | | | CGGTAACGCTAT |
| | CAGATACTGAGG | | | AGGCGGCTCTCA |
| | AAGAGTTCGGTA | | | CGGGGGTTCAAG |
| | ACGCTATAGGCG | | | CTACACGGTTTA |
| | GCTCTCACGGGG | | | CATTGACAAGAC |
| | GTTCAAGCTACA | | | ACGCTGATAATA |
| | CGGTTTACATTG | | | GGCTGGAGCCTC |
| | ACAAGACACGCT | | | GGTGGCCATGCT |
| | GATAATAGGCTG | | | TCTTGCCCCTTG |
| | GAGCCTCGGTGG | | | GGCCTCCCCCCA |
| | CCATGCTTCTTG | | | GCCCCTCCTCCC |
| | CCCCTTGGGCCT | | | CTTCCTGCACCC |
| | CCCCCCAGCCCC | | | GTACCCCCGTGG |
| | TCCTCCCCTTCC | | | TCTTTGAATAAA |
| | TGCACCCGTACC | | | GTCTGAGTGGGC |
| | CCCGTGGTCTTT | | | GGCAAAAAAAAA |
| | GAATAAAGTCTG | | | AAAAAAAAAAAA |
| | AGTGGGCGGC | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAATCTAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 17 | SEQ ID NO: 18 | SEQ ID NO: 19 | SEQ ID NO: 20 |
| VZV-GE- | TCAAGCTTTTGG | MGTVNKPVVGVLMGFGI | ATGGGGACAGTTAATAA | G*GGGAAATAAG |
| delete- | ACCCTCGTACAG | ITGTLRITNPVRASVLR | ACCTGTGGTGGGGGTAT | AGAGAAAAGAAG |
| 562 | AAGCTAATACGA | YDDFHIDEDKLDTNSVY | TGATGGGGTTCGGAATT | AGTAAGAAGAAA |
| | CTCACTATAGGG | EPYYHSDHAESSWVNRG | ATCACGGGAACGTTGCG | TATAAGAGCCAC |
| | AAATAAGAGAGA | ESSRKAYDHNSPYIWPR | TATAACGAATCCGGTCA | CATGGGGACAGT |
| | AAAGAAGAGTAA | NDYDGFLENAHEHHGVY | GAGCATCCGTCTTGCGA | TAATAAACCTGT |
| | GAAGAAATATAA | NQGRGIDSGERLMQPTQ | TACGATGATTTTCACAT | GGTGGGGGTATT |
| | GAGCCACCATGG | MSAQEDLGDDTGIHVIP | CGATGAAGACAAACTGG | GATGGGGTTCGG |
| | GGACAGTTAATA | TLNGDDRHKIVNVDQRQ | ATACAAACTCCGTATAT | AATTATCACGGG |
| | AACCTGTGGTGG | YGDVFKGDLNPKPQGQR | GAGCCTTACTACCATTC | AACGTTGCGTAT |
| | GGGTATTGATGG | LIEVSVEENHPFTLRAP | AGATCATGCGGAGTCTT | AACGAATCCGGT |
| | GGTTCGGAATTA | IQRIYGVRYTETWSFLP | CATGGGTAAATCGGGGA | CAGAGCATCCGT |
| | TCACGGGAACGT | SLTCTGDAAPAIQHICL | GAGTCTTCGCGAAAAGC | CTTGCGATACGA |
| | TGCGTATAACGA | KHTTCFQDVVVDVDCAE | GTACGATCATAACTCAC | TGATTTTCACAT |
| | ATCCGGTCAGAG | NTKEDQLAEISYRFQGK | CTTATATATGGCCACGT | CGATGAAGACAA |
| | CATCCGTCTTGC | KEADQPWIVVNTSTLFD | AATGATTATGATGGATT | ACTGGATACAAA |
| | GATACGATGATT | ELELDPPEIEPGVLKVL | TTTAGAGAACGCACACG | CTCCGTATATGA |
| | TTCACATCGATG | RTEKQYLGVYIWNMRGS | AACACCATGGGGTGTAT | GCCTTACTACCA |
| | AAGACAAACTGG | DGTSTYATFLVTWKGDE | AATCAGGGCCGTGGTAT | TTCAGATCATGC |
| | ATACAAACTCCG | KTRNPTPAVTPQPRGAE | CGATAGCGGGGAACGGT | GGAGTCTTCATG |
| | TATATGAGCCTT | FHMWNYHSHVFSVGDTF | TAATGCAACCCACACAA | GGTAAATCGGGG |
| | ACTACCATTCAG | SLAMHLQYKIHEAPFDL | ATGTCTGCACAGGAGGA | AGAGTCTTCGCG |
| | ATCATGCGGAGT | LLEWLYVPIDPTCQPMR | TCTTGGGGACGATACGG | AAAAGCGTACGA |
| | CTTCATGGGTAA | LYSTCLYHPNAPQCLSH | GCATCCACGTTATCCCT | TCATAACTCACC |
| | ATCGGGGAGAGT | MNSGCTFTSPHLAQRVA | ACGTTAAACGGCGATGA | TTATATATGGCC |
| | CTTCGCGAAAAG | STVYQNCEHADNYTAYC | CAGACATAAAATTGTAA | ACGTAATGATTA |
| | CGTACGATCATA | LGISHMEPSFGLILHDG | ATGTGGACCAACGTCAA | TGATGGATTTTT |
| | ACTCACCTTATA | GTTLKFVDTPESLSGLY | TACGGTGACGTGTTTAA | AGAGAACGCACA |
| | TATGGCCACGTA | VFVVYFNGHVEAVAYTV | AGGAGATCTTAATCCAA | CGAACACCATGG |
| | ATGATTATGATG | VSTVDHEVNAIEERGFP | AACCCCAAGGCCAAAGA | GGTGTATAATCA |
| | GATTTTTAGAGA | PTAGQPPATTKPKEITP | CTCATTGAGGTGTCAGT | GGGCCGTGGTAT |
| | ACGCACACGAAC | VNPGTSPLLRYAAWTGG | GGAAGAAAATCACCCGT | CGATAGCGGGGA |
| | ACCATGGGGTGT | LAAVVLLCLVIFLICTA | TTACTTTACGCGCACCG | ACGGTTAATGCA |
| | ATAATCAGGGCC | * | ATTCAGCGGATTTATGG | ACCCACACAAAT |
| | GTGGTATCGATA | | AGTCCGGTACACCGAGA | GTCTGCACAGGA |
| | GCGGGGAACGGT | | CTTGGAGCTTTTTGCCG | GGATCTTGGGGA |
| | TAATGCAACCCA | | TCATTAACCTGTACGGG | CGATACGGGCAT |
| | CACAAATGTCTG | | AGACGCAGCGCCCGCCA | CCACGTTATCCC |
| | CACAGGAGGATC | | TCCAGCATATATGTTTA | TACGTTAAACGG |
| | TTGGGGACGATA | | AAACATACAACATGCTT | CGATGACAGACA |
| | CGGGCATCCACG | | TCAAGACGTGGTGGTGG | TAAAATTGTAAA |
| | TTATCCCTACGT | | ATGTGGATTGCGCGGAA | TGTGGACCAACG |
| | TAAACGGCGATG | | AATACTAAAGAGGATCA | TCAATACGGTGA |
| | ACAGACATAAAA | | GTTGGCCGAAATCAGTT | CGTGTTTAAAGG |
| | TTGTAAATGTGG | | ACCGTTTTCAAGGTAAG | AGATCTTAATCC |
| | ACCAACGTCAAT | | AAGGAAGCGGACCAACC | AAAACCCCAAGG |
| | ACGGTGACGTGT | | GTGGATTGTTGTAAACA | CCAAAGACTCAT |
| | TTAAAGGAGATC | | CGAGCACACTGTTTGAT | TGAGGTGTCAGT |
| | TTAATCCAAAAC | | GAACTCGAATTAGACCC | GGAAGAAAATCA |
| | CCCAAGGCCAAA | | CCCCGAGATTGAACCGG | CCCGTTTACTTT |
| | GACTCATTGAGG | | GTGTCTTGAAAGTACTT | ACGCGCACCGAT |
| | TGTCAGTGGAAG | | CGGACAGAAAAACAATA | TCAGCGGATTTA |
| | AAAATCACCCGT | | CTTGGGTGTGTACATTT | TGGAGTCCGGTA |
| | TTACTTTACGCG | | GGAACATGCGCGGCTCC | CACCGAGACTTG |
| | CACCGATTCAGC | | GATGGTACGTCTACCTA | GAGCTTTTTGCC |
| | GGATTTATGGAG | | CGCCACGTTTTTGGTCA | GTCATTAACCTG |
| | TCCGGTACACCG | | CCTGGAAAGGGGATGAA | TACGGGAGACGC |
| | AGACTTGGAGCT | | AAAACAAGAAACCCTAC | AGCGCCCGCCAT |
| | TTTTGCCGTCAT | | GCCCGCAGTAACTCCTC | CCAGCATATATG |
| | TAACCTGTACGG | | AACCAAGAGGGGCTGAG | TTTAAAACATAC |
| | GAGACGCAGCGC | | TTTCATATGTGGAATTA | AACATGCTTTCA |
| | CCGCCATCCAGC | | CCACTCGCATGTATTTT | AGACGTGGTGGT |
| | ATATATGTTTAA | | CAGTTGGTGATACGTTT | GGATGTGGATTG |
| | AACATACAACAT | | AGCTTGGCAATGCATCT | CGCGGAAAATAC |
| | GCTTTCAAGACG | | TCAGTATAAGATACATG | TAAAGAGGATCA |
| | TGGTGGTGGATG | | AAGCGCCATTTGATTTG | GTTGGCCGAAAT |
| | TGGATTGCGCGG | | CTGTTAGAGTGGTTGTA | CAGTTACCGTTT |
| | AAAATACTAAAG | | TGTCCCCATCGATCCTA | TCAAGGTAAGAA |
| | AGGATCAGTTGG | | CATGTCAACCAATGCGG | GGAAGCGGACCA |
| | CCGAAATCAGTT | | TTATATTCTACGTGTTT | ACCGTGGATTGT |
| | ACCGTTTTCAAG | | GTATCATCCCAACGCAC | TGTAAACACGAG |
| | GTAAGAAGGAAG | | CCCAATGCCTCTCTCAT | CACACTGTTTGA |
| | CGGACCAACCGT | | ATGAATTCCGGTTGTAC | TGAACTCGAATT |
| | GGATTGTTGTAA | | ATTTACCTCGCCACATT | AGACCCCCCCGA |
| | ACACGAGCACAC | | TAGCCCAGCGTGTTGCA | GATTGAACCGGG |
| | TGTTTGATGAAC | | AGCACAGTGTATCAAAA | TGTCTTGAAAGT |
| | TCGAATTAGACC | | TTGTGAACATGCAGATA | ACTTCGGACAGA |
| | CCCCCGAGATTG | | ACTACACCGCATATTGT | AAAACAATACTT |
| | AACCGGGTGTCT | | CTGGGAATATCTCATAT | GGGTGTGTACAT |
| | TGAAAGTACTTC | | GGAGCCTAGCTTTGGTC | TTGGAACATGCG |
| | GGACAGAAAAAC | | TAATCTTACACGACGGG | CGGCTCCGATGG |
| | AATACTTGGGTG | | GGCACCACGTTAAAGTT | TACGTCTACCTA |
| | TGTACATTTGGA | | TGTAGATACACCCGAGA | CGCCACGTTTTT |
| | ACATGCGCGGCT | | GTTTGTCGGGATTATAC | GGTCACCTGGAA |
| | CCGATGGTACGT | | GTTTTTGTGGTGTATTT | AGGGGATGAAAA |
| | CTACCTACGCCA | | TAACGGGCATGTTGAAG | AACAAGAAACCC |
| | CGTTTTTGGTCA | | CCGTAGCATACACTGTT | TACGCCCGCAGT |
| | CCTGGAAAGGGG | | GTATCCACAGTAGATCA | AACTCCTCAACC |
| | ATGAAAAAACAA | | TTTTGTAAACGCAATTG | AAGAGGGGCTGA |
| | GAAACCCTACGC | | AAGAGCGTGGATTTCCG | GTTTCATATGTG |
| | CCGCAGTAACTC | | CCAACGGCCGGTCAGCC | GAATTACCACTC |
| | CTCAACCAAGAG | | ACCGGCGACTACTAAAC | GCATGTATTTTC |
| | GGGCTGAGTTTC | | CCAAGGAAATTACCCCC | AGTTGGTGATAC |
| | ATATGTGGAATT | | GTAAACCCCGGAACGTC | GTTTAGCTTGGC |
| | ACCACTCGCATG | | ACCACTTCTACGATATG | AATGCATCTTCA |
| | TATTTTCAGTTG | | CCGCATGGACCGGAGGG | GTATAAGATACA |
| | GTGATACGTTTA | | CTTGCAGCAGTAGTACT | TGAAGCGCCATT |
| | GCTTGGCAATGC | | TTTATGTCTCGTAATAT | TGATTTGCTGTT |
| | ATCTTCAGTATA | | TTTTAATCTGTACGGCT | AGAGTGGTTGTA |
| | AGATACATGAAG | | TGA | TGTCCCCATCGA |
| | CGCCATTTGATT | | | TCCTACATGTCA |
| | TGCTGTTAGAGT | | | ACCAATGCGGTT |
| | GGTTGTATGTCC | | | ATATTCTACGTG |
| | CCATCGATCCTA | | | TTTGTATCATCC |
| | CATGTCAACCAA | | | CAACGCACCCCA |
| | TGCGGTTATATT | | | ATGCCTCTCTCA |
| | CTACGTGTTTGT | | | TATGAATTCCGG |
| | ATCATCCCAACG | | | TTGTACATTTAC |
| | CACCCCAATGCC | | | CTCGCCACATTT |
| | TCTCTCATATGA | | | AGCCCAGCGTGT |
| | ATTCCGGTTGTA | | | TGCAAGCACAGT |
| | CATTTACCTCGC | | | GTATCAAAATTG |
| | CACATTTAGCCC | | | TGAACATGCAGA |
| | AGCGTGTTGCAA | | | TAACTACACCGC |
| | GCACAGTGTATC | | | ATATTGTCTGGG |
| | AAAATTGTGAAC | | | AATATCTCATAT |
| | ATGCAGATAACT | | | GGAGCCTAGCTT |
| | ACACCGCATATT | | | TGGTCTAATCTT |
| | GTCTGGGAATAT | | | ACACGACGGGGG |
| | CTCATATGGAGC | | | CACCACGTTAAA |
| | CTAGCTTTGGTC | | | GTTTGTAGATAC |
| | TAATCTTACACG | | | ACCCGAGAGTTT |
| | ACGGGGGCACCA | | | GTCGGGATTATA |
| | CGTTAAAGTTTG | | | CGTTTTTGTGGT |
| | TAGATACACCCG | | | GTATTTTAACGG |
| | AGAGTTTGTCGG | | | GCATGTTGAAGC |
| | GATTATACGTTT | | | CGTAGCATACAC |
| | TTGTGGTGTATT | | | TGTTGTATCCAC |
| | TTAACGGGCATG | | | AGTAGATCATTT |
| | TTGAAGCCGTAG | | | TGTAAACGCAAT |
| | CATACACTGTTG | | | TGAAGAGCGTGG |
| | TATCCACAGTAG | | | ATTTCCGCCAAC |
| | ATCATTTTGTAA | | | GGCCGGTCAGCC |
| | ACGCAATTGAAG | | | ACCGGCGACTAC |
| | AGCGTGGATTTC | | | TAAACCCAAGGA |
| | CGCCAACGGCCG | | | AATTACCCCCGT |
| | GTCAGCCACCGG | | | AAACCCCGGAAC |
| | CGACTACTAAAC | | | GTCACCACTTCT |
| | CCAAGGAAATTA | | | ACGATATGCCGC |
| | CCCCCGTAAACC | | | ATGGACCGGAGG |
| | CCGGAACGTCAC | | | GCTTGCAGCAGT |
| | CACTTCTACGAT | | | AGTACTTTTATG |
| | ATGCCGCATGGA | | | TCTCGTAATATT |
| | CCGGAGGGCTTG | | | TTTAATCTGTAC |
| | CAGCAGTAGTAC | | | GGCTTGATGATA |
| | TTTTATGTCTCG | | | ATAGGCTGGAGC |
| | TAATATTTTTAA | | | CTCGGTGGCCAT |
| | TCTGTACGGCTT | | | GCTTCTTGCCCC |
| | GATGATAATAGG | | | TTGGGCCTCCCC |
| | CTGGAGCCTCGG | | | CCAGCCCCTCCT |
| | TGGCCATGCTTC | | | CCCCTTCCTGCA |
| | TTGCCCCTTGGG | | | CCCGTACCCCCG |
| | CCTCCCCCCAGC | | | TGGTCTTTGAAT |
| | CCCTCCTCCCCT | | | AAAGTCTGAGTG |
| | TCCTGCACCCGT | | | GGCGGCAAAAAA |
| | ACCCCCGTGGTC | | | AAAAAAAAAAAA |
| | TTTGAATAAAGT | | | AAAAAAAAAAAA |
| | CTGAGTGGGCGG | | | AAAAAAAAAAAA |
| | C | | | AAAAAAAAAAAA |
| | | | | AAAAAAAAAAAA |
| | | | | AAAAAAAAAAAA |
| | | | | AAAAAAAAAAAA |
| | | | | AAAAAAAAAATC |
| | | | | TAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 21 | SEQ ID NO: 22 | SEQ ID NO: 23 | SEQ ID NO: 24 |
| VZV-GE- | TCAAGCTTTTGG | METPAQLLFLLLLWLPD | ATGGAAACCCCGGCGCA | G*GGGAAATAAG |
| delete- | ACCCTCGTACAG | TTGSVLRYDDFHIDEDK | GCTGCTGTTTCTGCTGC | AGAGAAAAGAAG |
| 562- | AAGCTAATACGA | LDTNSVYEPYYHSDHAE | TGCTGTGGCTGCCGGAT | AGTAAGAAGAAA |
| replace | CTCACTATAGGG | SSWVNRGESSRKAYDHN | ACCACCGGCTCCGTCTT | TATAAGAGCCAC |
| dsp- | AAATAAGAGAGA | SPYIWPRNDYDGFLENA | GCGATACGATGATTTTC | CATGGAAACCCC |
| with | AAAGAAGAGTAA | HEHHGVYNQGRGIDSGE | ACATCGATGAAGACAAA | GGCGCAGCTGCT |
| IgKappa | GAAGAAATATAA | RLMQPTQMSAQEDLGDD | CTGGATACAAACTCCGT | GTTTCTGCTGCT |
| | GAGCCACCATGG | TGIHVIPTLNGDDRHKI | ATATGAGCCTTACTACC | GCTGTGGCTGCC |
| | AAACCCCGGCGC | VNVDQRQYGDVFKGDLN | ATTCAGATCATGCGGAG | GGATACCACCGG |
| | AGCTGCTGTTTC | PKPQGQRLIEVSVEENH | TCTTCATGGGTAAATCG | CTCCGTCTTGCG |
| | TGCTGCTGCTGT | PFTLRAPIQRIYGVRYT | GGGAGAGTCTTCGCGAA | ATACGATGATTT |
| | GGCTGCCGGATA | ETWSFLPSLICTGDAAP | AAGCGTACGATCATAAC | TCACATCGATGA |
| | CCACCGGCTCCG | AIQHICLKHTTCFQDVV | TCACCTTATATATGGCC | AGACAAACTGGA |
| | TCTTGCGATACG | VDVDCAENTKEDQLAEI | ACGTAATGATTATGATG | TACAAACTCCGT |
| | ATGATTTTCACA | SYRFQGKKEADQPWIVV | GATTTTTAGAGAACGCA | ATATGAGCCTTA |
| | TCGATGAAGACA | NTSTLFDELELDPPEIE | CACGAACACCATGGGGT | CTACCATTCAGA |
| | AACTGGATACAA | PGVLKVLRTEKQYLGVY | GTATAATCAGGGCCGTG | TCATGCGGAGTC |
| | ACTCCGTATATG | IWNMRGSDGTSTYATFL | GTATCGATAGCGGGGAA | TTCATGGGTAAA |
| | AGCCTTACTACC | VTWKGDEKTRNPTPAVT | CGGTTAATGCAACCCAC | TCGGGGAGAGTC |
| | ATTCAGATCATG | PQPRGAEFHMWNYHSHV | ACAAATGTCTGCACAGG | TTCGCGAAAAGC |
| | CGGAGTCTTCAT | FSVGDTFSLAMHLQYKI | AGGATCTTGGGGACGAT | GTACGATCATAA |
| | GGGTAAATCGGG | HEAPFDLLLEWLYVPID | ACGGGCATCCACGTTAT | CTCACCTTATAT |
| | GAGAGTCTTCGC | PTCQPMRLYSTCLYHPN | CCCTACGTTAAACGGCG | ATGGCCACGTAA |
| | GAAAAGCGTACG | APQCLSHMNSGCTFTSP | ATGACAGACATAAAATT | TGATTATGATGG |
| | ATCATAACTCAC | HLAQRVASTVYQNCEHA | GTAAATGTGGACCAACG | ATTTTTAGAGAA |
| | CTTATATATGGC | DNYTAYCLGISHMEPSF | TCAATACGGTGACGTGT | CGCACACGAACA |
| | CACGTAATGATT | GLILHDGGTTLKFVDTP | TTAAAGGAGATCTTAAT | CCATGGGGTGTA |
| | ATGATGGATTTT | ESLSGLYVFVVYFNGHV | CCAAAACCCCAAGGCCA | TAATCAGGGCCG |
| | TAGAGAACGCAC | EAVAYTVVSTVDHFVNA | AAGACTCATTGAGGTGT | TGGTATCGATAG |
| | ACGAACACCATG | IEERGFPPTAGQPPATT | CAGTGGAAGAAAATCAC | CGGGGAACGGTT |
| | GGGTGTATAATC | KPKEITPVNPGTSPLLR | CCGTTTACTTTACGCGC | AATGCAACCCAC |
| | AGGGCCGTGGTA | YAAWTGGLAAVVLLCLV | ACCGATTCAGCGGATTT | ACAAATGTCTGC |
| | TCGATAGCGGGG | IFLICTA* | ATGGAGTCCGGTACACC | ACAGGAGGATCT |
| | AACGGTTAATGC | | GAGACTTGGAGCTTTTT | TGGGGACGATAC |
| | AACCCACACAAA | | GCCGTCATTAACCTGTA | GGGCATCCACGT |
| | TGTCTGCACAGG | | CGGGAGACGCAGCGCCC | TATCCCTACGTT |
| | AGGATCTTGGGG | | GCCATCCAGCATATATG | AAACGGCGATGA |
| | ACGATACGGGCA | | TTTAAAACATACAACAT | CAGACATAAAAT |
| | TCCACGTTATCC | | GCTTTCAAGACGTGGTG | TGTAAATGTGGA |
| | CTACGTTAAACG | | GTGGATGTGGATTGCGC | CCAACGTCAATA |
| | GCGATGACAGAC | | GGAAAATACTAAAGAGG | CGGTGACGTGTT |
| | ATAAAATTGTAA | | ATCAGTTGGCCGAAATC | TAAAGGAGATCT |
| | ATGTGGACCAAC | | AGTTACCGTTTTCAAGG | TAATCCAAAACC |
| | GTCAATACGGTG | | TAAGAAGGAAGCGGACC | CCAAGGCCAAAG |
| | ACGTGTTTAAAG | | AACCGTGGATTGTTGTA | ACTCATTGAGGT |
| | GAGATCTTAATC | | AACACGAGCACACTGTT | GTCAGTGGAAGA |
| | CAAAACCCCAAG | | TGATGAACTCGAATTAG | AAATCACCCGTT |
| | GCCAAAGACTCA | | ACCCCCCCGAGATTGAA | TACTTTACGCGC |
| | TTGAGGTGTCAG | | CCGGGTGTCTTGAAAGT | ACCGATTCAGCG |
| | TGGAAGAAAATC | | ACTTCGGACAGAAAAAC | GATTTATGGAGT |
| | ACCCGTTTACTT | | AATACTTGGGTGTGTAC | CCGGTACACCGA |
| | TACGCGCACCGA | | ATTTGGAACATGCGCGG | GACTTGGAGCTT |
| | TTCAGCGGATTT | | CTCCGATGGTACGTCTA | TTTGCCGTCATT |
| | ATGGAGTCCGGT | | CCTACGCCACGTTTTTG | AACCTGTACGGG |
| | ACACCGAGACTT | | GTCACCTGGAAAGGGGA | AGACGCAGCGCC |
| | GGAGCTTTTTGC | | TGAAAAAACAAGAAACC | CGCCATCCAGCA |
| | CGTCATTAACCT | | CTACGCCCGCAGTAACT | TATATGTTTAAA |
| | GTACGGGAGACG | | CCTCAACCAAGAGGGGC | ACATACAACATG |
| | CAGCGCCCGCCA | | TGAGTTTCATATGTGGA | CTTTCAAGACGT |
| | TCCAGCATATAT | | ATTACCACTCGCATGTA | GGTGGTGGATGT |
| | GTTTAAAACATA | | TTTTCAGTTGGTGATAC | GGATTGCGCGGA |
| | CAACATGCTTTC | | GTTTAGCTTGGCAATGC | AAATACTAAAGA |
| | AAGACGTGGTGG | | ATCTTCAGTATAAGATA | GGATCAGTTGGC |
| | TGGATGTGGATT | | CATGAAGCGCCATTTGA | CGAAATCAGTTA |
| | GCGCGGAAAATA | | TTTGCTGTTAGAGTGGT | CCGTTTTCAAGG |
| | CTAAAGAGGATC | | TGTATGTCCCCATCGAT | TAAGAAGGAAGC |
| | AGTTGGCCGAAA | | CCTACATGTCAACCAAT | GGACCAACCGTG |
| | TCAGTTACCGTT | | GCGGTTATATTCTACGT | GATTGTTGTAAA |
| | TTCAAGGTAAGA | | GTTTGTATCATCCCAAC | CACGAGCACACT |
| | AGGAAGCGGACC | | GCACCCCAATGCCTCTC | GTTTGATGAACT |
| | AACCGTGGATTG | | TCATATGAATTCCGGTT | CGAATTAGACCC |
| | TTGTAAACACGA | | GTACATTTACCTCGCCA | CCCCGAGATTGA |
| | GCACACTGTTTG | | CATTTAGCCCAGCGTGT | ACCGGGTGTCTT |
| | ATGAACTCGAAT | | TGCAAGCACAGTGTATC | GAAAGTACTTCG |
| | TAGACCCCCCCG | | AAAATTGTGAACATGCA | GACAGAAAAACA |
| | AGATTGAACCGG | | GATAACTACACCGCATA | ATACTTGGGTGT |
| | GTGTCTTGAAAG | | TTGTCTGGGAATATCTC | GTACATTTGGAA |
| | TACTTCGGACAG | | ATATGGAGCCTAGCTTT | CATGCGCGGCTC |
| | AAAAACAATACT | | GGTCTAATCTTACACGA | CGATGGTACGTC |
| | TGGGTGTGTACA | | CGGGGGCACCACGTTAA | TACCTACGCCAC |
| | TTTGGAACATGC | | AGTTTGTAGATACACCC | GTTTTTGGTCAC |
| | GCGGCTCCGATG | | GAGAGTTTGTCGGGATT | CTGGAAAGGGGA |
| | GTACGTCTACCT | | ATACGTTTTTGTGGTGT | TGAAAAAACAAG |
| | ACGCCACGTTTT | | ATTTTAACGGGCATGTT | AAACCCTACGCC |
| | TGGTCACCTGGA | | GAAGCCGTAGCATACAC | CGCAGTAACTCC |
| | AAGGGGATGAAA | | TGTTGTATCCACAGTAG | TCAACCAAGAGG |
| | AAACAAGAAACC | | ATCATTTTGTAAACGCA | GGCTGAGTTTCA |
| | CTACGCCCGCAG | | ATTGAAGAGCGTGGATT | TATGTGGAATTA |
| | TAACTCCTCAAC | | TCCGCCAACGGCCGGTC | CCACTCGCATGT |
| | CAAGAGGGGCTG | | AGCCACCGGCGACTACT | ATTTTCAGTTGG |
| | AGTTTCATATGT | | AAACCCAAGGAAATTAC | TGATACGTTTAG |
| | GGAATTACCACT | | CCCCGTAAACCCCGGAA | CTTGGCAATGCA |
| | CGCATGTATTTT | | CGTCACCACTTCTACGA | TCTTCAGTATAA |
| | CAGTTGGTGATA | | TATGCCGCATGGACCGG | GATACATGAAGC |
| | CGTTTAGCTTGG | | AGGGCTTGCAGCAGTAG | GCCATTTGATTT |
| | CAATGCATCTTC | | TACTTTTATGTCTCGTA | GCTGTTAGAGTG |
| | AGTATAAGATAC | | ATATTTTTAATCTGTAC | GTTGTATGTCCC |
| | ATGAAGCGCCAT | | GGCTTGA | CATCGATCCTAC |
| | TTGATTTGCTGT | | | ATGTCAACCAAT |
| | TAGAGTGGTTGT | | | GCGGTTATATTC |
| | ATGTCCCCATCG | | | TACGTGTTTGTA |
| | ATCCTACATGTC | | | TCATCCCAACGC |
| | AACCAATGCGGT | | | ACCCCAATGCCT |
| | TATATTCTACGT | | | CTCTCATATGAA |
| | GTTTGTATCATC | | | TTCCGGTTGTAC |
| | CCAACGCACCCC | | | ATTTACCTCGCC |
| | AATGCCTCTCTC | | | ACATTTAGCCCA |
| | ATATGAATTCCG | | | GCGTGTTGCAAG |
| | GTTGTACATTTA | | | CACAGTGTATCA |
| | CCTCGCCACATT | | | AAATTGTGAACA |
| | TAGCCCAGCGTG | | | TGCAGATAACTA |
| | TTGCAAGCACAG | | | CACCGCATATTG |
| | TGTATCAAAATT | | | TCTGGGAATATC |
| | GTGAACATGCAG | | | TCATATGGAGCC |
| | ATAACTACACCG | | | TAGCTTTGGTCT |
| | CATATTGTCTGG | | | AATCTTACACGA |
| | GAATATCTCATA | | | CGGGGGCACCAC |
| | TGGAGCCTAGCT | | | GTTAAAGTTTGT |
| | TTGGTCTAATCT | | | AGATACACCCGA |
| | TACACGACGGGG | | | GAGTTTGTCGGG |
| | GCACCACGTTAA | | | ATTATACGTTTT |
| | AGTTTGTAGATA | | | TGTGGTGTATTT |
| | CACCCGAGAGTT | | | TAACGGGCATGT |
| | TGTCGGGATTAT | | | TGAAGCCGTAGC |
| | ACGTTTTTGTGG | | | ATACACTGTTGT |
| | TGTATTTTAACG | | | ATCCACAGTAGA |
| | GGCATGTTGAAG | | | TCATTTTGTAAA |
| | CCGTAGCATACA | | | CGCAATTGAAGA |
| | CTGTTGTATCCA | | | GCGTGGATTTCC |
| | CAGTAGATCATT | | | GCCAACGGCCGG |
| | TTGTAAACGCAA | | | TCAGCCACCGGC |
| | TTGAAGAGCGTG | | | GACTACTAAACC |
| | GATTTCCGCCAA | | | CAAGGAAATTAC |
| | CGGCCGGTCAGC | | | CCCCGTAAACCC |
| | CACCGGCGACTA | | | CGGAACGTCACC |
| | CTAAACCCAAGG | | | ACTTCTACGATA |
| | AAATTACCCCCG | | | TGCCGCATGGAC |
| | TAAACCCCGGAA | | | CGGAGGGCTTGC |
| | CGTCACCACTTC | | | AGCAGTAGTACT |
| | TACGATATGCCG | | | TTTATGTCTCGT |
| | CATGGACCGGAG | | | AATATTTTTAAT |
| | GGCTTGCAGCAG | | | CTGTACGGCTTG |
| | TAGTACTTTTAT | | | ATGATAATAGGC |
| | GTCTCGTAATAT | | | TGGAGCCTCGGT |
| | TTTTAATCTGTA | | | GGCCATGCTTCT |
| | CGGCTTGATGAT | | | TGCCCCTTGGGC |
| | AATAGGCTGGAG | | | CTCCCCCCAGCC |
| | CCTCGGTGGCCA | | | CCTCCTCCCCTT |
| | TGCTTCTTGCCC | | | CCTGCACCCGTA |
| | CTTGGGCCTCCC | | | CCCCCGTGGTCT |
| | CCCAGCCCCTCC | | | TTGAATAAAGTC |
| | TCCCCTTCCTGC | | | TGAGTGGGCGGC |
| | ACCCGTACCCCC | | | AAAAAAAAAAAA |
| | GTGGTCTTTGAA | | | AAAAAAAAAAAA |
| | TAAAGTCTGAGT | | | AAAAAAAAAAAA |
| | GGGCGGC | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAATCTAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 25 | SEQ ID NO: 26 | SEQ ID NO: 27 | SEQ ID NO: 28 |
| VZV-GE- | TCAAGCTTTTGG | MGTVNKPVVGVLMGFGI | ATGGGGACAGTTAATAA | G*GGGAAATAAG |
| full_ | ACCCTCGTACAG | ITGTLRITNPVRASVLR | ACCTGIGGIGGGGGTAT | AGAGAAAAGAAG |
| with_ | AAGCTAATACGA | YDDFHIDEDKLDTNSVY | TGATGGGGTTCGGAATT | AGTAAGAAGAAA |
| AEAADA | CTCACTATAGGG | EPYYHSDHAESSWVNRG | ATCACGGGAACGTTGCG | TATAAGAGCCAC |
| (SEQ | AAATAAGAGAGA | ESSRKAYDHNSPYIWPR | TATAACGAATCCGGTCA | CATGGGGACAGT |
| ID NO: | AAAGAAGAGTAA | NDYDGFLENAHEHHGVY | GAGCATCCGTCTTGCGA | TAATAAACCTGT |
| 58) | GAAGAAATATAA | NQGRGIDSGERLMQPTQ | TACGATGATTTTCACAT | GGTGGGGGTATT |
| | GAGCCACCATGG | MSAQEDLGDDTGIHVIP | CGATGAAGACAAACTGG | GATGGGGTTCGG |
| | GGACAGTTAATA | TLNGDDRHKIVNVDQRQ | ATACAAACTCCGTATAT | AATTATCACGGG |
| | AACCTGTGGTGG | YGDVFKGDLNPKPQGQR | GAGCCTTACTACCATTC | AACGTTGCGTAT |
| | GGGTATTGATGG | LIEVSVEENHPFTLRAP | AGATCATGCGGAGTCTT | AACGAATCCGGT |
| | GGTTCGGAATTA | IQRIYGVRYTETWSFLP | CATGGGTAAATCGGGGA | CAGAGCATCCGT |
| | TCACGGGAACGT | SLTCTGDAAPAIQHICL | GAGTCTTCGCGAAAAGC | CTTGCGATACGA |
| | TGCGTATAACGA | KHTTCFQDVVVDVDCAE | GTACGATCATAACTCAC | TGATTTTCACAT |
| | ATCCGGTCAGAG | NTKEDQLAEISYRFQGK | CTTATATATGGCCACGT | CGATGAAGACAA |
| | CATCCGTCTTGC | KEADQPWIVVNTSTLFD | AATGATTATGATGGATT | ACTGGATACAAA |
| | GATACGATGATT | ELELDPPEIEPGVLKVL | TTTAGAGAACGCACACG | CTCCGTATATGA |
| | TTCACATCGATG | RTEKQYLGVYIWNMRGS | AACACCATGGGGTGTAT | GCCTTACTACCA |
| | AAGACAAACTGG | DGTSTYATFLVTWKGDE | AATCAGGGCCGTGGTAT | TTCAGATCATGC |
| | ATACAAACTCCG | KTRNPTPAVTPQPRGAE | CGATAGCGGGGAACGGT | GGAGTCTTCATG |
| | TATATGAGCCTT | FHMWNYHSHVFSVGDTF | TAATGCAACCCACACAA | GGTAAATCGGGG |
| | ACTACCATTCAG | SLAMHLQYKIHEAPFDL | ATGTCTGCACAGGAGGA | AGAGTCTTCGCG |
| | ATCATGCGGAGT | LLEWLYVPIDPTCQPMR | TCTTGGGGACGATACGG | AAAAGCGTACGA |
| | CTTCATGGGTAA | LYSTCLYHPNAPQCLSH | GCATCCACGTTATCCCT | TCATAACTCACC |
| | ATCGGGGAGAGT | MNSGCTFTSPHLAQRVA | ACGTTAAACGGCGATGA | TTATATATGGCC |
| | CTTCGCGAAAAG | STVYQNCEHADNYTAYC | CAGACATAAAATTGTAA | ACGTAATGATTA |
| | CGTACGATCATA | LGISHMEPSFGLILHDG | ATGTGGACCAACGTCAA | TGATGGATTTTT |
| | ACTCACCTTATA | GTTLKFVDTPESLSGLY | TACGGTGACGTGTTTAA | AGAGAACGCACA |
| | TATGGCCACGTA | VFVVYFNGHVEAVAYTV | AGGAGATCTTAATCCAA | CGAACACCATGG |
| | ATGATTATGATG | VSTVDHFVNAIEERGFP | AACCCCAAGGCCAAAGA | GGTGTATAATCA |
| | GATTTTTAGAGA | PTAGQPPATTKPKEITP | CTCATTGAGGTGTCAGT | GGGCCGTGGTAT |
| | ACGCACACGAAC | VNPGTSPLLRYAAWTGG | GGAAGAAAATCACCCGT | CGATAGCGGGGA |
| | ACCATGGGGTGT | LAAVVLLCLVIFLICTA | TTACTTTACGCGCACCG | ACGGTTAATGCA |
| | ATAATCAGGGCC | KRMRVKAYRVDKSPYNQ | ATTCAGCGGATTTATGG | ACCCACACAAAT |
| | GTGGTATCGATA | SMYYAGLPVDDFEDAEA | AGTCCGGTACACCGAGA | GTCTGCACAGGA |
| | GCGGGGAACGGT | ADAEEEFGNAIGGSHGG | CTTGGAGCTTTTTGCCG | GGATCTTGGGGA |
| | TAATGCAACCCA | SSYTVYIDKTR* | TCATTAACCTGTACGGG | CGATACGGGCAT |
| | CACAAATGTCTG | | AGACGCAGCGCCCGCCA | CCACGTTATCCC |
| | CACAGGAGGATC | | TCCAGCATATATGTTTA | TACGTTAAACGG |
| | TTGGGGACGATA | | AAACATACAACATGCTT | CGATGACAGACA |
| | CGGGCATCCACG | | TCAAGACGTGGTGGTGG | TAAAATTGTAAA |
| | TTATCCCTACGT | | ATGTGGATTGCGCGGAA | TGTGGACCAACG |
| | TAAACGGCGATG | | AATACTAAAGAGGATCA | TCAATACGGTGA |
| | ACAGACATAAAA | | GTTGGCCGAAATCAGTT | CGTGTTTAAAGG |
| | TTGTAAATGTGG | | ACCGTTTTCAAGGTAAG | AGATCTTAATCC |
| | ACCAACGTCAAT | | AAGGAAGCGGACCAACC | AAAACCCCAAGG |
| | ACGGTGACGTGT | | GTGGATTGTTGTAAACA | CCAAAGACTCAT |
| | TTAAAGGAGATC | | CGAGCACACTGTTTGAT | TGAGGTGTCAGT |
| | TTAATCCAAAAC | | GAACTCGAATTAGACCC | GGAAGAAAATCA |
| | CCCAAGGCCAAA | | CCCCGAGATTGAACCGG | CCCGTTTACTTT |
| | GACTCATTGAGG | | GTGTCTTGAAAGTACTT | ACGCGCACCGAT |
| | TGTCAGTGGAAG | | CGGACAGAAAAACAATA | TCAGCGGATTTA |
| | AAAATCACCCGT | | CTTGGGTGTGTACATTT | TGGAGTCCGGTA |
| | TTACTTTACGCG | | GGAACATGCGCGGCTCC | CACCGAGACTTG |
| | CACCGATTCAGC | | GATGGTACGTCTACCTA | GAGCTTTTTGCC |
| | GGATTTATGGAG | | CGCCACGTTTTTGGTCA | GTCATTAACCTG |
| | TCCGGTACACCG | | CCTGGAAAGGGGATGAA | TACGGGAGACGC |
| | AGACTTGGAGCT | | AAAACAAGAAACCCTAC | AGCGCCCGCCAT |
| | TTTTGCCGTCAT | | GCCCGCAGTAACTCCTC | CCAGCATATATG |
| | TAACCTGTACGG | | AACCAAGAGGGGCTGAG | TTTAAAACATAC |
| | GAGACGCAGCGC | | TTTCATATGTGGAATTA | AACATGCTTTCA |
| | CCGCCATCCAGC | | CCACTCGCATGTATTTT | AGACGTGGTGGT |
| | ATATATGTTTAA | | CAGTTGGTGATACGTTT | GGATGTGGATTG |
| | AACATACAACAT | | AGCTTGGCAATGCATCT | CGCGGAAAATAC |
| | GCTTTCAAGACG | | TCAGTATAAGATACATG | TAAAGAGGATCA |
| | TGGTGGTGGATG | | AAGCGCCATTTGATTTG | GTTGGCCGAAAT |
| | TGGATTGCGCGG | | CTGTTAGAGTGGTTGTA | CAGTTACCGTTT |
| | AAAATACTAAAG | | TGTCCCCATCGATCCTA | TCAAGGTAAGAA |
| | AGGATCAGTTGG | | CATGTCAACCAATGCGG | GGAAGCGGACCA |
| | CCGAAATCAGTT | | TTATATTCTACGTGTTT | ACCGTGGATTGT |
| | ACCGTTTTCAAG | | GTATCATCCCAACGCAC | TGTAAACACGAG |
| | GTAAGAAGGAAG | | CCCAATGCCTCTCTCAT | CACACTGTTTGA |
| | CGGACCAACCGT | | ATGAATTCCGGTTGTAC | TGAACTCGAATT |
| | GGATTGTTGTAA | | ATTTACCTCGCCACATT | AGACCCCCCCGA |
| | ACACGAGCACAC | | TAGCCCAGCGTGTTGCA | GATTGAACCGGG |
| | TGTTTGATGAAC | | AGCACAGTGTATCAAAA | TGTCTTGAAAGT |
| | TCGAATTAGACC | | TTGTGAACATGCAGATA | ACTTCGGACAGA |
| | CCCCCGAGATTG | | ACTACACCGCATATTGT | AAAACAATACTT |
| | AACCGGGTGTCT | | CTGGGAATATCTCATAT | GGGTGTGTACAT |
| | TGAAAGTACTTC | | GGAGCCTAGCTTTGGTC | TTGGAACATGCG |
| | GGACAGAAAAAC | | TAATCTTACACGACGGG | CGGCTCCGATGG |
| | AATACTTGGGTG | | GGCACCACGTTAAAGTT | TACGTCTACCTA |
| | TGTACATTTGGA | | TGTAGATACACCCGAGA | CGCCACGTTTTT |
| | ACATGCGCGGCT | | GTTTGTCGGGATTATAC | GGTCACCTGGAA |
| | CCGATGGTACGT | | GTTTTTGTGGTGTATTT | AGGGGATGAAAA |
| | CTACCTACGCCA | | TAACGGGCATGTTGAAG | AACAAGAAACCC |
| | CGTTTTTGGTCA | | CCGTAGCATACACTGTT | TACGCCCGCAGT |
| | CCTGGAAAGGGG | | GTATCCACAGTAGATCA | AACTCCTCAACC |
| | ATGAAAAAACAA | | TTTTGTAAACGCAATTG | AAGAGGGGCTGA |
| | GAAACCCTACGC | | AAGAGCGTGGATTTCCG | GTTTCATATGTG |
| | CCGCAGTAACTC | | CCAACGGCCGGTCAGCC | GAATTACCACTC |
| | CTCAACCAAGAG | | ACCGGCGACTACTAAAC | GCATGTATTTTC |
| | GGGCTGAGTTTC | | CCAAGGAAATTACCCCC | AGTTGGTGATAC |
| | ATATGTGGAATT | | GTAAACCCCGGAACGTC | GTTTAGCTTGGC |
| | ACCACTCGCATG | | ACCACTTCTACGATATG | AATGCATCTTCA |
| | TATTTTCAGTTG | | CCGCATGGACCGGAGGG | GTATAAGATACA |
| | GTGATACGTTTA | | CTTGCAGCAGTAGTACT | TGAAGCGCCATT |
| | GCTTGGCAATGC | | TTTATGTCTCGTAATAT | TGATTTGCTGTT |
| | ATCTTCAGTATA | | TTTTAATCTGTACGGCT | AGAGTGGTTGTA |
| | AGATACATGAAG | | AAACGAATGAGGGTTAA | TGTCCCCATCGA |
| | CGCCATTTGATT | | AGCCTATAGGGTAGACA | TCCTACATGTCA |
| | TGCTGTTAGAGT | | AGTCCCCGTATAACCAA | ACCAATGCGGTT |
| | GGTTGTATGTCC | | AGCATGTATTACGCTGG | ATATTCTACGTG |
| | CCATCGATCCTA | | CCTTCCAGTGGACGATT | TTTGTATCATCC |
| | CATGTCAACCAA | | TCGAGGACGCCGAAGCC | CAACGCACCCCA |
| | TGCGGTTATATT | | GCCGATGCCGAAGAAGA | ATGCCTCTCTCA |
| | CTACGTGTTTGT | | GTTTGGTAACGCGATTG | TATGAATTCCGG |
| | ATCATCCCAACG | | GAGGGAGTCACGGGGGT | TTGTACATTTAC |
| | CACCCCAATGCC | | TCGAGTTACACGGTGTA | CTCGCCACATTT |
| | TCTCTCATATGA | | TATAGATAAGACCCGGT | AGCCCAGCGTGT |
| | ATTCCGGTTGTA | | GA | TGCAAGCACAGT |
| | CATTTACCTCGC | | | GTATCAAAATTG |
| | CACATTTAGCCC | | | TGAACATGCAGA |
| | AGCGTGTTGCAA | | | TAACTACACCGC |
| | GCACAGTGTATC | | | ATATTGTCTGGG |
| | AAAATTGTGAAC | | | AATATCTCATAT |
| | ATGCAGATAACT | | | GGAGCCTAGCTT |
| | ACACCGCATATT | | | TGGTCTAATCTT |
| | GTCTGGGAATAT | | | ACACGACGGGGG |
| | CTCATATGGAGC | | | CACCACGTTAAA |
| | CTAGCTTTGGTC | | | GTTTGTAGATAC |
| | TAATCTTACACG | | | ACCCGAGAGTTT |
| | ACGGGGGCACCA | | | GTCGGGATTATA |
| | CGTTAAAGTTTG | | | CGTTTTTGTGGT |
| | TAGATACACCCG | | | GTATTTTAACGG |
| | AGAGTTTGTCGG | | | GCATGTTGAAGC |
| | GATTATACGTTT | | | CGTAGCATACAC |
| | TTGTGGTGTATT | | | TGTTGTATCCAC |
| | TTAACGGGCATG | | | AGTAGATCATTT |
| | TTGAAGCCGTAG | | | TGTAAACGCAAT |
| | CATACACTGTTG | | | TGAAGAGCGTGG |
| | TATCCACAGTAG | | | ATTTCCGCCAAC |
| | ATCATTTTGTAA | | | GGCCGGTCAGCC |
| | ACGCAATTGAAG | | | ACCGGCGACTAC |
| | AGCGTGGATTTC | | | TAAACCCAAGGA |
| | CGCCAACGGCCG | | | AATTACCCCCGT |
| | GTCAGCCACCGG | | | AAACCCCGGAAC |
| | CGACTACTAAAC | | | GTCACCACTTCT |
| | CCAAGGAAATTA | | | ACGATATGCCGC |
| | CCCCCGTAAACC | | | ATGGACCGGAGG |
| | CCGGAACGTCAC | | | GCTTGCAGCAGT |
| | CACTTCTACGAT | | | AGTACTTTTATG |
| | ATGCCGCATGGA | | | TCTCGTAATATT |
| | CCGGAGGGCTTG | | | TTTAATCTGTAC |
| | CAGCAGTAGTAC | | | GGCTAAACGAAT |
| | TTTTATGTCTCG | | | GAGGGTTAAAGC |
| | TAATATTTTTAA | | | CTATAGGGTAGA |
| | TCTGTACGGCTA | | | CAAGTCCCCGTA |
| | AACGAATGAGGG | | | TAACCAAAGCAT |
| | TTAAAGCCTATA | | | GTATTACGCTGG |
| | GGGTAGACAAGT | | | CCTTCCAGTGGA |
| | CCCCGTATAACC | | | CGATTTCGAGGA |
| | AAAGCATGTATT | | | CGCCGAAGCCGC |
| | ACGCTGGCCTTC | | | CGATGCCGAAGA |
| | CAGTGGACGATT | | | AGAGTTTGGTAA |
| | TCGAGGACGCCG | | | CGCGATTGGAGG |
| | AAGCCGCCGATG | | | GAGTCACGGGGG |
| | CCGAAGAAGAGT | | | TTCGAGTTACAC |
| | TTGGTAACGCGA | | | GGTGTATATAGA |
| | TTGGAGGGAGTC | | | TAAGACCCGGTG |
| | ACGGGGGTTCGA | | | ATGATAATAGGC |
| | GTTACACGGTGT | | | TGGAGCCTCGGT |
| | ATATAGATAAGA | | | GGCCATGCTTCT |
| | CCCGGTGATGAT | | | TGCCCCTTGGGC |
| | AATAGGCTGGAG | | | CTCCCCCCAGCC |
| | CCTCGGTGGCCA | | | CCTCCTCCCCTT |
| | TGCTTCTTGCCC | | | CCTGCACCCGTA |
| | CTTGGGCCTCCC | | | CCCCCGTGGTCT |
| | CCCAGCCCCTCC | | | TTGAATAAAGTC |
| | TCCCCTTCCTGC | | | TGAGTGGGCGGC |
| | ACCCGTACCCCC | | | AAAAAAAAAAAA |
| | GTGGTCTTTGAA | | | AAAAAAAAAAAA |
| | TAAAGTCTGAGT | | | AAAAAAAAAAAA |
| | GGGCGGC | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAATCTAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 29 | SEQ ID NO: 30 | SEQ ID NO: 31 | SEQ ID NO: 32 |
| VZV-GE- | TCAAGCTTTTGG | MGTVNKPVVGVLMGFGI | ATGGGGACAGTTAATAA | G*GGGAAATAAG |
| full_ | ACCCTCGTACAG | ITGTLRITNPVRASVLR | ACCTGTGGTGGGGGTAT | AGAGAAAAGAAG |
| with_ | AAGCTAATACGA | YDDFHIDEDKLDTNSVY | TGATGGGGTTCGGAATT | AGTAAGAAGAAA |
| AEAADA | CTCACTATAGGG | EPYYHSDHAESSWVNRG | ATCACGGGAACGTTGCG | TATAAGAGCCAC |
| (SEQ | AAATAAGAGAGA | ESSRKAYDHNSPYIWPR | TATAACGAATCCGGTCA | CATGGGGACAGT |
| ID NO: | AAAGAAGAGTAA | NDYDGFLENAHEHHGVY | GAGCATCCGTCTTGCGA | TAATAAACCTGT |
| 58)_and | GAAGAAATATAA | NQGRGIDSGERLMQPTQ | TACGATGATTTTCACAT | GGTGGGGGTATT |
| Y582G | GAGCCACCATGG | MSAQEDLGDDTGIHVIP | CGATGAAGACAAACTGG | GATGGGGTTCGG |
| | GGACAGTTAATA | TLNGDDRHKIVNVDQRQ | ATACAAACTCCGTATAT | AATTATCACGGG |
| | AACCTGTGGTGG | YGDVFKGDLNPKPQGQR | GAGCCTTACTACCATTC | AACGTTGCGTAT |
| | GGGTATTGATGG | LIEVSVEENHPFTLRAP | AGATCATGCGGAGTCTT | AACGAATCCGGT |
| | GGTTCGGAATTA | IQRIYGVRYTETWSFLP | CATGGGTAAATCGGGGA | CAGAGCATCCGT |
| | TCACGGGAACGT | SLTCTGDAAPAIQHICL | GAGTCTTCGCGAAAAGC | CTTGCGATACGA |
| | TGCGTATAACGA | KHTTCFQDVVVDVDCAE | GTACGATCATAACTCAC | TGATTTTCACAT |
| | ATCCGGTCAGAG | NTKEDQLAEISYRFQGK | CTTATATATGGCCACGT | CGATGAAGACAA |
| | CATCCGTCTTGC | KEADQPWIVVNISTLFD | AATGATTATGATGGATT | ACTGGATACAAA |
| | GATACGATGATT | ELELDPPEIEPGVLKVL | TTTAGAGAACGCACACG | CTCCGTATATGA |
| | TTCACATCGATG | RTEKQYLGVYIWNMRGS | AACACCATGGGGTGTAT | GCCTTACTACCA |
| | AAGACAAACTGG | DGTSTYATFLVTWKGDE | AATCAGGGCCGTGGTAT | TTCAGATCATGC |
| | ATACAAACTCCG | KTRNPTPAVTPQPRGAE | CGATAGCGGGGAACGGT | GGAGTCTTCATG |
| | TATATGAGCCTT | FHMWNYHSHVFSVGDTF | TAATGCAACCCACACAA | GGTAAATCGGGG |
| | ACTACCATTCAG | SLAMHLQYKIHEAPFDL | ATGTCTGCACAGGAGGA | AGAGTCTTCGCG |
| | ATCATGCGGAGT | LLEWLYVPIDPTCQPMR | TCTTGGGGACGATACGG | AAAAGCGTACGA |
| | CTTCATGGGTAA | LYSTCLYHPNAPQCLSH | GCATCCACGTTATCCCT | TCATAACTCACC |
| | ATCGGGGAGAGT | MNSGCTFTSPHLAQRVA | ACGTTAAACGGCGATGA | TTATATATGGCC |
| | CTTCGCGAAAAG | STVYQNCEHADNYTAYC | CAGACATAAAATTGTAA | ACGTAATGATTA |
| | CGTACGATCATA | LGISHMEPSFGLILHDG | ATGTGGACCAACGTCAA | TGATGGATTTTT |
| | ACTCACCTTATA | GTTLKFVDTPESLSGLY | TACGGTGACGTGTTTAA | AGAGAACGCACA |
| | TATGGCCACGTA | VFVVYFNGHVEAVAYTV | AGGAGATCTTAATCCAA | CGAACACCATGG |
| | ATGATTATGATG | VSTVDHFVNAIEERGFP | AACCCCAAGGCCAAAGA | GGTGTATAATCA |
| | GATTTTTAGAGA | PTAGQPPATTKPKEITP | CTCATTGAGGTGTCAGT | GGGCCGTGGTAT |
| | ACGCACACGAAC | VNPGTSPLLRYAAWTGG | GGAAGAAAATCACCCGT | CGATAGCGGGGA |
| | ACCATGGGGTGT | LAAVVLLCLVIFLICTA | TTACTTTACGCGCACCG | ACGGTTAATGCA |
| | ATAATCAGGGCC | KRMRVKAYRVDKSPYNQ | ATTCAGCGGATTTATGG | ACCCACACAAAT |
| | GTGGTATCGATA | SMYGAGLPVDDFEDAEA | AGTCCGGTACACCGAGA | GTCTGCACAGGA |
| | GCGGGGAACGGT | ADAEEEFGNAIGGSHGG | CTTGGAGCTTTTTGCCG | GGATCTTGGGGA |
| | TAATGCAACCCA | SSYTVYIDKTR* | TCATTAACCTGTACGGG | CGATACGGGCAT |
| | CACAAATGTCTG | | AGACGCAGCGCCCGCCA | CCACGTTATCCC |
| | CACAGGAGGATC | | TCCAGCATATATGTTTA | TACGTTAAACGG |
| | TTGGGGACGATA | | AAACATACAACATGCTT | CGATGACAGACA |
| | CGGGCATCCACG | | TCAAGACGTGGTGGTGG | TAAAATTGTAAA |
| | TTATCCCTACGT | | ATGTGGATTGCGCGGAA | TGTGGACCAACG |
| | TAAACGGCGATG | | AATACTAAAGAGGATCA | TCAATACGGTGA |
| | ACAGACATAAAA | | GTTGGCCGAAATCAGTT | CGTGTTTAAAGG |
| | TTGTAAATGTGG | | ACCGTTTTCAAGGTAAG | AGATCTTAATCC |
| | ACCAACGTCAAT | | AAGGAAGCGGACCAACC | AAAACCCCAAGG |
| | ACGGTGACGTGT | | GTGGATTGTTGTAAACA | CCAAAGACTCAT |
| | TTAAAGGAGATC | | CGAGCACACTGTTTGAT | TGAGGTGTCAGT |
| | TTAATCCAAAAC | | GAACTCGAATTAGACCC | GGAAGAAAATCA |
| | CCCAAGGCCAAA | | CCCCGAGATTGAACCGG | CCCGTTTACTTT |
| | GACTCATTGAGG | | GTGTCTTGAAAGTACTT | ACGCGCACCGAT |
| | TGTCAGTGGAAG | | CGGACAGAAAAACAATA | TCAGCGGATTTA |
| | AAAATCACCCGT | | CTTGGGTGTGTACATTT | TGGAGTCCGGTA |
| | TTACTTTACGCG | | GGAACATGCGCGGCTCC | CACCGAGACTTG |
| | CACCGATTCAGC | | GATGGTACGTCTACCTA | GAGCTTTTTGCC |
| | GGATTTATGGAG | | CGCCACGTTTTTGGTCA | GTCATTAACCTG |
| | TCCGGTACACCG | | CCTGGAAAGGGGATGAA | TACGGGAGACGC |
| | AGACTTGGAGCT | | AAAACAAGAAACCCTAC | AGCGCCCGCCAT |
| | TTTTGCCGTCAT | | GCCCGCAGTAACTCCTC | CCAGCATATATG |
| | TAACCTGTACGG | | AACCAAGAGGGGCTGAG | TTTAAAACATAC |
| | GAGACGCAGCGC | | TTTCATATGTGGAATTA | AACATGCTTTCA |
| | CCGCCATCCAGC | | CCACTCGCATGTATTTT | AGACGTGGTGGT |
| | ATATATGTTTAA | | CAGTTGGTGATACGTTT | GGATGTGGATTG |
| | AACATACAACAT | | AGCTTGGCAATGCATCT | CGCGGAAAATAC |
| | GCTTTCAAGACG | | TCAGTATAAGATACATG | TAAAGAGGATCA |
| | TGGTGGTGGATG | | AAGCGCCATTTGATTTG | GTTGGCCGAAAT |
| | TGGATTGCGCGG | | CTGTTAGAGTGGTTGTA | CAGTTACCGTTT |
| | AAAATACTAAAG | | TGTCCCCATCGATCCTA | TCAAGGTAAGAA |
| | AGGATCAGTTGG | | CATGTCAACCAATGCGG | GGAAGCGGACCA |
| | CCGAAATCAGTT | | TTATATTCTACGTGTTT | ACCGTGGATTGT |
| | ACCGTTTTCAAG | | GTATCATCCCAACGCAC | TGTAAACACGAG |
| | GTAAGAAGGAAG | | CCCAATGCCTCTCTCAT | CACACTGTTTGA |
| | CGGACCAACCGT | | ATGAATTCCGGTTGTAC | TGAACTCGAATT |
| | GGATTGTTGTAA | | ATTTACCTCGCCACATT | AGACCCCCCCGA |
| | ACACGAGCACAC | | TAGCCCAGCGTGTTGCA | GATTGAACCGGG |
| | TGTTTGATGAAC | | AGCACAGTGTATCAAAA | TGTCTTGAAAGT |
| | TCGAATTAGACC | | TTGTGAACATGCAGATA | ACTTCGGACAGA |
| | CCCCCGAGATTG | | ACTACACCGCATATTGT | AAAACAATACTT |
| | AACCGGGTGTCT | | CTGGGAATATCTCATAT | GGGTGTGTACAT |
| | TGAAAGTACTTC | | GGAGCCTAGCTTTGGTC | TTGGAACATGCG |
| | GGACAGAAAAAC | | TAATCTTACACGACGGG | CGGCTCCGATGG |
| | AATACTTGGGTG | | GGCACCACGTTAAAGTT | TACGTCTACCTA |
| | TGTACATTTGGA | | TGTAGATACACCCGAGA | CGCCACGTTTTT |
| | ACATGCGCGGCT | | GTTTGTCGGGATTATAC | GGTCACCTGGAA |
| | CCGATGGTACGT | | GTTTTTGTGGTGTATTT | AGGGGATGAAAA |
| | CTACCTACGCCA | | TAACGGGCATGTTGAAG | AACAAGAAACCC |
| | CGTTTTTGGTCA | | CCGTAGCATACACTGTT | TACGCCCGCAGT |
| | CCTGGAAAGGGG | | GTATCCACAGTAGATCA | AACTCCTCAACC |
| | ATGAAAAAACAA | | TTTTGTAAACGCAATTG | AAGAGGGGCTGA |
| | GAAACCCTACGC | | AAGAGCGTGGATTTCCG | GTTTCATATGTG |
| | CCGCAGTAACTC | | CCAACGGCCGGTCAGCC | GAATTACCACTC |
| | CTCAACCAAGAG | | ACCGGCGACTACTAAAC | GCATGTATTTTC |
| | GGGCTGAGTTTC | | CCAAGGAAATTACCCCC | AGTTGGTGATAC |
| | ATATGTGGAATT | | GTAAACCCCGGAACGTC | GTTTAGCTTGGC |
| | ACCACTCGCATG | | ACCACTTCTACGATATG | AATGCATCTTCA |
| | TATTTTCAGTTG | | CCGCATGGACCGGAGGG | GTATAAGATACA |
| | GTGATACGTTTA | | CTTGCAGCAGTAGTACT | TGAAGCGCCATT |
| | GCTTGGCAATGC | | TTTATGTCTCGTAATAT | TGATTTGCTGTT |
| | ATCTTCAGTATA | | TTTTAATCTGTACGGCT | AGAGTGGTTGTA |
| | AGATACATGAAG | | AAACGAATGAGGGTTAA | TGTCCCCATCGA |
| | CGCCATTTGATT | | AGCCTATAGGGTAGACA | TCCTACATGTCA |
| | TGCTGTTAGAGT | | AGTCCCCGTATAACCAA | ACCAATGCGGTT |
| | GGTTGTATGTCC | | AGCATGTATGGCGCTGG | ATATTCTACGTG |
| | CCATCGATCCTA | | CCTTCCAGTGGACGATT | TTTGTATCATCC |
| | CATGTCAACCAA | | TCGAGGACGCCGAAGCC | CAACGCACCCCA |
| | TGCGGTTATATT | | GCCGATGCCGAAGAAGA | ATGCCTCTCTCA |
| | CTACGTGTTTGT | | GTTTGGTAACGCGATTG | TATGAATTCCGG |
| | ATCATCCCAACG | | GAGGGAGTCACGGGGGT | TTGTACATTTAC |
| | CACCCCAATGCC | | TCGAGTTACACGGTGTA | CTCGCCACATTT |
| | TCTCTCATATGA | | TATAGATAAGACCCGGT | AGCCCAGCGTGT |
| | ATTCCGGTTGTA | | GA | TGCAAGCACAGT |
| | CATTTACCTCGC | | | GTATCAAAATTG |
| | CACATTTAGCCC | | | TGAACATGCAGA |
| | AGCGTGTTGCAA | | | TAACTACACCGC |
| | GCACAGTGTATC | | | ATATTGTCTGGG |
| | AAAATTGTGAAC | | | AATATCTCATAT |
| | ATGCAGATAACT | | | GGAGCCTAGCTT |
| | ACACCGCATATT | | | TGGTCTAATCTT |
| | GTCTGGGAATAT | | | ACACGACGGGGG |
| | CTCATATGGAGC | | | CACCACGTTAAA |
| | CTAGCTTTGGTC | | | GTTTGTAGATAC |
| | TAATCTTACACG | | | ACCCGAGAGTTT |
| | ACGGGGGCACCA | | | GTCGGGATTATA |
| | CGTTAAAGTTTG | | | CGTTTTTGTGGT |
| | TAGATACACCCG | | | GTATTTTAACGG |
| | AGAGTTTGTCGG | | | GCATGTTGAAGC |
| | GATTATACGTTT | | | CGTAGCATACAC |
| | TTGTGGTGTATT | | | TGTTGTATCCAC |
| | TTAACGGGCATG | | | AGTAGATCATTT |
| | TTGAAGCCGTAG | | | TGTAAACGCAAT |
| | CATACACTGTTG | | | TGAAGAGCGTGG |
| | TATCCACAGTAG | | | ATTTCCGCCAAC |
| | ATCATTTTGTAA | | | GGCCGGTCAGCC |
| | ACGCAATTGAAG | | | ACCGGCGACTAC |
| | AGCGTGGATTTC | | | TAAACCCAAGGA |
| | CGCCAACGGCCG | | | AATTACCCCCGT |
| | GTCAGCCACCGG | | | AAACCCCGGAAC |
| | CGACTACTAAAC | | | GTCACCACTTCT |
| | CCAAGGAAATTA | | | ACGATATGCCGC |
| | CCCCCGTAAACC | | | ATGGACCGGAGG |
| | CCGGAACGTCAC | | | GCTTGCAGCAGT |
| | CACTTCTACGAT | | | AGTACTTTTATG |
| | ATGCCGCATGGA | | | TCTCGTAATATT |
| | CCGGAGGGCTTG | | | TTTAATCTGTAC |
| | CAGCAGTAGTAC | | | GGCTAAACGAAT |
| | TTTTATGTCTCG | | | GAGGGTTAAAGC |
| | TAATATTTTTAA | | | CTATAGGGTAGA |
| | TCTGTACGGCTA | | | CAAGTCCCCGTA |
| | AACGAATGAGGG | | | TAACCAAAGCAT |
| | TTAAAGCCTATA | | | GTATGGCGCTGG |
| | GGGTAGACAAGT | | | CCTTCCAGTGGA |
| | CCCCGTATAACC | | | CGATTTCGAGGA |
| | AAAGCATGTATG | | | CGCCGAAGCCGC |
| | GCGCTGGCCTTC | | | CGATGCCGAAGA |
| | CAGTGGACGATT | | | AGAGTTTGGTAA |
| | TCGAGGACGCCG | | | CGCGATTGGAGG |
| | AAGCCGCCGATG | | | GAGTCACGGGGG |
| | CCGAAGAAGAGT | | | TTCGAGTTACAC |
| | TTGGTAACGCGA | | | GGTGTATATAGA |
| | TTGGAGGGAGTC | | | TAAGACCCGGTG |
| | ACGGGGGTTCGA | | | ATGATAATAGGC |
| | GTTACACGGTGT | | | TGGAGCCTCGGT |
| | ATATAGATAAGA | | | GGCCATGCTTCT |
| | CCCGGTGATGAT | | | TGCCCCTTGGGC |
| | AATAGGCTGGAG | | | CTCCCCCCAGCC |
| | CCTCGGTGGCCA | | | CCTCCTCCCCTT |
| | TGCTTCTTGCCC | | | CCTGCACCCGTA |
| | CTTGGGCCTCCC | | | CCCCCGTGGTCT |
| | CCCAGCCCCTCC | | | TTGAATAAAGTC |
| | TCCCCTTCCTGC | | | TGAGTGGGCGGC |
| | ACCCGTACCCCC | | | AAAAAAAAAAAA |
| | GTGGTCTTTGAA | | | AAAAAAAAAAAA |
| | TAAAGTCTGAGT | | | AAAAAAAAAAAA |
| | GGGCGGC | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAATCTAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 33 | SEQ ID NO: 34 | SEQ ID NO: 35 | SEQ ID NO: 36 |
| VZV-GE- | TCAAGCTTTTGG | MGTVNKPVVGVLMGFGI | ATGGGGACAGTTAATAA | G*GGGAAATAAG |
| truncated- | ACCCTCGTACAG | ITGTLRITNPVRASVLR | ACCTGTGGTGGGGGTAT | AGAGAAAAGAAG |
| delete_ | AAGCTAATACGA | YDDFHIDEDKLDTNSVY | TGATGGGGTTCGGAATT | AGTAAGAAGAAA |
| from_574 | CTCACTATAGGG | EPYYHSDHAESSWVNRG | ATCACGGGAACGTTGCG | TATAAGAGCCAC |
| | AAATAAGAGAGA | ESSRKAYDHNSPYIWPR | TATAACGAATCCGGTCA | CATGGGGACAGT |
| | AAAGAAGAGTAA | NDYDGFLENAHEHHGVY | GAGCATCCGTCTTGCGA | TAATAAACCTGT |
| | GAAGAAATATAA | NQGRGIDSGERLMQPTQ | TACGATGATTTTCACAT | GGTGGGGGTATT |
| | GAGCCACCATGG | MSAQEDLGDDTGIHVIP | CGATGAAGACAAACTGG | GATGGGGTTCGG |
| | GGACAGTTAATA | TLNGDDRHKIVNVDQRQ | ATACAAACTCCGTATAT | AATTATCACGGG |
| | AACCTGTGGTGG | YGDVFKGDLNPKPQGQR | GAGCCTTACTACCATTC | AACGTTGCGTAT |
| | GGGTATTGATGG | LIEVSVEENHPFTLRAP | AGATCATGCGGAGTCTT | AACGAATCCGGT |
| | GGTTCGGAATTA | IQRIYGVRYTETWSFLP | CATGGGTAAATCGGGGA | CAGAGCATCCGT |
| | TCACGGGAACGT | SLTCTGDAAPAIQHICL | GAGTCTTCGCGAAAAGC | CTTGCGATACGA |
| | TGCGTATAACGA | KHTTCFQDVVVDVDCAE | GTACGATCATAACTCAC | TGATTTTCACAT |
| | ATCCGGTCAGAG | NTKEDQLAEISYRFQGK | CTTATATATGGCCACGT | CGATGAAGACAA |
| | CATCCGTCTTGC | KEADQPWIVVNTSTLFD | AATGATTATGATGGATT | ACTGGATACAAA |
| | GATACGATGATT | ELELDPPEIEPGVLKVL | TTTAGAGAACGCACACG | CTCCGTATATGA |
| | TTCACATCGATG | RTEKQYLGVYIWNMRGS | AACACCATGGGGTGTAT | GCCTTACTACCA |
| | AAGACAAACTGG | DGTSTYATFLVTWKGDE | AATCAGGGCCGTGGTAT | TTCAGATCATGC |
| | ATACAAACTCCG | KTRNPTPAVTPQPRGAE | CGATAGCGGGGAACGGT | GGAGTCTTCATG |
| | TATATGAGCCTT | FHMWNYHSHVFSVGDTF | TAATGCAACCCACACAA | GGTAAATCGGGG |
| | ACTACCATTCAG | SLAMHLQYKIHEAPFDL | ATGTCTGCACAGGAGGA | AGAGTCTTCGCG |
| | ATCATGCGGAGT | LLEWLYVPIDPTCQPMR | TCTTGGGGACGATACGG | AAAAGCGTACGA |
| | CTTCATGGGTAA | LYSTCLYHPNAPQCLSH | GCATCCACGTTATCCCT | TCATAACTCACC |
| | ATCGGGGAGAGT | MNSGCTFTSPHLAQRVA | ACGTTAAACGGCGATGA | TTATATATGGCC |
| | CTTCGCGAAAAG | STVYQNCEHADNYTAYC | CAGACATAAAATTGTAA | ACGTAATGATTA |
| | CGTACGATCATA | LGISHMEPSFGLILHDG | ATGTGGACCAACGTCAA | TGATGGATTTTT |
| | ACTCACCTTATA | GTTLKFVDTPESLSGLY | TACGGTGACGTGTTTAA | AGAGAACGCACA |
| | TATGGCCACGTA | VFVVYFNGHVEAVAYTV | AGGAGATCTTAATCCAA | CGAACACCATGG |
| | ATGATTATGATG | VSTVDHFVNAIEERGFP | AACCCCAAGGCCAAAGA | GGTGTATAATCA |
| | GATTTTTAGAGA | PTAGQPPATTKPKEITP | CTCATTGAGGTGTCAGT | GGGCCGTGGTAT |
| | ACGCACACGAAC | VNPGTSPLLRYAAWTGG | GGAAGAAAATCACCCGT | CGATAGCGGGGA |
| | ACCATGGGGTGT | LAAVVLLCLVIFLICTA | TTACTTTACGCGCACCG | ACGGTTAATGCA |
| | ATAATCAGGGCC | KRMRVKAYRVDK* | ATTCAGCGGATTTATGG | ACCCACACAAAT |
| | GTGGTATCGATA | | AGTCCGGTACACCGAGA | GTCTGCACAGGA |
| | GCGGGGAACGGT | | CTTGGAGCTTTTTGCCG | GGATCTTGGGGA |
| | TAATGCAACCCA | | TCATTAACCTGTACGGG | CGATACGGGCAT |
| | CACAAATGTCTG | | AGACGCAGCGCCCGCCA | CCACGTTATCCC |
| | CACAGGAGGATC | | TCCAGCATATATGTTTA | TACGTTAAACGG |
| | TTGGGGACGATA | | AAACATACAACATGCTT | CGATGACAGACA |
| | CGGGCATCCACG | | TCAAGACGTGGTGGTGG | TAAAATTGTAAA |
| | TTATCCCTACGT | | ATGTGGATTGCGCGGAA | TGTGGACCAACG |
| | TAAACGGCGATG | | AATACTAAAGAGGATCA | TCAATACGGTGA |
| | ACAGACATAAAA | | GTTGGCCGAAATCAGTT | CGTGTTTAAAGG |
| | TTGTAAATGTGG | | ACCGTTTTCAAGGTAAG | AGATCTTAATCC |
| | ACCAACGTCAAT | | AAGGAAGCGGACCAACC | AAAACCCCAAGG |
| | ACGGTGACGTGT | | GTGGATTGTTGTAAACA | CCAAAGACTCAT |
| | TTAAAGGAGATC | | CGAGCACACTGTTTGAT | TGAGGTGTCAGT |
| | TTAATCCAAAAC | | GAACTCGAATTAGACCC | GGAAGAAAATCA |
| | CCCAAGGCCAAA | | CCCCGAGATTGAACCGG | CCCGTTTACTTT |
| | GACTCATTGAGG | | GTGTCTTGAAAGTACTT | ACGCGCACCGAT |
| | TGTCAGTGGAAG | | CGGACAGAAAAACAATA | TCAGCGGATTTA |
| | AAAATCACCCGT | | CTTGGGTGTGTACATTT | TGGAGTCCGGTA |
| | TTACTTTACGCG | | GGAACATGCGCGGCTCC | CACCGAGACTTG |
| | CACCGATTCAGC | | GATGGTACGTCTACCTA | GAGCTTTTTGCC |
| | GGATTTATGGAG | | CGCCACGTTTTTGGTCA | GTCATTAACCTG |
| | TCCGGTACACCG | | CCTGGAAAGGGGATGAA | TACGGGAGACGC |
| | AGACTTGGAGCT | | AAAACAAGAAACCCTAC | AGCGCCCGCCAT |
| | TTTTGCCGTCAT | | GCCCGCAGTAACTCCTC | CCAGCATATATG |
| | TAACCTGTACGG | | AACCAAGAGGGGCTGAG | TTTAAAACATAC |
| | GAGACGCAGCGC | | TTTCATATGTGGAATTA | AACATGCTTTCA |
| | CCGCCATCCAGC | | CCACTCGCATGTATTTT | AGACGTGGTGGT |
| | ATATATGTTTAA | | CAGTTGGTGATACGTTT | GGATGTGGATTG |
| | AACATACAACAT | | AGCTTGGCAATGCATCT | CGCGGAAAATAC |
| | GCTTTCAAGACG | | TCAGTATAAGATACATG | TAAAGAGGATCA |
| | TGGTGGTGGATG | | AAGCGCCATTTGATTTG | GTTGGCCGAAAT |
| | TGGATTGCGCGG | | CTGTTAGAGTGGTTGTA | CAGTTACCGTTT |
| | AAAATACTAAAG | | TGTCCCCATCGATCCTA | TCAAGGTAAGAA |
| | AGGATCAGTTGG | | CATGTCAACCAATGCGG | GGAAGCGGACCA |
| | CCGAAATCAGTT | | TTATATTCTACGTGTTT | ACCGTGGATTGT |
| | ACCGTTTTCAAG | | GTATCATCCCAACGCAC | TGTAAACACGAG |
| | GTAAGAAGGAAG | | CCCAATGCCTCTCTCAT | CACACTGTTTGA |
| | CGGACCAACCGT | | ATGAATTCCGGTTGTAC | TGAACTCGAATT |
| | GGATTGTTGTAA | | ATTTACCTCGCCACATT | AGACCCCCCCGA |
| | ACACGAGCACAC | | TAGCCCAGCGTGTTGCA | GATTGAACCGGG |
| | TGTTTGATGAAC | | AGCACAGTGTATCAAAA | TGTCTTGAAAGT |
| | TCGAATTAGACC | | TTGTGAACATGCAGATA | ACTTCGGACAGA |
| | CCCCCGAGATTG | | ACTACACCGCATATTGT | AAAACAATACTT |
| | AACCGGGTGTCT | | CTGGGAATATCTCATAT | GGGTGTGTACAT |
| | TGAAAGTACTTC | | GGAGCCTAGCTTTGGTC | TTGGAACATGCG |
| | GGACAGAAAAAC | | TAATCTTACACGACGGG | CGGCTCCGATGG |
| | AATACTTGGGTG | | GGCACCACGTTAAAGTT | TACGTCTACCTA |
| | TGTACATTTGGA | | TGTAGATACACCCGAGA | CGCCACGTTTTT |
| | ACATGCGCGGCT | | GTTTGTCGGGATTATAC | GGTCACCTGGAA |
| | CCGATGGTACGT | | GTTTTTGTGGTGTATTT | AGGGGATGAAAA |
| | CTACCTACGCCA | | TAACGGGCATGTTGAAG | AACAAGAAACCC |
| | CGTTTTTGGTCA | | CCGTAGCATACACTGTT | TACGCCCGCAGT |
| | CCTGGAAAGGGG | | GTATCCACAGTAGATCA | AACTCCTCAACC |
| | ATGAAAAAACAA | | TTTTGTAAACGCAATTG | AAGAGGGGCTGA |
| | GAAACCCTACGC | | AAGAGCGTGGATTTCCG | GTTTCATATGTG |
| | CCGCAGTAACTC | | CCAACGGCCGGTCAGCC | GAATTACCACTC |
| | CTCAACCAAGAG | | ACCGGCGACTACTAAAC | GCATGTATTTTC |
| | GGGCTGAGTTTC | | CCAAGGAAATTACCCCC | AGTTGGTGATAC |
| | ATATGTGGAATT | | GTAAACCCCGGAACGTC | GTTTAGCTTGGC |
| | ACCACTCGCATG | | ACCACTTCTACGATATG | AATGCATCTTCA |
| | TATTTTCAGTTG | | CCGCATGGACCGGAGGG | GTATAAGATACA |
| | GTGATACGTTTA | | CTTGCAGCAGTAGTACT | TGAAGCGCCATT |
| | GCTTGGCAATGC | | TTTATGTCTCGTAATAT | TGATTTGCTGTT |
| | ATCTTCAGTATA | | TTTTAATCTGTACGGCT | AGAGTGGTTGTA |
| | AGATACATGAAG | | AAACGAATGAGGGTTAA | TGTCCCCATCGA |
| | CGCCATTTGATT | | AGCCTATAGGGTAGACA | TCCTACATGTCA |
| | TGCTGTTAGAGT | | AGTGA | ACCAATGCGGTT |
| | GGTTGTATGTCC | | | ATATTCTACGTG |
| | CCATCGATCCTA | | | TTTGTATCATCC |
| | CATGTCAACCAA | | | CAACGCACCCCA |
| | TGCGGTTATATT | | | ATGCCTCTCTCA |
| | CTACGTGTTTGT | | | TATGAATTCCGG |
| | ATCATCCCAACG | | | TTGTACATTTAC |
| | CACCCCAATGCC | | | CTCGCCACATTT |
| | TCTCTCATATGA | | | AGCCCAGCGTGT |
| | ATTCCGGTTGTA | | | TGCAAGCACAGT |
| | CATTTACCTCGC | | | GTATCAAAATTG |
| | CACATTTAGCCC | | | TGAACATGCAGA |
| | AGCGTGTTGCAA | | | TAACTACACCGC |
| | GCACAGTGTATC | | | ATATTGTCTGGG |
| | AAAATTGTGAAC | | | AATATCTCATAT |
| | ATGCAGATAACT | | | GGAGCCTAGCTT |
| | ACACCGCATATT | | | TGGTCTAATCTT |
| | GTCTGGGAATAT | | | ACACGACGGGGG |
| | CTCATATGGAGC | | | CACCACGTTAAA |
| | CTAGCTTTGGTC | | | GTTTGTAGATAC |
| | TAATCTTACACG | | | ACCCGAGAGTTT |
| | ACGGGGGCACCA | | | GTCGGGATTATA |
| | CGTTAAAGTTTG | | | CGTTTTTGTGGT |
| | TAGATACACCCG | | | GTATTTTAACGG |
| | AGAGTTTGTCGG | | | GCATGTTGAAGC |
| | GATTATACGTTT | | | CGTAGCATACAC |
| | TTGTGGTGTATT | | | TGTTGTATCCAC |
| | TTAACGGGCATG | | | AGTAGATCATTT |
| | TTGAAGCCGTAG | | | TGTAAACGCAAT |
| | CATACACTGTTG | | | TGAAGAGCGTGG |
| | TATCCACAGTAG | | | ATTTCCGCCAAC |
| | ATCATTTTGTAA | | | GGCCGGTCAGCC |
| | ACGCAATTGAAG | | | ACCGGCGACTAC |
| | AGCGTGGATTTC | | | TAAACCCAAGGA |
| | CGCCAACGGCCG | | | AATTACCCCCGT |
| | GTCAGCCACCGG | | | AAACCCCGGAAC |
| | CGACTACTAAAC | | | GTCACCACTTCT |
| | CCAAGGAAATTA | | | ACGATATGCCGC |
| | CCCCCGTAAACC | | | ATGGACCGGAGG |
| | CCGGAACGTCAC | | | GCTTGCAGCAGT |
| | CACTTCTACGAT | | | AGTACTTTTATG |
| | ATGCCGCATGGA | | | TCTCGTAATATT |
| | CCGGAGGGCTTG | | | TTTAATCTGTAC |
| | CAGCAGTAGTAC | | | GGCTAAACGAAT |
| | TTTTATGTCTCG | | | GAGGGTTAAAGC |
| | TAATATTTTTAA | | | CTATAGGGTAGA |
| | TCTGTACGGCTA | | | CAAGTGATGATA |
| | AACGAATGAGGG | | | ATAGGCTGGAGC |
| | TTAAAGCCTATA | | | CTCGGTGGCCAT |
| | GGGTAGACAAGT | | | GCTTCTTGCCCC |
| | GATGATAATAGG | | | TTGGGCCTCCCC |
| | CTGGAGCCTCGG | | | CCAGCCCCTCCT |
| | TGGCCATGCTTC | | | CCCCTTCCTGCA |
| | TTGCCCCTTGGG | | | CCCGTACCCCCG |
| | CCTCCCCCCAGC | | | TGGTCTTTGAAT |
| | CCCTCCTCCCCT | | | AAAGTCTGAGTG |
| | TCCTGCACCCGT | | | GGCGGCAAAAAA |
| | ACCCCCGTGGTC | | | AAAAAAAAAAAA |
| | TTTGAATAAAGT | | | AAAAAAAAAAAA |
| | CTGAGTGGGCGG | | | AAAAAAAAAAAA |
| | C | | | AAAAAAAAAAAA |
| | | | | AAAAAAAAAAAA |
| | | | | AAAAAAAAAAAA |
| | | | | AAAAAAAAAAAA |
| | | | | AAAAAAAAAATC |
| | | | | TAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 37 | SEQ ID NO: 38 | SEQ ID NO: 39 | SEQ ID NO: 40 |
| VZV-GE- | TCAAGCTTTTGG | MGTVNKPVVGVLMGFGI | ATGGGGACAGTTAATAA | G*GGGAAATAAG |
| truncated- | ACCCTCGTACAG | ITGTLRITNPVRASVLR | ACCTGTGGTGGGGGTAT | AGAGAAAAGAAG |
| delete_ | AAGCTAATACGA | YDDFHIDEDKLDTNSVY | TGATGGGGTTCGGAATT | AGTAAGAAGAAA |
| from_ | CTCACTATAGGG | EPYYHSDHAESSWVNRG | ATCACGGGAACGTTGCG | TATAAGAGCCAC |
| 574_-_ | AAATAAGAGAGA | ESSRKAYDHNSPYIWPR | TATAACGAATCCGGTCA | CATGGGGACAGT |
| Y569A | AAAGAAGAGTAA | NDYDGFLENAHEHHGVY | GAGCATCCGTCTTGCGA | TAATAAACCTGT |
| | GAAGAAATATAA | NQGRGIDSGERLMQPTQ | TACGATGATTTTCACAT | GGTGGGGGTATT |
| | GAGCCACCATGG | MSAQEDLGDDTGIHVIP | CGATGAAGACAAACTGG | GATGGGGTTCGG |
| | GGACAGTTAATA | TLNGDDRHKIVNVDQRQ | ATACAAACTCCGTATAT | AATTATCACGGG |
| | AACCTGTGGTGG | YGDVFKGDLNPKPQGQR | GAGCCTTACTACCATTC | AACGTTGCGTAT |
| | GGGTATTGATGG | LIEVSVEENHPFTLRAP | AGATCATGCGGAGTCTT | AACGAATCCGGT |
| | GGTTCGGAATTA | IQRIYGVRYTETWSFLP | CATGGGTAAATCGGGGA | CAGAGCATCCGT |
| | TCACGGGAACGT | SLTCTGDAAPAIQHICL | GAGTCTTCGCGAAAAGC | CTTGCGATACGA |
| | TGCGTATAACGA | KHTTCFQDVVVDVDCAE | GTACGATCATAACTCAC | TGATTTTCACAT |
| | ATCCGGTCAGAG | NTKEDQLAEISYRFQGK | CTTATATATGGCCACGT | CGATGAAGACAA |
| | CATCCGTCTTGC | KEADQPWIVVNTSTLFD | AATGATTATGATGGATT | ACTGGATACAAA |
| | GATACGATGATT | ELELDPPEIEPGVLKVL | TTTAGAGAACGCACACG | CTCCGTATATGA |
| | TTCACATCGATG | RTEKQYLGVYIWNMRGS | AACACCATGGGGTGTAT | GCCTTACTACCA |
| | AAGACAAACTGG | DGTSTYATFLVTWKGDE | AATCAGGGCCGTGGTAT | TTCAGATCATGC |
| | ATACAAACTCCG | KTRNPTPAVTPQPRGAE | CGATAGCGGGGAACGGT | GGAGTCTTCATG |
| | TATATGAGCCTT | FHMWNYHSHVFSVGDTF | TAATGCAACCCACACAA | GGTAAATCGGGG |
| | ACTACCATTCAG | SLAMHLQYKIHEAPFDL | ATGTCTGCACAGGAGGA | AGAGTCTTCGCG |
| | ATCATGCGGAGT | LLEWLYVPIDPTCQPMR | TCTTGGGGACGATACGG | AAAAGCGTACGA |
| | CTTCATGGGTAA | LYSTCLYHPNAPQCLSH | GCATCCACGTTATCCCT | TCATAACTCACC |
| | ATCGGGGAGAGT | MNSGCTFTSPHLAQRVA | ACGTTAAACGGCGATGA | TTATATATGGCC |
| | CTTCGCGAAAAG | STVYQNCEHADNYTAYC | CAGACATAAAATTGTAA | ACGTAATGATTA |
| | CGTACGATCATA | LGISHMEPSFGLILHDG | ATGTGGACCAACGTCAA | TGATGGATTTTT |
| | ACTCACCTTATA | GTTLKFVDTPESLSGLY | TACGGTGACGTGTTTAA | AGAGAACGCACA |
| | TATGGCCACGTA | VFVVYFNGHVEAVAYTV | AGGAGATCTTAATCCAA | CGAACACCATGG |
| | ATGATTATGATG | VSTVDHFVNAIEERGFP | AACCCCAAGGCCAAAGA | GGTGTATAATCA |
| | GATTTTTAGAGA | PTAGQPPATTKPKEITP | CTCATTGAGGTGTCAGT | GGGCCGTGGTAT |
| | ACGCACACGAAC | VNPGTSPLLRYAAWTGG | GGAAGAAAATCACCCGT | CGATAGCGGGGA |
| | ACCATGGGGTGT | LAAVVLLCLVIFLICTA | TTACTTTACGCGCACCG | ACGGTTAATGCA |
| | ATAATCAGGGCC | KRMRVKAARVDK* | ATTCAGCGGATTTATGG | ACCCACACAAAT |
| | GTGGTATCGATA | | AGTCCGGTACACCGAGA | GTCTGCACAGGA |
| | GCGGGGAACGGT | | CTTGGAGCTTTTTGCCG | GGATCTTGGGGA |
| | TAATGCAACCCA | | TCATTAACCTGTACGGG | CGATACGGGCAT |
| | CACAAATGTCTG | | AGACGCAGCGCCCGCCA | CCACGTTATCCC |
| | CACAGGAGGATC | | TCCAGCATATATGTTTA | TACGTTAAACGG |
| | TTGGGGACGATA | | AAACATACAACATGCTT | CGATGACAGACA |
| | CGGGCATCCACG | | TCAAGACGTGGTGGTGG | TAAAATTGTAAA |
| | TTATCCCTACGT | | ATGTGGATTGCGCGGAA | TGTGGACCAACG |
| | TAAACGGCGATG | | AATACTAAAGAGGATCA | TCAATACGGTGA |
| | ACAGACATAAAA | | GTTGGCCGAAATCAGTT | CGTGTTTAAAGG |
| | TTGTAAATGTGG | | ACCGTTTTCAAGGTAAG | AGATCTTAATCC |
| | ACCAACGTCAAT | | AAGGAAGCGGACCAACC | AAAACCCCAAGG |
| | ACGGTGACGTGT | | GTGGATTGTTGTAAACA | CCAAAGACTCAT |
| | TTAAAGGAGATC | | CGAGCACACTGTTTGAT | TGAGGTGTCAGT |
| | TTAATCCAAAAC | | GAACTCGAATTAGACCC | GGAAGAAAATCA |
| | CCCAAGGCCAAA | | CCCCGAGATTGAACCGG | CCCGTTTACTTT |
| | GACTCATTGAGG | | GTGTCTTGAAAGTACTT | ACGCGCACCGAT |
| | TGTCAGTGGAAG | | CGGACAGAAAAACAATA | TCAGCGGATTTA |
| | AAAATCACCCGT | | CTTGGGTGTGTACATTT | TGGAGTCCGGTA |
| | TTACTTTACGCG | | GGAACATGCGCGGCTCC | CACCGAGACTTG |
| | CACCGATTCAGC | | GATGGTACGTCTACCTA | GAGCTTTTTGCC |
| | GGATTTATGGAG | | CGCCACGTTTTTGGTCA | GTCATTAACCTG |
| | TCCGGTACACCG | | CCTGGAAAGGGGATGAA | TACGGGAGACGC |
| | AGACTTGGAGCT | | AAAACAAGAAACCCTAC | AGCGCCCGCCAT |
| | TTTTGCCGTCAT | | GCCCGCAGTAACTCCTC | CCAGCATATATG |
| | TAACCTGTACGG | | AACCAAGAGGGGCTGAG | TTTAAAACATAC |
| | GAGACGCAGCGC | | TTTCATATGTGGAATTA | AACATGCTTTCA |
| | CCGCCATCCAGC | | CCACTCGCATGTATTTT | AGACGTGGTGGT |
| | ATATATGTTTAA | | CAGTTGGTGATACGTTT | GGATGTGGATTG |
| | AACATACAACAT | | AGCTTGGCAATGCATCT | CGCGGAAAATAC |
| | GCTTTCAAGACG | | TCAGTATAAGATACATG | TAAAGAGGATCA |
| | TGGTGGTGGATG | | AAGCGCCATTTGATTTG | GTTGGCCGAAAT |
| | TGGATTGCGCGG | | CTGTTAGAGTGGTTGTA | CAGTTACCGTTT |
| | AAAATACTAAAG | | TGTCCCCATCGATCCTA | TCAAGGTAAGAA |
| | AGGATCAGTTGG | | CATGTCAACCAATGCGG | GGAAGCGGACCA |
| | CCGAAATCAGTT | | TTATATTCTACGTGTTT | ACCGTGGATTGT |
| | ACCGTTTTCAAG | | GTATCATCCCAACGCAC | TGTAAACACGAG |
| | GTAAGAAGGAAG | | CCCAATGCCTCTCTCAT | CACACTGTTTGA |
| | CGGACCAACCGT | | ATGAATTCCGGTTGTAC | TGAACTCGAATT |
| | GGATTGTTGTAA | | ATTTACCTCGCCACATT | AGACCCCCCCGA |
| | ACACGAGCACAC | | TAGCCCAGCGTGTTGCA | GATTGAACCGGG |
| | TGTTTGATGAAC | | AGCACAGTGTATCAAAA | TGTCTTGAAAGT |
| | TCGAATTAGACC | | TTGTGAACATGCAGATA | ACTTCGGACAGA |
| | CCCCCGAGATTG | | ACTACACCGCATATTGT | AAAACAATACTT |
| | AACCGGGTGTCT | | CTGGGAATATCTCATAT | GGGTGTGTACAT |
| | TGAAAGTACTTC | | GGAGCCTAGCTTTGGTC | TTGGAACATGCG |
| | GGACAGAAAAAC | | TAATCTTACACGACGGG | CGGCTCCGATGG |
| | AATACTTGGGTG | | GGCACCACGTTAAAGTT | TACGTCTACCTA |
| | TGTACATTTGGA | | TGTAGATACACCCGAGA | CGCCACGTTTTT |
| | ACATGCGCGGCT | | GTTTGTCGGGATTATAC | GGTCACCTGGAA |
| | CCGATGGTACGT | | GTTTTTGTGGTGTATTT | AGGGGATGAAAA |
| | CTACCTACGCCA | | TAACGGGCATGTTGAAG | AACAAGAAACCC |
| | CGTTTTTGGTCA | | CCGTAGCATACACTGTT | TACGCCCGCAGT |
| | CCTGGAAAGGGG | | GTATCCACAGTAGATCA | AACTCCTCAACC |
| | ATGAAAAAACAA | | TTTTGTAAACGCAATTG | AAGAGGGGCTGA |
| | GAAACCCTACGC | | AAGAGCGTGGATTTCCG | GTTTCATATGTG |
| | CCGCAGTAACTC | | CCAACGGCCGGTCAGCC | GAATTACCACTC |
| | CTCAACCAAGAG | | ACCGGCGACTACTAAAC | GCATGTATTTTC |
| | GGGCTGAGTTTC | | CCAAGGAAATTACCCCC | AGTTGGTGATAC |
| | ATATGTGGAATT | | GTAAACCCCGGAACGTC | GTTTAGCTTGGC |
| | ACCACTCGCATG | | ACCACTTCTACGATATG | AATGCATCTTCA |
| | TATTTTCAGTTG | | CCGCATGGACCGGAGGG | GTATAAGATACA |
| | GTGATACGTTTA | | CTTGCAGCAGTAGTACT | TGAAGCGCCATT |
| | GCTTGGCAATGC | | TTTATGTCTCGTAATAT | TGATTTGCTGTT |
| | ATCTTCAGTATA | | TTTTAATCTGTACGGCT | AGAGTGGTTGTA |
| | AGATACATGAAG | | AAACGAATGAGGGTTAA | TGTCCCCATCGA |
| | CGCCATTTGATT | | AGCCGCCAGGGTAGACA | TCCTACATGTCA |
| | TGCTGTTAGAGT | | AGTGA | ACCAATGCGGTT |
| | GGTTGTATGTCC | | | ATATTCTACGTG |
| | CCATCGATCCTA | | | TTTGTATCATCC |
| | CATGTCAACCAA | | | CAACGCACCCCA |
| | TGCGGTTATATT | | | ATGCCTCTCTCA |
| | CTACGTGTTTGT | | | TATGAATTCCGG |
| | ATCATCCCAACG | | | TTGTACATTTAC |
| | CACCCCAATGCC | | | CTCGCCACATTT |
| | TCTCTCATATGA | | | AGCCCAGCGTGT |
| | ATTCCGGTTGTA | | | TGCAAGCACAGT |
| | CATTTACCTCGC | | | GTATCAAAATTG |
| | CACATTTAGCCC | | | TGAACATGCAGA |
| | AGCGTGTTGCAA | | | TAACTACACCGC |
| | GCACAGTGTATC | | | ATATTGTCTGGG |
| | AAAATTGTGAAC | | | AATATCTCATAT |
| | ATGCAGATAACT | | | GGAGCCTAGCTT |
| | ACACCGCATATT | | | TGGTCTAATCTT |
| | GTCTGGGAATAT | | | ACACGACGGGGG |
| | CTCATATGGAGC | | | CACCACGTTAAA |
| | CTAGCTTTGGTC | | | GTTTGTAGATAC |
| | TAATCTTACACG | | | ACCCGAGAGTTT |
| | ACGGGGGCACCA | | | GTCGGGATTATA |
| | CGTTAAAGTTTG | | | CGTTTTTGTGGT |
| | TAGATACACCCG | | | GTATTTTAACGG |
| | AGAGTTTGTCGG | | | GCATGTTGAAGC |
| | GATTATACGTTT | | | CGTAGCATACAC |
| | TTGTGGTGTATT | | | TGTTGTATCCAC |
| | TTAACGGGCATG | | | AGTAGATCATTT |
| | TTGAAGCCGTAG | | | TGTAAACGCAAT |
| | CATACACTGTTG | | | TGAAGAGCGTGG |
| | TATCCACAGTAG | | | ATTTCCGCCAAC |
| | ATCATTTTGTAA | | | GGCCGGTCAGCC |
| | ACGCAATTGAAG | | | ACCGGCGACTAC |
| | AGCGTGGATTTC | | | TAAACCCAAGGA |
| | CGCCAACGGCCG | | | AATTACCCCCGT |
| | GTCAGCCACCGG | | | AAACCCCGGAAC |
| | CGACTACTAAAC | | | GTCACCACTTCT |
| | CCAAGGAAATTA | | | ACGATATGCCGC |
| | CCCCCGTAAACC | | | ATGGACCGGAGG |
| | CCGGAACGTCAC | | | GCTTGCAGCAGT |
| | CACTTCTACGAT | | | AGTACTTTTATG |
| | ATGCCGCATGGA | | | TCTCGTAATATT |
| | CCGGAGGGCTTG | | | TTTAATCTGTAC |
| | CAGCAGTAGTAC | | | GGCTAAACGAAT |
| | TTTTATGTCTCG | | | GAGGGTTAAAGC |
| | TAATATTTTTAA | | | CGCCAGGGTAGA |
| | TCTGTACGGCTA | | | CAAGTGATGATA |
| | AACGAATGAGGG | | | ATAGGCTGGAGC |
| | TTAAAGCCGCCA | | | CTCGGTGGCCAT |
| | GGGTAGACAAGT | | | GCTTCTTGCCCC |
| | GATGATAATAGG | | | TTGGGCCTCCCC |
| | CTGGAGCCTCGG | | | CCAGCCCCTCCT |
| | TGGCCATGCTTC | | | CCCCTTCCTGCA |
| | TTGCCCCTTGGG | | | CCCGTACCCCCG |
| | CCTCCCCCCAGC | | | TGGTCTTTGAAT |
| | CCCTCCTCCCCT | | | AAAGTCTGAGTG |
| | TCCTGCACCCGT | | | GGCGGCAAAAAA |
| | ACCCCCGTGGTC | | | AAAAAAAAAAAA |
| | TTTGAATAAAGT | | | AAAAAAAAAAAA |
| | CTGAGTGGGCGG | | | AAAAAAAAAAAA |
| | C | | | AAAAAAAAAAAA |
| | | | | AAAAAAAAAAAA |
| | | | | AAAAAAAAAAAA |
| | | | | AAAAAAAAAAAA |
| | | | | AAAAAAAAAATC |
| | | | | TAG |
| | Sequence, NT | | | mRNA Sequence |
| | (5′ UTR, ORF, | | | (assumes T100 |
| mRNA | 3′ UTR) | ORF Sequence, AA | ORF Sequence, NT | tail) |
| Name(s) | SEQ ID NO: 2 | SEQ ID NO: 42 | SEQ ID NO: 43 | SEQ ID NO: 44 |
| VZV-GI- | TCAAGCTTTTGG | MFLIQCLISAVIFYIQV | ATGTTTTTAATCCAATG | G*GGGAAATAAG |
| full | ACCCTCGTACAG | TNALIFKGDHVSLQVNS | TTTGATATCGGCCGTTA | AGAGAAAAGAAG |
| | AAGCTAATACGA | SLTSILIPMQNDNYTEI | TATTTTACATACAAGTG | AGTAAGAAGAAA |
| | CTCACTATAGGG | KGQLVFigEQLPTGTNY | ACCAACGCTTTGATCTT | TATAAGAGCCAC |
| | AAATAAGAGAGA | SGTLELLYADTVAFCFR | CAAGGGCGACCACGTGA | CATGTTTTTAAT |
| | AAAGAAGAGTAA | SVQVIRYDGCPRIRTSA | GCTTGCAAGTTAACAGC | CCAATGTTTGAT |
| | GAAGAAATATAA | FISCRYKHSWHYGNSTD | AGTCTCACGTCTATCCT | ATCGGCCGTTAT |
| | GAGCCACCATGT | RISTEPDAGVMLKITKP | TATTCCCATGCAAAATG | ATTTTACATACA |
| | TTTTAATCCAAT | GINDAGVYVLLVRLDHS | ATAATTATACAGAGATA | AGTGACCAACGC |
| | GTTTGATATCGG | RSTDGFILGVNVYTAGS | AAAGGACAGCTTGTCTT | TTTGATCTTCAA |
| | CCGTTATATTTT | HHNIHGVIYTSPSLQNG | TATTGGAGAGCAACTAC | GGGCGACCACGT |
| | ACATACAAGTGA | YSTRALFQQARLCDLPA | CTACCGGGACAAACTAT | GAGCTTGCAAGT |
| | CCAACGCTTTGA | TPKGSGTSLFQHMLDLR | AGCGGAACACTGGAACT | TAACAGCAGTCT |
| | TCTTCAAGGGCG | AGKSLEDNPWLHEDVVT | GTTATACGCGGATACGG | CACGTCTATCCT |
| | ACCACGTGAGCT | TETKSVVKEGIENHVYP | TGGCGTTTTGTTTCCGG | TATTCCCATGCA |
| | TGCAAGTTAACA | TDMSTLPEKSLNDPPEN | TCAGTACAAGTAATAAG | AAATGATAATTA |
| | GCAGTCTCACGT | LLIIIPIVASVMILTAM | ATACGACGGATGTCCCC | TACAGAGATAAA |
| | CTATCCTTATTC | VIVIVISVKRRRIKKHP | GGATTAGAACGAGCGCT | AGGACAGCTTGT |
| | CCATGCAAAATG | IYRPNTKTRRGIQNATP | TTTATTTCGTGTAGGTA | CTTTATTGGAGA |
| | ATAATTATACAG | ESDVMLEAAIAQLATIR | CAAACATTCGTGGCATT | GCAACTACCTAC |
| | AGATAAAAGGAC | EESPPHSVVNPFVK* | ATGGTAACTCAACGGAT | CGGGACAAACTA |
| | AGCTTGTCTTTA | | CGGATATCAACAGAGCC | TAGCGGAACACT |
| | TTGGAGAGCAAC | | GGATGCTGGTGTAATGT | GGAACTGTTATA |
| | TACCTACCGGGA | | TGAAAATTACCAAACCG | CGCGGATACGGT |
| | CAAACTATAGCG | | GGAATAAATGATGCTGG | GGCGTTTTGTTT |
| | GAACACTGGAAC | | TGTGTATGTACTTCTTG | CCGGTCAGTACA |
| | TGTTATACGCGG | | TTCGGTTAGACCATAGC | AGTAATAAGATA |
| | ATACGGTGGCGT | | AGATCCACCGATGGTTT | CGACGGATGTCC |
| | TTTGTTTCCGGT | | CATTCTTGGTGTAAATG | CCGGATTAGAAC |
| | CAGTACAAGTAA | | TATATACAGCGGGCTCG | GAGCGCTTTTAT |
| | TAAGATACGACG | | CATCACAACATTCACGG | TTCGTGTAGGTA |
| | GATGTCCCCGGA | | GGTTATCTACACTTCTC | CAAACATTCGTG |
| | TTAGAACGAGCG | | CATCTCTACAGAATGGA | GCATTATGGTAA |
| | CTTTTATTTCGT | | TATTCTACAAGAGCCCT | CTCAACGGATCG |
| | GTAGGTACAAAC | | TTTTCAACAAGCTCGTT | GATATCAACAGA |
| | ATTCGTGGCATT | | TGTGTGATTTACCCGCG | GCCGGATGCTGG |
| | ATGGTAACTCAA | | ACACCCAAAGGGTCCGG | TGTAATGTTGAA |
| | CGGATCGGATAT | | TACCTCCCTGTTTCAAC | AATTACCAAACC |
| | CAACAGAGCCGG | | ATATGCTTGATCTTCGT | GGGAATAAATGA |
| | ATGCTGGTGTAA | | GCCGGTAAATCGTTAGA | TGCTGGTGTGTA |
| | TGTTGAAAATTA | | GGATAACCCTTGGTTAC | TGTACTTCTTGT |
| | CCAAACCGGGAA | | ATGAGGACGTTGTTACG | TCGGTTAGACCA |
| | TAAATGATGCTG | | ACAGAAACTAAGTCCGT | TAGCAGATCCAC |
| | GTGTGTATGTAC | | TGTTAAGGAGGGGATAG | CGATGGTTTCAT |
| | TTCTTGTTCGGT | | AAAATCACGTATATCCA | TCTTGGTGTAAA |
| | TAGACCATAGCA | | ACGGATATGTCCACGTT | TGTATATACAGC |
| | GATCCACCGATG | | ACCCGAAAAGTCCCTTA | GGGCTCGCATCA |
| | GTTTCATTCTTG | | ATGATCCTCCAGAAAAT | CAACATTCACGG |
| | GTGTAAATGTAT | | CTACTTATAATTATTCC | GGTTATCTACAC |
| | ATACAGCGGGCT | | TATAGTAGCGTCTGTCA | TTCTCCATCTCT |
| | CGCATCACAACA | | TGATCCTCACCGCCATG | ACAGAATGGATA |
| | TTCACGGGGTTA | | GTTATTGTTATTGTAAT | TTCTACAAGAGC |
| | TCTACACTTCTC | | AAGCGTTAAGCGACGTA | CCTTTTTCAACA |
| | CATCTCTACAGA | | GAATTAAAAAACATCCA | AGCTCGTTTGTG |
| | ATGGATATTCTA | | ATTTATCGCCCAAATAC | TGATTTACCCGC |
| | CAAGAGCCCTTT | | AAAAACAAGAAGGGGCA | GACACCCAAAGG |
| | TTCAACAAGCTC | | TACAAAATGCGACACCA | GTCCGGTACCTC |
| | GTTTGTGTGATT | | GAATCCGATGTGATGTT | CCTGTTTCAACA |
| | TACCCGCGACAC | | GGAGGCCGCCATTGCAC | TATGCTTGATCT |
| | CCAAAGGGTCCG | | AACTAGCAACGATTCGC | TCGTGCCGGTAA |
| | GTACCTCCCTGT | | GAAGAATCCCCCCCACA | ATCGTTAGAGGA |
| | TTCAACATATGC | | TTCCGTTGTAAACCCGT | TAACCCTTGGTT |
| | TTGATCTTCGTG | | TTGTTAAATAG | ACATGAGGACGT |
| | CCGGTAAATCGT | | | TGTTACGACAGA |
| | TAGAGGATAACC | | | AACTAAGTCCGT |
| | CTTGGTTACATG | | | TGTTAAGGAGGG |
| | AGGACGTTGTTA | | | GATAGAAAATCA |
| | CGACAGAAACTA | | | CGTATATCCAAC |
| | AGTCCGTTGTTA | | | GGATATGTCCAC |
| | AGGAGGGGATAG | | | GTTACCCGAAAA |
| | AAAATCACGTAT | | | GTCCCTTAATGA |
| | ATCCAACGGATA | | | TCCTCCAGAAAA |
| | TGTCCACGTTAC | | | TCTACTTATAAT |
| | CCGAAAAGTCCC | | | TATTCCTATAGT |
| | TTAATGATCCTC | | | AGCGTCTGTCAT |
| | CAGAAAATCTAC | | | GATCCTCACCGC |
| | TTATAATTATTC | | | CATGGTTATTGT |
| | CTATAGTAGCGT | | | TATTGTAATAAG |
| | CTGTCATGATCC | | | CGTTAAGCGACG |
| | TCACCGCCATGG | | | TAGAATTAAAAA |
| | TTATTGTTATTG | | | ACATCCAATTTA |
| | TAATAAGCGTTA | | | TCGCCCAAATAC |
| | AGCGACGTAGAA | | | AAAAACAAGAAG |
| | TTAAAAAACATC | | | GGGCATACAAAA |
| | CAATTTATCGCC | | | TGCGACACCAGA |
| | CAAATACAAAAA | | | ATCCGATGTGAT |
| | CAAGAAGGGGCA | | | GTTGGAGGCCGC |
| | TACAAAATGCGA | | | CATTGCACAACT |
| | CACCAGAATCCG | | | AGCAACGATTCG |
| | ATGTGATGTTGG | | | CGAAGAATCCCC |
| | AGGCCGCCATTG | | | CCCACATTCCGT |
| | CACAACTAGCAA | | | TGTAAACCCGTT |
| | CGATTCGCGAAG | | | TGTTAAATAGTG |
| | AATCCCCCCCAC | | | ATAATAGGCTGG |
| | ATTCCGTTGTAA | | | AGCCTCGGTGGC |
| | ACCCGTTTGTTA | | | CATGCTTCTTGC |
| | AATAGTGATAAT | | | CCCTTGGGCCTC |
| | AGGCTGGAGCCT | | | CCCCCAGCCCCT |
| | CGGTGGCCATGC | | | CCTCCCCTTCCT |
| | TTCTTGCCCCTT | | | GCACCCGTACCC |
| | GGGCCTCCCCCC | | | CCGTGGTCTTTG |
| | AGCCCCTCCTCC | | | AATAAAGTCTGA |
| | CCTTCCTGCACC | | | GTGGGCGGCAAA |
| | CGTACCCCCGTG | | | AAAAAAAAAAAA |
| | GTCTTTGAATAA | | | AAAAAAAAAAAA |
| | AGTCTGAGTGGG | | | AAAAAAAAAAAA |
| | CGGC | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | ATCTAG |
| | Sequence, NT | | | mRNA Sequence |
| | (5′ UTR, ORF, | | | (assumes |
| mRNA | 3′ UTR) | ORF Sequence, AA | ORF Sequence, NT | T100 tail) |
| Name(s) | SEQ ID NO: 60 | SEQ ID NO: 61 | SEQ ID NO: 62 | SEQ ID NO: 63 |
| VZV-GE- | GGGAAATAAGAG | MGTVNKPVVGVLMGFGI | ATGGGCACCGTGAACAA | GGGAAATAAGAG |
| truncated- | AGAAAAGAAGAG | ITGTLRITNPVRASVLR | GCCCGTCGTGGGCGTGC | AGAAAAGAAGAG |
| delete_ | TAAGAAGAAATA | YDDFHIDEDKLDTNSVY | TGATGGGCTTCGGCATC | TAAGAAGAAATA |
| from_ | TAAGAGCCACCA | EPYYHSDHAESSWVNRG | ATCACCGGCACCCTGCG | TAAGAGCCACCA |
| 574_-_ | TGGGCACCGTGA | ESSRKAYDHNSPYIWPR | GATCACCAATCCTGTGC | TGGGCACCGTGA |
| Y569A | ACAAGCCCGTCG | NDYDGFLENAHEHHGVY | GGGCCAGCGTGCTGAGA | ACAAGCCCGTCG |
| Variant | TGGGCGTGCTGA | NQGRGIDSGERLMQPTQ | TACGACGACTTCCACAT | TGGGCGTGCTGA |
| 1 | TGGGCTTCGGCA | MSAQEDLGDDTGIHVIP | CGACGAGGACAAGCTGG | TGGGCTTCGGCA |
| | TCATCACCGGCA | TLNGDDRHKIVNVDQRQ | ACACCAACAGCGTGTAC | TCATCACCGGCA |
| | CCCTGCGGATCA | YGDVFKGDLNPKPQGQR | GAGCCCTACTACCACAG | CCCTGCGGATCA |
| | CCAATCCTGTGC | LIEVSVEENHPFTLRAP | CGACCACGCCGAGAGCA | CCAATCCTGTGC |
| | GGGCCAGCGTGC | IQRIYGVRYTETWSFLP | GCTGGGTCAACAGAGGC | GGGCCAGCGTGC |
| | TGAGATACGACG | SLTCTGDAAPAIQHICL | GAGTCCAGCCGGAAGGC | TGAGATACGACG |
| | ACTTCCACATCG | KHTTCFQDVVVDVDCAE | CTACGACCACAACAGCC | ACTTCCACATCG |
| | ACGAGGACAAGC | NTKEDQLAEISYRFQGK | CCTACATCTGGCCCCGG | ACGAGGACAAGC |
| | TGGACACCAACA | KEADQPWIVVNTSTLFD | AACGACTACGACGGCTT | TGGACACCAACA |
| | GCGTGTACGAGC | ELELDPPEIEPGVLKVL | CCTGGAAAATGCCCACG | GCGTGTACGAGC |
| | CCTACTACCACA | RTEKQYLGVYIWNMRGS | AGCACCACGGCGTGTAC | CCTACTACCACA |
| | GCGACCACGCCG | DGTSTYATFLVTWKGDE | AACCAGGGCAGAGGCAT | GCGACCACGCCG |
| | AGAGCAGCTGGG | KTRNPTPAVTPQPRGAE | CGACAGCGGCGAGAGAC | AGAGCAGCTGGG |
| | TCAACAGAGGCG | FHMWNYHSHVFSVGDTF | TGATGCAGCCCACCCAG | TCAACAGAGGCG |
| | AGTCCAGCCGGA | SLAMHLQYKIHEAPFDL | ATGAGCGCCCAGGAAGA | AGTCCAGCCGGA |
| | AGGCCTACGACC | LLEWLYVPIDPTCQPMR | TCTGGGCGACGACACCG | AGGCCTACGACC |
| | ACAACAGCCCCT | LYSTCLYHPNAPQCLSH | GCATCCACGTGATCCCT | ACAACAGCCCCT |
| | ACATCTGGCCCC | MNSGCTFTSPHLAQRVA | ACCCTGAACGGCGACGA | ACATCTGGCCCC |
| | GGAACGACTACG | STVYQNCEHADNYTAYC | CCGGCACAAGATCGTGA | GGAACGACTACG |
| | ACGGCTTCCTGG | LGISHMEPSFGLILHDG | ACGTGGACCAGCGGCAG | ACGGCTTCCTGG |
| | AAAATGCCCACG | GTTLKFVDTPESLSGLY | TACGGCGACGTGTTCAA | AAAATGCCCACG |
| | AGCACCACGGCG | VFVVYFNGHVEAVAYTV | GGGCGACCTGAACCCCA | AGCACCACGGCG |
| | TGTACAACCAGG | VSTVDHFVNAIEERGFP | AGCCCCAGGGACAGCGG | TGTACAACCAGG |
| | GCAGAGGCATCG | PTAGQPPATTKPKEITP | CTGATTGAGGTGTCCGT | GCAGAGGCATCG |
| | ACAGCGGCGAGA | VNPGTSPLLRYAAWTGG | GGAAGAGAACCACCCCT | ACAGCGGCGAGA |
| | GACTGATGCAGC | LAAVVLLCLVIFLICTA | TCACCCTGAGAGCCCCT | GACTGATGCAGC |
| | CCACCCAGATGA | KRMRVKAARVDK | ATCCAGCGGATCTACGG | CCACCCAGATGA |
| | GCGCCCAGGAAG | | CGTGCGCTATACCGAGA | GCGCCCAGGAAG |
| | ATCTGGGCGACG | | CTTGGAGCTTCCTGCCC | ATCTGGGCGACG |
| | ACACCGGCATCC | | AGCCTGACCTGTACTGG | ACACCGGCATCC |
| | ACGTGATCCCTA | | CGACGCCGCTCCTGCCA | ACGTGATCCCTA |
| | CCCTGAACGGCG | | TCCAGCACATCTGCCTG | CCCTGAACGGCG |
| | ACGACCGGCACA | | AAGCACACCACCTGTTT | ACGACCGGCACA |
| | AGATCGTGAACG | | CCAGGACGTGGTGGTGG | AGATCGTGAACG |
| | TGGACCAGCGGC | | ACGTGGACTGCGCCGAG | TGGACCAGCGGC |
| | AGTACGGCGACG | | AACACCAAAGAGGACCA | AGTACGGCGACG |
| | TGTTCAAGGGCG | | GCTGGCCGAGATCAGCT | TGTTCAAGGGCG |
| | ACCTGAACCCCA | | ACCGGTTCCAGGGCAAG | ACCTGAACCCCA |
| | AGCCCCAGGGAC | | AAAGAGGCCGACCAGCC | AGCCCCAGGGAC |
| | AGCGGCTGATTG | | CTGGATCGTCGTGAACA | AGCGGCTGATTG |
| | AGGTGTCCGTGG | | CCAGCACCCTGTTCGAC | AGGTGTCCGTGG |
| | AAGAGAACCACC | | GAGCTGGAACTGGACCC | AAGAGAACCACC |
| | CCTTCACCCTGA | | TCCCGAGATCGAACCCG | CCTTCACCCTGA |
| | GAGCCCCTATCC | | GGGTGCTGAAGGTGCTG | GAGCCCCTATCC |
| | AGCGGATCTACG | | CGGACCGAGAAGCAGTA | AGCGGATCTACG |
| | GCGTGCGCTATA | | CCTGGGAGTGTACATCT | GCGTGCGCTATA |
| | CCGAGACTTGGA | | GGAACATGCGGGGCAGC | CCGAGACTTGGA |
| | GCTTCCTGCCCA | | GACGGCACCTCTACCTA | GCTTCCTGCCCA |
| | GCCTGACCTGTA | | CGCCACCTTCCTCGTGA | GCCTGACCTGTA |
| | CTGGCGACGCCG | | CCTGGAAGGGCGACGAG | CTGGCGACGCCG |
| | CTCCTGCCATCC | | AAAACCCGGAACCCTAC | CTCCTGCCATCC |
| | AGCACATCTGCC | | CCCTGCCGTGACCCCTC | AGCACATCTGCC |
| | TGAAGCACACCA | | AGCCTAGAGGCGCCGAG | TGAAGCACACCA |
| | CCTGTTTCCAGG | | TTTCACATGTGGAATTA | CCTGTTTCCAGG |
| | ACGTGGTGGTGG | | CCACAGCCACGTGTTCA | ACGTGGTGGTGG |
| | ACGTGGACTGCG | | GCGTGGGCGACACCTTC | ACGTGGACTGCG |
| | CCGAGAACACCA | | TCCCTGGCCATGCATCT | CCGAGAACACCA |
| | AAGAGGACCAGC | | GCAGTACAAGATCCACG | AAGAGGACCAGC |
| | TGGCCGAGATCA | | AGGCCCCTTTCGACCTG | TGGCCGAGATCA |
| | GCTACCGGTTCC | | CTGCTGGAATGGCTGTA | GCTACCGGTTCC |
| | AGGGCAAGAAAG | | CGTGCCCATCGACCCTA | AGGGCAAGAAAG |
| | AGGCCGACCAGC | | CCTGCCAGCCCATGCGG | AGGCCGACCAGC |
| | CCTGGATCGTCG | | CTGTACTCCACCTGTCT | CCTGGATCGTCG |
| | TGAACACCAGCA | | GTACCACCCCAACGCCC | TGAACACCAGCA |
| | CCCTGTTCGACG | | CTCAGTGCCTGAGCCAC | CCCTGTTCGACG |
| | AGCTGGAACTGG | | ATGAATAGCGGCTGCAC | AGCTGGAACTGG |
| | ACCCTCCCGAGA | | CTTCACCAGCCCTCACC | ACCCTCCCGAGA |
| | TCGAACCCGGGG | | TGGCTCAGAGGGTGGCC | TCGAACCCGGGG |
| | TGCTGAAGGTGC | | AGCACCGTGTACCAGAA | TGCTGAAGGTGC |
| | TGCGGACCGAGA | | TTGCGAGCACGCCGACA | TGCGGACCGAGA |
| | AGCAGTACCTGG | | ACTACACCGCCTACTGC | AGCAGTACCTGG |
| | GAGTGTACATCT | | CTGGGCATCAGCCACAT | GAGTGTACATCT |
| | GGAACATGCGGG | | GGAACCCAGCTTCGGCC | GGAACATGCGGG |
| | GCAGCGACGGCA | | TGATCCTGCACGATGGC | GCAGCGACGGCA |
| | CCTCTACCTACG | | GGCACCACCCTGAAGTT | CCTCTACCTACG |
| | CCACCTTCCTCG | | CGTGGACACCCCTGAGT | CCACCTTCCTCG |
| | TGACCTGGAAGG | | CCCTGAGCGGCCTGTAC | TGACCTGGAAGG |
| | GCGACGAGAAAA | | GTGTTCGTGGTGTACTT | GCGACGAGAAAA |
| | CCCGGAACCCTA | | CAACGGCCACGTGGAAG | CCCGGAACCCTA |
| | CCCCTGCCGTGA | | CCGTGGCCTACACCGTG | CCCCTGCCGTGA |
| | CCCCTCAGCCTA | | GTGTCCACCGTGGACCA | CCCCTCAGCCTA |
| | GAGGCGCCGAGT | | CTTCGTGAACGCCATCG | GAGGCGCCGAGT |
| | TTCACATGTGGA | | AGGAACGGGGCTTCCCT | TTCACATGTGGA |
| | ATTACCACAGCC | | CCAACTGCTGGACAGCC | ATTACCACAGCC |
| | ACGTGTTCAGCG | | TCCTGCCACCACCAAGC | ACGTGTTCAGCG |
| | TGGGCGACACCT | | CCAAAGAAATCACCCCT | TGGGCGACACCT |
| | TCTCCCTGGCCA | | GTGAACCCCGGCACCAG | TCTCCCTGGCCA |
| | TGCATCTGCAGT | | CCCACTGCTGCGCTATG | TGCATCTGCAGT |
| | ACAAGATCCACG | | CTGCTTGGACAGGCGGA | ACAAGATCCACG |
| | AGGCCCCTTTCG | | CTGGCTGCTGTGGTGCT | AGGCCCCTTTCG |
| | ACCTGCTGCTGG | | GCTGTGCCTCGTGATTT | ACCTGCTGCTGG |
| | AATGGCTGTACG | | TCCTGATCTGCACCGCC | AATGGCTGTACG |
| | TGCCCATCGACC | | AAGCGGATGAGAGTGAA | TGCCCATCGACC |
| | CTACCTGCCAGC | | GGCCGCCAGAGTGGACA | CTACCTGCCAGC |
| | CCATGCGGCTGT | | AG | CCATGCGGCTGT |
| | ACTCCACCTGTC | | | ACTCCACCTGTC |
| | TGTACCACCCCA | | | TGTACCACCCCA |
| | ACGCCCCTCAGT | | | ACGCCCCTCAGT |
| | GCCTGAGCCACA | | | GCCTGAGCCACA |
| | TGAATAGCGGCT | | | TGAATAGCGGCT |
| | GCACCTTCACCA | | | GCACCTTCACCA |
| | GCCCTCACCTGG | | | GCCCTCACCTGG |
| | CTCAGAGGGTGG | | | CTCAGAGGGTGG |
| | CCAGCACCGTGT | | | CCAGCACCGTGT |
| | ACCAGAATTGCG | | | ACCAGAATTGCG |
| | AGCACGCCGACA | | | AGCACGCCGACA |
| | ACTACACCGCCT | | | ACTACACCGCCT |
| | ACTGCCTGGGCA | | | ACTGCCTGGGCA |
| | TCAGCCACATGG | | | TCAGCCACATGG |
| | AACCCAGCTTCG | | | AACCCAGCTTCG |
| | GCCTGATCCTGC | | | GCCTGATCCTGC |
| | ACGATGGCGGCA | | | ACGATGGCGGCA |
| | CCACCCTGAAGT | | | CCACCCTGAAGT |
| | TCGTGGACACCC | | | TCGTGGACACCC |
| | CTGAGTCCCTGA | | | CTGAGTCCCTGA |
| | GCGGCCTGTACG | | | GCGGCCTGTACG |
| | TGTTCGTGGTGT | | | TGTTCGTGGTGT |
| | ACTTCAACGGCC | | | ACTTCAACGGCC |
| | ACGTGGAAGCCG | | | ACGTGGAAGCCG |
| | TGGCCTACACCG | | | TGGCCTACACCG |
| | TGGTGTCCACCG | | | TGGTGTCCACCG |
| | TGGACCACTTCG | | | TGGACCACTTCG |
| | TGAACGCCATCG | | | TGAACGCCATCG |
| | AGGAACGGGGCT | | | AGGAACGGGGCT |
| | TCCCTCCAACTG | | | TCCCTCCAACTG |
| | CTGGACAGCCTC | | | CTGGACAGCCTC |
| | CTGCCACCACCA | | | CTGCCACCACCA |
| | AGCCCAAAGAAA | | | AGCCCAAAGAAA |
| | TCACCCCTGTGA | | | TCACCCCTGTGA |
| | ACCCCGGCACCA | | | ACCCCGGCACCA |
| | GCCCACTGCTGC | | | GCCCACTGCTGC |
| | GCTATGCTGCTT | | | GCTATGCTGCTT |
| | GGACAGGCGGAC | | | GGACAGGCGGAC |
| | TGGCTGCTGTGG | | | TGGCTGCTGTGG |
| | TGCTGCTGTGCC | | | TGCTGCTGTGCC |
| | TCGTGATTTTCC | | | TCGTGATTTTCC |
| | TGATCTGCACCG | | | TGATCTGCACCG |
| | CCAAGCGGATGA | | | CCAAGCGGATGA |
| | GAGTGAAGGCCG | | | GAGTGAAGGCCG |
| | CCAGAGTGGACA | | | CCAGAGTGGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |
| | Sequence, NT | | | mRNA Sequence |
| | (5′ UTR, ORF, | | | (assumes |
| mRNA | 3′ UTR) | ORF Sequence, AA | ORF Sequence, NT | T100 tail) |
| Name(s) | SEQ ID NO: 64 | SEQ ID NO: 65 | SEQ ID NO: 66 | SEQ ID NO: 67 |
| VZV-GE- | GGGAAATAAGAG | MGTVNKPVVGVLMGFGI | ATGGGGACAGTTAATAA | GGGAAATAAGAG |
| truncated- | AGAAAAGAAGAG | ITGTLRITNPVRASVLR | ACCTGTGGTGGGGGTAT | AGAAAAGAAGAG |
| delete_ | TAAGAAGAAATA | YDDFHIDEDKLDTNSVY | TGATGGGGTTCGGAATT | TAAGAAGAAATA |
| from_ | TAAGAGCCACCA | EPYYHSDHAESSWVNRG | ATCACGGGAACGTTGCG | TAAGAGCCACCA |
| 574_-_ | TGGGGACAGTTA | ESSRKAYDHNSPYIWPR | TATAACGAATCCGGTCA | TGGGGACAGTTA |
| Y569A | ATAAACCTGTGG | NDYDGFLENAHEHHGVY | GAGCATCCGTCTTGCGA | ATAAACCTGTGG |
| Variant | TGGGGGTATTGA | NQGRGIDSGERLMQPTQ | TACGATGATTTTCACAT | TGGGGGTATTGA |
| 2 | TGGGGTTCGGAA | MSAQEDLGDDTGIHVIP | CGATGAAGACAAACTGG | TGGGGTTCGGAA |
| | TTATCACGGGAA | TLNGDDRHKIVNVDQRQ | ATACAAACTCCGTATAT | TTATCACGGGAA |
| | CGTTGCGTATAA | YGDVFKGDLNPKPQGQR | GAGCCTTACTACCATTC | CGTTGCGTATAA |
| | CGAATCCGGTCA | LIEVSVEENHPFTLRAP | AGATCATGCGGAGTCTT | CGAATCCGGTCA |
| | GAGCATCCGTCT | IQRIYGVRYTETWSFLP | CATGGGTAAATCGGGGA | GAGCATCCGTCT |
| | TGCGATACGATG | SLTCTGDAAPAIQHICL | GAGTCTTCGCGAAAAGC | TGCGATACGATG |
| | ATTTTCACATCG | KHTTCFQDVVVDVDCAE | GTACGATCATAACTCAC | ATTTTCACATCG |
| | ATGAAGACAAAC | NTKEDQLAEISYRFQGK | CTTATATATGGCCACGT | ATGAAGACAAAC |
| | TGGATACAAACT | KEADQPWIVVNTSTLFD | AATGATTATGATGGATT | TGGATACAAACT |
| | CCGTATATGAGC | ELELDPPEIEPGVLKVL | TTTAGAGAACGCACACG | CCGTATATGAGC |
| | CTTACTACCATT | RTEKQYLGVYIWNMRGS | AACACCATGGGGTGTAT | CTTACTACCATT |
| | CAGATCATGCGG | DGTSTYATFLVTWKGDE | AATCAGGGCCGTGGTAT | CAGATCATGCGG |
| | AGTCTTCATGGG | KTRNPTPAVTPQPRGAE | CGATAGCGGGGAACGGT | AGTCTTCATGGG |
| | TAAATCGGGGAG | FHMWNYHSHVFSVGDTF | TAATGCAACCCACACAA | TAAATCGGGGAG |
| | AGTCTTCGCGAA | SLAMHLQYKIHEAPFDL | ATGTCTGCACAGGAGGA | AGTCTTCGCGAA |
| | AAGCGTACGATC | LLEWLYVPIDPTCQPMR | TCTTGGGGACGATACGG | AAGCGTACGATC |
| | ATAACTCACCTT | LYSTCLYHPNAPQCLSH | GCATCCACGTTATCCCT | ATAACTCACCTT |
| | ATATATGGCCAC | MNSGCTFTSPHLAQRVA | ACGTTAAACGGCGATGA | ATATATGGCCAC |
| | GTAATGATTATG | STVYQNCEHADNYTAYC | CAGACATAAAATTGTAA | GTAATGATTATG |
| | ATGGATTTTTAG | LGISHMEPSFGLILHDG | ATGTGGACCAACGTCAA | ATGGATTTTTAG |
| | AGAACGCACACG | GTTLKFVDTPESLSGLY | TACGGTGACGTGTTTAA | AGAACGCACACG |
| | AACACCATGGGG | VFVVYFNGHVEAVAYTV | AGGAGATCTTAATCCAA | AACACCATGGGG |
| | TGTATAATCAGG | VSTVDHFVNAIEERGFP | AACCCCAAGGCCAAAGA | TGTATAATCAGG |
| | GCCGTGGTATCG | PTAGQPPATTKPKEITP | CTCATTGAGGTGTCAGT | GCCGTGGTATCG |
| | ATAGCGGGGAAC | VNPGTSPLLRYAAWTGG | GGAAGAAAATCACCCGT | ATAGCGGGGAAC |
| | GGTTAATGCAAC | LAAVVLLCLVIFLICTA | TTACTTTACGCGCACCG | GGTTAATGCAAC |
| | CCACACAAATGT | KRMRVKAARVDK | ATTCAGCGGATTTATGG | CCACACAAATGT |
| | CTGCACAGGAGG | | AGTCCGGTACACCGAGA | CTGCACAGGAGG |
| | ATCTTGGGGACG | | CTTGGAGCTTTTTGCCG | ATCTTGGGGACG |
| | ATACGGGCATCC | | TCATTAACCTGTACGGG | ATACGGGCATCC |
| | ACGTTATCCCTA | | AGACGCAGCGCCCGCCA | ACGTTATCCCTA |
| | CGTTAAACGGCG | | TCCAGCATATATGTTTA | CGTTAAACGGCG |
| | ATGACAGACATA | | AAACATACAACATGCTT | ATGACAGACATA |
| | AAATTGTAAATG | | TCAAGACGTGGTGGTGG | AAATTGTAAATG |
| | TGGACCAACGTC | | ATGTGGATTGCGCGGAA | TGGACCAACGTC |
| | AATACGGTGACG | | AATACTAAAGAGGATCA | AATACGGTGACG |
| | TGTTTAAAGGAG | | GTTGGCCGAAATCAGTT | TGTTTAAAGGAG |
| | ATCTTAATCCAA | | ACCGTTTTCAAGGTAAG | ATCTTAATCCAA |
| | AACCCCAAGGCC | | AAGGAAGCGGACCAACC | AACCCCAAGGCC |
| | AAAGACTCATTG | | GTGGATTGTTGTAAACA | AAAGACTCATTG |
| | AGGTGTCAGTGG | | CGAGCACACTGTTTGAT | AGGTGTCAGTGG |
| | AAGAAAATCACC | | GAACTCGAATTAGACCC | AAGAAAATCACC |
| | CGTTTACTTTAC | | CCCCGAGATTGAACCGG | CGTTTACTTTAC |
| | GCGCACCGATTC | | GTGTCTTGAAAGTACTT | GCGCACCGATTC |
| | AGCGGATTTATG | | CGGACAGAGAAACAATA | AGCGGATTTATG |
| | GAGTCCGGTACA | | CTTGGGTGTGTACATTT | GAGTCCGGTACA |
| | CCGAGACTTGGA | | GGAACATGCGCGGCTCC | CCGAGACTTGGA |
| | GCTTTTTGCCGT | | GATGGTACGTCTACCTA | GCTTTTTGCCGT |
| | CATTAACCTGTA | | CGCCACGTTTTTGGTCA | CATTAACCTGTA |
| | CGGGAGACGCAG | | CCTGGAAAGGGGATGAG | CGGGAGACGCAG |
| | CGCCCGCCATCC | | AAGACAAGAAACCCTAC | CGCCCGCCATCC |
| | AGCATATATGTT | | GCCCGCAGTAACTCCTC | AGCATATATGTT |
| | TAAAACATACAA | | AACCAAGAGGGGCTGAG | TAAAACATACAA |
| | CATGCTTTCAAG | | TTTCATATGTGGAATTA | CATGCTTTCAAG |
| | ACGTGGTGGTGG | | CCACTCGCATGTATTTT | ACGTGGTGGTGG |
| | ATGTGGATTGCG | | CAGTTGGTGATACGTTT | ATGTGGATTGCG |
| | CGGAAAATACTA | | AGCTTGGCAATGCATCT | CGGAAAATACTA |
| | AAGAGGATCAGT | | TCAGTATAAGATACATG | AAGAGGATCAGT |
| | TGGCCGAAATCA | | AAGCGCCATTTGATTTG | TGGCCGAAATCA |
| | GTTACCGTTTTC | | CTGTTAGAGTGGTTGTA | GTTACCGTTTTC |
| | AAGGTAAGAAGG | | TGTCCCCATCGATCCTA | AAGGTAAGAAGG |
| | AAGCGGACCAAC | | CATGTCAACCAATGCGG | AAGCGGACCAAC |
| | CGTGGATTGTTG | | TTATATTCTACGTGTTT | CGTGGATTGTTG |
| | TAAACACGAGCA | | GTATCATCCCAACGCAC | TAAACACGAGCA |
| | CACTGTTTGATG | | CCCAATGCCTCTCTCAT | CACTGTTTGATG |
| | AACTCGAATTAG | | ATGAATTCCGGTTGTAC | AACTCGAATTAG |
| | ACCCCCCCGAGA | | ATTTACCTCGCCACATT | ACCCCCCCGAGA |
| | TTGAACCGGGTG | | TAGCCCAGCGTGTTGCA | TTGAACCGGGTG |
| | TCTTGAAAGTAC | | AGCACAGTGTATCAAAA | TCTTGAAAGTAC |
| | TTCGGACAGAGA | | TTGTGAACATGCAGATA | TTCGGACAGAGA |
| | AACAATACTTGG | | ACTACACCGCATATTGT | AACAATACTTGG |
| | GTGTGTACATTT | | CTGGGAATATCTCATAT | GTGTGTACATTT |
| | GGAACATGCGCG | | GGAGCCTAGCTTTGGTC | GGAACATGCGCG |
| | GCTCCGATGGTA | | TAATCTTACACGACGGG | GCTCCGATGGTA |
| | CGTCTACCTACG | | GGCACCACGTTAAAGTT | CGTCTACCTACG |
| | CCACGTTTTTGG | | TGTAGATACACCCGAGA | CCACGTTTTTGG |
| | TCACCTGGAAAG | | GTTTGTCGGGATTATAC | TCACCTGGAAAG |
| | GGGATGAGAAGA | | GTTTTTGTGGTGTATTT | GGGATGAGAAGA |
| | CAAGAAACCCTA | | TAACGGGCATGTTGAAG | CAAGAAACCCTA |
| | CGCCCGCAGTAA | | CCGTAGCATACACTGTT | CGCCCGCAGTAA |
| | CTCCTCAACCAA | | GTATCCACAGTAGATCA | CTCCTCAACCAA |
| | GAGGGGCTGAGT | | TTTTGTAAACGCAATTG | GAGGGGCTGAGT |
| | TTCATATGTGGA | | AAGAGCGTGGATTTCCG | TTCATATGTGGA |
| | ATTACCACTCGC | | CCAACGGCCGGTCAGCC | ATTACCACTCGC |
| | ATGTATTTTCAG | | ACCGGCGACTACTAAAC | ATGTATTTTCAG |
| | TTGGTGATACGT | | CCAAGGAAATTACCCCC | TTGGTGATACGT |
| | TTAGCTTGGCAA | | GTAAACCCCGGAACGTC | TTAGCTTGGCAA |
| | TGCATCTTCAGT | | ACCACTTCTACGATATG | TGCATCTTCAGT |
| | ATAAGATACATG | | CCGCATGGACCGGAGGG | ATAAGATACATG |
| | AAGCGCCATTTG | | CTTGCAGCAGTAGTACT | AAGCGCCATTTG |
| | ATTTGCTGTTAG | | TTTATGTCTCGTAATAT | ATTTGCTGTTAG |
| | AGTGGTTGTATG | | TTTTAATCTGTACGGCT | AGTGGTTGTATG |
| | TCCCCATCGATC | | AAACGAATGAGGGTTAA | TCCCCATCGATC |
| | CTACATGTCAAC | | AGCCGCCAGGGTAGACA | CTACATGTCAAC |
| | CAATGCGGTTAT | | AG | CAATGCGGTTAT |
| | ATTCTACGTGTT | | | ATTCTACGTGTT |
| | TGTATCATCCCA | | | TGTATCATCCCA |
| | ACGCACCCCAAT | | | ACGCACCCCAAT |
| | GCCTCTCTCATA | | | GCCTCTCTCATA |
| | TGAATTCCGGTT | | | TGAATTCCGGTT |
| | GTACATTTACCT | | | GTACATTTACCT |
| | CGCCACATTTAG | | | CGCCACATTTAG |
| | CCCAGCGTGTTG | | | CCCAGCGTGTTG |
| | CAAGCACAGTGT | | | CAAGCACAGTGT |
| | ATCAAAATTGTG | | | ATCAAAATTGTG |
| | AACATGCAGATA | | | AACATGCAGATA |
| | ACTACACCGCAT | | | ACTACACCGCAT |
| | ATTGTCTGGGAA | | | ATTGTCTGGGAA |
| | TATCTCATATGG | | | TATCTCATATGG |
| | AGCCTAGCTTTG | | | AGCCTAGCTTTG |
| | GTCTAATCTTAC | | | GTCTAATCTTAC |
| | ACGACGGGGGCA | | | ACGACGGGGGCA |
| | CCACGTTAAAGT | | | CCACGTTAAAGT |
| | TTGTAGATACAC | | | TTGTAGATACAC |
| | CCGAGAGTTTGT | | | CCGAGAGTTTGT |
| | CGGGATTATACG | | | CGGGATTATACG |
| | TTTTTGTGGTGT | | | TTTTTGTGGTGT |
| | ATTTTAACGGGC | | | ATTTTAACGGGC |
| | ATGTTGAAGCCG | | | ATGTTGAAGCCG |
| | TAGCATACACTG | | | TAGCATACACTG |
| | TTGTATCCACAG | | | TTGTATCCACAG |
| | TAGATCATTTTG | | | TAGATCATTTTG |
| | TAAACGCAATTG | | | TAAACGCAATTG |
| | AAGAGCGTGGAT | | | AAGAGCGTGGAT |
| | TTCCGCCAACGG | | | TTCCGCCAACGG |
| | CCGGTCAGCCAC | | | CCGGTCAGCCAC |
| | CGGCGACTACTA | | | CGGCGACTACTA |
| | AACCCAAGGAAA | | | AACCCAAGGAAA |
| | TTACCCCCGTAA | | | TTACCCCCGTAA |
| | ACCCCGGAACGT | | | ACCCCGGAACGT |
| | CACCACTTCTAC | | | CACCACTTCTAC |
| | GATATGCCGCAT | | | GATATGCCGCAT |
| | GGACCGGAGGGC | | | GGACCGGAGGGC |
| | TTGCAGCAGTAG | | | TTGCAGCAGTAG |
| | TACTTTTATGTC | | | TACTTTTATGTC |
| | TCGTAATATTTT | | | TCGTAATATTTT |
| | TAATCTGTACGG | | | TAATCTGTACGG |
| | CTAAACGAATGA | | | CTAAACGAATGA |
| | GGGTTAAAGCCG | | | GGGTTAAAGCCG |
| | CCAGGGTAGACA | | | CCAGGGTAGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |
| | Sequence, NT | | | mRNA Sequence |
| | (5′ UTR, ORF, | | | (assumes |
| mRNA | 3′ UTR) | ORF Sequence, AA | ORF Sequence, NT | T100 tail) |
| Name(s) | SEQ ID NO: 68 | SEQ ID NO: 69 | SEQ ID NO: 70 | SEQ ID NO: 71 |
| VZV-GE- | GGGAAATAAGAG | MGTVNKPVVGVLMGFGI | ATGGGGACAGTTAATAA | GGGAAATAAGAG |
| truncated- | AGAAAAGAAGAG | ITGTLRITNPVRASVLR | ACCTGTGGTGGGGGTAT | AGAAAAGAAGAG |
| delete_ | TAAGAAGAAATA | YDDFHIDEDKLDTNSVY | TGATGGGGTTCGGAATT | TAAGAAGAAATA |
| from_ | TAAGAGCCACCA | EPYYHSDHAESSWVNRG | ATCACGGGAACGTTGCG | TAAGAGCCACCA |
| 574_-_ | TGGGGACAGTTA | ESSRKAYDHNSPYIWPR | TATAACGAATCCGGTCA | TGGGGACAGTTA |
| Y569A | ATAAACCTGTGG | NDYDGFLENAHEHHGVY | GAGCATCCGTCTTGCGA | ATAAACCTGTGG |
| Variant | TGGGGGTATTGA | NQGRGIDSGERLMQPTQ | TACGATGATTTTCACAT | TGGGGGTATTGA |
| 3 | TGGGGTTCGGAA | MSAQEDLGDDTGIHVIP | CGATGAAGACAAACTGG | TGGGGTTCGGAA |
| | TTATCACGGGAA | TLNGDDRHKIVNVDQRQ | ATACAAACTCCGTATAT | TTATCACGGGAA |
| | CGTTGCGTATAA | YGDVFKGDLNPKPQGQR | GAGCCTTACTACCATTC | CGTTGCGTATAA |
| | CGAATCCGGTCA | LIEVSVEENHPFTLRAP | AGATCATGCGGAGTCTT | CGAATCCGGTCA |
| | GAGCATCCGTCT | IQRIYGVRYTETWSFLP | CATGGGTAAATCGGGGA | GAGCATCCGTCT |
| | TGCGATACGATG | SLTCTGDAAPAIQHICL | GAGTCTTCGCGAAAAGC | TGCGATACGATG |
| | ATTTTCACATCG | KHTTCFQDVVVDVDCAE | GTACGATCATAACTCAC | ATTTTCACATCG |
| | ATGAAGACAAAC | NTKEDQLAEISYRFQGK | CTTATATATGGCCACGT | ATGAAGACAAAC |
| | TGGATACAAACT | KEADQPWIVVNTSTLFD | AATGATTATGATGGATT | TGGATACAAACT |
| | CCGTATATGAGC | ELELDPPEIEPGVLKVL | TTTAGAGAACGCACACG | CCGTATATGAGC |
| | CTTACTACCATT | RTEKQYLGVYIWNMRGS | AACACCATGGGGTGTAT | CTTACTACCATT |
| | CAGATCATGCGG | DGTSTYATFLVTWKGDE | AATCAGGGCCGTGGTAT | CAGATCATGCGG |
| | AGTCTTCATGGG | KTRNPTPAVTPQPRGAE | CGATAGCGGGGAACGGT | AGTCTTCATGGG |
| | TAAATCGGGGAG | FHMWNYHSHVFSVGDTF | TAATGCAACCCACACAA | TAAATCGGGGAG |
| | AGTCTTCGCGAA | SLAMHLQYKIHEAPFDL | ATGTCTGCACAGGAGGA | AGTCTTCGCGAA |
| | AAGCGTACGATC | LLEWLYVPIDPTCQPMR | TCTTGGGGACGATACGG | AAGCGTACGATC |
| | ATAACTCACCTT | LYSTCLYHPNAPQCLSH | GCATCCACGTTATCCCT | ATAACTCACCTT |
| | ATATATGGCCAC | MNSGCTFTSPHLAQRVA | ACGTTAAACGGCGATGA | ATATATGGCCAC |
| | GTAATGATTATG | STVYQNCEHADNYTAYC | CAGACATAAAATTGTAA | GTAATGATTATG |
| | ATGGATTTTTAG | LGISHMEPSFGLILHDG | ATGTGGACCAACGTCAA | ATGGATTTTTAG |
| | AGAACGCACACG | GTTLKFVDTPESLSGLY | TACGGTGACGTGTTTAA | AGAACGCACACG |
| | AACACCATGGGG | VFVVYFNGHVEAVAYTV | AGGAGATCTTAATCCAA | AACACCATGGGG |
| | TGTATAATCAGG | VSTVDHFVNAIEERGFP | AACCCCAAGGCCAAAGA | TGTATAATCAGG |
| | GCCGTGGTATCG | PTAGQPPATTKPKEITP | CTCATTGAGGTGTCAGT | GCCGTGGTATCG |
| | ATAGCGGGGAAC | VNPGTSPLLRYAAWTGG | GGAAGAAAATCACCCGT | ATAGCGGGGAAC |
| | GGTTAATGCAAC | LAAVVLLCLVIFLICTA | TTACTTTACGCGCACCG | GGTTAATGCAAC |
| | CCACACAAATGT | KRMRVKAARVDK | ATTCAGCGGATTTATGG | CCACACAAATGT |
| | CTGCACAGGAGG | | AGTCCGGTACACCGAGA | CTGCACAGGAGG |
| | ATCTTGGGGACG | | CTTGGAGCTTTTTGCCG | ATCTTGGGGACG |
| | ATACGGGCATCC | | TCATTAACCTGTACGGG | ATACGGGCATCC |
| | ACGTTATCCCTA | | AGACGCAGCGCCCGCCA | ACGTTATCCCTA |
| | CGTTAAACGGCG | | TCCAGCATATATGTTTA | CGTTAAACGGCG |
| | ATGACAGACATA | | AAACATACAACATGCTT | ATGACAGACATA |
| | AAATTGTAAATG | | TCAAGACGTGGTGGTGG | AAATTGTAAATG |
| | TGGACCAACGTC | | ATGTGGATTGCGCGGAA | TGGACCAACGTC |
| | AATACGGTGACG | | AATACTAAAGAGGATCA | AATACGGTGACG |
| | TGTTTAAAGGAG | | GTTGGCCGAAATCAGTT | TGTTTAAAGGAG |
| | ATCTTAATCCAA | | ACCGTTTTCAAGGTAAG | ATCTTAATCCAA |
| | AACCCCAAGGCC | | AAGGAAGCGGACCAACC | AACCCCAAGGCC |
| | AAAGACTCATTG | | GTGGATTGTTGTAAACA | AAAGACTCATTG |
| | AGGTGTCAGTGG | | CGAGCACACTGTTTGAT | AGGTGTCAGTGG |
| | AAGAAAATCACC | | GAACTCGAATTAGACCC | AAGAAAATCACC |
| | CGTTTACTTTAC | | ACCCGAGATTGAACCGG | CGTTTACTTTAC |
| | GCGCACCGATTC | | GTGTCTTGAAAGTACTT | GCGCACCGATTC |
| | AGCGGATTTATG | | CGGACAGAGAAACAATA | AGCGGATTTATG |
| | GAGTCCGGTACA | | CTTGGGTGTGTACATTT | GAGTCCGGTACA |
| | CCGAGACTTGGA | | GGAACATGCGCGGCTCC | CCGAGACTTGGA |
| | GCTTTTTGCCGT | | GATGGTACGTCTACCTA | GCTTTTTGCCGT |
| | CATTAACCTGTA | | CGCCACGTTTTTGGTCA | CATTAACCTGTA |
| | CGGGAGACGCAG | | CCTGGAAAGGGGATGAG | CGGGAGACGCAG |
| | CGCCCGCCATCC | | AAGACAAGAAACCCTAC | CGCCCGCCATCC |
| | AGCATATATGTT | | GCCCGCAGTAACTCCTC | AGCATATATGTT |
| | TAAAACATACAA | | AACCAAGAGGGGCTGAG | TAAAACATACAA |
| | CATGCTTTCAAG | | TTTCATATGTGGAATTA | CATGCTTTCAAG |
| | ACGTGGTGGTGG | | CCACTCGCATGTATTTT | ACGTGGTGGTGG |
| | ATGTGGATTGCG | | CAGTTGGTGATACGTTT | ATGTGGATTGCG |
| | CGGAAAATACTA | | AGCTTGGCAATGCATCT | CGGAAAATACTA |
| | AAGAGGATCAGT | | TCAGTATAAGATACATG | AAGAGGATCAGT |
| | TGGCCGAAATCA | | AAGCGCCATTTGATTTG | TGGCCGAAATCA |
| | GTTACCGTTTTC | | CTGTTAGAGTGGTTGTA | GTTACCGTTTTC |
| | AAGGTAAGAAGG | | TGTCCCCATCGATCCTA | AAGGTAAGAAGG |
| | AAGCGGACCAAC | | CATGTCAACCAATGCGG | AAGCGGACCAAC |
| | CGTGGATTGTTG | | TTATATTCTACGTGTTT | CGTGGATTGTTG |
| | TAAACACGAGCA | | GTATCATCCCAACGCAC | TAAACACGAGCA |
| | CACTGTTTGATG | | CCCAATGCCTCTCTCAT | CACTGTTTGATG |
| | AACTCGAATTAG | | ATGAATTCCGGTTGTAC | AACTCGAATTAG |
| | ACCCACCCGAGA | | ATTTACCTCGCCACATT | ACCCACCCGAGA |
| | TTGAACCGGGTG | | TAGCCCAGCGTGTTGCA | TTGAACCGGGTG |
| | TCTTGAAAGTAC | | AGCACAGTGTATCAAAA | TCTTGAAAGTAC |
| | TTCGGACAGAGA | | TTGTGAACATGCAGATA | TTCGGACAGAGA |
| | AACAATACTTGG | | ACTACACCGCATATTGT | AACAATACTTGG |
| | GTGTGTACATTT | | CTGGGAATATCTCATAT | GTGTGTACATTT |
| | GGAACATGCGCG | | GGAGCCTAGCTTTGGTC | GGAACATGCGCG |
| | GCTCCGATGGTA | | TAATCTTACACGACGGG | GCTCCGATGGTA |
| | CGTCTACCTACG | | GGCACCACGTTAAAGTT | CGTCTACCTACG |
| | CCACGTTTTTGG | | TGTAGATACACCCGAGA | CCACGTTTTTGG |
| | TCACCTGGAAAG | | GTTTGTCGGGATTATAC | TCACCTGGAAAG |
| | GGGATGAGAAGA | | GTTTTTGTGGTGTATTT | GGGATGAGAAGA |
| | CAAGAAACCCTA | | TAACGGGCATGTTGAAG | CAAGAAACCCTA |
| | CGCCCGCAGTAA | | CCGTAGCATACACTGTT | CGCCCGCAGTAA |
| | CTCCTCAACCAA | | GTATCCACAGTAGATCA | CTCCTCAACCAA |
| | GAGGGGCTGAGT | | TTTTGTAAACGCAATTG | GAGGGGCTGAGT |
| | TTCATATGTGGA | | AAGAGCGTGGATTTCCG | TTCATATGTGGA |
| | ATTACCACTCGC | | CCAACGGCCGGTCAGCC | ATTACCACTCGC |
| | ATGTATTTTCAG | | ACCGGCGACTACTAAAC | ATGTATTTTCAG |
| | TTGGTGATACGT | | CCAAGGAAATTACCCCC | TTGGTGATACGT |
| | TTAGCTTGGCAA | | GTAAACCCCGGAACGTC | TTAGCTTGGCAA |
| | TGCATCTTCAGT | | ACCACTTCTACGATATG | TGCATCTTCAGT |
| | ATAAGATACATG | | CCGCATGGACCGGAGGG | ATAAGATACATG |
| | AAGCGCCATTTG | | CTTGCAGCAGTAGTACT | AAGCGCCATTTG |
| | ATTTGCTGTTAG | | TTTATGTCTCGTAATAT | ATTTGCTGTTAG |
| | AGTGGTTGTATG | | TTTTAATCTGTACGGCT | AGTGGTTGTATG |
| | TCCCCATCGATC | | AAACGAATGAGGGTTAA | TCCCCATCGATC |
| | CTACATGTCAAC | | AGCCGCCAGGGTAGACA | CTACATGTCAAC |
| | CAATGCGGTTAT | | AG | CAATGCGGTTAT |
| | ATTCTACGTGTT | | | ATTCTACGTGTT |
| | TGTATCATCCCA | | | TGTATCATCCCA |
| | ACGCACCCCAAT | | | ACGCACCCCAAT |
| | GCCTCTCTCATA | | | GCCTCTCTCATA |
| | TGAATTCCGGTT | | | TGAATTCCGGTT |
| | GTACATTTACCT | | | GTACATTTACCT |
| | CGCCACATTTAG | | | CGCCACATTTAG |
| | CCCAGCGTGTTG | | | CCCAGCGTGTTG |
| | CAAGCACAGTGT | | | CAAGCACAGTGT |
| | ATCAAAATTGTG | | | ATCAAAATTGTG |
| | AACATGCAGATA | | | AACATGCAGATA |
| | ACTACACCGCAT | | | ACTACACCGCAT |
| | ATTGTCTGGGAA | | | ATTGTCTGGGAA |
| | TATCTCATATGG | | | TATCTCATATGG |
| | AGCCTAGCTTTG | | | AGCCTAGCTTTG |
| | GTCTAATCTTAC | | | GTCTAATCTTAC |
| | ACGACGGGGGCA | | | ACGACGGGGGCA |
| | CCACGTTAAAGT | | | CCACGTTAAAGT |
| | TTGTAGATACAC | | | TTGTAGATACAC |
| | CCGAGAGTTTGT | | | CCGAGAGTTTGT |
| | CGGGATTATACG | | | CGGGATTATACG |
| | TTTTTGTGGTGT | | | TTTTTGTGGTGT |
| | ATTTTAACGGGC | | | ATTTTAACGGGC |
| | ATGTTGAAGCCG | | | ATGTTGAAGCCG |
| | TAGCATACACTG | | | TAGCATACACTG |
| | TTGTATCCACAG | | | TTGTATCCACAG |
| | TAGATCATTTTG | | | TAGATCATTTTG |
| | TAAACGCAATTG | | | TAAACGCAATTG |
| | AAGAGCGTGGAT | | | AAGAGCGTGGAT |
| | TTCCGCCAACGG | | | TTCCGCCAACGG |
| | CCGGTCAGCCAC | | | CCGGTCAGCCAC |
| | CGGCGACTACTA | | | CGGCGACTACTA |
| | AACCCAAGGAAA | | | AACCCAAGGAAA |
| | TTACCCCCGTAA | | | TTACCCCCGTAA |
| | ACCCCGGAACGT | | | ACCCCGGAACGT |
| | CACCACTTCTAC | | | CACCACTTCTAC |
| | GATATGCCGCAT | | | GATATGCCGCAT |
| | GGACCGGAGGGC | | | GGACCGGAGGGC |
| | TTGCAGCAGTAG | | | TTGCAGCAGTAG |
| | TACTTTTATGTC | | | TACTTTTATGTC |
| | TCGTAATATTTT | | | TCGTAATATTTT |
| | TAATCTGTACGG | | | TAATCTGTACGG |
| | CTAAACGAATGA | | | CTAAACGAATGA |
| | GGGTTAAAGCCG | | | GGGTTAAAGCCG |
| | CCAGGGTAGACA | | | CCAGGGTAGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |
| | Sequence, NT | | | mRNA Sequence |
| | (5′ UTR, ORF, | | | (assumes |
| mRNA | 3′ UTR) | ORF Sequence, AA | ORF Sequence, NT | T100 tail) |
| Name(s) | SEQ ID NO: 72 | SEQ ID NO: 73 | SEQ ID NO: 74 | SEQ ID NO: 75 |
| VZV-GE- | GGGAAATAAGAG | MGTVNKPVVGVLMGFGI | ATGGGGACAGTTAATAA | GGGAAATAAGAG |
| truncated- | AGAAAAGAAGAG | ITGTLRITNPVRASVLR | ACCTGTGGTGGGGGTAT | AGAAAAGAAGAG |
| delete_ | TAAGAAGAAATA | YDDFHIDEDKLDTNSVY | TGATGGGGTTCGGAATT | TAAGAAGAAATA |
| from_ | TAAGAGCCACCA | EPYYHSDHAESSWVNRG | ATCACGGGAACGTTGCG | TAAGAGCCACCA |
| 574_-_ | TGGGGACAGTTA | ESSRKAYDHNSPYIWPR | TATAACGAATCCGGTCA | TGGGGACAGTTA |
| Y569A | ATAAACCTGTGG | NDYDGFLENAHEHHGVY | GAGCATCCGTCTTGCGA | ATAAACCTGTGG |
| Variant 4 | TGGGGGTATTGA | NQGRGIDSGERLMQPTQ | TACGATGATTTTCACAT | TGGGGGTATTGA |
| | TGGGGTTCGGAA | MSAQEDLGDDTGIHVIP | CGATGAAGACAAACTGG | TGGGGTTCGGAA |
| | TTATCACGGGAA | TLNGDDRHKIVNVDQRQ | ATACAAACTCCGTATAT | TTATCACGGGAA |
| | CGTTGCGTATAA | YGDVFKGDLNPKPQGQR | GAGCCTTACTACCATTC | CGTTGCGTATAA |
| | CGAATCCGGTCA | LIEVSVEENHPFTLRAP | AGATCATGCGGAGTCTT | CGAATCCGGTCA |
| | GAGCATCCGTCT | IQRIYGVRYTETWSFLP | CATGGGTAAATCGGGGA | GAGCATCCGTCT |
| | TGCGATACGATG | SLTCTGDAAPAIQHICL | GAGTCTTCGCGAAAGGC | TGCGATACGATG |
| | ATTTTCACATCG | KHTTCFQDVVVDVDCAE | GTACGATCATAACTCAC | ATTTTCACATCG |
| | ATGAAGACAAAC | NTKEDQLAEISYRFQGK | CTTATATATGGCCACGT | ATGAAGACAAAC |
| | TGGATACAAACT | KEADQPWIVVNTSTLFD | AATGATTATGATGGATT | TGGATACAAACT |
| | CCGTATATGAGC | ELELDPPEIEPGVLKVL | TTTAGAGAACGCACACG | CCGTATATGAGC |
| | CTTACTACCATT | RTEKQYLGVYIWNMRGS | AACACCATGGGGTGTAT | CTTACTACCATT |
| | CAGATCATGCGG | DGTSTYATFLVTWKGDE | AATCAGGGCCGTGGTAT | CAGATCATGCGG |
| | AGTCTTCATGGG | KTRNPTPAVTPQPRGAE | CGATAGCGGGGAACGGT | AGTCTTCATGGG |
| | TAAATCGGGGAG | FHMWNYHSHVFSVGDTF | TAATGCAACCCACACAA | TAAATCGGGGAG |
| | AGTCTTCGCGAA | SLAMHLQYKIHEAPFDL | ATGTCTGCACAGGAGGA | AGTCTTCGCGAA |
| | AGGCGTACGATC | LLEWLYVPIDPTCQPMR | TCTTGGGGACGATACGG | AGGCGTACGATC |
| | ATAACTCACCTT | LYSTCLYHPNAPQCLSH | GCATCCACGTTATCCCT | ATAACTCACCTT |
| | ATATATGGCCAC | MNSGCTFTSPHLAQRVA | ACGTTAAACGGCGATGA | ATATATGGCCAC |
| | GTAATGATTATG | STVYQNCEHADNYTAYC | CAGACATAAGATTGTAA | GTAATGATTATG |
| | ATGGATTTTTAG | LGISHMEPSFGLILHDG | ATGTGGACCAACGTCAA | ATGGATTTTTAG |
| | AGAACGCACACG | GTTLKFVDTPESLSGLY | TACGGTGACGTGTTTAA | AGAACGCACACG |
| | AACACCATGGGG | VFVVYFNGHVEAVAYTV | AGGAGATCTTAATCCAA | AACACCATGGGG |
| | TGTATAATCAGG | VSTVDHFVNAIEERGFP | AGCCCCAAGGCCAAAGA | TGTATAATCAGG |
| | GCCGTGGTATCG | PTAGQPPATTKPKEITP | CTCATTGAGGTGTCAGT | GCCGTGGTATCG |
| | ATAGCGGGGAAC | VNPGTSPLLRYAAWTGG | GGAAGAGAATCACCCGT | ATAGCGGGGAAC |
| | GGTTAATGCAAC | LAAVVLLCLVIFLICTA | TTACTTTACGCGCACCG | GGTTAATGCAAC |
| | CCACACAAATGT | KRMRVKAARVDK | ATTCAGCGGATTTATGG | CCACACAAATGT |
| | CTGCACAGGAGG | | AGTCCGGTACACCGAGA | CTGCACAGGAGG |
| | ATCTTGGGGACG | | CTTGGAGCTTTTTGCCG | ATCTTGGGGACG |
| | ATACGGGCATCC | | TCATTAACCTGTACGGG | ATACGGGCATCC |
| | ACGTTATCCCTA | | AGACGCAGCGCCCGCCA | ACGTTATCCCTA |
| | CGTTAAACGGCG | | TCCAGCATATATGTTTA | CGTTAAACGGCG |
| | ATGACAGACATA | | AAGCATACAACATGCTT | ATGACAGACATA |
| | AGATTGTAAATG | | TCAAGACGTGGTGGTGG | AGATTGTAAATG |
| | TGGACCAACGTC | | ATGTGGATTGCGCGGAG | TGGACCAACGTC |
| | AATACGGTGACG | | AATACTAAAGAGGATCA | AATACGGTGACG |
| | TGTTTAAAGGAG | | GTTGGCCGAAATCAGTT | TGTTTAAAGGAG |
| | ATCTTAATCCAA | | ACCGTTTTCAAGGTAAG | ATCTTAATCCAA |
| | AGCCCCAAGGCC | | AAGGAAGCGGACCAACC | AGCCCCAAGGCC |
| | AAAGACTCATTG | | GTGGATTGTTGTAAACA | AAAGACTCATTG |
| | AGGTGTCAGTGG | | CGAGCACACTGTTTGAT | AGGTGTCAGTGG |
| | AAGAGAATCACC | | GAACTCGAATTAGACCC | AAGAGAATCACC |
| | CGTTTACTTTAC | | CCCCGAGATTGAACCGG | CGTTTACTTTAC |
| | GCGCACCGATTC | | GTGTCTTGAAAGTACTT | GCGCACCGATTC |
| | AGCGGATTTATG | | CGGACAGAGAAACAATA | AGCGGATTTATG |
| | GAGTCCGGTACA | | CTTGGGTGTGTACATTT | GAGTCCGGTACA |
| | CCGAGACTTGGA | | GGAACATGCGCGGCTCC | CCGAGACTTGGA |
| | GCTTTTTGCCGT | | GATGGTACGTCTACCTA | GCTTTTTGCCGT |
| | CATTAACCTGTA | | CGCCACGTTTTTGGTCA | CATTAACCTGTA |
| | CGGGAGACGCAG | | CCTGGAAAGGGGATGAG | CGGGAGACGCAG |
| | CGCCCGCCATCC | | AAGACAAGAAACCCTAC | CGCCCGCCATCC |
| | AGCATATATGTT | | GCCCGCAGTAACTCCTC | AGCATATATGTT |
| | TAAAGCATACAA | | AACCAAGAGGGGCTGAG | TAAAGCATACAA |
| | CATGCTTTCAAG | | TTTCATATGTGGAATTA | CATGCTTTCAAG |
| | ACGTGGTGGTGG | | CCACTCGCATGTATTTT | ACGTGGTGGTGG |
| | ATGTGGATTGCG | | CAGTTGGTGATACGTTT | ATGTGGATTGCG |
| | CGGAGAATACTA | | AGCTTGGCAATGCATCT | CGGAGAATACTA |
| | AAGAGGATCAGT | | TCAGTATAAGATACATG | AAGAGGATCAGT |
| | TGGCCGAAATCA | | AAGCGCCATTTGATTTG | TGGCCGAAATCA |
| | GTTACCGTTTTC | | CTGTTAGAGTGGTTGTA | GTTACCGTTTTC |
| | AAGGTAAGAAGG | | TGTCCCCATCGATCCTA | AAGGTAAGAAGG |
| | AAGCGGACCAAC | | CATGTCAACCAATGCGG | AAGCGGACCAAC |
| | CGTGGATTGTTG | | TTATATTCTACGTGTTT | CGTGGATTGTTG |
| | TAAACACGAGCA | | GTATCATCCCAACGCAC | TAAACACGAGCA |
| | CACTGTTTGATG | | CCCAATGCCTCTCTCAT | CACTGTTTGATG |
| | AACTCGAATTAG | | ATGAATTCCGGTTGTAC | AACTCGAATTAG |
| | ACCCCCCCGAGA | | ATTTACCTCGCCACATT | ACCCCCCCGAGA |
| | TTGAACCGGGTG | | TAGCCCAGCGTGTTGCA | TTGAACCGGGTG |
| | TCTTGAAAGTAC | | AGCACAGTGTATCAGAA | TCTTGAAAGTAC |
| | TTCGGACAGAGA | | TTGTGAACATGCAGATA | TTCGGACAGAGA |
| | AACAATACTTGG | | ACTACACCGCATATTGT | AACAATACTTGG |
| | GTGTGTACATTT | | CTGGGAATATCTCATAT | GTGTGTACATTT |
| | GGAACATGCGCG | | GGAGCCTAGCTTTGGTC | GGAACATGCGCG |
| | GCTCCGATGGTA | | TAATCTTACACGACGGG | GCTCCGATGGTA |
| | CGTCTACCTACG | | GGCACCACGTTAAAGTT | CGTCTACCTACG |
| | CCACGTTTTTGG | | TGTAGATACACCCGAGA | CCACGTTTTTGG |
| | TCACCTGGAAAG | | GTTTGTCGGGATTATAC | TCACCTGGAAAG |
| | GGGATGAGAAGA | | GTTTTTGTGGTGTATTT | GGGATGAGAAGA |
| | CAAGAAACCCTA | | TAACGGGCATGTTGAAG | CAAGAAACCCTA |
| | CGCCCGCAGTAA | | CCGTAGCATACACTGTT | CGCCCGCAGTAA |
| | CTCCTCAACCAA | | GTATCCACAGTAGATCA | CTCCTCAACCAA |
| | GAGGGGCTGAGT | | TTTTGTAAACGCAATTG | GAGGGGCTGAGT |
| | TTCATATGTGGA | | AAGAGCGTGGATTTCCG | TTCATATGTGGA |
| | ATTACCACTCGC | | CCAACGGCCGGTCAGCC | ATTACCACTCGC |
| | ATGTATTTTCAG | | ACCGGCGACTACTAAAC | ATGTATTTTCAG |
| | TTGGTGATACGT | | CCAAGGAAATTACCCCC | TTGGTGATACGT |
| | TTAGCTTGGCAA | | GTAAACCCCGGAACGTC | TTAGCTTGGCAA |
| | TGCATCTTCAGT | | ACCACTTCTACGATATG | TGCATCTTCAGT |
| | ATAAGATACATG | | CCGCATGGACCGGAGGG | ATAAGATACATG |
| | AAGCGCCATTTG | | CTTGCAGCAGTAGTACT | AAGCGCCATTTG |
| | ATTTGCTGTTAG | | TTTATGTCTCGTAATAT | ATTTGCTGTTAG |
| | AGTGGTTGTATG | | TTTTAATCTGTACGGCT | AGTGGTTGTATG |
| | TCCCCATCGATC | | AAACGAATGAGGGTTAA | TCCCCATCGATC |
| | CTACATGTCAAC | | AGCCGCCAGGGTAGACA | CTACATGTCAAC |
| | CAATGCGGTTAT | | AG | CAATGCGGTTAT |
| | ATTCTACGTGTT | | | ATTCTACGTGTT |
| | TGTATCATCCCA | | | TGTATCATCCCA |
| | ACGCACCCCAAT | | | ACGCACCCCAAT |
| | GCCTCTCTCATA | | | GCCTCTCTCATA |
| | TGAATTCCGGTT | | | TGAATTCCGGTT |
| | GTACATTTACCT | | | GTACATTTACCT |
| | CGCCACATTTAG | | | CGCCACATTTAG |
| | CCCAGCGTGTTG | | | CCCAGCGTGTTG |
| | CAAGCACAGTGT | | | CAAGCACAGTGT |
| | ATCAGAATTGTG | | | ATCAGAATTGTG |
| | AACATGCAGATA | | | AACATGCAGATA |
| | ACTACACCGCAT | | | ACTACACCGCAT |
| | ATTGTCTGGGAA | | | ATTGTCTGGGAA |
| | TATCTCATATGG | | | TATCTCATATGG |
| | AGCCTAGCTTTG | | | AGCCTAGCTTTG |
| | GTCTAATCTTAC | | | GTCTAATCTTAC |
| | ACGACGGGGGCA | | | ACGACGGGGGCA |
| | CCACGTTAAAGT | | | CCACGTTAAAGT |
| | TTGTAGATACAC | | | TTGTAGATACAC |
| | CCGAGAGTTTGT | | | CCGAGAGTTTGT |
| | CGGGATTATACG | | | CGGGATTATACG |
| | TTTTTGTGGTGT | | | TTTTTGTGGTGT |
| | ATTTTAACGGGC | | | ATTTTAACGGGC |
| | ATGTTGAAGCCG | | | ATGTTGAAGCCG |
| | TAGCATACACTG | | | TAGCATACACTG |
| | TTGTATCCACAG | | | TTGTATCCACAG |
| | TAGATCATTTTG | | | TAGATCATTTTG |
| | TAAACGCAATTG | | | TAAACGCAATTG |
| | AAGAGCGTGGAT | | | AAGAGCGTGGAT |
| | TTCCGCCAACGG | | | TTCCGCCAACGG |
| | CCGGTCAGCCAC | | | CCGGTCAGCCAC |
| | CGGCGACTACTA | | | CGGCGACTACTA |
| | AACCCAAGGAAA | | | AACCCAAGGAAA |
| | TTACCCCCGTAA | | | TTACCCCCGTAA |
| | ACCCCGGAACGT | | | ACCCCGGAACGT |
| | CACCACTTCTAC | | | CACCACTTCTAC |
| | GATATGCCGCAT | | | GATATGCCGCAT |
| | GGACCGGAGGGC | | | GGACCGGAGGGC |
| | TTGCAGCAGTAG | | | TTGCAGCAGTAG |
| | TACTTTTATGTC | | | TACTTTTATGTC |
| | TCGTAATATTTT | | | TCGTAATATTTT |
| | TAATCTGTACGG | | | TAATCTGTACGG |
| | CTAAACGAATGA | | | CTAAACGAATGA |
| | GGGTTAAAGCCG | | | GGGTTAAAGCCG |
| | CCAGGGTAGACA | | | CCAGGGTAGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |
| | Sequence, NT | | | mRNA Sequence |
| | (5′ UTR, | | | (assumes |
| mRNA | ORF, 3′ UTR) | ORF Sequence, AA | ORF Sequence, NT | T100 tail) |
| Name(s) | SEQ ID NO: 76 | SEQ ID NO: 77 | SEQ ID NO: 78 | SEQ ID NO: 79 |
| VZV-GE- | GGGAAATAAGAG | MGTVNKPVVGVLMGFGI | ATGGGGACAGTTAATAA | GGGAAATAAGAG |
| truncated- | AGAAAAGAAGAG | ITGTLRITNPVRASVLR | ACCTGTGGTGGGGGTAT | AGAAAAGAAGAG |
| delete_ | TAAGAAGAAATA | YDDFHIDEDKLDTNSVY | TGATGGGGTTCGGAATT | TAAGAAGAAATA |
| from_ | TAAGAGCCACCA | EPYYHSDHAESSWVNRG | ATCACGGGAACGTTGCG | TAAGAGCCACCA |
| 574_-_ | TGGGGACAGTTA | ESSRKAYDHNSPYIWPR | TATAACGAATCCGGTCA | TGGGGACAGTTA |
| Y569A | ATAAACCTGTGG | NDYDGFLENAHEHHGVY | GAGCATCCGTCTTGCGA | ATAAACCTGTGG |
| Variant | TGGGGGTATTGA | NQGRGIDSGERLMQPTQ | TACGATGATTTTCACAT | TGGGGGTATTGA |
| 5 | TGGGGTTCGGAA | MSAQEDLGDDTGIHVIP | CGATGAAGACAAACTGG | TGGGGTTCGGAA |
| | TTATCACGGGAA | TLNGDDRHKIVNVDQRQ | ATACAAACTCCGTATAT | TTATCACGGGAA |
| | CGTTGCGTATAA | YGDVFKGDLNPKPQGQR | GAGCCTTACTACCATTC | CGTTGCGTATAA |
| | CGAATCCGGTCA | LIEVSVEENHPFTLRAP | AGATCATGCGGAGTCTT | CGAATCCGGTCA |
| | GAGCATCCGTCT | IQRIYGVRYTETWSFLP | CATGGGTAAATCGGGGA | GAGCATCCGTCT |
| | TGCGATACGATG | SLTCTGDAAPAIQHICL | GAGTCTTCGCGAAAGGC | TGCGATACGATG |
| | ATTTTCACATCG | KHTTCFQDVVVDVDCAE | GTACGATCATAACTCAC | ATTTTCACATCG |
| | ATGAAGACAAAC | NTKEDQLAEISYRFQGK | CTTATATATGGCCACGT | ATGAAGACAAAC |
| | TGGATACAAACT | KEADQPWIVVNTSTLFD | AATGATTATGATGGATT | TGGATACAAACT |
| | CCGTATATGAGC | ELELDPPEIEPGVLKVL | TTTAGAGAACGCACACG | CCGTATATGAGC |
| | CTTACTACCATT | RTEKQYLGVYIWNMRGS | AACACCATGGGGTGTAT | CTTACTACCATT |
| | CAGATCATGCGG | DGTSTYATFLVTWKGDE | AATCAGGGCCGTGGTAT | CAGATCATGCGG |
| | AGTCTTCATGGG | KTRNPTPAVTPQPRGAE | CGATAGCGGGGAACGGT | AGTCTTCATGGG |
| | TAAATCGGGGAG | FHMWNYHSHVFSVGDTF | TAATGCAACCCACACAA | TAAATCGGGGAG |
| | AGTCTTCGCGAA | SLAMHLQYKIHEAPFDL | ATGTCTGCACAGGAGGA | AGTCTTCGCGAA |
| | AGGCGTACGATC | LLEWLYVPIDPTCQPMR | TCTTGGGGACGATACGG | AGGCGTACGATC |
| | ATAACTCACCTT | LYSTCLYHPNAPQCLSH | GCATCCACGTTATCCCT | ATAACTCACCTT |
| | ATATATGGCCAC | MNSGCTFTSPHLAQRVA | ACGTTAAACGGCGATGA | ATATATGGCCAC |
| | GTAATGATTATG | STVYQNCEHADNYTAYC | CAGACATAAGATTGTAA | GTAATGATTATG |
| | ATGGATTTTTAG | LGISHMEPSFGLILHDG | ATGTGGACCAACGTCAA | ATGGATTTTTAG |
| | AGAACGCACACG | GTTLKFVDTPESLSGLY | TACGGTGACGTGTTTAA | AGAACGCACACG |
| | AACACCATGGGG | VFVVYFNGHVEAVAYTV | AGGAGATCTTAATCCAA | AACACCATGGGG |
| | TGTATAATCAGG | VSTVDHEVNAIEERGFP | AGCCCCAAGGCCAAAGA | TGTATAATCAGG |
| | GCCGTGGTATCG | PTAGQPPATTKPKEITP | CTCATTGAGGTGTCAGT | GCCGTGGTATCG |
| | ATAGCGGGGAAC | VNPGTSPLLRYAAWTGG | GGAAGAGAATCACCCGT | ATAGCGGGGAAC |
| | GGTTAATGCAAC | LAAVVLLCLVIFLICTA | TTACTTTACGCGCACCG | GGTTAATGCAAC |
| | CCACACAAATGT | KRMRVKAARVDK | ATTCAGCGGATTTATGG | CCACACAAATGT |
| | CTGCACAGGAGG | | AGTCCGGTACACCGAGA | CTGCACAGGAGG |
| | ATCTTGGGGACG | | CTTGGAGCTTTTTGCCG | ATCTTGGGGACG |
| | ATACGGGCATCC | | TCATTAACCTGTACGGG | ATACGGGCATCC |
| | ACGTTATCCCTA | | AGACGCAGCGCCCGCCA | ACGTTATCCCTA |
| | CGTTAAACGGCG | | TCCAGCATATATGTTTA | CGTTAAACGGCG |
| | ATGACAGACATA | | AAGCATACAACATGCTT | ATGACAGACATA |
| | AGATTGTAAATG | | TCAAGACGTGGTGGTGG | AGATTGTAAATG |
| | TGGACCAACGTC | | ATGTGGATTGCGCGGAG | TGGACCAACGTC |
| | AATACGGTGACG | | AATACTAAAGAGGATCA | AATACGGTGACG |
| | TGTTTAAAGGAG | | GTTGGCCGAAATCAGTT | TGTTTAAAGGAG |
| | ATCTTAATCCAA | | ACCGTTTTCAAGGTAAG | ATCTTAATCCAA |
| | AGCCCCAAGGCC | | AAGGAAGCGGACCAACC | AGCCCCAAGGCC |
| | AAAGACTCATTG | | GTGGATTGTTGTAAACA | AAAGACTCATTG |
| | AGGTGTCAGTGG | | CGAGCACACTGTTTGAT | AGGTGTCAGTGG |
| | AAGAGAATCACC | | GAACTCGAATTAGACCC | AAGAGAATCACC |
| | CGTTTACTTTAC | | ACCCGAGATTGAACCGG | CGTTTACTTTAC |
| | GCGCACCGATTC | | GTGTCTTGAAAGTACTT | GCGCACCGATTC |
| | AGCGGATTTATG | | CGGACAGAGAAACAATA | AGCGGATTTATG |
| | GAGTCCGGTACA | | CTTGGGTGTGTACATTT | GAGTCCGGTACA |
| | CCGAGACTTGGA | | GGAACATGCGCGGCTCC | CCGAGACTTGGA |
| | GCTTTTTGCCGT | | GATGGTACGTCTACCTA | GCTTTTTGCCGT |
| | CATTAACCTGTA | | CGCCACGTTTTTGGTCA | CATTAACCTGTA |
| | CGGGAGACGCAG | | CCTGGAAAGGGGATGAG | CGGGAGACGCAG |
| | CGCCCGCCATCC | | AAGACAAGAAACCCTAC | CGCCCGCCATCC |
| | AGCATATATGTT | | GCCCGCAGTAACTCCTC | AGCATATATGTT |
| | TAAAGCATACAA | | AACCAAGAGGGGCTGAG | TAAAGCATACAA |
| | CATGCTTTCAAG | | TTTCATATGTGGAATTA | CATGCTTTCAAG |
| | ACGTGGTGGTGG | | CCACTCGCATGTATTTT | ACGTGGTGGTGG |
| | ATGTGGATTGCG | | CAGTTGGTGATACGTTT | ATGTGGATTGCG |
| | CGGAGAATACTA | | AGCTTGGCAATGCATCT | CGGAGAATACTA |
| | AAGAGGATCAGT | | TCAGTATAAGATACATG | AAGAGGATCAGT |
| | TGGCCGAAATCA | | AAGCGCCATTTGATTTG | TGGCCGAAATCA |
| | GTTACCGTTTTC | | CTGTTAGAGTGGTTGTA | GTTACCGTTTTC |
| | AAGGTAAGAAGG | | TGTCCCCATCGATCCTA | AAGGTAAGAAGG |
| | AAGCGGACCAAC | | CATGTCAACCAATGCGG | AAGCGGACCAAC |
| | CGTGGATTGTTG | | TTATATTCTACGTGTTT | CGTGGATTGTTG |
| | TAAACACGAGCA | | GTATCATCCCAACGCAC | TAAACACGAGCA |
| | CACTGTTTGATG | | CCCAATGCCTCTCTCAT | CACTGTTTGATG |
| | AACTCGAATTAG | | ATGAATTCCGGTTGTAC | AACTCGAATTAG |
| | ACCCACCCGAGA | | ATTTACCTCGCCACATT | ACCCACCCGAGA |
| | TTGAACCGGGTG | | TAGCCCAGCGTGTTGCA | TTGAACCGGGTG |
| | TCTTGAAAGTAC | | AGCACAGTGTATCAGAA | TCTTGAAAGTAC |
| | TTCGGACAGAGA | | TTGTGAACATGCAGATA | TTCGGACAGAGA |
| | AACAATACTTGG | | ACTACACCGCATATTGT | AACAATACTTGG |
| | GTGTGTACATTT | | CTGGGAATATCTCATAT | GTGTGTACATTT |
| | GGAACATGCGCG | | GGAGCCTAGCTTTGGTC | GGAACATGCGCG |
| | GCTCCGATGGTA | | TAATCTTACACGACGGG | GCTCCGATGGTA |
| | CGTCTACCTACG | | GGCACCACGTTAAAGTT | CGTCTACCTACG |
| | CCACGTTTTTGG | | TGTAGATACACCCGAGA | CCACGTTTTTGG |
| | TCACCTGGAAAG | | GTTTGTCGGGATTATAC | TCACCTGGAAAG |
| | GGGATGAGAAGA | | GTTTTTGTGGTGTATTT | GGGATGAGAAGA |
| | CAAGAAACCCTA | | TAACGGGCATGTTGAAG | CAAGAAACCCTA |
| | CGCCCGCAGTAA | | CCGTAGCATACACTGTT | CGCCCGCAGTAA |
| | CTCCTCAACCAA | | GTATCCACAGTAGATCA | CTCCTCAACCAA |
| | GAGGGGCTGAGT | | TTTTGTAAACGCAATTG | GAGGGGCTGAGT |
| | TTCATATGTGGA | | AAGAGCGTGGATTTCCG | TTCATATGTGGA |
| | ATTACCACTCGC | | CCAACGGCCGGTCAGCC | ATTACCACTCGC |
| | ATGTATTTTCAG | | ACCGGCGACTACTAAAC | ATGTATTTTCAG |
| | TTGGTGATACGT | | CCAAGGAAATTACCCCC | TTGGTGATACGT |
| | TTAGCTTGGCAA | | GTAAACCCCGGAACGTC | TTAGCTTGGCAA |
| | TGCATCTTCAGT | | ACCACTTCTACGATATG | TGCATCTTCAGT |
| | ATAAGATACATG | | CCGCATGGACCGGAGGG | ATAAGATACATG |
| | AAGCGCCATTTG | | CTTGCAGCAGTAGTACT | AAGCGCCATTTG |
| | ATTTGCTGTTAG | | TTTATGTCTCGTAATAT | ATTTGCTGTTAG |
| | AGTGGTTGTATG | | TTTTAATCTGTACGGCT | AGTGGTTGTATG |
| | TCCCCATCGATC | | AAACGAATGAGGGTTAA | TCCCCATCGATC |
| | CTACATGTCAAC | | AGCCGCCAGGGTAGACA | CTACATGTCAAC |
| | CAATGCGGTTAT | | AG | CAATGCGGTTAT |
| | ATTCTACGTGTT | | | ATTCTACGTGTT |
| | TGTATCATCCCA | | | TGTATCATCCCA |
| | ACGCACCCCAAT | | | ACGCACCCCAAT |
| | GCCTCTCTCATA | | | GCCTCTCTCATA |
| | TGAATTCCGGTT | | | TGAATTCCGGTT |
| | GTACATTTACCT | | | GTACATTTACCT |
| | CGCCACATTTAG | | | CGCCACATTTAG |
| | CCCAGCGTGTTG | | | CCCAGCGTGTTG |
| | CAAGCACAGTGT | | | CAAGCACAGTGT |
| | ATCAGAATTGTG | | | ATCAGAATTGTG |
| | AACATGCAGATA | | | AACATGCAGATA |
| | ACTACACCGCAT | | | ACTACACCGCAT |
| | ATTGTCTGGGAA | | | ATTGTCTGGGAA |
| | TATCTCATATGG | | | TATCTCATATGG |
| | AGCCTAGCTTTG | | | AGCCTAGCTTTG |
| | GTCTAATCTTAC | | | GTCTAATCTTAC |
| | ACGACGGGGGCA | | | ACGACGGGGGCA |
| | CCACGTTAAAGT | | | CCACGTTAAAGT |
| | TTGTAGATACAC | | | TTGTAGATACAC |
| | CCGAGAGTTTGT | | | CCGAGAGTTTGT |
| | CGGGATTATACG | | | CGGGATTATACG |
| | TTTTTGTGGTGT | | | TTTTTGTGGTGT |
| | ATTTTAACGGGC | | | ATTTTAACGGGC |
| | ATGTTGAAGCCG | | | ATGTTGAAGCCG |
| | TAGCATACACTG | | | TAGCATACACTG |
| | TTGTATCCACAG | | | TTGTATCCACAG |
| | TAGATCATTTTG | | | TAGATCATTTTG |
| | TAAACGCAATTG | | | TAAACGCAATTG |
| | AAGAGCGTGGAT | | | AAGAGCGTGGAT |
| | TTCCGCCAACGG | | | TTCCGCCAACGG |
| | CCGGTCAGCCAC | | | CCGGTCAGCCAC |
| | CGGCGACTACTA | | | CGGCGACTACTA |
| | AACCCAAGGAAA | | | AACCCAAGGAAA |
| | TTACCCCCGTAA | | | TTACCCCCGTAA |
| | ACCCCGGAACGT | | | ACCCCGGAACGT |
| | CACCACTTCTAC | | | CACCACTTCTAC |
| | GATATGCCGCAT | | | GATATGCCGCAT |
| | GGACCGGAGGGC | | | GGACCGGAGGGC |
| | TTGCAGCAGTAG | | | TTGCAGCAGTAG |
| | TACTTTTATGTC | | | TACTTTTATGTC |
| | TCGTAATATTTT | | | TCGTAATATTTT |
| | TAATCTGTACGG | | | TAATCTGTACGG |
| | CTAAACGAATGA | | | CTAAACGAATGA |
| | GGGTTAAAGCCG | | | GGGTTAAAGCCG |
| | CCAGGGTAGACA | | | CCAGGGTAGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |
| | Sequence, NT | | | mRNA Sequence |
| | (5′ UTR, | | | (assumes |
| mRNA | ORF, 3′ UTR) | ORF Sequence, AA | ORF Sequence, NT | T100 tail) |
| Name(s) | SEQ ID NO: 80 | SEQ ID NO: 81 | SEQ ID NO: 82 | SEQ ID NO: 83 |
| VZV-GE- | GGGAAATAAGAG | MGTVNKPVVGVLMGFGI | ATGGGGACAGTTAATAA | GGGAAATAAGAG |
| truncated- | AGAAAAGAAGAG | ITGTLRITNPVRASVLR | ACCTGTGGTGGGGGTAT | AGAAAAGAAGAG |
| delete_ | TAAGAAGAAATA | YDDFHIDEDKLDTNSVY | TGATGGGGTTCGGAATT | TAAGAAGAAATA |
| from_ | TAAGAGCCACCA | EPYYHSDHAESSWVNRG | ATCACGGGAACGTTGCG | TAAGAGCCACCA |
| 574_-_ | TGGGGACAGTTA | ESSRKAYDHNSPYIWPR | TATAACGAATCCGGTCA | TGGGGACAGTTA |
| Y569A | ATAAACCTGTGG | NDYDGFLENAHEHHGVY | GAGCATCCGTCTTGCGA | ATAAACCTGTGG |
| Variant | TGGGGGTATTGA | NQGRGIDSGERLMQPTQ | TACGATGATTTTCACAT | TGGGGGTATTGA |
| 6 | TGGGGTTCGGAA | MSAQEDLGDDTGIHVIP | CGATGAAGACAAACTGG | TGGGGTTCGGAA |
| | TTATCACGGGAA | TLNGDDRHKIVNVDQRQ | ATACAAACTCCGTATAT | TTATCACGGGAA |
| | CGTTGCGTATAA | YGDVFKGDLNPKPQGQR | GAGCCTTACTACCATTC | CGTTGCGTATAA |
| | CGAATCCGGTCA | LIEVSVEENHPFTLRAP | AGATCATGCGGAGTCTT | CGAATCCGGTCA |
| | GAGCATCCGTCT | IQRIYGVRYTETWSFLP | CATGGGTAAATCGGGGA | GAGCATCCGTCT |
| | TGCGATACGATG | SLTCTGDAAPAIQHICL | GAGTCTTCGCGAAAAGC | TGCGATACGATG |
| | ATTTTCACATCG | KHTTCFQDVVVDVDCAE | GTACGATCATAACTCAC | ATTTTCACATCG |
| | ATGAAGACAAAC | NTKEDQLAEISYRFQGK | CTTATATATGGCCACGT | ATGAAGACAAAC |
| | TGGATACAAACT | KEADQPWIVVNTSTLFD | AATGATTATGATGGATT | TGGATACAAACT |
| | CCGTATATGAGC | ELELDPPEIEPGVLKVL | TTTAGAGAACGCACACG | CCGTATATGAGC |
| | CTTACTACCATT | RTEKQYLGVYIWNMRGS | AACACCATGGGGTGTAT | CTTACTACCATT |
| | CAGATCATGCGG | DGTSTYATFLVTWKGDE | AATCAGGGCCGTGGTAT | CAGATCATGCGG |
| | AGTCTTCATGGG | KTRNPTPAVTPQPRGAE | CGATAGCGGGGAACGGT | AGTCTTCATGGG |
| | TAAATCGGGGAG | FHMWNYHSHVFSVGDTF | TAATGCAACCCACACAA | TAAATCGGGGAG |
| | AGTCTTCGCGAA | SLAMHLQYKIHEAPFDL | ATGTCTGCACAGGAGGA | AGTCTTCGCGAA |
| | AAGCGTACGATC | LLEWLYVPIDPTCQPMR | TCTTGGGGACGATACGG | AAGCGTACGATC |
| | ATAACTCACCTT | LYSTCLYHPNAPQCLSH | GCATCCACGTTATCCCT | ATAACTCACCTT |
| | ATATATGGCCAC | MNSGCTFTSPHLAQRVA | ACGTTAAACGGCGATGA | ATATATGGCCAC |
| | GTAATGATTATG | STVYQNCEHADNYTAYC | CAGACATAAAATTGTAA | GTAATGATTATG |
| | ATGGATTTTTAG | LGISHMEPSFGLILHDG | ATGTGGACCAACGTCAA | ATGGATTTTTAG |
| | AGAACGCACACG | GTTLKFVDTPESLSGLY | TACGGTGACGTGTTTAA | AGAACGCACACG |
| | AACACCATGGGG | VFVVYFNGHVEAVAYTV | AGGAGATCTTAATCCAA | AACACCATGGGG |
| | TGTATAATCAGG | VSTVDHFVNAIEERGFP | AACCCCAAGGCCAAAGA | TGTATAATCAGG |
| | GCCGTGGTATCG | PTAGQPPATTKPKEITP | CTCATTGAGGTGTCAGT | GCCGTGGTATCG |
| | ATAGCGGGGAAC | VNPGTSPLLRYAAWTGG | GGAAGAAAATCACCCGT | ATAGCGGGGAAC |
| | GGTTAATGCAAC | LAAVVLLCLVIFLICTA | TTACTTTACGCGCACCG | GGTTAATGCAAC |
| | CCACACAAATGT | KRMRVKAARVDK | ATTCAGCGGATTTATGG | CCACACAAATGT |
| | CTGCACAGGAGG | | AGTCCGGTACACCGAGA | CTGCACAGGAGG |
| | ATCTTGGGGACG | | CTTGGAGCTTTTTGCCG | ATCTTGGGGACG |
| | ATACGGGCATCC | | TCATTAACCTGTACGGG | ATACGGGCATCC |
| | ACGTTATCCCTA | | AGACGCAGCGCCCGCCA | ACGTTATCCCTA |
| | CGTTAAACGGCG | | TCCAGCATATATGTTTA | CGTTAAACGGCG |
| | ATGACAGACATA | | AAGCATACAACATGCTT | ATGACAGACATA |
| | AAATTGTAAATG | | TCAAGACGTGGTGGTGG | AAATTGTAAATG |
| | TGGACCAACGTC | | ATGTGGATTGCGCGGAA | TGGACCAACGTC |
| | AATACGGTGACG | | AATACTAAAGAGGATCA | AATACGGTGACG |
| | TGTTTAAAGGAG | | GTTGGCCGAAATCAGTT | TGTTTAAAGGAG |
| | ATCTTAATCCAA | | ACCGTTTTCAAGGTAAG | ATCTTAATCCAA |
| | AACCCCAAGGCC | | AAGGAAGCGGACCAACC | AACCCCAAGGCC |
| | AAAGACTCATTG | | GTGGATTGTTGTAAACA | AAAGACTCATTG |
| | AGGTGTCAGTGG | | CGAGCACACTGTTTGAT | AGGTGTCAGTGG |
| | AAGAAAATCACC | | GAACTCGAATTAGACCC | AAGAAAATCACC |
| | CGTTTACTTTAC | | CCCCGAGATTGAACCGG | CGTTTACTTTAC |
| | GCGCACCGATTC | | GTGTCTTGAAAGTACTT | GCGCACCGATTC |
| | AGCGGATTTATG | | CGGACAGAGAAACAATA | AGCGGATTTATG |
| | GAGTCCGGTACA | | CTTGGGTGTGTACATTT | GAGTCCGGTACA |
| | CCGAGACTTGGA | | GGAACATGCGCGGCTCC | CCGAGACTTGGA |
| | GCTTTTTGCCGT | | GATGGTACGTCTACCTA | GCTTTTTGCCGT |
| | CATTAACCTGTA | | CGCCACGTTTTTGGTCA | CATTAACCTGTA |
| | CGGGAGACGCAG | | CCTGGAAAGGGGATGAG | CGGGAGACGCAG |
| | CGCCCGCCATCC | | AAGACAAGAAACCCTAC | CGCCCGCCATCC |
| | AGCATATATGTT | | GCCCGCAGTAACTCCTC | AGCATATATGTT |
| | TAAAGCATACAA | | AACCAAGAGGGGCTGAG | TAAAGCATACAA |
| | CATGCTTTCAAG | | TTTCATATGTGGAATTA | CATGCTTTCAAG |
| | ACGTGGTGGTGG | | CCACTCGCATGTATTTT | ACGTGGTGGTGG |
| | ATGTGGATTGCG | | CAGTTGGTGATACGTTT | ATGTGGATTGCG |
| | CGGAAAATACTA | | AGCTTGGCAATGCATCT | CGGAAAATACTA |
| | AAGAGGATCAGT | | TCAGTATAAGATACATG | AAGAGGATCAGT |
| | TGGCCGAAATCA | | AAGCGCCATTTGATTTG | TGGCCGAAATCA |
| | GTTACCGTTTTC | | CTGTTAGAGTGGTTGTA | GTTACCGTTTTC |
| | AAGGTAAGAAGG | | TGTCCCCATCGATCCTA | AAGGTAAGAAGG |
| | AAGCGGACCAAC | | CATGTCAACCAATGCGG | AAGCGGACCAAC |
| | CGTGGATTGTTG | | TTATATTCTACGTGTTT | CGTGGATTGTTG |
| | TAAACACGAGCA | | GTATCATCCCAACGCAC | TAAACACGAGCA |
| | CACTGTTTGATG | | CCCAATGCCTCTCTCAT | CACTGTTTGATG |
| | AACTCGAATTAG | | ATGAATTCCGGTTGTAC | AACTCGAATTAG |
| | ACCCCCCCGAGA | | ATTTACCTCGCCACATT | ACCCCCCCGAGA |
| | TTGAACCGGGTG | | TAGCCCAGCGTGTTGCA | TTGAACCGGGTG |
| | TCTTGAAAGTAC | | AGCACAGTGTATCAGAA | TCTTGAAAGTAC |
| | TTCGGACAGAGA | | TTGTGAACATGCAGATA | TTCGGACAGAGA |
| | AACAATACTTGG | | ACTACACCGCATATTGT | AACAATACTTGG |
| | GTGTGTACATTT | | CTGGGAATATCTCATAT | GTGTGTACATTT |
| | GGAACATGCGCG | | GGAGCCTAGCTTTGGTC | GGAACATGCGCG |
| | GCTCCGATGGTA | | TAATCTTACACGACGGG | GCTCCGATGGTA |
| | CGTCTACCTACG | | GGCACCACGTTAAAGTT | CGTCTACCTACG |
| | CCACGTTTTTGG | | TGTAGATACACCCGAGA | CCACGTTTTTGG |
| | TCACCTGGAAAG | | GTTTGTCGGGATTATAC | TCACCTGGAAAG |
| | GGGATGAGAAGA | | GTTTTTGTGGTGTATTT | GGGATGAGAAGA |
| | CAAGAAACCCTA | | TAACGGGCATGTTGAAG | CAAGAAACCCTA |
| | CGCCCGCAGTAA | | CCGTAGCATACACTGTT | CGCCCGCAGTAA |
| | CTCCTCAACCAA | | GTATCCACAGTAGATCA | CTCCTCAACCAA |
| | GAGGGGCTGAGT | | TTTTGTAAACGCAATTG | GAGGGGCTGAGT |
| | TTCATATGTGGA | | AAGAGCGTGGATTTCCG | TTCATATGTGGA |
| | ATTACCACTCGC | | CCAACGGCCGGTCAGCC | ATTACCACTCGC |
| | ATGTATTTTCAG | | ACCGGCGACTACTAAAC | ATGTATTTTCAG |
| | TTGGTGATACGT | | CCAAGGAAATTACCCCC | TTGGTGATACGT |
| | TTAGCTTGGCAA | | GTAAACCCCGGAACGTC | TTAGCTTGGCAA |
| | TGCATCTTCAGT | | ACCACTTCTACGATATG | TGCATCTTCAGT |
| | ATAAGATACATG | | CCGCATGGACCGGAGGG | ATAAGATACATG |
| | AAGCGCCATTTG | | CTTGCAGCAGTAGTACT | AAGCGCCATTTG |
| | ATTTGCTGTTAG | | TTTATGTCTCGTAATAT | ATTTGCTGTTAG |
| | AGTGGTTGTATG | | TTTTAATCTGTACGGCT | AGTGGTTGTATG |
| | TCCCCATCGATC | | AAACGAATGAGGGTTAA | TCCCCATCGATC |
| | CTACATGTCAAC | | AGCCGCCAGGGTAGACA | CTACATGTCAAC |
| | CAATGCGGTTAT | | AG | CAATGCGGTTAT |
| | ATTCTACGTGTT | | | ATTCTACGTGTT |
| | TGTATCATCCCA | | | TGTATCATCCCA |
| | ACGCACCCCAAT | | | ACGCACCCCAAT |
| | GCCTCTCTCATA | | | GCCTCTCTCATA |
| | TGAATTCCGGTT | | | TGAATTCCGGTT |
| | GTACATTTACCT | | | GTACATTTACCT |
| | CGCCACATTTAG | | | CGCCACATTTAG |
| | CCCAGCGTGTTG | | | CCCAGCGTGTTG |
| | CAAGCACAGTGT | | | CAAGCACAGTGT |
| | ATCAGAATTGTG | | | ATCAGAATTGTG |
| | AACATGCAGATA | | | AACATGCAGATA |
| | ACTACACCGCAT | | | ACTACACCGCAT |
| | ATTGTCTGGGAA | | | ATTGTCTGGGAA |
| | TATCTCATATGG | | | TATCTCATATGG |
| | AGCCTAGCTTTG | | | AGCCTAGCTTTG |
| | GTCTAATCTTAC | | | GTCTAATCTTAC |
| | ACGACGGGGGCA | | | ACGACGGGGGCA |
| | CCACGTTAAAGT | | | CCACGTTAAAGT |
| | TTGTAGATACAC | | | TTGTAGATACAC |
| | CCGAGAGTTTGT | | | CCGAGAGTTTGT |
| | CGGGATTATACG | | | CGGGATTATACG |
| | TTTTTGTGGTGT | | | TTTTTGTGGTGT |
| | ATTTTAACGGGC | | | ATTTTAACGGGC |
| | ATGTTGAAGCCG | | | ATGTTGAAGCCG |
| | TAGCATACACTG | | | TAGCATACACTG |
| | TTGTATCCACAG | | | TTGTATCCACAG |
| | TAGATCATTTTG | | | TAGATCATTTTG |
| | TAAACGCAATTG | | | TAAACGCAATTG |
| | AAGAGCGTGGAT | | | AAGAGCGTGGAT |
| | TTCCGCCAACGG | | | TTCCGCCAACGG |
| | CCGGTCAGCCAC | | | CCGGTCAGCCAC |
| | CGGCGACTACTA | | | CGGCGACTACTA |
| | AACCCAAGGAAA | | | AACCCAAGGAAA |
| | TTACCCCCGTAA | | | TTACCCCCGTAA |
| | ACCCCGGAACGT | | | ACCCCGGAACGT |
| | CACCACTTCTAC | | | CACCACTTCTAC |
| | GATATGCCGCAT | | | GATATGCCGCAT |
| | GGACCGGAGGGC | | | GGACCGGAGGGC |
| | TTGCAGCAGTAG | | | TTGCAGCAGTAG |
| | TACTTTTATGTC | | | TACTTTTATGTC |
| | TCGTAATATTTT | | | TCGTAATATTTT |
| | TAATCTGTACGG | | | TAATCTGTACGG |
| | CTAAACGAATGA | | | CTAAACGAATGA |
| | GGGTTAAAGCCG | | | GGGTTAAAGCCG |
| | CCAGGGTAGACA | | | CCAGGGTAGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |
| | Sequence, NT | | | mRNA Sequence |
| | (5′ UTR, | | | (assumes |
| mRNA | ORF, 3′ UTR) | ORF Sequence, AA | ORF Sequence, NT | T100 tail) |
| Name(s) | SEQ ID NO: 84 | SEQ ID NO: 85 | SEQ ID NO: 86 | SEQ ID NO: 87 |
| VZV-GE- | GGGAAATAAGAG | MGTVNKPVVGVLMGFGI | ATGGGGACAGTTAATAA | GGGAAATAAGAG |
| truncated- | AGAAAAGAAGAG | ITGTLRITNPVRASVLR | ACCTGTGGTGGGGGTAT | AGAAAAGAAGAG |
| delete_ | TAAGAAGAAATA | YDDFHIDEDKLDTNSVY | TGATGGGGTTCGGAATT | TAAGAAGAAATA |
| from_ | TAAGAGCCACCA | EPYYHSDHAESSWVNRG | ATCACGGGAACGTTGCG | TAAGAGCCACCA |
| 574_-_ | TGGGGACAGTTA | ESSRKAYDHNSPYIWPR | TATAACGAATCCGGTCA | TGGGGACAGTTA |
| Y569A | ATAAACCTGTGG | NDYDGFLENAHEHHGVY | GAGCATCCGTCTTGCGA | ATAAACCTGTGG |
| Variant | TGGGGGTATTGA | NQGRGIDSGERLMQPTQ | TACGATGATTTTCACAT | TGGGGGTATTGA |
| 7 | TGGGGTTCGGAA | MSAQEDLGDDTGIHVIP | CGATGAAGACAAACTGG | TGGGGTTCGGAA |
| | TTATCACGGGAA | TLNGDDRHKIVNVDQRQ | ATACAAACTCCGTATAT | TTATCACGGGAA |
| | CGTTGCGTATAA | YGDVFKGDLNPKPQGQR | GAGCCTTACTACCATTC | CGTTGCGTATAA |
| | CGAATCCGGTCA | LIEVSVEENHPFTLRAP | AGATCATGCGGAGTCTT | CGAATCCGGTCA |
| | GAGCATCCGTCT | IQRIYGVRYTETWSFLP | CATGGGTAAATCGGGGA | GAGCATCCGTCT |
| | TGCGATACGATG | SLTCTGDAAPAIQHICL | GAGTCTTCGCGAAAAGC | TGCGATACGATG |
| | ATTTTCACATCG | KHTTCFQDVVVDVDCAE | GTACGATCATAACTCAC | ATTTTCACATCG |
| | ATGAAGACAAAC | NTKEDQLAEISYRFQGK | CTTATATATGGCCACGT | ATGAAGACAAAC |
| | TGGATACAAACT | KEADQPWIVVNTSTLFD | AATGATTATGATGGATT | TGGATACAAACT |
| | CCGTATATGAGC | ELELDPPEIEPGVLKVL | TTTAGAGAACGCACACG | CCGTATATGAGC |
| | CTTACTACCATT | RTEKQYLGVYIWNMRGS | AACACCATGGGGTGTAT | CTTACTACCATT |
| | CAGATCATGCGG | DGTSTYATFLVTWKGDE | AATCAGGGCCGTGGTAT | CAGATCATGCGG |
| | AGTCTTCATGGG | KTRNPTPAVTPQPRGAE | CGATAGCGGGGAACGGT | AGTCTTCATGGG |
| | TAAATCGGGGAG | FHMWNYHSHVFSVGDTF | TAATGCAACCCACACAA | TAAATCGGGGAG |
| | AGTCTTCGCGAA | SLAMHLQYKIHEAPFDL | ATGTCTGCACAGGAGGA | AGTCTTCGCGAA |
| | AAGCGTACGATC | LLEWLYVPIDPTCQPMR | TCTTGGGGACGATACGG | AAGCGTACGATC |
| | ATAACTCACCTT | LYSTCLYHPNAPQCLSH | GCATCCACGTTATCCCT | ATAACTCACCTT |
| | ATATATGGCCAC | MNSGCTFTSPHLAQRVA | ACGTTAAACGGCGATGA | ATATATGGCCAC |
| | GTAATGATTATG | STVYQNCEHADNYTAYC | CAGACATAAAATTGTAA | GTAATGATTATG |
| | ATGGATTTTTAG | LGISHMEPSFGLILHDG | ATGTGGACCAACGTCAA | ATGGATTTTTAG |
| | AGAACGCACACG | GTTLKFVDTPESLSGLY | TACGGTGACGTGTTTAA | AGAACGCACACG |
| | AACACCATGGGG | VFVVYFNGHVEAVAYTV | AGGAGATCTTAATCCAA | AACACCATGGGG |
| | TGTATAATCAGG | VSTVDHEVNAIEERGFP | AACCCCAAGGCCAAAGA | TGTATAATCAGG |
| | GCCGTGGTATCG | PTAGQPPATTKPKEITP | CTCATTGAGGTGTCAGT | GCCGTGGTATCG |
| | ATAGCGGGGAAC | VNPGTSPLLRYAAWTGG | GGAAGAAAATCACCCGT | ATAGCGGGGAAC |
| | GGTTAATGCAAC | LAAVVLLCLVIFLICTA | TTACTTTACGCGCACCG | GGTTAATGCAAC |
| | CCACACAAATGT | KRMRVKAARVDK | ATTCAGCGGATTTATGG | CCACACAAATGT |
| | CTGCACAGGAGG | | AGTCCGGTACACCGAGA | CTGCACAGGAGG |
| | ATCTTGGGGACG | | CTTGGAGCTTTTTGCCG | ATCTTGGGGACG |
| | ATACGGGCATCC | | TCATTAACCTGTACGGG | ATACGGGCATCC |
| | ACGTTATCCCTA | | AGACGCAGCGCCCGCCA | ACGTTATCCCTA |
| | CGTTAAACGGCG | | TCCAGCATATATGTTTA | CGTTAAACGGCG |
| | ATGACAGACATA | | AAGCATACAACATGCTT | ATGACAGACATA |
| | AAATTGTAAATG | | TCAAGACGTGGTGGTGG | AAATTGTAAATG |
| | TGGACCAACGTC | | ATGTGGATTGCGCGGAA | TGGACCAACGTC |
| | AATACGGTGACG | | AATACTAAAGAGGATCA | AATACGGTGACG |
| | TGTTTAAAGGAG | | GTTGGCCGAAATCAGTT | TGTTTAAAGGAG |
| | ATCTTAATCCAA | | ACCGTTTTCAAGGTAAG | ATCTTAATCCAA |
| | AACCCCAAGGCC | | AAGGAAGCGGACCAACC | AACCCCAAGGCC |
| | AAAGACTCATTG | | GTGGATTGTTGTAAACA | AAAGACTCATTG |
| | AGGTGTCAGTGG | | CGAGCACACTGTTTGAT | AGGTGTCAGTGG |
| | AAGAAAATCACC | | GAACTCGAATTAGACCC | AAGAAAATCACC |
| | CGTTTACTTTAC | | ACCCGAGATTGAACCGG | CGTTTACTTTAC |
| | GCGCACCGATTC | | GTGTCTTGAAAGTACTT | GCGCACCGATTC |
| | AGCGGATTTATG | | CGGACAGAGAAACAATA | AGCGGATTTATG |
| | GAGTCCGGTACA | | CTTGGGTGTGTACATTT | GAGTCCGGTACA |
| | CCGAGACTTGGA | | GGAACATGCGCGGCTCC | CCGAGACTTGGA |
| | GCTTTTTGCCGT | | GATGGTACGTCTACCTA | GCTTTTTGCCGT |
| | CATTAACCTGTA | | CGCCACGTTTTTGGTCA | CATTAACCTGTA |
| | CGGGAGACGCAG | | CCTGGAAAGGGGATGAG | CGGGAGACGCAG |
| | CGCCCGCCATCC | | AAGACAAGAAACCCTAC | CGCCCGCCATCC |
| | AGCATATATGTT | | GCCCGCAGTAACTCCTC | AGCATATATGTT |
| | TAAAGCATACAA | | AACCAAGAGGGGCTGAG | TAAAGCATACAA |
| | CATGCTTTCAAG | | TTTCATATGTGGAATTA | CATGCTTTCAAG |
| | ACGTGGTGGTGG | | CCACTCGCATGTATTTT | ACGTGGTGGTGG |
| | ATGTGGATTGCG | | CAGTTGGTGATACGTTT | ATGTGGATTGCG |
| | CGGAAAATACTA | | AGCTTGGCAATGCATCT | CGGAAAATACTA |
| | AAGAGGATCAGT | | TCAGTATAAGATACATG | AAGAGGATCAGT |
| | TGGCCGAAATCA | | AAGCGCCATTTGATTTG | TGGCCGAAATCA |
| | GTTACCGTTTTC | | CTGTTAGAGTGGTTGTA | GTTACCGTTTTC |
| | AAGGTAAGAAGG | | TGTCCCCATCGATCCTA | AAGGTAAGAAGG |
| | AAGCGGACCAAC | | CATGTCAACCAATGCGG | AAGCGGACCAAC |
| | CGTGGATTGTTG | | TTATATTCTACGTGTTT | CGTGGATTGTTG |
| | TAAACACGAGCA | | GTATCATCCCAACGCAC | TAAACACGAGCA |
| | CACTGTTTGATG | | CCCAATGCCTCTCTCAT | CACTGTTTGATG |
| | AACTCGAATTAG | | ATGAATTCCGGTTGTAC | AACTCGAATTAG |
| | ACCCACCCGAGA | | ATTTACCTCGCCACATT | ACCCACCCGAGA |
| | TTGAACCGGGTG | | TAGCCCAGCGTGTTGCA | TTGAACCGGGTG |
| | TCTTGAAAGTAC | | AGCACAGTGTATCAGAA | TCTTGAAAGTAC |
| | TTCGGACAGAGA | | TTGTGAACATGCAGATA | TTCGGACAGAGA |
| | AACAATACTTGG | | ACTACACCGCATATTGT | AACAATACTTGG |
| | GTGTGTACATTT | | CTGGGAATATCTCATAT | GTGTGTACATTT |
| | GGAACATGCGCG | | GGAGCCTAGCTTTGGTC | GGAACATGCGCG |
| | GCTCCGATGGTA | | TAATCTTACACGACGGG | GCTCCGATGGTA |
| | CGTCTACCTACG | | GGCACCACGTTAAAGTT | CGTCTACCTACG |
| | CCACGTTTTTGG | | TGTAGATACACCCGAGA | CCACGTTTTTGG |
| | TCACCTGGAAAG | | GTTTGTCGGGATTATAC | TCACCTGGAAAG |
| | GGGATGAGAAGA | | GTTTTTGTGGTGTATTT | GGGATGAGAAGA |
| | CAAGAAACCCTA | | TAACGGGCATGTTGAAG | CAAGAAACCCTA |
| | CGCCCGCAGTAA | | CCGTAGCATACACTGTT | CGCCCGCAGTAA |
| | CTCCTCAACCAA | | GTATCCACAGTAGATCA | CTCCTCAACCAA |
| | GAGGGGCTGAGT | | TTTTGTAAACGCAATTG | GAGGGGCTGAGT |
| | TTCATATGTGGA | | AAGAGCGTGGATTTCCG | TTCATATGTGGA |
| | ATTACCACTCGC | | CCAACGGCCGGTCAGCC | ATTACCACTCGC |
| | ATGTATTTTCAG | | ACCGGCGACTACTAAAC | ATGTATTTTCAG |
| | TTGGTGATACGT | | CCAAGGAAATTACCCCC | TTGGTGATACGT |
| | TTAGCTTGGCAA | | GTAAACCCCGGAACGTC | TTAGCTTGGCAA |
| | TGCATCTTCAGT | | ACCACTTCTACGATATG | TGCATCTTCAGT |
| | ATAAGATACATG | | CCGCATGGACCGGAGGG | ATAAGATACATG |
| | AAGCGCCATTTG | | CTTGCAGCAGTAGTACT | AAGCGCCATTTG |
| | ATTTGCTGTTAG | | TTTATGTCTCGTAATAT | ATTTGCTGTTAG |
| | AGTGGTTGTATG | | TTTTAATCTGTACGGCT | AGTGGTTGTATG |
| | TCCCCATCGATC | | AAACGAATGAGGGTTAA | TCCCCATCGATC |
| | CTACATGTCAAC | | AGCCGCCAGGGTAGACA | CTACATGTCAAC |
| | CAATGCGGTTAT | | AG | CAATGCGGTTAT |
| | ATTCTACGTGTT | | | ATTCTACGTGTT |
| | TGTATCATCCCA | | | TGTATCATCCCA |
| | ACGCACCCCAAT | | | ACGCACCCCAAT |
| | GCCTCTCTCATA | | | GCCTCTCTCATA |
| | TGAATTCCGGTT | | | TGAATTCCGGTT |
| | GTACATTTACCT | | | GTACATTTACCT |
| | CGCCACATTTAG | | | CGCCACATTTAG |
| | CCCAGCGTGTTG | | | CCCAGCGTGTTG |
| | CAAGCACAGTGT | | | CAAGCACAGTGT |
| | ATCAGAATTGTG | | | ATCAGAATTGTG |
| | AACATGCAGATA | | | AACATGCAGATA |
| | ACTACACCGCAT | | | ACTACACCGCAT |
| | ATTGTCTGGGAA | | | ATTGTCTGGGAA |
| | TATCTCATATGG | | | TATCTCATATGG |
| | AGCCTAGCTTTG | | | AGCCTAGCTTTG |
| | GTCTAATCTTAC | | | GTCTAATCTTAC |
| | ACGACGGGGGCA | | | ACGACGGGGGCA |
| | CCACGTTAAAGT | | | CCACGTTAAAGT |
| | TTGTAGATACAC | | | TTGTAGATACAC |
| | CCGAGAGTTTGT | | | CCGAGAGTTTGT |
| | CGGGATTATACG | | | CGGGATTATACG |
| | TTTTTGTGGTGT | | | TTTTTGTGGTGT |
| | ATTTTAACGGGC | | | ATTTTAACGGGC |
| | ATGTTGAAGCCG | | | ATGTTGAAGCCG |
| | TAGCATACACTG | | | TAGCATACACTG |
| | TTGTATCCACAG | | | TTGTATCCACAG |
| | TAGATCATTTTG | | | TAGATCATTTTG |
| | TAAACGCAATTG | | | TAAACGCAATTG |
| | AAGAGCGTGGAT | | | AAGAGCGTGGAT |
| | TTCCGCCAACGG | | | TTCCGCCAACGG |
| | CCGGTCAGCCAC | | | CCGGTCAGCCAC |
| | CGGCGACTACTA | | | CGGCGACTACTA |
| | AACCCAAGGAAA | | | AACCCAAGGAAA |
| | TTACCCCCGTAA | | | TTACCCCCGTAA |
| | ACCCCGGAACGT | | | ACCCCGGAACGT |
| | CACCACTTCTAC | | | CACCACTTCTAC |
| | GATATGCCGCAT | | | GATATGCCGCAT |
| | GGACCGGAGGGC | | | GGACCGGAGGGC |
| | TTGCAGCAGTAG | | | TTGCAGCAGTAG |
| | TACTTTTATGTC | | | TACTTTTATGTC |
| | TCGTAATATTTT | | | TCGTAATATTTT |
| | TAATCTGTACGG | | | TAATCTGTACGG |
| | CTAAACGAATGA | | | CTAAACGAATGA |
| | GGGTTAAAGCCG | | | GGGTTAAAGCCG |
| | CCAGGGTAGACA | | | CCAGGGTAGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |
| | Sequence, NT | | | mRNA Sequence |
| | (5′ UTR, | | | (assumes |
| mRNA | ORF, 3′ UTR) | ORF Sequence, AA | ORF Sequence, NT | T100 tail) |
| Name(s) | SEQ ID NO: 88 | SEQ ID NO: 89 | SEQ ID NO: 90 | SEQ ID NO: 91 |
| VZV-GE- | GGGAAATAAGAG | MGTVNKPVVGVLMGFGI | ATGGGGACAGTTAATAA | GGGAAATAAGAG |
| truncated- | AGAAAAGAAGAG | ITGTLRITNPVRASVLR | ACCTGTGGTGGGCGTAT | AGAAAAGAAGAG |
| delete_ | TAAGAAGAAATA | YDDFHIDEDKLDTNSVY | TGATGGGGTTCGGAATT | TAAGAAGAAATA |
| from_ | TAAGAGCCACCA | EPYYHSDHAESSWVNRG | ATCACGGGAACGTTGCG | TAAGAGCCACCA |
| 574_-_ | TGGGGACAGTTA | ESSRKAYDHNSPYIWPR | TATAACGAATCCGGTCA | TGGGGACAGTTA |
| Y569A | ATAAACCTGTGG | NDYDGFLENAHEHHGVY | GAGCATCCGTCTTGCGA | ATAAACCTGTGG |
| Variant | TGGGCGTATTGA | NQGRGIDSGERLMQPTQ | TACGATGATTTTCACAT | TGGGCGTATTGA |
| 8 | TGGGGTTCGGAA | MSAQEDLGDDTGIHVIP | CGATGAAGACAAACTGG | TGGGGTTCGGAA |
| | TTATCACGGGAA | TLNGDDRHKIVNVDQRQ | ATACAAACTCCGTATAT | TTATCACGGGAA |
| | CGTTGCGTATAA | YGDVFKGDLNPKPQGQR | GAGCCTTACTACCATTC | CGTTGCGTATAA |
| | CGAATCCGGTCA | LIEVSVEENHPFTLRAP | AGATCATGCGGAGTCTT | CGAATCCGGTCA |
| | GAGCATCCGTCT | IQRIYGVRYTETWSFLP | CATGGGTAAATCGGGGA | GAGCATCCGTCT |
| | TGCGATACGATG | SLTCTGDAAPAIQHICL | GAGTCTTCGCGAAAGGC | TGCGATACGATG |
| | ATTTTCACATCG | KHTTCFQDVVVDVDCAE | GTACGATCATAACTCAC | ATTTTCACATCG |
| | ATGAAGACAAAC | NTKEDQLAEISYRFQGK | CTTATATATGGCCACGT | ATGAAGACAAAC |
| | TGGATACAAACT | KEADQPWIVVNTSTLFD | AATGATTATGATGGATT | TGGATACAAACT |
| | CCGTATATGAGC | ELELDPPEIEPGVLKVL | CTTAGAGAACGCACACG | CCGTATATGAGC |
| | CTTACTACCATT | RTEKQYLGVYIWNMRGS | AACACCATGGGGTGTAT | CTTACTACCATT |
| | CAGATCATGCGG | DGTSTYATFLVTWKGDE | AATCAGGGCCGTGGTAT | CAGATCATGCGG |
| | AGTCTTCATGGG | KTRNPTPAVTPQPRGAE | CGATAGCGGGGAACGGT | AGTCTTCATGGG |
| | TAAATCGGGGAG | FHMWNYHSHVFSVGDTF | TAATGCAACCCACACAA | TAAATCGGGGAG |
| | AGTCTTCGCGAA | SLAMHLQYKIHEAPFDL | ATGTCTGCACAGGAGGA | AGTCTTCGCGAA |
| | AGGCGTACGATC | LLEWLYVPIDPTCQPMR | TCTTGGGGACGATACGG | AGGCGTACGATC |
| | ATAACTCACCTT | LYSTCLYHPNAPQCLSH | GCATCCACGTTATCCCT | ATAACTCACCTT |
| | ATATATGGCCAC | MNSGCTFTSPHLAQRVA | ACGTTAAACGGCGATGA | ATATATGGCCAC |
| | GTAATGATTATG | STVYQNCEHADNYTAYC | CAGACATAAGATTGTAA | GTAATGATTATG |
| | ATGGATTCTTAG | LGISHMEPSFGLILHDG | ATGTGGACCAACGTCAA | ATGGATTCTTAG |
| | AGAACGCACACG | GTTLKFVDTPESLSGLY | TACGGTGACGTGTTTAA | AGAACGCACACG |
| | AACACCATGGGG | VFVVYFNGHVEAVAYTV | AGGAGATCTTAATCCAA | AACACCATGGGG |
| | TGTATAATCAGG | VSTVDHFVNAIEERGFP | AGCCCCAAGGCCAAAGA | TGTATAATCAGG |
| | GCCGTGGTATCG | PTAGQPPATTKPKEITP | CTCATTGAGGTGTCAGT | GCCGTGGTATCG |
| | ATAGCGGGGAAC | VNPGTSPLLRYAAWTGG | GGAAGAGAATCACCCGT | ATAGCGGGGAAC |
| | GGTTAATGCAAC | LAAVVLLCLVIFLICTA | TTACTTTACGCGCACCG | GGTTAATGCAAC |
| | CCACACAAATGT | KRMRVKAARVDK | ATTCAGCGGATTTATGG | CCACACAAATGT |
| | CTGCACAGGAGG | | AGTCCGGTACACCGAGA | CTGCACAGGAGG |
| | ATCTTGGGGACG | | CTTGGAGCTTCTTGCCG | ATCTTGGGGACG |
| | ATACGGGCATCC | | TCATTAACCTGTACGGG | ATACGGGCATCC |
| | ACGTTATCCCTA | | AGACGCAGCGCCCGCCA | ACGTTATCCCTA |
| | CGTTAAACGGCG | | TCCAGCATATATGTTTA | CGTTAAACGGCG |
| | ATGACAGACATA | | AAGCATACAACATGCTT | ATGACAGACATA |
| | AGATTGTAAATG | | TCAAGACGTGGTGGTGG | AGATTGTAAATG |
| | TGGACCAACGTC | | ATGTGGATTGCGCGGAG | TGGACCAACGTC |
| | AATACGGTGACG | | AATACTAAAGAGGATCA | AATACGGTGACG |
| | TGTTTAAAGGAG | | GTTGGCCGAAATCAGTT | TGTTTAAAGGAG |
| | ATCTTAATCCAA | | ACCGTTTTCAAGGTAAG | ATCTTAATCCAA |
| | AGCCCCAAGGCC | | AAGGAAGCGGACCAACC | AGCCCCAAGGCC |
| | AAAGACTCATTG | | GTGGATTGTTGTAAACA | AAAGACTCATTG |
| | AGGTGTCAGTGG | | CGAGCACACTGTTTGAT | AGGTGTCAGTGG |
| | AAGAGAATCACC | | GAACTCGAATTAGACCC | AAGAGAATCACC |
| | CGTTTACTTTAC | | ACCCGAGATTGAACCGG | CGTTTACTTTAC |
| | GCGCACCGATTC | | GTGTCTTGAAAGTACTT | GCGCACCGATTC |
| | AGCGGATTTATG | | CGGACAGAGAAACAATA | AGCGGATTTATG |
| | GAGTCCGGTACA | | CTTGGGTGTGTACATTT | GAGTCCGGTACA |
| | CCGAGACTTGGA | | GGAACATGCGCGGCTCC | CCGAGACTTGGA |
| | GCTTCTTGCCGT | | GATGGTACGTCTACCTA | GCTTCTTGCCGT |
| | CATTAACCTGTA | | CGCCACGTTCTTGGTCA | CATTAACCTGTA |
| | CGGGAGACGCAG | | CCTGGAAAGGGGATGAG | CGGGAGACGCAG |
| | CGCCCGCCATCC | | AAGACAAGAAACCCTAC | CGCCCGCCATCC |
| | AGCATATATGTT | | GCCCGCAGTAACTCCTC | AGCATATATGTT |
| | TAAAGCATACAA | | AACCAAGAGGGGCTGAG | TAAAGCATACAA |
| | CATGCTTTCAAG | | TTTCATATGTGGAATTA | CATGCTTTCAAG |
| | ACGTGGTGGTGG | | CCACTCGCATGTATTTT | ACGTGGTGGTGG |
| | ATGTGGATTGCG | | CAGTTGGTGATACGTTT | ATGTGGATTGCG |
| | CGGAGAATACTA | | AGCTTGGCAATGCATCT | CGGAGAATACTA |
| | AAGAGGATCAGT | | TCAGTATAAGATACATG | AAGAGGATCAGT |
| | TGGCCGAAATCA | | AAGCGCCATTTGATTTG | TGGCCGAAATCA |
| | GTTACCGTTTTC | | CTGTTAGAGTGGTTGTA | GTTACCGTTTTC |
| | AAGGTAAGAAGG | | TGTCCCCATCGATCCTA | AAGGTAAGAAGG |
| | AAGCGGACCAAC | | CATGTCAACCAATGCGG | AAGCGGACCAAC |
| | CGTGGATTGTTG | | TTATATTCTACGTGTTT | CGTGGATTGTTG |
| | TAAACACGAGCA | | GTATCATCCCAACGCAC | TAAACACGAGCA |
| | CACTGTTTGATG | | CCCAATGCCTCTCTCAT | CACTGTTTGATG |
| | AACTCGAATTAG | | ATGAATTCCGGTTGTAC | AACTCGAATTAG |
| | ACCCACCCGAGA | | ATTTACCTCGCCACATT | ACCCACCCGAGA |
| | TTGAACCGGGTG | | TAGCCCAGCGTGTTGCA | TTGAACCGGGTG |
| | TCTTGAAAGTAC | | AGCACAGTGTATCAGAA | TCTTGAAAGTAC |
| | TTCGGACAGAGA | | TTGTGAACATGCAGATA | TTCGGACAGAGA |
| | AACAATACTTGG | | ACTACACCGCATATTGT | AACAATACTTGG |
| | GTGTGTACATTT | | CTGGGAATATCTCATAT | GTGTGTACATTT |
| | GGAACATGCGCG | | GGAGCCTAGCTTTGGTC | GGAACATGCGCG |
| | GCTCCGATGGTA | | TAATCTTACACGACGGA | GCTCCGATGGTA |
| | CGTCTACCTACG | | GGCACCACGTTAAAGTT | CGTCTACCTACG |
| | CCACGTTCTTGG | | TGTAGATACACCCGAGA | CCACGTTCTTGG |
| | TCACCTGGAAAG | | GTTTGTCGGGATTATAC | TCACCTGGAAAG |
| | GGGATGAGAAGA | | GTCTTTGTGGTGTATTT | GGGATGAGAAGA |
| | CAAGAAACCCTA | | TAACGGGCATGTTGAAG | CAAGAAACCCTA |
| | CGCCCGCAGTAA | | CCGTAGCATACACTGTT | CGCCCGCAGTAA |
| | CTCCTCAACCAA | | GTATCCACAGTAGATCA | CTCCTCAACCAA |
| | GAGGGGCTGAGT | | TTTTGTAAACGCAATTG | GAGGGGCTGAGT |
| | TTCATATGTGGA | | AAGAGCGTGGATTTCCG | TTCATATGTGGA |
| | ATTACCACTCGC | | CCAACGGCCGGTCAGCC | ATTACCACTCGC |
| | ATGTATTTTCAG | | ACCGGCGACTACTAAAC | ATGTATTTTCAG |
| | TTGGTGATACGT | | CCAAGGAAATTACGCCC | TTGGTGATACGT |
| | TTAGCTTGGCAA | | GTAAACCCCGGAACGTC | TTAGCTTGGCAA |
| | TGCATCTTCAGT | | ACCACTTCTACGATATG | TGCATCTTCAGT |
| | ATAAGATACATG | | CCGCATGGACCGGAGGG | ATAAGATACATG |
| | AAGCGCCATTTG | | CTTGCAGCAGTAGTACT | AAGCGCCATTTG |
| | ATTTGCTGTTAG | | TTTATGTCTCGTAATAT | ATTTGCTGTTAG |
| | AGTGGTTGTATG | | TCTTAATCTGTACGGCT | AGTGGTTGTATG |
| | TCCCCATCGATC | | AAACGAATGAGGGTTAA | TCCCCATCGATC |
| | CTACATGTCAAC | | AGCCGCCAGGGTAGACA | CTACATGTCAAC |
| | CAATGCGGTTAT | | AG | CAATGCGGTTAT |
| | ATTCTACGTGTT | | | ATTCTACGTGTT |
| | ACGCACCCCAAT | | | TGTATCATCCCA |
| | TGTATCATCCCA | | | ACGCACCCCAAT |
| | GCCTCTCTCATA | | | GCCTCTCTCATA |
| | TGAATTCCGGTT | | | TGAATTCCGGTT |
| | GTACATTTACCT | | | GTACATTTACCT |
| | CGCCACATTTAG | | | CGCCACATTTAG |
| | CCCAGCGTGTTG | | | CCCAGCGTGTTG |
| | CAAGCACAGTGT | | | CAAGCACAGTGT |
| | ATCAGAATTGTG | | | ATCAGAATTGTG |
| | AACATGCAGATA | | | AACATGCAGATA |
| | ACTACACCGCAT | | | ACTACACCGCAT |
| | ATTGTCTGGGAA | | | ATTGTCTGGGAA |
| | TATCTCATATGG | | | TATCTCATATGG |
| | AGCCTAGCTTTG | | | AGCCTAGCTTTG |
| | GTCTAATCTTAC | | | GTCTAATCTTAC |
| | ACGACGGAGGCA | | | ACGACGGAGGCA |
| | CCACGTTAAAGT | | | CCACGTTAAAGT |
| | TTGTAGATACAC | | | TTGTAGATACAC |
| | CCGAGAGTTTGT | | | CCGAGAGTTTGT |
| | CGGGATTATACG | | | CGGGATTATACG |
| | TCTTTGTGGTGT | | | TCTTTGTGGTGT |
| | ATTTTAACGGGC | | | ATTTTAACGGGC |
| | ATGTTGAAGCCG | | | ATGTTGAAGCCG |
| | TAGCATACACTG | | | TAGCATACACTG |
| | TTGTATCCACAG | | | TTGTATCCACAG |
| | TAGATCATTTTG | | | TAGATCATTTTG |
| | TAAACGCAATTG | | | TAAACGCAATTG |
| | AAGAGCGTGGAT | | | AAGAGCGTGGAT |
| | TTCCGCCAACGG | | | TTCCGCCAACGG |
| | CCGGTCAGCCAC | | | CCGGTCAGCCAC |
| | CGGCGACTACTA | | | CGGCGACTACTA |
| | AACCCAAGGAAA | | | AACCCAAGGAAA |
| | TTACGCCCGTAA | | | TTACGCCCGTAA |
| | ACCCCGGAACGT | | | ACCCCGGAACGT |
| | CACCACTTCTAC | | | CACCACTTCTAC |
| | GATATGCCGCAT | | | GATATGCCGCAT |
| | GGACCGGAGGGC | | | GGACCGGAGGGC |
| | TTGCAGCAGTAG | | | TTGCAGCAGTAG |
| | TACTTTTATGTC | | | TACTTTTATGTC |
| | TCGTAATATTCT | | | TCGTAATATTCT |
| | TAATCTGTACGG | | | TAATCTGTACGG |
| | CTAAACGAATGA | | | CTAAACGAATGA |
| | GGGTTAAAGCCG | | | GGGTTAAAGCCG |
| | CCAGGGTAGACA | | | CCAGGGTAGACA |
| | AGTGATAATAGG | | | AGTGATAATAGG |
| | CTGGAGCCTCGG | | | CTGGAGCCTCGG |
| | TGGCCATGCTTC | | | TGGCCATGCTTC |
| | TTGCCCCTTGGG | | | TTGCCCCTTGGG |
| | CCTCCCCCCAGC | | | CCTCCCCCCAGC |
| | CCCTCCTCCCCT | | | CCCTCCTCCCCT |
| | TCCTGCACCCGT | | | TCCTGCACCCGT |
| | ACCCCCGTGGTC | | | ACCCCCGTGGTC |
| | TTTGAATAAAGT | | | TTTGAATAAAGT |
| | CTGAGTGGGCGG | | | CTGAGTGGGCGG |
| | C | | | CAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAAAAAAAAA |
| | | | | AAAAATCTAG |

VZV mRNA Sequences

| mRNA | | SEQ ID |
|---|---|---|
| Name(s) | mRNA Sequence (assumes T100 tail) | NO |
| VZV_gE_ | G*GGGAAAUAAGAGAGAAAAGAAGAGUAAGAAGAAAUAUAAGA | 92 |
| Oka | GCCACCAUGGGGACAGUGAAUAAGCCGGUUGUGGGCGUGCUUAU | |
| | GGGCUUUGGGAUUAUUACCGGUACAUUACGAAUUACCAAUCCAG | |
| | UGCGCGCCAGUGUGCUGCGUUACGACGACUUUCACAUUGACGAG | |
| | GAUAAGCUGGAUACUAACAGCGUGUACGAACCUUAUUACCACUC | |
| | AGAUCAUGCCGAAUCAAGCUGGGUUAAUAGAGGAGAAAGCAGC | |
| | CGAAAAGCCUACGACCACAACUCACCUUAUAUUUGGCCCAGAAA | |
| | CGAUUAUGACGGUUUCCUGGAAAACGCACAUGAACACCAUGGAG | |
| | UCUACAACCAAGGCAGGGGAAUCGACAGUGGCGAGCGUCUUAUG | |
| | CAGCCAACACAGAUGUCGGCACAGGAGGAUCUCGGUGAUGACAC | |
| | CGGCAUACACGUGAUUCCCACAUUAAACGGCGACGACAGACAUA | |
| | AGAUCGUCAAUGUGGAUCAGCGUCAGUAUGGGGAUGUCUUUAA | |
| | AGGCGAUUUGAAUCCAAAGCCCCAAGGACAGAGACUGAUCGAGG | |
| | UCUCUGUAGAAGAAAAUCACCCCUUCACUUUGCGCGCUCCAAUC | |
| | CAGAGGAUUUACGGGGUGCGUUAUACCGAAACUUGGAGUUUCU | |
| | UGCCGUCACUGACGUGUACGGGGGAUGCCGCCCCCGCAAUCCAG | |
| | CACAUCUGUCUGAAACACACCACAUGCUUUCAGGACGUGGUUGU | |
| | GGAUGUGGAUUGCGCGGAAAACACAAAAGAAGACCAACUCGCCG | |
| | AAAUCAGCUAUCGUUUUCAGGGUAAAAAAGAGGCCGACCAACCG | |
| | UGGAUUGUUGUGAAUACGAGCACGCUCUUCGAUGAGCUUGAAC | |
| | UCGAUCCCCCGGAAAUCGAGCCUGGGGUUCUAAAAGUGUUGAGG | |
| | ACCGAGAAGCAGUACCUCGGGGUUUAUAUCUGGAAUAUGAGAG | |
| | GCUCCGAUGGCACCUCUACCUACGCAACGUUUCUGGUUACCUGG | |
| | AAGGGAGACGAGAAGACACGGAAUCCAACGCCCGCUGUGACCCC | |
| | UCAGCCUAGGGGAGCCGAAUUCCACAUGUGGAACUAUCACUCCC | |
| | AUGUAUUCAGUGUGGGUGACACUUUCAGCCUGGCCAUGCACCUG | |
| | CAGUAUAAGAUUCACGAGGCACCCUUCGACCUCCUGCUGGAGUG | |
| | GUUGUACGUACCUAUUGAUCCCACUUGUCAGCCCAUGCGCCUGU | |
| | ACUCCACUUGCUUGUACCACCCCAAUGCACCACAGUGUCUAUCA | |
| | CACAUGAACUCCGGGUGUACCUUUACUUCACCCCAUCUUGCCCA | |
| | GCGGGUCGCCAGCACAGUGUAUCAGAACUGUGAGCAUGCUGACA | |
| | ACUAUACUGCUUAUUGCCUCGGAAUAUCCCAUAUGGAGCCAAGC | |
| | UUCGGGCUCAUACUGCACGAUGGUGGUACGACACUCAAGUUCGU | |
| | GGACACCCCCGAAAGCCUUUCUGGCUUGUACGUGUUCGUGGUCU | |
| | ACUUCAAUGGACAUGUGGAGGCAGUGGCUUACACAGUGGUUUC | |
| | GACAGUUGAUCACUUUGUAAAUGCCAUUGAGGAACGCGGCUUCC | |
| | CGCCUACAGCGGGCCAGCCCCCUGCGACAACAAAACCAAAAGAG | |
| | AUUACGCCCGUUAAUCCUGGGACUAGUCCAUUGCUGAGGUAUGC | |
| | CGCCUGGACUGGCGGUCUGGCGGCCGUGGUACUUCUGUGUUUAG | |
| | UCAUAUUUCUGAUCUGUACCGCUAAACGUAUGCGGGUCAAGGCU | |
| | UACCGUGUUGACAAGUCUCCUUACAAUCAGUCAAUGUACUAUGC | |
| | AGGACUCCCUGUUGACGAUUUCGAAGACUCAGAGAGUACAGACA | |
| | CAGAAGAAGAAUUCGGAAACGCUAUAGGUGGCUCUCACGGAGG | |
| | UAGCUCGUAUACAGUGUACAUCGAUAAAACCAGAUGAUAAUAG | |
| | GCUGGAGCCUCGGUGGCCAUGCUUCUUGCCCCUUGGGCCUCCCC | |
| | CCAGCCCCUCCUCCCCUUCCUGCACCCGUACCCCCGUGGUCUUUG | |
| | AAUAAAGUCUGAGUGGGCGGCAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAUCUAG | |
| VZV_gE_ | G*GGGAAAUAAGAGAGAAAAGAAGAGUAAGAAGAAAUAUAAGA | 93 |
| Oka_ | GCCACCAUGGAGACUCCCGCUCAGCUACUGUUCCUCCUGCUCCU | |
| hIgkappa | UUGGCUGCCUGAUACUACAGGCUCUGUUUUGCGGUACGACGACU | |
| | UUCACAUCGAUGAGGACAAGCUCGACACUAAUAGCGUGUAUGAG | |
| | CCCUACUACCAUUCAGAUCACGCCGAGUCCUCUUGGGUGAACAG | |
| | GGGUGAAAGUUCUAGGAAAGCCUAUGAUCACAACAGCCCUUAUA | |
| | UUUGGCCACGGAAUGAUUACGACGGAUUUCUCGAAAAUGCCCAC | |
| | GAGCAUCACGGAGUGUACAACCAGGGCCGUGGAAUCGACUCUGG | |
| | GGAGAGAUUGAUGCAACCUACACAGAUGAGCGCCCAGGAAGAUC | |
| | UCGGGGAUGAUACAGGAAUUCACGUUAUCCCUACAUUAAACGGA | |
| | GAUGACCGCCACAAAAUCGUCAAUGUCGAUCAAAGACAGUAUGG | |
| | AGAUGUGUUCAAAGGCGAUCUCAACCCUAAGCCGCAGGGCCAGA | |
| | GACUCAUUGAGGUGUCUGUCGAAGAGAACCACCCUUUCACUCUG | |
| | CGCGCUCCCAUUCAGAGAAUCUAUGGAGUUCGCUAUACGGAGAC | |
| | UUGGUCAUUCCUUCCUUCCCUGACAUGCACCGGAGACGCCGCCC | |
| | CUGCCAUUCAGCACAUAUGCCUGAAACAUACCACCUGUUUCCAG | |
| | GAUGUGGUGGUUGAUGUUGAUUGUGCUGAAAAUACCAAGGAAG | |
| | ACCAACUGGCCGAGAUUAGUUACCGGUUCCAAGGGAAAAAGGAA | |
| | GCCGACCAGCCAUGGAUUGUGGUUAAUACAAGCACUCUGUUCGA | |
| | UGAGCUCGAGCUGGAUCCCCCCGAGAUAGAACCCGGAGUUCUGA | |
| | AAGUGCUCCGGACAGAAAAACAAUAUCUGGGAGUCUACAUAUG | |
| | GAACAUGCGCGGUUCCGAUGGGACCUCCACUUAUGCAACCUUUC | |
| | UCGUCACGUGGAAGGGAGAUGAGAAAACUAGGAAUCCCACACCC | |
| | GCUGUCACACCACAGCCAAGAGGGGCUGAGUUCCAUAUGUGGAA | |
| | CUAUCAUAGUCACGUGUUUAGUGUCGGAGAUACGUUUUCAUUG | |
| | GCUAUGCAUCUCCAGUACAAGAUUCAUGAGGCUCCCUUCGAUCU | |
| | GUUGCUUGAGUGGUUGUACGUCCCGAUUGACCCGACCUGCCAGC | |
| | CCAUGCGACUGUACAGCACCUGUCUCUACCAUCCAAACGCUCCG | |
| | CAAUGUCUGAGCCACAUGAACUCUGGGUGUACUUUCACCAGUCC | |
| | CCACCUCGCCCAGCGGGUGGCCUCUACUGUUUACCAGAACUGUG | |
| | AGCACGCCGACAACUACACCGCAUACUGCCUCGGUAUUUCUCAC | |
| | AUGGAACCCUCCUUCGGACUCAUCCUGCACGAUGGGGGCACUAC | |
| | CCUGAAGUUCGUUGAUACGCCAGAAUCUCUGUCUGGGCUCUAUG | |
| | UUUUCGUGGUCUACUUCAAUGGCCAUGUCGAGGCCGUGGCCUAU | |
| | ACUGUCGUUUCUACCGUGGAUCAUUUUGUGAACGCCAUCGAAGA | |
| | ACGGGGAUUCCCCCCUACGGCAGGCCAGCCGCCUGCAACCACCA | |
| | AGCCCAAGGAAAUAACACCAGUGAACCCUGGCACCUCACCUCUC | |
| | CUAAGAUAUGCCGCGUGGACAGGGGGACUGGCGGCAGUGGUGCU | |
| | CCUCUGUCUCGUGAUCUUUCUGAUCUGUACAGCCAAGAGGAUGA | |
| | GGGUCAAGGCUUAUAGAGUGGACAAGUCCCCCUACAAUCAGUCA | |
| | AUGUACUACGCCGGCCUUCCCGUUGAUGAUUUUGAGGAUUCCGA | |
| | GUCCACAGAUACUGAGGAAGAGUUCGGUAACGCUAUAGGCGGCU | |
| | CUCACGGGGGUUCAAGCUACACGGUUUACAUUGACAAGACACGC | |
| | UGAUAAUAGGCUGGAGCCUCGGUGGCCAUGCUUCUUGCCCCUUG | |
| | GGCCUCCCCCCAGCCCCUCCUCCCCUUCCUGCACCCGUACCCCCG | |
| | UGGUCUUUGAAUAAAGUCUGAGUGGGCGGCAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAAAAAAAAA | |
| | AUCUAG | |
| VZV-GE- | G*GGGAAAUAAGAGAGAAAAGAAGAGUAAGAAGAAAUAUAAGA | 94 |
| delete- | GCCACCAUGGGGACAGUUAAUAAACCUGUGGUGGGGGUAUUGA | |
| 562 | UGGGGUUCGGAAUUAUCACGGGAACGUUGCGUAUAACGAAUCC | |
| | GGUCAGAGCAUCCGUCUUGCGAUACGAUGAUUUUCACAUCGAUG | |
| | AAGACAAACUGGAUACAAACUCCGUAUAUGAGCCUUACUACCAU | |
| | UCAGAUCAUGCGGAGUCUUCAUGGGUAAAUCGGGGAGAGUCUU | |
| | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAACAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAAAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAAAAAACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAAAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUUGAUGAUAAUAG | |
| | GCUGGAGCCUCGGUGGCCAUGCUUCUUGCCCCUUGGGCCUCCCC | |
| | CCAGCCCCUCCUCCCCUUCCUGCACCCGUACCCCCGUGGUCUUUG | |
| | AAUAAAGUCUGAGUGGGCGGCAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAUCUA | |
| VZV-GE- | G*GGGAAAUAAGAGAGAAAAGAAGAGUAAGAAGAAAUAUAAGA | 95 |
| delete- | GCCACCAUGGAAACCCCGGCGCAGCUGCUGUUUCUGCUGCUGCU | |
| 562- | GUGGCUGCCGGAUACCACCGGCUCCGUCUUGCGAUACGAUGAUU | |
| replaced | UUCACAUCGAUGAAGACAAACUGGAUACAAACUCCGUAUAUGAG | |
| SP- | CCUUACUACCAUUCAGAUCAUGCGGAGUCUUCAUGGGUAAAUCG | |
| with | GGGAGAGUCUUCGCGAAAAGCGUACGAUCAUAACUCACCUUAUA | |
| IgKappa | UAUGGCCACGUAAUGAUUAUGAUGGAUUUUUAGAGAACGCACA | |
| | CGAACACCAUGGGGUGUAUAAUCAGGGCCGUGGUAUCGAUAGCG | |
| | GGGAACGGUUAAUGCAACCCACACAAAUGUCUGCACAGGAGGAU | |
| | CUUGGGGACGAUACGGGCAUCCACGUUAUCCCUACGUUAAACGG | |
| | CGAUGACAGACAUAAAAUUGUAAAUGUGGACCAACGUCAAUAC | |
| | GGUGACGUGUUUAAAGGAGAUCUUAAUCCAAAACCCCAAGGCCA | |
| | AAGACUCAUUGAGGUGUCAGUGGAAGAAAAUCACCCGUUUACU | |
| | UUACGCGCACCGAUUCAGCGGAUUUAUGGAGUCCGGUACACCGA | |
| | GACUUGGAGCUUUUUGCCGUCAUUAACCUGUACGGGAGACGCAG | |
| | CGCCCGCCAUCCAGCAUAUAUGUUUAAAACAUACAACAUGCUUU | |
| | CAAGACGUGGUGGUGGAUGUGGAUUGCGCGGAAAAUACUAAAG | |
| | AGGAUCAGUUGGCCGAAAUCAGUUACCGUUUUCAAGGUAAGAA | |
| | GGAAGCGGACCAACCGUGGAUUGUUGUAAACACGAGCACACUGU | |
| | UUGAUGAACUCGAAUUAGACCCCCCCGAGAUUGAACCGGGUGUC | |
| | UUGAAAGUACUUCGGACAGAAAAACAAUACUUGGGUGUGUACA | |
| | UUUGGAACAUGCGCGGCUCCGAUGGUACGUCUACCUACGCCACG | |
| | UUUUUGGUCACCUGGAAAGGGGAUGAAAAAACAAGAAACCCUA | |
| | CGCCCGCAGUAACUCCUCAACCAAGAGGGGCUGAGUUUCAUAUG | |
| | UGGAAUUACCACUCGCAUGUAUUUUCAGUUGGUGAUACGUUUA | |
| | GCUUGGCAAUGCAUCUUCAGUAUAAGAUACAUGAAGCGCCAUUU | |
| | GAUUUGCUGUUAGAGUGGUUGUAUGUCCCCAUCGAUCCUACAUG | |
| | UCAACCAAUGCGGUUAUAUUCUACGUGUUUGUAUCAUCCCAACG | |
| | CACCCCAAUGCCUCUCUCAUAUGAAUUCCGGUUGUACAUUUACC | |
| | UCGCCACAUUUAGCCCAGCGUGUUGCAAGCACAGUGUAUCAAAA | |
| | UUGUGAACAUGCAGAUAACUACACCGCAUAUUGUCUGGGAAUA | |
| | UCUCAUAUGGAGCCUAGCUUUGGUCUAAUCUUACACGACGGGGG | |
| | CACCACGUUAAAGUUUGUAGAUACACCCGAGAGUUUGUCGGGAU | |
| | UAUACGUUUUUGUGGUGUAUUUUAACGGGCAUGUUGAAGCCGU | |
| | AGCAUACACUGUUGUAUCCACAGUAGAUCAUUUUGUAAACGCAA | |
| | UUGAAGAGCGUGGAUUUCCGCCAACGGCCGGUCAGCCACCGGCG | |
| | ACUACUAAACCCAAGGAAAUUACCCCCGUAAACCCCGGAACGUC | |
| | ACCACUUCUACGAUAUGCCGCAUGGACCGGAGGGCUUGCAGCAG | |
| | UAGUACUUUUAUGUCUCGUAAUAUUUUUAAUCUGUACGGCUUG | |
| | AUGAUAAUAGGCUGGAGCCUCGGUGGCCAUGCUUCUUGCCCCUU | |
| | GGGCCUCCCCCCAGCCCCUCCUCCCCUUCCUGCACCCGUACCCCC | |
| | GUGGUCUUUGAAUAAAGUCUGAGUGGGCGGCAAAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAAAAAAAAA | |
| | AAUCUAG | |
| VZV-GE- | G*GGGAAAUAAGAGAGAAAAGAAGAGUAAGAAGAAAUAUAAGA | 96 |
| full_ | GCCACCAUGGGGACAGUUAAUAAACCUGUGGUGGGGGUAUUGA | |
| with_ | UGGGGUUCGGAAUUAUCACGGGAACGUUGCGUAUAACGAAUCC | |
| AEAADA | GGUCAGAGCAUCCGUCUUGCGAUACGAUGAUUUUCACAUCGAUG | |
| (SEQ | AAGACAAACUGGAUACAAACUCCGUAUAUGAGCCUUACUACCAU | |
| ID NO: | UCAGAUCAUGCGGAGUCUUCAUGGGUAAAUCGGGGAGAGUCUU | |
| 58) | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAACAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAAAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAAAAAACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAAAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCUAUAGGGUAGACAAGUCCCCGUAUAACCAAAGCAU | |
| | GUAUUACGCUGGCCUUCCAGUGGACGAUUUCGAGGACGCCGAAG | |
| | CCGCCGAUGCCGAAGAAGAGUUUGGUAACGCGAUUGGAGGGAG | |
| | UCACGGGGGUUCGAGUUACACGGUGUAUAUAGAUAAGACCCGG | |
| | UGAUGAUAAUAGGCUGGAGCCUCGGUGGCCAUGCUUCUUGCCCC | |
| | UUGGGCCUCCCCCCAGCCCCUCCUCCCCUUCCUGCACCCGUACCC | |
| | CCGUGGUCUUUGAAUAAAGUCUGAGUGGGCGGCAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAUCUAG | |
| VZV-GE- | G*GGGAAAUAAGAGAGAAAAGAAGAGUAAGAAGAAAUAUAAGA | 97 |
| full_ | GCCACCAUGGGGACAGUUAAUAAACCUGUGGUGGGGGUAUUGA | |
| with_ | UGGGGUUCGGAAUUAUCACGGGAACGUUGCGUAUAACGAAUCC | |
| AEAADA | GGUCAGAGCAUCCGUCUUGCGAUACGAUGAUUUUCACAUCGAUG | |
| (SEQ | AAGACAAACUGGAUACAAACUCCGUAUAUGAGCCUUACUACCAU | |
| ID NO: | UCAGAUCAUGCGGAGUCUUCAUGGGUAAAUCGGGGAGAGUCUU | |
| 58)_and_ | CGCGAAAAGCGUACGAUCAUAACUCACCUUAUAUAUGGCCACGU | |
| Y582G | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAACAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAAAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAAAAAACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAAAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCUAUAGGGUAGACAAGUCCCCGUAUAACCAAAGCAU | |
| | GUAUGGCGCUGGCCUUCCAGUGGACGAUUUCGAGGACGCCGAAG | |
| | CCGCCGAUGCCGAAGAAGAGUUUGGUAACGCGAUUGGAGGGAG | |
| | UCACGGGGGUUCGAGUUACACGGUGUAUAUAGAUAAGACCCGG | |
| | UGAUGAUAAUAGGCUGGAGCCUCGGUGGCCAUGCUUCUUGCCCC | |
| | UUGGGCCUCCCCCCAGCCCCUCCUCCCCUUCCUGCACCCGUACCC | |
| | CCGUGGUCUUUGAAUAAAGUCUGAGUGGGCGGCAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAUCUAG | |
| VZV-GE- | G*GGGAAAUAAGAGAGAAAAGAAGAGUAAGAAGAAAUAUAAGA | 98 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_574 | GGUCAGAGCAUCCGUCUUGCGAUACGAUGAUUUUCACAUCGAUG | |
| | AAGACAAACUGGAUACAAACUCCGUAUAUGAGCCUUACUACCAU | |
| | UCAGAUCAUGCGGAGUCUUCAUGGGUAAAUCGGGGAGAGUCUU | |
| | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAACAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAAAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAAAAAACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAAAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCUAUAGGGUAGACAAGUGAUGAUAAUAGGCUGGAG | |
| | CCUCGGUGGCCAUGCUUCUUGCCCCUUGGGCCUCCCCCCAGCCCC | |
| | UCCUCCCCUUCCUGCACCCGUACCCCCGUGGUCUUUGAAUAAAG | |
| | UCUGAGUGGGCGGCAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAUCUAG | |
| VZV-GE- | G*GGGAAAUAAGAGAGAAAAGAAGAGUAAGAAGAAAUAUAAGA | 99 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_ | GGUCAGAGCAUCCGUCUUGCGAUACGAUGAUUUUCACAUCGAUG | |
| 574_-_ | AAGACAAACUGGAUACAAACUCCGUAUAUGAGCCUUACUACCAU | |
| Y569A | UCAGAUCAUGCGGAGUCUUCAUGGGUAAAUCGGGGAGAGUCUU | |
| | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAACAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAAAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAAAAAACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAAAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCGCCAGGGUAGACAAGUGAUGAUAAUAGGCUGGAG | |
| | CCUCGGUGGCCAUGCUUCUUGCCCCUUGGGCCUCCCCCCAGCCCC | |
| | UCCUCCCCUUCCUGCACCCGUACCCCCGUGGUCUUUGAAUAAAG | |
| | UCUGAGUGGGCGGCAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAUCUAG | |
| VZV-GE- | AUGGGGACAGUUAAUAAACCUGUGGUGGGGGUAUUGAUGGGGU | 133 |
| Truncated- | UCGGAAUUAUCACGGGAACGUUGCGUAUAACGAAUCCGGUCAGA | |
| delete_ | GCAUCCGUCUUGCGAUACGAUGAUUUUCACAUCGAUGAAGACAA | |
| from_ | ACUGGAUACAAACUCCGUAUAUGAGCCUUACUACCAUUCAGAUC | |
| 574-_ | AUGCGGAGUCUUCAUGGGUAAAUCGGGGAGAGUCUUCGCGAAA | |
| Y569A | AGCGUACGAUAAUCAUAACUCACCUUAUAUAUGGCCACGUAAUG | |
| (ORF) | AUUAUGAUGGAUUUUUAGAGAACGCACACGAACACCAUGGGGU | |
| | GUAUAAUCAGGGCCGUGGUAUCGAUAGCGGGGAACGGUUAAUG | |
| | CAACCCACACAAAUGUCUGCACAGGAGGAUCUUGGGGACGAUAC | |
| | GGGCAUCCACGUUAUCCCUACGUUAAACGGCGAUGACAGACAUA | |
| | AAAUUGUAAAUGUGGACCAACGUCAAUACGGUGACGUGUUUAA | |
| | AGGAGAUCUUAAUCCAAAACCCCAAGGCCAAAGACUCAUUGAGG | |
| | UGUCAGUGGAAGAAAAUCACCCGUUUACUUUACGCGCACCGAUU | |
| | CAGCGGAUUUAUGGAGUCCGGUACACCGAGACUUGGAGCUUUUU | |
| | GCCGUCAUUAACCUGUACGGGAGACGCAGCGCCCGCCAUCCAGC | |
| | AUAUAUGUUUAAAACAUACAACAUGCUUUCAAGACGUGGUGGU | |
| | GGAUGUGGAUUGCGCGGAAAAUACUAAAGAGGAUCAGUUGGCC | |
| | GAAAUCAGUUACCGUUUUCAAGGUAAGAAGGAAGCGGACCAACC | |
| | GUGGAUUGUUGUAAACACGAGCACACUGUUUGAUGAACUCGAA | |
| | UUAGACCCCCCCGAGAUUGAACCGGGUGUCUUGAAAGUACUUCG | |
| | GACAGAAAAACAAUACUUGGGUGUGUACAUUUGGAACAUGCGC | |
| | GGCUCCGAUGGUACGUCUACCUACGCCACGUUUUUGGUCACCUG | |
| | GAAAGGGGAUGAAAAAACAAGAAACCCUACGCCCGCAGUAACUC | |
| | CUCAACCAAGAGGGGCUGAGUUUCAUAUGUGGAAUUACCACUCG | |
| | CAUGUAUUUUCAGUUGGUGAUACGUUUAGCUUGGCAAUGCAUC | |
| | UUCAGUAUAAGAUACAUGAAGCGCCAUUUGAUUUGCUGUUAGA | |
| | GUGGUUGUAUGUCCCCAUCGAUCCUACAUGUCAACCAAUGCGGU | |
| | UAUAUUCUACGUGUUUGUAUCAUCCCAACGCACCCCAAUGCCUC | |
| | UCUCAUAUGAAUUCCGGUUGUACAUUUACCUCGCCACAUUUAGC | |
| | CCAGCGUGUUGCAAGCACAGUGUAUCAAAAUUGUGAACAUGCAG | |
| | AUAACUACACCGCAUAUUGUCUGGGAAUAUCUCAUAUGGAGCCU | |
| | AGCUUUGGUCUAAUCUUACACGACGGGGGCACCACGUUAAAGUU | |
| | UGUAGAUACACCCGAGAGUUUGUCGGGAUUAUACGUUUUUGUG | |
| | GUGUAUUUUAACGGGCAUGUUGAAGCCGUAGCAUACACUGUUG | |
| | UAUCCACAGUAGAUCAUUUUGUAAACGCAAUUGAAGAGCGUGG | |
| | AUUUCCGCCAACGGCCGGUCAGCCACCGGCGACUACUAAACCCA | |
| | AGGAAAUUACCCCCGUAAACCCCGGAACGUCACCACUUCUACGA | |
| | UAUGCCGCAUGGACCGGAGGGCUUGCAGCAGUAGUACUUUUAUG | |
| | UCUCGUAAUAUUUUUAAUCUGUACGGCUAAACGAAUGAGGGUU | |
| | AAAGCCGCCAGGGUAGACAAGUGAUGAUAAUAGGCUGGAGCCUC | |
| | GGUGGCCAUGCUUCUUGCCCCUUGGGCCUCCCCCCAGCCCCUCCU | |
| | CCCCUUCCUGCACCCGUACCCCCGUGGUCUUUGAAUAAAGUCUG | |
| | AGUGGGCGGC | |
| VZV-GI- | G*GGGAAAUAAGAGAGAAAAGAAGAGUAAGAAGAAAUAUAAGA | 100 |
| full | GCCACCAUGUUUUUAAUCCAAUGUUUGAUAUCGGCCGUUAUAUU | |
| | UUACAUACAAGUGACCAACGCUUUGAUCUUCAAGGGCGACCACG | |
| | UGAGCUUGCAAGUUAACAGCAGUCUCACGUCUAUCCUUAUUCCC | |
| | AUGCAAAAUGAUAAUUAUACAGAGAUAAAAGGACAGCUUGUCU | |
| | UUAUUGGAGAGCAACUACCUACCGGGACAAACUAUAGCGGAACA | |
| | CUGGAACUGUUAUACGCGGAUACGGUGGCGUUUUGUUUCCGGUC | |
| | AGUACAAGUAAUAAGAUACGACGGAUGUCCCCGGAUUAGAACG | |
| | AGCGCUUUUAUUUCGUGUAGGUACAAACAUUCGUGGCAUUAUG | |
| | GUAACUCAACGGAUCGGAUAUCAACAGAGCCGGAUGCUGGUGUA | |
| | AUGUUGAAAAUUACCAAACCGGGAAUAAAUGAUGCUGGUGUGU | |
| | AUGUACUUCUUGUUCGGUUAGACCAUAGCAGAUCCACCGAUGGU | |
| | UUCAUUCUUGGUGUAAAUGUAUAUACAGCGGGCUCGCAUCACAA | |
| | CAUUCACGGGGUUAUCUACACUUCUCCAUCUCUACAGAAUGGAU | |
| | AUUCUACAAGAGCCCUUUUUCAACAAGCUCGUUUGUGUGAUUUA | |
| | CCCGCGACACCCAAAGGGUCCGGUACCUCCCUGUUUCAACAUAU | |
| | GCUUGAUCUUCGUGCCGGUAAAUCGUUAGAGGAUAACCCUUGGU | |
| | UACAUGAGGACGUUGUUACGACAGAAACUAAGUCCGUUGUUAA | |
| | GGAGGGGAUAGAAAAUCACGUAUAUCCAACGGAUAUGUCCACG | |
| | UUACCCGAAAAGUCCCUUAAUGAUCCUCCAGAAAAUCUACUUAU | |
| | AAUUAUUCCUAUAGUAGCGUCUGUCAUGAUCCUCACCGCCAUGG | |
| | UUAUUGUUAUUGUAAUAAGCGUUAAGCGACGUAGAAUUAAAAA | |
| | ACAUCCAAUUUAUCGCCCAAAUACAAAAACAAGAAGGGGCAUAC | |
| | AAAAUGCGACACCAGAAUCCGAUGUGAUGUUGGAGGCCGCCAUU | |
| | GCACAACUAGCAACGAUUCGCGAAGAAUCCCCCCCACAUUCCGU | |
| | UGUAAACCCGUUUGUUAAAUAGUGAUAAUAGGCUGGAGCCUCG | |
| | GUGGCCAUGCUUCUUGCCCCUUGGGCCUCCCCCCAGCCCCUCCUC | |
| | CCCUUCCUGCACCCGUACCCCCGUGGUCUUUGAAUAAAGUCUGA | |
| | GUGGGCGGCAAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAUCUAG | |
| VZV-GE- | G*GGGAAAUAAGAGAGAAAAGAAGAGUAAGAAGAAAUAUAAGA | 101 |
| Truncated- | GCCACCAUGGGCACCGUGAACAAGCCCGUCGUGGGCGUGCUGAU | |
| delete_ | GGGCUUCGGCAUCAUCACCGGCACCCUGCGGAUCACCAAUCCUG | |
| from_ | UGCGGGCCAGCGUGCUGAGAUACGACGACUUCCACAUCGACGAG | |
| 574_-_ | GACAAGCUGGACACCAACAGCGUGUACGAGCCCUACUACCACAG | |
| Y569A | CGACCACGCCGAGAGCAGCUGGGUCAACAGAGGCGAGUCCAGCC | |
| Variant 1 | GGAAGGCCUACGACCACAACAGCCCCUACAUCUGGCCCCGGAAC | |
| | GACUACGACGGCUUCCUGGAAAAUGCCCACGAGCACCACGGCGU | |
| | GUACAACCAGGGCAGAGGCAUCGACAGCGGCGAGAGACUGAUGC | |
| | AGCCCACCCAGAUGAGCGCCCAGGAAGAUCUGGGCGACGACACC | |
| | GGCAUCCACGUGAUCCCUACCCUGAACGGCGACGACCGGCACAA | |
| | GAUCGUGAACGUGGACCAGCGGCAGUACGGCGACGUGUUCAAGG | |
| | GCGACCUGAACCCCAAGCCCCAGGGACAGCGGCUGAUUGAGGUG | |
| | UCCGUGGAAGAGAACCACCCCUUCACCCUGAGAGCCCCUAUCCA | |
| | GCGGAUCUACGGCGUGCGCUAUACCGAGACUUGGAGCUUCCUGC | |
| | CCAGCCUGACCUGUACUGGCGACGCCGCUCCUGCCAUCCAGCAC | |
| | AUCUGCCUGAAGCACACCACCUGUUUCCAGGACGUGGUGGUGGA | |
| | CGUGGACUGCGCCGAGAACACCAAAGAGGACCAGCUGGCCGAGA | |
| | UCAGCUACCGGUUCCAGGGCAAGAAAGAGGCCGACCAGCCCUGG | |
| | AUCGUCGUGAACACCAGCACCCUGUUCGACGAGCUGGAACUGGA | |
| | CCCUCCCGAGAUCGAACCCGGGGUGCUGAAGGUGCUGCGGACCG | |
| | AGAAGCAGUACCUGGGAGUGUACAUCUGGAACAUGCGGGGCAGC | |
| | GACGGCACCUCUACCUACGCCACCUUCCUCGUGACCUGGAAGGG | |
| | CGACGAGAAAACCCGGAACCCUACCCCUGCCGUGACCCCUCAGC | |
| | CUAGAGGCGCCGAGUUUCACAUGUGGAAUUACCACAGCCACGUG | |
| | UUCAGCGUGGGCGACACCUUCUCCCUGGCCAUGCAUCUGCAGUA | |
| | CAAGAUCCACGAGGCCCCUUUCGACCUGCUGCUGGAAUGGCUGU | |
| | ACGUGCCCAUCGACCCUACCUGCCAGCCCAUGCGGCUGUACUCC | |
| | ACCUGUCUGUACCACCCCAACGCCCCUCAGUGCCUGAGCCACAU | |
| | GAAUAGCGGCUGCACCUUCACCAGCCCUCACCUGGCUCAGAGGG | |
| | UGGCCAGCACCGUGUACCAGAAUUGCGAGCACGCCGACAACUAC | |
| | ACCGCCUACUGCCUGGGCAUCAGCCACAUGGAACCCAGCUUCGG | |
| | CCUGAUCCUGCACGAUGGCGGCACCACCCUGAAGUUCGUGGACA | |
| | CCCCUGAGUCCCUGAGCGGCCUGUACGUGUUCGUGGUGUACUUC | |
| | AACGGCCACGUGGAAGCCGUGGCCUACACCGUGGUGUCCACCGU | |
| | GGACCACUUCGUGAACGCCAUCGAGGAACGGGGCUUCCCUCCAA | |
| | CUGCUGGACAGCCUCCUGCCACCACCAAGCCCAAAGAAAUCACC | |
| | CCUGUGAACCCCGGCACCAGCCCACUGCUGCGCUAUGCUGCUUG | |
| | GACAGGCGGACUGGCUGCUGUGGUGCUGCUGUGCCUCGUGAUUU | |
| | UCCUGAUCUGCACCGCCAAGCGGAUGAGAGUGAAGGCCGCCAGA | |
| | GUGGACAAGUGAUAAUAGGCUGGAGCCUCGGUGGCCAUGCUUCU | |
| | UGCCCCUUGGGCCUCCCCCCAGCCCCUCCUCCCCUUCCUGCACCC | |
| | GUACCCCCGUGGUCUUUGAAUAAAGUCUGAGUGGGCGGCAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAUCUAG | |
| VZV-GE- | G*GGGAAAUAAGAGAGAAAAGAAGAGUAAGAAGAAAUAUAAGA | 102 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_ | GGUCAGAGCAUCCGUCUUGCGAUACGAUGAUUUUCACAUCGAUG | |
| 574-_ | AAGACAAACUGGAUACAAACUCCGUAUAUGAGCCUUACUACCAU | |
| Y569A | UCAGAUCAUGCGGAGUCUUCAUGGGUAAAUCGGGGAGAGUCUU | |
| Variant 2 | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAACAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAGAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAGAAGACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAAAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCGCCAGGGUAGACAAGUGAUAAUAGGCUGGAGCCUC | |
| | GGUGGCCAUGCUUCUUGCCCCUUGGGCCUCCCCCCAGCCCCUCCU | |
| | CCCCUUCCUGCACCCGUACCCCCGUGGUCUUUGAAUAAAGUCUG | |
| | AGUGGGCGGCAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAUCUAG | |
| VZV-GE- | G*GGGAAAUAAGAGAGAAAAGAAGAGUAAGAAGAAAUAUAAGA | 103 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_ | GGUCAGAGCAUCCGUCUUGCGAUACGAUGAUUUUCACAUCGAUG | |
| 574_-_ | AAGACAAACUGGAUACAAACUCCGUAUAUGAGCCUUACUACCAU | |
| Y569A | UCAGAUCAUGCGGAGUCUUCAUGGGUAAAUCGGGGAGAGUCUU | |
| Variant 3 | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAACAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCACCCGAGAUUGAACCGGGUGUCUUGAAAGUACU | |
| | UCGGACAGAGAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAGAAGACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAAAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCGCCAGGGUAGACAAGUGAUAAUAGGCUGGAGCCUC | |
| | GGUGGCCAUGCUUCUUGCCCCUUGGGCCUCCCCCCAGCCCCUCCU | |
| | CCCCUUCCUGCACCCGUACCCCCGUGGUCUUUGAAUAAAGUCUG | |
| | AGUGGGCGGCAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAUCUAG | |
| VZV-GE- | G*GGGAAAUAAGAGAGAAAAGAAGAGUAAGAAGAAAUAUAAGA | 104 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_ | GGUCAGAGCAUCCGUCUUGCGAUACGAUGAUUUUCACAUCGAUG | |
| 574_-_ | AAGACAAACUGGAUACAAACUCCGUAUAUGAGCCUUACUACCAU | |
| Y569A | UCAGAUCAUGCGGAGUCUUCAUGGGUAAAUCGGGGAGAGUCUU | |
| Variant 4 | CGCGAAAGGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAGAUUGUAAAUGUGGACCAACGUCAAUACGGUGACGUGU | |
| | UUAAAGGAGAUCUUAAUCCAAAGCCCCAAGGCCAAAGACUCAUU | |
| | GAGGUGUCAGUGGAAGAGAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAGCAUACAACAUGCUUUCAAGACGUGG | |
| | UGGUGGAUGUGGAUUGCGCGGAGAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAGAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAGAAGACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAGAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCGCCAGGGUAGACAAGUGAUAAUAGGCUGGAGCCUC | |
| | GGUGGCCAUGCUUCUUGCCCCUUGGGCCUCCCCCCAGCCCCUCCU | |
| | CCCCUUCCUGCACCCGUACCCCCGUGGUCUUUGAAUAAAGUCUG | |
| | AGUGGGCGGCAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAUCUAG | |
| VZV-GE- | G*GGGAAAUAAGAGAGAAAAGAAGAGUAAGAAGAAAUAUAAGA | 105 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_ | GGUCAGAGCAUCCGUCUUGCGAUACGAUGAUUUUCACAUCGAUG | |
| 574-_ | AAGACAAACUGGAUACAAACUCCGUAUAUGAGCCUUACUACCAU | |
| Y569A | UCAGAUCAUGCGGAGUCUUCAUGGGUAAAUCGGGGAGAGUCUU | |
| Variant 5 | CGCGAAAGGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAGAUUGUAAAUGUGGACCAACGUCAAUACGGUGACGUGU | |
| | UUAAAGGAGAUCUUAAUCCAAAGCCCCAAGGCCAAAGACUCAUU | |
| | GAGGUGUCAGUGGAAGAGAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAGCAUACAACAUGCUUUCAAGACGUGG | |
| | UGGUGGAUGUGGAUUGCGCGGAGAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCACCCGAGAUUGAACCGGGUGUCUUGAAAGUACU | |
| | UCGGACAGAGAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAGAAGACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAGAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCGCCAGGGUAGACAAGUGAUAAUAGGCUGGAGCCUC | |
| | GGUGGCCAUGCUUCUUGCCCCUUGGGCCUCCCCCCAGCCCCUCCU | |
| | CCCCUUCCUGCACCCGUACCCCCGUGGUCUUUGAAUAAAGUCUG | |
| | AGUGGGCGGCAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAUCUAG | |
| VZV-GE- | G*GGGAAAUAAGAGAGAAAAGAAGAGUAAGAAGAAAUAUAAGA | 106 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_ | GGUCAGAGCAUCCGUCUUGCGAUACGAUGAUUUUCACAUCGAUG | |
| 574_-_ | AAGACAAACUGGAUACAAACUCCGUAUAUGAGCCUUACUACCAU | |
| Y569A | UCAGAUCAUGCGGAGUCUUCAUGGGUAAAUCGGGGAGAGUCUU | |
| Variant 6 | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAGCAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCCCCCGAGAUUGAACCGGGUGUCUUGAAAGUACU | |
| | UCGGACAGAGAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAGAAGACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAGAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCGCCAGGGUAGACAAGUGAUAAUAGGCUGGAGCCUC | |
| | GGUGGCCAUGCUUCUUGCCCCUUGGGCCUCCCCCCAGCCCCUCCU | |
| | CCCCUUCCUGCACCCGUACCCCCGUGGUCUUUGAAUAAAGUCUG | |
| | AGUGGGCGGCAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAUCUAG | |
| VZV-GE- | G*GGGAAAUAAGAGAGAAAAGAAGAGUAAGAAGAAAUAUAAGA | 107 |
| Truncated- | GCCACCAUGGGGACAGUUAAUAAACCUGUGGUGGGGGUAUUGA | |
| delete_ | UGGGGUUCGGAAUUAUCACGGGAACGUUGCGUAUAACGAAUCC | |
| from_ | GGUCAGAGCAUCCGUCUUGCGAUACGAUGAUUUUCACAUCGAUG | |
| 574_-_ | AAGACAAACUGGAUACAAACUCCGUAUAUGAGCCUUACUACCAU | |
| Y569A | UCAGAUCAUGCGGAGUCUUCAUGGGUAAAUCGGGGAGAGUCUU | |
| Variant 7 | CGCGAAAAGCGUACGAUCAUAACUCACCUUAUAUAUGGCCACGU | |
| | AAUGAUUAUGAUGGAUUUUUAGAGAACGCACACGAACACCAUG | |
| | GGGUGUAUAAUCAGGGCCGUGGUAUCGAUAGCGGGGAACGGUU | |
| | AAUGCAACCCACACAAAUGUCUGCACAGGAGGAUCUUGGGGACG | |
| | AUACGGGCAUCCACGUUAUCCCUACGUUAAACGGCGAUGACAGA | |
| | CAUAAAAUUGUAAAUGUGGACCAACGUCAAUACGGUGACGUGU | |
| | UUAAAGGAGAUCUUAAUCCAAAACCCCAAGGCCAAAGACUCAUU | |
| | GAGGUGUCAGUGGAAGAAAAUCACCCGUUUACUUUACGCGCACC | |
| | GAUUCAGCGGAUUUAUGGAGUCCGGUACACCGAGACUUGGAGCU | |
| | UUUUGCCGUCAUUAACCUGUACGGGAGACGCAGCGCCCGCCAUC | |
| | CAGCAUAUAUGUUUAAAGCAUACAACAUGCUUUCAAGACGUGG | |
| | UGGUGGAUGUGGAUUGCGCGGAAAAUACUAAAGAGGAUCAGUU | |
| | GGCCGAAAUCAGUUACCGUUUUCAAGGUAAGAAGGAAGCGGACC | |
| | AACCGUGGAUUGUUGUAAACACGAGCACACUGUUUGAUGAACUC | |
| | GAAUUAGACCCACCCGAGAUUGAACCGGGUGUCUUGAAAGUACU | |
| | UCGGACAGAGAAACAAUACUUGGGUGUGUACAUUUGGAACAUG | |
| | CGCGGCUCCGAUGGUACGUCUACCUACGCCACGUUUUUGGUCAC | |
| | CUGGAAAGGGGAUGAGAAGACAAGAAACCCUACGCCCGCAGUAA | |
| | CUCCUCAACCAAGAGGGGCUGAGUUUCAUAUGUGGAAUUACCAC | |
| | UCGCAUGUAUUUUCAGUUGGUGAUACGUUUAGCUUGGCAAUGC | |
| | AUCUUCAGUAUAAGAUACAUGAAGCGCCAUUUGAUUUGCUGUU | |
| | AGAGUGGUUGUAUGUCCCCAUCGAUCCUACAUGUCAACCAAUGC | |
| | GGUUAUAUUCUACGUGUUUGUAUCAUCCCAACGCACCCCAAUGC | |
| | CUCUCUCAUAUGAAUUCCGGUUGUACAUUUACCUCGCCACAUUU | |
| | AGCCCAGCGUGUUGCAAGCACAGUGUAUCAGAAUUGUGAACAUG | |
| | CAGAUAACUACACCGCAUAUUGUCUGGGAAUAUCUCAUAUGGAG | |
| | CCUAGCUUUGGUCUAAUCUUACACGACGGGGGCACCACGUUAAA | |
| | GUUUGUAGAUACACCCGAGAGUUUGUCGGGAUUAUACGUUUUU | |
| | GUGGUGUAUUUUAACGGGCAUGUUGAAGCCGUAGCAUACACUG | |
| | UUGUAUCCACAGUAGAUCAUUUUGUAAACGCAAUUGAAGAGCG | |
| | UGGAUUUCCGCCAACGGCCGGUCAGCCACCGGCGACUACUAAAC | |
| | CCAAGGAAAUUACCCCCGUAAACCCCGGAACGUCACCACUUCUA | |
| | CGAUAUGCCGCAUGGACCGGAGGGCUUGCAGCAGUAGUACUUUU | |
| | AUGUCUCGUAAUAUUUUUAAUCUGUACGGCUAAACGAAUGAGG | |
| | GUUAAAGCCGCCAGGGUAGACAAGUGAUAAUAGGCUGGAGCCUC | |
| | GGUGGCCAUGCUUCUUGCCCCUUGGGCCUCCCCCCAGCCCCUCCU | |
| | CCCCUUCCUGCACCCGUACCCCCGUGGUCUUUGAAUAAAGUCUG | |
| | AGUGGGCGGCAAAAAAAAAAAAAAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAUCUAG | |
| VZV-GE- | AUGGGGACAGUUAAUAAACCUGUGGUGGGGGUAUUGAUGGGGU | 134 |
| Truncated- | UCGGAAUUAUCACGGGAACGUUGCGUAUAACGAAUCCGGUCAGA | |
| delete_ | GCAUCCGUCUUGCGAUACGAUGAUUUUCACAUCGAUGAAGACAA | |
| from_ | ACUGGAUACAAACUCCGUAUAUGAGCCUUACUACCAUUCAGAUC | |
| 574_-_ | AUGCGGAGUCUUCAUGGGUAAAUCGGGGAGAGUCUUCGCGAAA | |
| Y569A | AGCGUACGAUCAUAACUCACCUUAUAUAUGGCCACGUAAUGAUU | |
| Variant 7 | AUGAUGGAUUUUUAGAGAACGCACACGAACACCAUGGGGUGUA | |
| | UAAUCAGGGCCGUGGUAUCGAUAGCGGGGAACGGUUAAUGCAA | |
| | CCCACACAAAUGUCUGCACAGGAGGAUCUUGGGGACGAUACGGG | |
| | CAUCCACGUUAUCCCUACGUUAAACGGCGAUGACAGACAUAAAA | |
| | UUGUAAAUGUGGACCAACGUCAAUACGGUGACGUGUUUAAAGG | |
| | AGAUCUUAAUCCAAAACCCCAAGGCCAAAGACUCAUUGAGGUGU | |
| | CAGUGGAAGAAAAUCACCCGUUUACUUUACGCGCACCGAUUCAG | |
| | CGGAUUUAUGGAGUCCGGUACACCGAGACUUGGAGCUUUUUGCC | |
| | GUCAUUAACCUGUACGGGAGACGCAGCGCCCGCCAUCCAGCAUA | |
| | UAUGUUUAAAGCAUACAACAUGCUUUCAAGACGUGGUGGUGGA | |
| | UGUGGAUUGCGCGGAAAAUACUAAAGAGGAUCAGUUGGCCGAA | |
| | AUCAGUUACCGUUUUCAAGGUAAGAAGGAAGCGGACCAACCGUG | |
| | GAUUGUUGUAAACACGAGCACACUGUUUGAUGAACUCGAAUUA | |
| | GACCCACCCGAGAUUGAACCGGGUGUCUUGAAAGUACUUCGGAC | |
| | AGAGAAACAAUACUUGGGUGUGUACAUUUGGAACAUGCGCGGC | |
| | UCCGAUGGUACGUCUACCUACGCCACGUUUUUGGUCACCUGGAA | |
| | AGGGGAUGAGAAGACAAGAAACCCUACGCCCGCAGUAACUCCUC | |
| | AACCAAGAGGGGCUGAGUUUCAUAUGUGGAAUUACCACUCGCAU | |
| | GUAUUUUCAGUUGGUGAUACGUUUAGCUUGGCAAUGCAUCUUC | |
| | AGUAUAAGAUACAUGAAGCGCCAUUUGAUUUGCUGUUAGAGUG | |
| | GUUGUAUGUCCCCAUCGAUCCUACAUGUCAACCAAUGCGGUUAU | |
| | AUUCUACGUGUUUGUAUCAUCCCAACGCACCCCAAUGCCUCUCU | |
| | CAUAUGAAUUCCGGUUGUACAUUUACCUCGCCACAUUUAGCCCA | |
| | GCGUGUUGCAAGCACAGUGUAUCAGAAUUGUGAACAUGCAGAU | |
| | AACUACACCGCAUAUUGUCUGGGAAUAUCUCAUAUGGAGCCUAG | |
| | CUUUGGUCUAAUCUUACACGACGGGGGCACCACGUUAAAGUUUG | |
| | UAGAUACACCCGAGAGUUUGUCGGGAUUAUACGUUUUUGUGGU | |
| | GUAUUUUAACGGGCAUGUUGAAGCCGUAGCAUACACUGUUGUA | |
| | UCCACAGUAGAUCAUUUUGUAAACGCAAUUGAAGAGCGUGGAU | |
| | UUCCGCCAACGGCCGGUCAGCCACCGGCGACUACUAAACCCAAG | |
| | GAAAUUACCCCCGUAAACCCCGGAACGUCACCACUUCUACGAUA | |
| | UGCCGCAUGGACCGGAGGGCUUGCAGCAGUAGUACUUUUAUGUC | |
| | UCGUAAUAUUUUUAAUCUGUACGGCUAAACGAAUGAGGGUUAA | |
| | AGCCGCCAGGGUAGACAAGUGAUAAUAGGCUGGAGCCUCGGUGG | |
| | CCAUGCUUCUUGCCCCUUGGGCCUCCCCCCAGCCCCUCCUCCCCU | |
| | UCCUGCACCCGUACCCCCGUGGUCUUUGAAUAAAGUCUGAGUGG | |
| | GCGGC | |
| VZV-GE- | G*AGAAGAAAUAUAAGAGCCACCAUGGGGACAGUUAAUAAACCU | 108 |
| Truncated- | GUGGUGGGCGUAUUGAUGGGGUUCGGAAUUAUCACGGGAACGU | |
| delete_ | UGCGUAUAACGAAUCCGGUCAGAGCAUCCGUCUUGCGAUACGAU | |
| from_ | GAUUUUCACAUCGAUGAAGACAAACUGGAUACAAACUCCGUAUA | |
| 574_-_ | UGAGCCUUACUACCAUUCAGAUCAUGCGGAGUCUUCAUGGGUAA | |
| Y569A | AUCGGGGAGAGUCUUCGCGAAAGGCGUACGAUCAUAACUCACCU | |
| Variant 8 | UAUAUAUGGCCACGUAAUGAUUAUGAUGGAUUCUUAGAGAACG | |
| | CACACGAACACCAUGGGGUGUAUAAUCAGGGCCGUGGUAUCGAU | |
| | AGCGGGGAACGGUUAAUGCAACCCACACAAAUGUCUGCACAGGA | |
| | GGAUCUUGGGGACGAUACGGGCAUCCACGUUAUCCCUACGUUAA | |
| | ACGGCGAUGACAGACAUAAGAUUGUAAAUGUGGACCAACGUCA | |
| | AUACGGUGACGUGUUUAAAGGAGAUCUUAAUCCAAAGCCCCAAG | |
| | GCCAAAGACUCAUUGAGGUGUCAGUGGAAGAGAAUCACCCGUUU | |
| | ACUUUACGCGCACCGAUUCAGCGGAUUUAUGGAGUCCGGUACAC | |
| | CGAGACUUGGAGCUUCUUGCCGUCAUUAACCUGUACGGGAGACG | |
| | CAGCGCCCGCCAUCCAGCAUAUAUGUUUAAAGCAUACAACAUGC | |
| | UUUCAAGACGUGGUGGUGGAUGUGGAUUGCGCGGAGAAUACUA | |
| | AAGAGGAUCAGUUGGCCGAAAUCAGUUACCGUUUUCAAGGUAA | |
| | GAAGGAAGCGGACCAACCGUGGAUUGUUGUAAACACGAGCACAC | |
| | UGUUUGAUGAACUCGAAUUAGACCCACCCGAGAUUGAACCGGGU | |
| | GUCUUGAAAGUACUUCGGACAGAGAAACAAUACUUGGGUGUGU | |
| | ACAUUUGGAACAUGCGCGGCUCCGAUGGUACGUCUACCUACGCC | |
| | ACGUUCUUGGUCACCUGGAAAGGGGAUGAGAAGACAAGAAACCC | |
| | UACGCCCGCAGUAACUCCUCAACCAAGAGGGGCUGAGUUUCAUA | |
| | UGUGGAAUUACCACUCGCAUGUAUUUUCAGUUGGUGAUACGUU | |
| | UAGCUUGGCAAUGCAUCUUCAGUAUAAGAUACAUGAAGCGCCAU | |
| | UUGAUUUGCUGUUAGAGUGGUUGUAUGUCCCCAUCGAUCCUACA | |
| | UGUCAACCAAUGCGGUUAUAUUCUACGUGUUUGUAUCAUCCCAA | |
| | CGCACCCCAAUGCCUCUCUCAUAUGAAUUCCGGUUGUACAUUUA | |
| | CCUCGCCACAUUUAGCCCAGCGUGUUGCAAGCACAGUGUAUCAG | |
| | AAUUGUGAACAUGCAGAUAACUACACCGCAUAUUGUCUGGGAA | |
| | UAUCUCAUAUGGAGCCUAGCUUUGGUCUAAUCUUACACGACGGA | |
| | GGCACCACGUUAAAGUUUGUAGAUACACCCGAGAGUUUGUCGGG | |
| | AUUAUACGUCUUUGUGGUGUAUUUUAACGGGCAUGUUGAAGCC | |
| | GUAGCAUACACUGUUGUAUCCACAGUAGAUCAUUUUGUAAACGC | |
| | AAUUGAAGAGCGUGGAUUUCCGCCAACGGCCGGUCAGCCACCGG | |
| | CGACUACUAAACCCAAGGAAAUUACGCCCGUAAACCCCGGAACG | |
| | UCACCACUUCUACGAUAUGCCGCAUGGACCGGAGGGCUUGCAGC | |
| | AGUAGUACUUUUAUGUCUCGUAAUAUUCUUAAUCUGUACGGCU | |
| | AAACGAAUGAGGGUUAAAGCCGCCAGGGUAGACAAGUGAUAAU | |
| | AGGCUGGAGCCUCGGUGGCCAUGCUUCUUGCCCCUUGGGCCUCC | |
| | CCCCAGCCCCUCCUCCCCUUCCUGCACCCGUACCCCCGUGGUCUU | |
| | UGAAUAAAGUCUGAGUGGGCGGCAAAAAAAAAAAAAAAAAAAA | |
| | AAAAAAAAAAAAAAAAAAAAAAAAAAAAAAAAAAAAAAAAAAA | |
| | AAAAAAAAAAAAAAAAAAAAAAAAAAAAAAAAAAAAAUCUAG | |

G* represents a 5′ terminal cap, e.g., 7mG(5′)ppp(5′)NlmpNp

### Example 14: Variant gE Antigen Distribution in Vero and Mewo Cells

The expression and trafficking of VZV gE antigens having different C terminal variants was investigated in Vero cells and Mewo cells.

Vero cells are lineages of cells used in cell cultures. The ‘Vero’ lineage was isolated from kidney epithelial cells extracted from an African green monkey. MeWo cells are human malignant melanoma cells that are susceptible to VZV infection. Vero cells or Mewo cells were transfected with the constructs indicated below in Table 3. The tranfected cells were stained with antibodies for gE and for golgi markers GM 130 and golgin. Confocal microscopy was used to visualize the stained cells. The results for the constructs are described in Table 3 (“Cellular localization” column). FIG. 9 provides an exemplary experiment, which shows the results of the following transfected constructs: (1) VZV gE mRNA encoding a VZV gE polypeptide having a 62 amino acid deletion at the C-terminus (encoded by SEQ ID NO: 3); (2) full-length VZV gE mRNA encoding a VZV gE polypeptide having the AEAADA sequence (SEQ ID NO: 58) (encoded by SEQ ID NO: 7); or (3) PBS (as negative control). Using an anti-gE antibody, FIG. 9 shows that the truncated VZV gE polypeptide (having the 62 amino acid C-terminal deletion) localizes to a perinuclear location and organelles. The full-length VZV gE polypeptide having AEAADA sequence (SEQ ID NO: 58) was localized to the golgi and a perinuclear location. Importantly, several of the constructs, e.g., gE-truncated-delete_from_574_Y569A, gE full length with AEAADA (SEQ ID NO: 58), gE full length with AEAADA (SEQ ID NO: 58) and Y582C mutation, gE-truncated-delete_from_574, and gE-truncated-delete_from_574 with Y569A mutation each encoded polypeptides that localized to the cell membrane, indicating that these polypeptides may have enhanced antigenicity.

TABLE 3
Summary of Results for Cellular Trafficking of Variant VZV gE
Polypeptides

| | | | Cellular |
|---|---|---|---|
| Construct | Experimental conditions | Expression | localization |
| Full length gE | Vero cells—500 ng/well, | + | shows Golgi |
| | transfected 24 h transfection | | localization |
| | (C8) Construct = Full length | | |
| | GE | | |
| GE-full with | Vero cells—500 ng/well, | +++ | shows Golgi |
| AEAADA (SEQ | transfected 24 h transfection | | localization |
| ID NO: 58) | (C1)-Construct = VZV-GE- | | and diffuse |
| | full with AEAADA (SEQ | | perinuclear |
| | ID NO: 58) | | |
| GE-full with | Vero cells—500 ng/well, | low | shows |
| AEAADA (SEQ | transfected 24 h transfection | | organelles |
| ID NO: 58) and | (C6) Construct = VZV-GE | | and |
| Y582C | full_with_AEAADA (SEQ | | cytoplasmic |
| | ID NO: 58)_and_Y582G | | localization |
| GE-delete-562 | Vero cells—500 ng/well, | + | shows |
| | transfected 24 h transfection | | perinuclear |
| | (C2)-Construct = C2 VZV- | | and |
| | GE-delete-562 | | organelles |
| GE-delete-562- | Vero cells—500 ng/well, | +++ | shows golgi |
| replaced SP-with | transfected 24 h transfection | | localization |
| IgKappa | (C5) VZV-GE-delete-562- | | and |
| | replacedSignal Pepetide- | | cytoplasmic |
| | with IgKappa | | |
| GE-truncated- | Vero cells—500 ng/well, | ++ | shows Golgi |
| delete_from_574 | transfected 24 h transfection | | and |
| | (C4)-Construct = VZV-GE- | | cytoplasmic |
| | truncated-delete_from_574 | | localization |
| GE-truncated- | Vero cells—500 ng/well, | +++ | shows Golgi |
| delete_from_ | transfected 24 h transfection | | and cell |
| 574_Y569A | (C3)-Construct = VZV-GE- | | membrane |
| | truncated-delete_from_574_ | | localization |
| | Y569A | | |
| Full length gE | MeWo cells—500 ng/well, | +++ | shows Golgi |
| | transfected 24 h transfection | | localization |
| | (C8) Construct = Full length | | |
| | GE | | |
| GE-full with | MeWo cells—500 ng/well, | +++ | shows Golgi |
| AEAADA (SEQ | transfected 24 h transfection | | and |
| ID NO: 58) | (C1)-Construct = VZV-GE- | | Membrane |
| | full with AEAADA (SEQ | | localization |
| | ID NO: 58) | | |
| GE-full with | MeWo cells—500 ng/well, | ++ | shows golgi |
| AEAADA (SEQ | transfected 24 h transfection | | and cell |
| ID NO: 58) and | (C6) Construct = SE-VZV- | | membrane |
| Y582C | GE full_with_AEAADA | | localization |
| | (SEQ ID NO: 58)_and_ | | |
| | Y582G | | |
| GE-delete-562 | MeWo cells—500 ng/well, | +++ | shows |
| | transfected 24 h transfection | | perinuclear |
| | (C2)-Construct = C2 VZV- | | and |
| | GE-delete-562 | | cytoplasmic |
| | | | localization |
| GE-delete-562- | MeWo cells—500 ng/well, | +++ | shows golgi |
| replaced SP— | transfected 24 h transfection | | localization |
| with Ig Kappa | (C5) VZV-GE-delete-562- | | and |
| | replacedSignal Peptide with | | cytoplasmic |
| | IgKappa | | |
| GE-truncated- | MeWo cells—500 ng/well, | +++ | shows Golgi |
| delete_from_574 | transfected 24 h transfection | | and cell |
| | (C4)-Construct = -VZV-GE- | | membrane |
| | truncated-delete_from_574 | | localization |
| GE-truncated- | MeWo cells—500 ng/well, | +++ | shows Golgi |
| delete_from_ | transfected 24 h transfection | | and cell |
| 574_Y569A | (C3)-Construct = VZV-GE- | | membrane |
| | truncated-delete_from_574_ | | localization |
| | Y569A | | |

### Example 15: Immunization of BALB/C Mice with MC3 Formulated mRNA Encoded VZV gE Antigens

An immunization study was conducted as an initial evaluation of the effect of MC3-formulated mRNAs encoding VZV antigens as vaccine candidates to achieve immunization in BALB/C mice post intramuscular or intradermal administration.

The candidate vaccines were as follows:

(1) MC3 formulated VZVgE-hlg kappa mRNA having 5′ cap: m⁷G (5′) ppp (5′) G-2′-O-methyl, N1-methylpseudouridine chemical modification, and the additional hlg Kappa sequence.

(2) MC3 formulated VZV gE mRNA having 5′ cap: m⁷G (5′) ppp (5′) G-2′-O-methyl and N1-methylpseudouridine chemical modification.

(3) MC3 formulated VZVgE mRNA having 5′ cap: m⁷G (5′) ppp (5′) G-2′-O-methyl and no chemical modification.

All of the VZV gE mRNAs were strain Oka.

BALB/C mice were given a single 10 μg dose or two 10 μg doses (at day 28) of MC3 formulated VZV gE mRNA (either vaccine (1), (2), or (3) described above) either intramuscularly or intradermally. G5 refers mRNA having N1-methylpseudouridine chemical modification. GO refers to unmodified mRNA. Cap 1 refers to 5′ cap: m⁷G (5′) ppp (5′) G-2′-O-methyl. Each treatment group contained eight mice. The positive control was VARIVAX® vaccine and the negative control was PBS.

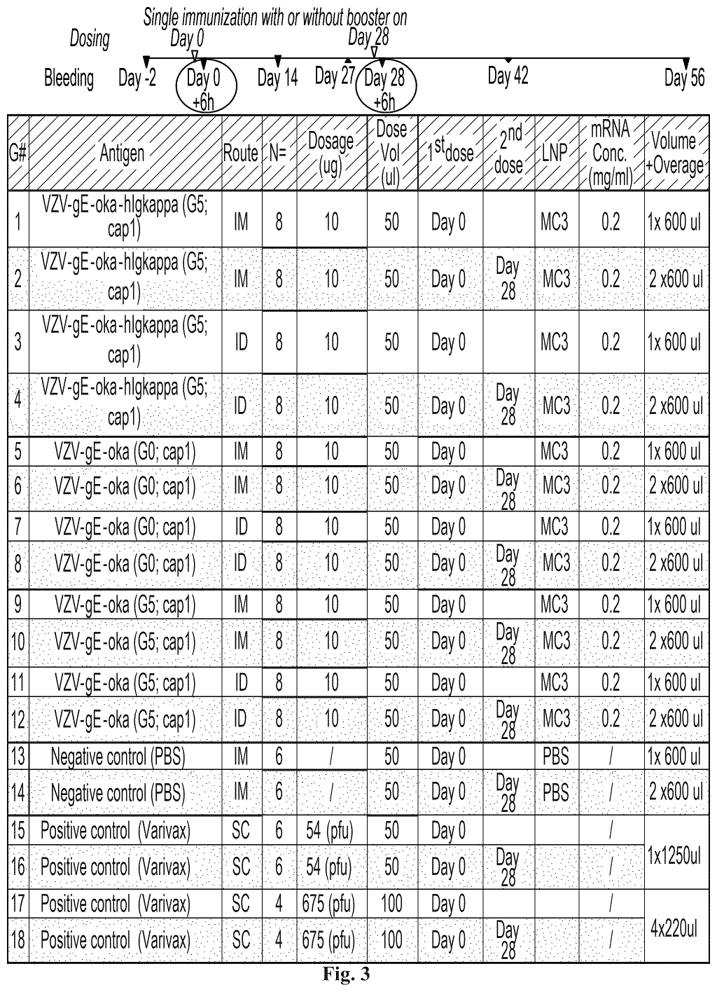

Blood samples were taken to determine the presence/level of serum protein and antibodies. Western blots were performed to detect VZV-gE protein expression at six hours and ELISAs were performed to detect mouse IgGs. Schematics of the constructs encoding VZV gE (strain Oka) are shown in FIG. 2. Schematics of the study's design and schedule of injection are shown in FIG. 3 and Table 3. Table 4 shows the various time points for collection of different samples. Blood was collected for serum protein and antibody determination, while VZV protein expression was surveyed 6 hours post-dosing on day 0 for groups 1-4, 13, and 14, and 6 hours post-dosing on day 28 for groups 2, 4, and 14. Antibody detection assays were performed on day-3, day 14, day 27, day 42, and day 56.

TABLE 4
Injection Schedule

| | | | | | Dose | | | | mRNA | |
|---|---|---|---|---|---|---|---|---|---|---|
| | | | | Dose | Vol | 1^{st} | 2^{nd} | | Conc. | Volume + |
| G# | Antigen | Route | N= | (μg) | (μl) | dose | dose | LNP | (mg/ml) | Overage |
| 1 | VZV-gE-oka- | IM | 8 | 10 | 50 | Day | | MC3 | 0.2 | 1 × 600 μl |
| | hIgkappa (G5; | | | | | 0 | | | | |
| | cap1) | | | | | | | | | |
| 2 | VZV-gE-oka- | IM | 8 | 10 | 50 | Day | Day | MC3 | 0.2 | 2 × 600 μl |
| | hIgkappa (G5; | | | | | 0 | 28 | | | |
| | cap1) | | | | | | | | | |
| 3 | VZV-gE-oka- | ID | 8 | 10 | 50 | Day | | MC3 | 0.2 | 1 × 600 μl |
| | hIgkappa (G5; | | | | | 0 | | | | |
| | cap1) | | | | | | | | | |
| 4 | VZV-gE-oka- | ID | 8 | 10 | 50 | Day | Day | MC3 | 0.2 | 2 × 600 μl |
| | hIgkappa (G5; | | | | | 0 | 28 | | | |
| | cap1) | | | | | | | | | |
| 5 | VZV-gE-oka | IM | 8 | 10 | 50 | Day | | MC3 | 0.2 | 1 × 600 μl |
| | (G0; cap1) | | | | | 0 | | | | |
| 6 | VZV-gE-oka | IM | 8 | 10 | 50 | Day | Day | MC3 | 0.2 | 2 × 600 μl |
| | (G0; cap1) | | | | | 0 | 28 | | | |
| 7 | VZV-gE-oka | ID | 8 | 10 | 50 | Day | | MC3 | 0.2 | 1 × 600 μl |
| | (G0; cap1) | | | | | 0 | | | | |
| 8 | VZV-gE-oka | ID | 8 | 10 | 50 | Day | Day | MC3 | 0.2 | 2 × 600 μl |
| | (G0; cap1) | | | | | 0 | 28 | | | |
| 9 | VZV-gE-oka | IM | 8 | 10 | 50 | Day | | MC3 | 0.2 | 1 × 600 μl |
| | (G5; cap1) | | | | | 0 | | | | |
| 10 | VZV-gE-oka | IM | 8 | 10 | 50 | Day | Day | MC3 | 0.2 | 2 × 600 μl |
| | (G5; cap1) | | | | | 0 | 28 | | | |
| 11 | VZV-gE-oka | ID | 8 | 10 | 50 | Day | | MC3 | 0.2 | 1 × 600 μl |
| | (G5; cap1) | | | | | 0 | | | | |
| 12 | VZV-gE-oka | ID | 8 | 10 | 50 | Day | Day | MC3 | 0.2 | 2 × 600 μl |
| | (G5; cap1) | | | | | 0 | 28 | | | |
| 13 | Negative | IM | 6 | / | 50 | Day | | PBS | / | 1 × 600 μl |
| | control (PBS) | | | | | 0 | | | | |
| 14 | Negative | IM | 6 | / | 50 | Day | Day | PBS | / | 2 × 600 μl |
| | control (PBS) | | | | | 0 | 28 | | | |
| 15 | Positive control | SC | 6 | 54 | 50 | Day | | | / | 1 × 1250 μl |
| | (VARIVAX ®) | | | (pfu) | | 0 | | | | |
| 16 | Positive control | SC | 6 | 54 | 50 | Day | Day | | / | |
| | (VARIVAX ®) | | | (pfu) | | 0 | 28 | | | |
| 17 | Positive control | SC | 4 | 675 | 100 | Day | | | / | 4 × 220 μl |
| | (VARIVAX ®) | | | (pfu) | | 0 | | | | |
| 18 | Positive control | SC | 4 | 675 | 100 | Day | Day | | / | |
| | (VARIVAX ®) | | | (pfu) | | 0 | 28 | | | |

TABLE 5
Schedule of Sample Collection

| | | Pre- | Day | Day | Day | Day | Day | Day |
|---|---|---|---|---|---|---|---|---|
| G# | Antigen | bleed | 0 + 6 h | 14 | 27 | 28 + 6 h | 42 | 56 |
| 1 | VZV-gE-oka- | √ | √ | √ | √ | | √ | √ |
| | hIgkappa | | | | | | | |
| | (G5; cap1) | | | | | | | |
| 2 | VZV-gE-oka- | √ | √ | √ | √ | √ | √ | √ |
| | hIgkappa | | | | | | | |
| | (G5; cap1) | | | | | | | |
| 3 | VZV-gE-oka- | √ | √ | √ | √ | | √ | √ |
| | hIgkappa | | | | | | | |
| | (G5; cap1) | | | | | | | |
| 4 | VZV-gE-oka- | √ | √ | √ | √ | √ | √ | √ |
| | hIgkappa | | | | | | | |
| | (G5; cap1) | | | | | | | |
| 5 | VZV-gE-oka | √ | | √ | √ | | √ | √ |
| | (G0; cap1) | | | | | | | |
| 6 | VZV-gE-oka | √ | | √ | √ | | √ | √ |
| | (G0; cap1) | | | | | | | |
| 7 | VZV-gE-oka | √ | | √ | √ | | √ | √ |
| | (G0; cap1) | | | | | | | |
| 8 | VZV-gE-oka | √ | | √ | √ | | √ | √ |
| | (G0; cap1) | | | | | | | |
| 9 | VZV-gE-oka | √ | | √ | √ | | √ | √ |
| | (G5; cap1) | | | | | | | |
| 10 | VZV-gE-oka | √ | | √ | √ | | √ | √ |
| | (G5; cap1) | | | | | | | |
| 11 | VZV-gE-oka | √ | | √ | √ | | √ | √ |
| | (G5; cap1) | | | | | | | |
| 12 | VZV-gE-oka | √ | | √ | √ | | √ | √ |
| | (G5; cap1) | | | | | | | |
| 13 | PBS | √ | √ | √ | √ | | √ | √ |
| 14 | PBS | √ | √ | √ | √ | √ | √ | √ |
| 15 | Positive control | √ | | √ | √ | | √ | √ |
| 16 | Positive control | √ | | √ | √ | | √ | √ |
| 17 | Positive control | √ | | √ | √ | | √ | √ |
| 18 | Positive control | √ | | √ | √ | | √ | √ |

### Example 16: Immunogenicity Study-ELISA

The instant studies were designed to test the immunogenicity in BALB/C mice of candidate VZV vaccines comprising a mRNA polynucleotide encoding glycoprotein gE from VZV. Mice were immunized with various VZV mRNA vaccine formulations at set intervals, and sera were collected after each immunization. The immunization schedule is provided in Table 2 of Example 15. The sera collection schedule is set forth in Table 4 of Example 15. Enzyme-linked immunosorbent assay (ELISA)

Serum antibody titers against VZV glycoprotein E were determined by Enzyme-linked immunosorbent assay (ELISA) using standard methods. In one study, the amount of anti-VZV gE mouse IgG was measured in the pre-bleed and in serum collected at day 14 and day 42 post vaccination in mice vaccinated intramuscularly with two 10 μg doses of either:

(1) VZV-gE-hlgkappa having 5′ cap: m⁷G (5′) ppp (5′) G-2′-O-methyl and N1-methylpseudouridine chemical modification (#1 in Tables 2 and 3); (2) VZV-gE having 5′ cap: m⁷G (5′) ppp (5′) G-2′-O-methyl, and no chemical modification (#6 in Tables 2 and 3); (3) VZV-gE having 5′ cap: m⁷G (5′) ppp (5′) G-2′-O-methyl and N1-methylpseudouridine chemical modification (#10 in Tables 2 and 3); (4) VARIVAX® vaccine (positive control); or (5) PBS (negative control).

FIGS. 5-7 show that there was a very strong immune response with all mRNA encoded VZV-gE vaccines tested relative to the current VARIVAX® vaccine. FIG. 5 shows that at day 14, the titer for anti-VZV-gE IgG was about 10 μg/mL in the serum of mice vaccinated with vaccine candidate (1) VZV-gE-hlgkappa having 5′ cap: m⁷G (5′) ppp (5′) G-2′-O-methyl and

NI-methylpseudouridine chemical modification and about 50 μg/mL in the serum of mice vaccinated with vaccine candidate (3) VZV-gE 5′ cap: m⁷G (5′) ppp (5′) G-2′-O-methyl and N1-methylpseudouridine chemical modification. The level of anti-VZV-gE IgG in the serum of mice vaccinated with VARIVAX® was not detectable at day 14. At day 42, the amount of anti-VZV-gE IgG present in the serum of mice vaccinated with vaccine candidate (1) VZV-gE-hlgkappa having 5′ cap: m⁷G (5′) ppp (5′) G-2′-O-methyl and N1-methylpseudouridine chemical modification or vaccine candidate (3) VZV-gE having 5′ cap: m⁷G (5′) ppp (5′) G-2′-O-methyl and N1-methylpseudouridine chemical modification was almost 1000-fold greater than the amount of anti-VZV-gE IgG present in the serum of mice vaccinated with VARIVAX®. The amount of anti-VZV gE IgG present in the serum of mice vaccinated with vaccine candidate (2) VZV-gE having 5′ cap: m⁷G (5′) ppp (5′) G-2′-O-methyl and no chemical modification was almost 100-fold greater than the amount of anti-VZV-gE IgG present in the serum of mice vaccinated with VARIVAX®. This study indicates that each of the VZV gE mRNA vaccines tested is a more immunogenic vaccine that the current VARIVAX® VZV vaccine.

### Example 17: Immunogenicity Study-ELISA

The instant studies were designed to test the immunogenicity in BALB/C mice of candidate VZV vaccines comprising a mRNA polynucleotide encoding glycoprotein gE from VZV. Mice were immunized with various VZV mRNA vaccine formulations at set intervals, and sera were collected after each immunization. The immunization schedule is provided in Table 4 of Example 15. The sera collection schedule is set forth in Table 5 of Example 15.

Serum antibody titers against VZV glycoprotein E was determined by Enzyme-linked immunosorbent assay (ELISA) using standard methods. In a second expanded study, the serum samples were serially diluted to bring the signal within the scope of detectability using ELISA. The amount of anti-VZV gE mouse IgG was measured in serum collected at day 42 post vaccination in mice vaccinated intramuscularly with two 10 μg doses of either: (1) VZV-gE-hlgkappa having 5′ cap: m⁷G (5′) ppp (5′) G-2′-O-methyl and N1-methylpseudouridine chemical modification (#1 in Tables 2 and 3); (2) VZV-gE having 5′ cap: m⁷G (5′) ppp (5′) G-2′-O-methyl and no chemical modification (#6 in Tables 2 and 3); (3) VZV-gE having 5′ cap: m⁷G (5′) ppp (5′) G-2′-O-methyl and N1-methylpseudouridine chemical modification (#10 in Tables 2 and 3); (4) VARIVAX® vaccine (positive control); or (5) PBS (negative control). The concentration of anti-VZV-gE mouse IgG was measured in 10-fold serial dilutions.

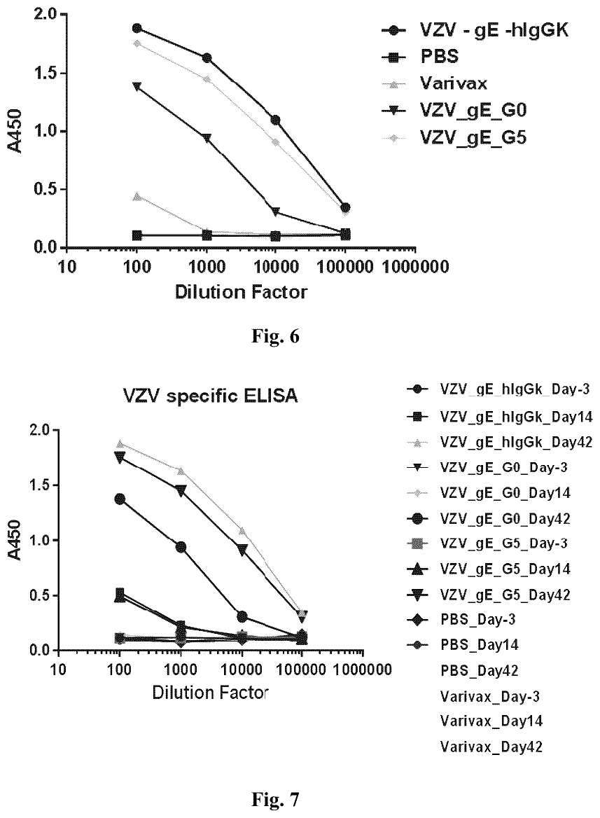

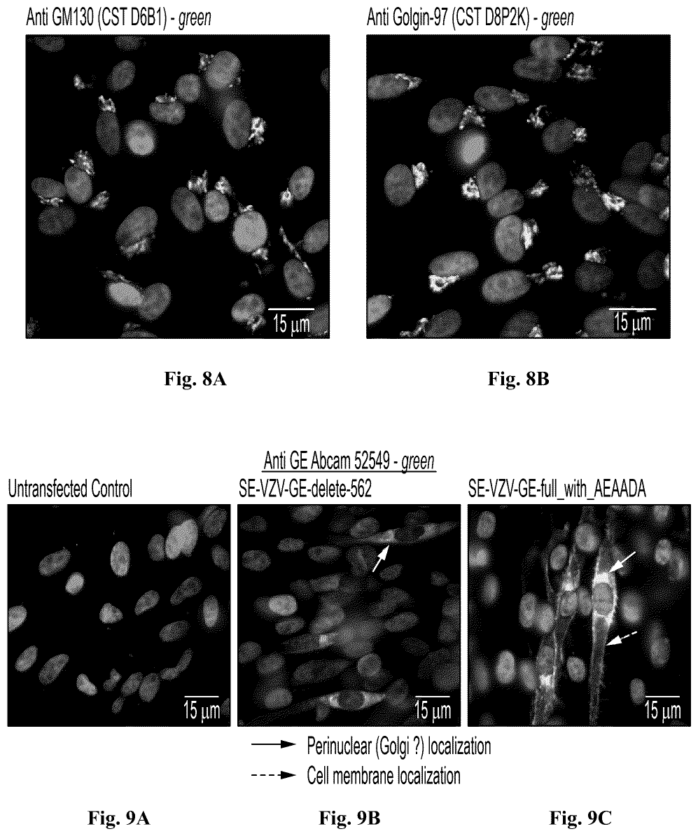

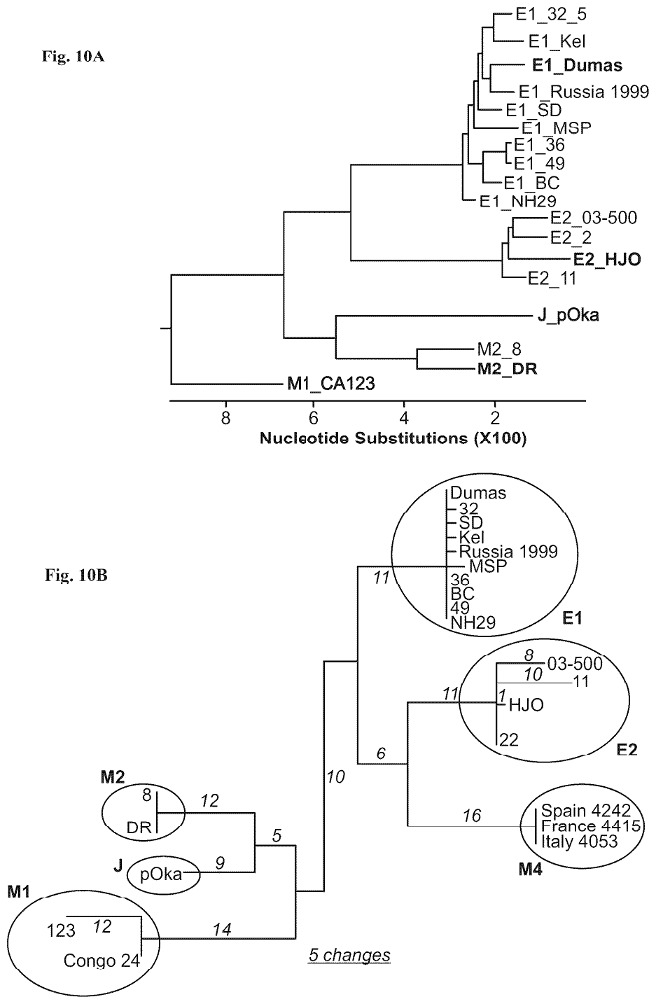

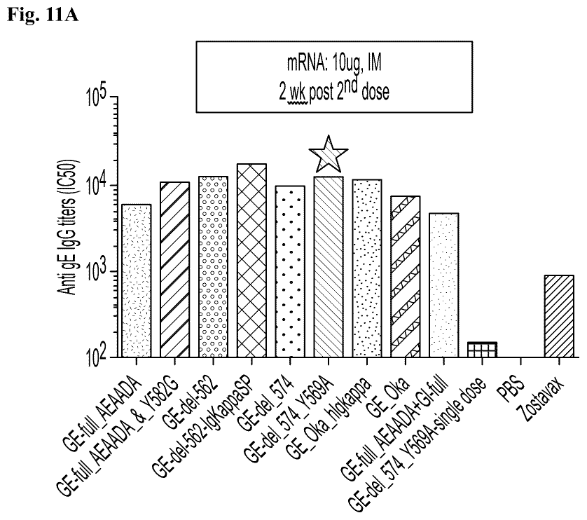

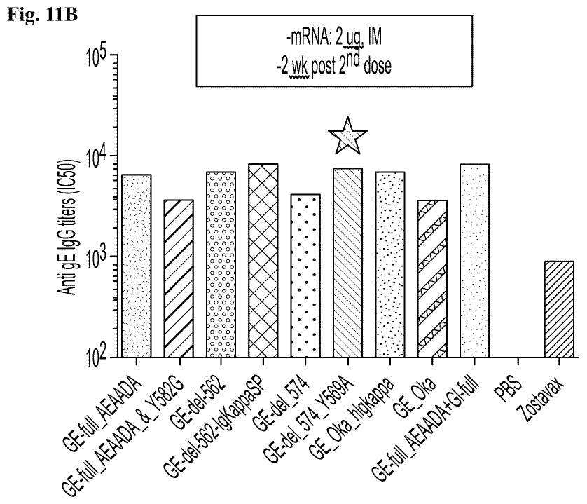

FIG. 6 shows that the strongest immune response was found in mice vaccinated with vaccine candidate (1) VZV-gE-hlgkappa having 5′ cap: m⁷G (5′) ppp (5′) G-2′-O-methyl and N1-methylpseudouridine chemical modification. The second strongest response was found in mice vaccinated with vaccine candidate (3) VZV-gE 5′ cap: m⁷G (5′) ppp (5′) G-2′-O-methyl and N1-methylpseudouridine chemical modification. The third strongest response was found in mice vaccinated with vaccine candidate (2) VZV-gE having 5′ cap: m⁷G (5′) ppp (5′) G-2′-O-methyl and no chemical modification. All three VZV gE mRNA vaccines generated a significantly greater immune response than VARIVAX® vaccine.

FIG. 7 shows the amount of anti-VZV-gE mouse IgG present in mice vaccinated with vaccines (1)-(4) and (5) negative control at day 3, day 14, and day 42 post-vaccination.

### Example 18: Immunogenicity Study

The instant studies are designed to test the immunogenicity in BALB/C mice of candidate VZV vaccines comprising a mRNA polynucleotide encoding variant glycoprotein gE from VZV. Mice were immunized with various VZV mRNA vaccine formulations at set intervals, and sera were collected after each immunization at indicated time points. The immunization schedule is provided in Table 6 below. The sera collection schedule is set forth in Table 7 below.

The amount of anti-VZV gE mouse IgG is measured in serum collected at the times indicated in Table 7 post vaccination in mice vaccinated intramuscularly with two 10 μg or 2 μg doses of the indicated constructs. All mRNAs used have the 5′ cap: m⁷G (5′) ppp (5′) G-2′-O-methyl and N1-methylpseudouridine chemical modification. ZOSTAVAX® was used as a positive control and was injected into mice intramuscularlarly with twice clinical dose of 19400 pfu SC. PBS was used as negative control.

Antibody titers against the VZV gE variant polypeptides in the sera of mice immunized with VZV gE variant mRNA vaccines indicated in Table 6 were determined by enzyme-linked immunosorbent assay (ELISA). To perform the ELISA, wells of a plate were coated with VZV gE antigen (Abcam: ab43050) in PBS. 100 μl of the VZV gE antigen at a concentration of 1, 2, or 4 μg/ml were used for coating overnight at 4° C. The wells were then washed with 300 μl of PBST (PBS with 0.05% tween) 3 times. The VZV gE-coated wells were blocked with 200 μl of blocking butter containing 1% Blotto in PBS for 30 minutes at room temperature. Mice sera containing anti-VZV gE antibodies were diluted 1:2000 and then subject to 1:3 serial dilutions using PBST. The diluted sera were added to the VZV gE-coated wells and incubated for 1 hour at room temperature. A secondary antibody, rabbit anti-mouse conjugated to horseradish peroxidase (HRP, Abcam: ab6728) was diluted 1:1000 in PBST and 100 μl of the secondary antibody containing solution was added to the wells and incubated for 45 minutes at room temperature. 100 μl of HRP substrates, KPL TMB, were added the wells and incubated for 3 minutes at room temperature before 100 μl of a stop solution (2M H₂SO₄) was added to stop the HRP reaction. Signals generated from the HRP substrates were measured at A450. The results were shown in FIGS. 11A, 11B, 12A, 12B, 13 and Tables 8 and 9.

FIG. 11A shows that all gE variants induced much stronger immune response than ZOSTAVAX® after the two 10 μg doses. FIG. 11B show that all gE variants induced much stronger immune response than ZOSTAVAX® after the two 2 μg doses. With both dosages, the gE variants GE-del_574_Y569A and GE-del-562-IgKappaSP induced the strongest immune response and the antibody titer measured in the sera of mice immunized with this gE variant is over 10 times more than the antibody titer measured in the sera of mice immunized with ZOSTAVAX®, indicating that the GE-del_574_Y569A and GE-del_562-IgKappaSP mRNAs are superior vaccine candidates against VZV.

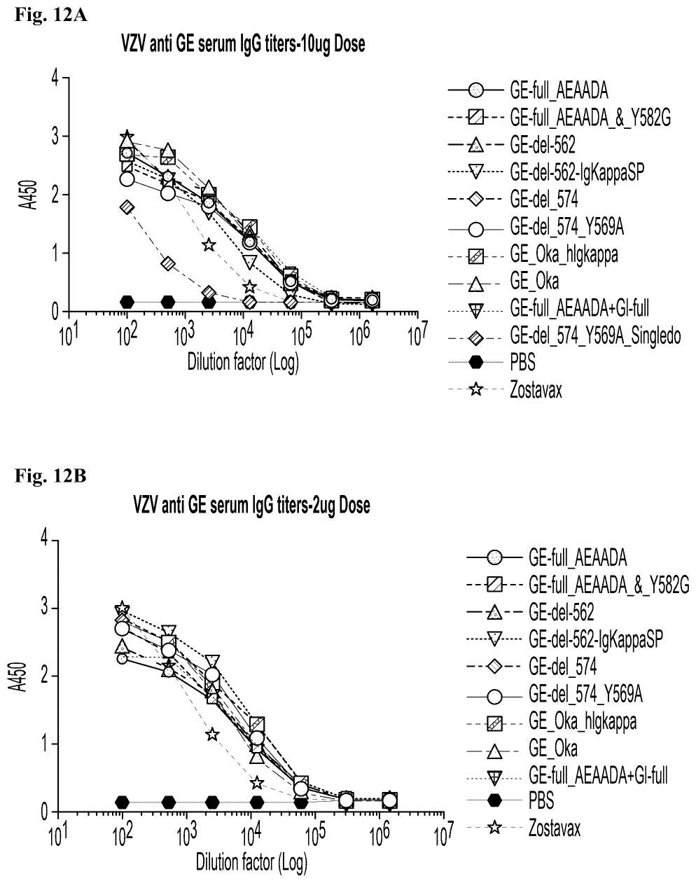

FIG. 12A shows the amount of antibodies in titrated sera collected from mice immunized twice with 10 μg of VZV gE mRNA variants described in Table 6. FIG. 12B shows the amount of antibodies in titrated sera collected from mice immunized twice with 2 μg of VZV gE mRNA variants described in Table 6. When the sera were diluted more than 100 fold, the antibody titer is higher in VZV gE variants vaccinated mice sera than in ZOSTAVAX® vaccinated mice sera, suggesting that the VZV gE mRNA variants induced much stronger immune response than ZOSTAVAX® in mice. All the VZV gE mRNA variants tested showed comparable ability in inducing immune response in mice.

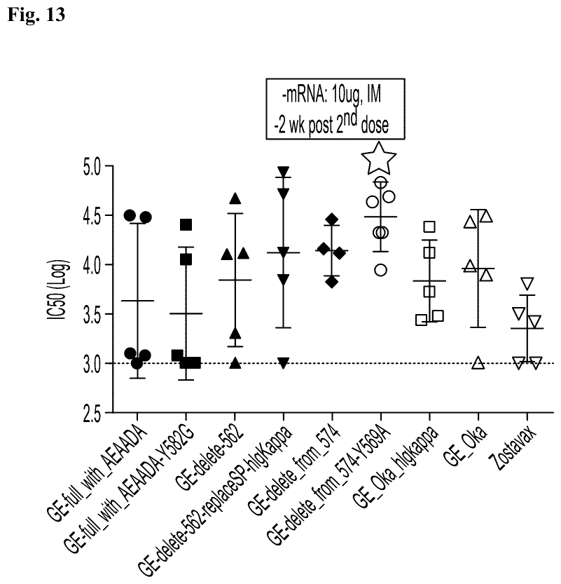

FIG. 13 is a graph showing the anti-VZV gE immune response induced by the VZV gE variant mRNA vaccines compared to ZOSTAVAX®. The VZV gE variant GE-delete_from_574-Y569A induced immune response in mice that is about 1 log greater than ZOSTAVAX®.

Table 9 summarizes the reciprocal IgG titer (IC50) in the sera collected from mice immunized with 10 μg or 2 μg of the respective VZV gE mRNAs twice. GE-delete_from_574-Y569A induced strong immune response with either 10 μg or 2 μg dosages. The Geometric Mean Titer (GMT) was used to indicate the immunogenic potential of the VZV gE variant mRNA vaccines. GE-delete_from_574-Y569A showed the highest GMT value, indicating that it is the most efficacious in inducing immune response against VZV gE.

TABLE 6
Injection Schedule

| | | | | | | Dose | | | | |
|---|---|---|---|---|---|---|---|---|---|---|
| | | | | | Dosage | Vol | 1^{st} | 2^{nd} | MC3/ | |
| G# | Antigen | mRNA# | Route | N | (μg) | (μl) | dose | dose | conc | |
| 1 | SE-VZV-GE-full_ | 1704271 | IM | 5 | 10 | 50 | Day | Day | 0.2 | 2 × 500 |
| | with_AEAADA | | | | | | 0 | 28 | mg/ml | |
| | (SEQ ID NO: 58) | | | | | | | | | |
| 2 | SE-VZV-GE-full_ | 1704271 | IM | 5 | 2 | 50 | Day | Day | 0.04 | 2 × 500 |
| | with_AEAADA | | | | | | 0 | 28 | mg/ml | |
| | (SEQ ID NO: 58) | | | | | | | | | |
| 3 | SE-VZV-GE-full_ | 1704265 | IM | 5 | 10 | 50 | Day | Day | 0.2 | 2 × 500 |
| | with_AEAADA | | | | | | 0 | 28 | mg/ml | |
| | (SEQ ID NO: 58)_ | | | | | | | | | |
| | and_Y582G | | | | | | | | | |
| 4 | SE-VZV-GE-full_ | 1704265 | IM | 5 | 2 | 50 | Day | Day | 0.04 | 2 × 500 |
| | with_AEAADA | | | | | | 0 | 28 | mg/ml | |
| | (SEQ ID NO: 58)_ | | | | | | | | | |
| | and_Y582G | | | | | | | | | |
| 5 | SE-VZV-GE- | 1704270 | IM | 5 | 10 | 50 | Day | Day | 0.2 | 2 × 500 |
| | delete-562 | | | | | | 0 | 28 | mg/ml | |
| 6 | SE-VZV-GE- | 1704270 | IM | 5 | 2 | 50 | Day | Day | 0.04 | 2 × 500 |
| | delete-562 | | | | | | 0 | 28 | mg/ml | |
| 7 | SE-VZV-GE- | 1704266 | IM | 5 | 10 | 50 | Day | Day | 0.2 | 2 × 500 |
| | delete-562- | | | | | | 0 | 28 | mg/ml | |
| | replacedSP- | | | | | | | | | |
| | withIgKappa | | | | | | | | | |
| 8 | SE-VZV-GE- | 1704266 | IM | 5 | 2 | 50 | Day | Day | 0.04 | 2 × 500 |
| | delete-562- | | | | | | 0 | 28 | mg/ml | |
| | replacedSP- | | | | | | | | | |
| | withIgKappa | | | | | | | | | |
| 9 | SE-VZV-GE- | 1704267 | IM | 5 | 10 | 50 | Day | Day | 0.2 | 2 × 500 |
| | truncated- | | | | | | 0 | 28 | mg/ml | |
| | delete_from_574 | | | | | | | | | |
| 10 | SE-VZV-GE- | 1704267 | IM | 5 | 2 | 50 | Day | Day | 0.04 | 2 × 500 |
| | truncated- | | | | | | 0 | 28 | mg/ml | |
| | delete_from_574 | | | | | | | | | |
| 11 | SE-VZV-GE- | 1704268 | IM | 5 | 10 | 50 | Day | Day | 0.2 | 2 × 500 |
| | truncated- | | | | | | 0 | 28 | mg/ml | |
| | delete_from_574_-_ | | | | | | | | | |
| | Y569A | | | | | | | | | |
| 12 | SE-VZV-GE- | 1704268 | IM | 5 | 2 | 50 | Day | Day | 0.04 | 2 × 500 |
| | truncated- | | | | | | 0 | 28 | mg/ml | |
| | delete_from_574_-_ | | | | | | | | | |
| | Y569A | | | | | | | | | |
| 13 | KB_VZV_gE_ | 1703872 | IM | 5 | 10 | 50 | Day | Day | 0.2 | 2 × 500 |
| | Oka_hIgkappa | | | | | | 0 | 28 | mg/ml | |
| 14 | KB_VZV_gE_ | 1703872 | IM | 5 | 2 | 50 | Day | Day | 0.04 | 2 × 500 |
| | Oka_hIgkappa | | | | | | 0 | 28 | mg/ml | |
| 15 | KB_VZV_gE_Oka | 1703869 | IM | 5 | 10 | 50 | Day | Day | 0.2 | 2 × 500 |
| | | | | | | | 0 | 28 | mg/ml | |
| 16 | KB_VZV_gE_Oka | 1703869 | IM | 5 | 2 | 50 | Day | Day | 0.04 | 2 × 500 |
| | | | | | | | 0 | 28 | mg/ml | |
| 17 | SE-VZV-GI-full | 1704269 | IM | 5 | 10 | 50 | Day | Day | 0.2 | 2 × 500 |
| | | | | | | | 0 | 28 | mg/ml | |
| 18 | SE-VZV-GI-full | 1704269 | IM | 5 | 2 | 50 | Day | Day | 0.04 | 2 × 500 |
| | | | | | | | 0 | 28 | mg/ml | |
| 19 | SE-VZV-GE-full_ | 1704271 + | IM | 5 | 10 | 50 | Day | Day | 0.2 | 2 × 500 |
| | with_AEAADA | 1704269 | | | | | 0 | 28 | mg/ml | |
| | (SEQ ID NO: 58) + | | | | | | | | | |
| | SE-VZV-GI-full | | | | | | | | | |
| 20 | SE-VZV-GE-full_ | 1704271 + | IM | 5 | 2 | 50 | Day | Day | 0.04 | 2 × 500 |
| | with_AEAADA | 1704269 | | | | | 0 | 28 | mg/ml | |
| | (SEQ ID NO: 58) + | | | | | | | | | |
| | SE-VZV-GI-full | | | | | | | | | |
| 21 | SE-VZV-GE- | 1704268 | IM | 5 | 10 | 50 | Day | No | 0.2 | 500 |
| | truncated-delete_ | | | | | | 0 | dosing | mg/ml | |
| | from_574_-_ | | | | | | | | | |
| | Y569A | | | | | | | | | |
| 22 | PBS | | IM | 5 | — | 50 | Day | Day | | 2 × 500 |
| | | | | | | | 0 | 28 | | |
| 23 | Positive control | ZOSTAVAX ® | SC | 5 | 19400 | 100 | Day | Day | | 2 × 500 |
| | | | | | PFUs | | 0 | 28 | | |

TABLE 7
Schedule of Sample Collection

| | | Pre- | Day | Day | Day | Day |
|---|---|---|---|---|---|---|
| G# | Antigen | bleed | 14 | 27 | 42 | 56 |
| 1 | SE-VZV-GE-full_with_AEAADA | ✓ | ✓ | ✓ | ✓ | ✓ |
| | (SEQ ID NO: 58) | | | | | |
| 2 | SE-VZV-GE-full_with_AEAADA | ✓ | ✓ | ✓ | ✓ | ✓ |
| | (SEQ ID NO: 58) | | | | | |
| 3 | SE-VZV-GE-full_with_AEAADA | ✓ | ✓ | ✓ | ✓ | ✓ |
| | (SEQ ID NO: 58)_and_Y582G | | | | | |
| 4 | SE-VZV-GE-full_with_AEAADA | ✓ | ✓ | ✓ | ✓ | ✓ |
| | (SEQ ID NO: 58)_and_Y582G | | | | | |
| 5 | SE-VZV-GE-delete-562 | ✓ | ✓ | ✓ | ✓ | ✓ |
| 6 | SE-VZV-GE-delete-562 | ✓ | ✓ | ✓ | ✓ | ✓ |
| 7 | SE-VZV-GE-delete-562-replacedSP- | ✓ | ✓ | ✓ | ✓ | ✓ |
| | withIgKappa | | | | | |
| 8 | SE-VZV-GE-delete-562-replacedSP- | ✓ | ✓ | ✓ | ✓ | ✓ |
| | withIgKappa | | | | | |
| 9 | SE-VZV-GE-truncated-delete_from_574 | ✓ | ✓ | ✓ | ✓ | ✓ |
| 10 | SE-VZV-GE-truncated-delete_from_574 | ✓ | ✓ | ✓ | ✓ | ✓ |
| 11 | SE-VZV-GE-truncated-delete_from_ | ✓ | ✓ | ✓ | ✓ | ✓ |
| | 574_-_Y569A | | | | | |
| 12 | SE-VZV-GE-truncated-delete_from_ | ✓ | ✓ | ✓ | ✓ | ✓ |
| | 574_-_Y569A | | | | | |
| 13 | KB_VZV_gE_Oka_hIgkappa | ✓ | ✓ | ✓ | ✓ | ✓ |
| 14 | KB_VZV_gE_Oka_hIgkappa | ✓ | ✓ | ✓ | ✓ | ✓ |
| 15 | KB_VZV_gE_Oka | ✓ | ✓ | ✓ | ✓ | ✓ |
| 16 | KB_VZV_gE_Oka | ✓ | ✓ | ✓ | ✓ | ✓ |
| 17 | SE-VZV-GI-full | ✓ | ✓ | ✓ | ✓ | ✓ |
| 18 | SE-VZV-GI-full | ✓ | ✓ | ✓ | ✓ | ✓ |
| 19 | SE-VZV-GE-full_with_AEAADA | ✓ | ✓ | ✓ | ✓ | ✓ |
| | (SEQ ID NO: 58) + SE-VZV-GI-full | | | | | |
| 20 | SE-VZV-GE-full_with_AEAADA | ✓ | ✓ | ✓ | ✓ | ✓ |
| | (SEQ ID NO: 58) + SE-VZV-GI-full | | | | | |
| 21 | SE-VZV-GE-truncated-delete_from_ | ✓ | ✓ | ✓ | ✓ | ✓ |
| | 574_-_Y569A | | | | | |
| 22 | PBS | ✓ | ✓ | ✓ | ✓ | ✓ |
| 23 | Positive control | ✓ | ✓ | ✓ | ✓ | ✓ |

TABLE 8
Summary of IC₅₀ of the different VZV constructs
Reciprocal IgG titer (IC50)

| Name | 10 ug | 2 ug | | | | |
|---|---|---|---|---|---|---|
| GE-FULL_AEAADA | 5741 | 6378 | | | | |
| GE-FULL_AEAADA_ | 10306 | 3556 | | | | |
| &_Y582G | | | | | | |
| GE-del-562 | 11672 | 6445 | | | | |
| GE-del-562-IaKappaSP | 16490 | 7939 | | | | |
| GE-del_574 | 9031 | 4082 | | | | |
| GE-del_574_Y569A | 11704 | 7291 | | | | |
| GE_Oka_hIgkappa | 11708 | 6448 | | | | |
| GE_Oka_hIgkappa | 7045 | 3672 | | | | |
| GE-full_AEAADA_ | 4457 | 8242 | | | | |
| GI-full | | | | | | |
| GE-del_574_Y569A | NA | | | | | |
| PBS | NA | NA | | | | |
| Zostavax | 860 | 860 | | | | |
| Assay controls | plate 1 | plate 2 | % CV | std | mean | CV |
| VZV_gE_Oka_ | 12803 | 11078 | 7.22 | 862.5 | 11940.5 | 0.07 |
| hIgkappa | | | | | | |

TABLE 9
Reciprocal anti-gE IgG titer (IC50) measured by ELISA

| Name | IC50 | GMT |
|---|---|---|
| GE-full_AEAADA | 1188.5 | 4291.4 |
| | 31915.4 | |
| | 1261.8 | |
| | 30408.9 | |
| | 1000 | |
| full_AEAADA_&_Y582G | 1150.8 | 3181.3 |
| | 25351.3 | |
| | 1000 | |
| | 1000 | |
| | 11168.6 | |
| GE-del-562 | 1000 | 6921.5 |
| | 47752.9 | |
| | 12676.5 | |
| | 12912.2 | |
| | 2032.4 | |
| GE-del-562-IgKappaSP | 1000 | 13140.1 |
| | 13122 | |
| | 51760.7 | |
| | 84918.1 | |
| | 6792 | |
| GE-del_574 | 13091.8 | 13795.9 |
| | 6760.8 | |
| | 14223.3 | |
| | 28774 | |
| GE-del_574_Y569A | 20941.1 | 30549.2 |
| | 48865.2 | |
| | 8810.5 | |
| | 43351.1 | |
| | 68076.9 | |
| GE_Oka_hIgkappa | 24266.1 | 6763.9 |
| | 3026.9 | |
| | 13213 | |
| | 5236 | |
| | 2786.1 | |
| GE-Oka | 27227 | 9078.2 |
| | 30903 | |
| | 7638.4 | |
| | 1000 | |
| | 9594 | |
| Zostavax | 6397.3 | 2228.4 |
| | 1000 | |
| | 1000 | |
| | 2660.7 | |
| | 3228.5 | |

### Example 19: VZV In Vitro Neutralization Assay

A VZV in vitro neutralization assay was performed to evaluate the anti-VZV gE antibodies in neutralizing VZV. The anti-VZV gE antibodies were obtained by collecting the sera of mice vaccinated with VZV gE variant mRNA vaccines. Mice were vaccinated with VZV gE variant mRNA vaccines at dosages or 10 μg or 2 μg as described in Table 6 and sera were collected 2 weeks post 2nd immunization.

To perform the assay, mice sera were diluted 1:5 and then subjected to 1:2 serial dilutions. VZV virus were added to the sera and neutralization was allowed to continue for 1 hour at room temperature.ARPE-19 cells were seeded in 96-wells one day before and the virus/serum mixtures were added to ARPE-19 cells at 50-100 pfu per well. The ARPE-19 cells were fixed on the next day and VZV-specific staining was performed. The plates were scanned and analyzed. Results of the VZV in vitro neutralization assay were summarized in Table 10. Values in Table 10 are serum dilutions showing 50% reduction in well-area coverage by VZV virus plaques. No reduction in plaque number was observed. As shown in Table 10, one replicate of serum from mice immunized with GE-delete_from_574-Y569A variant mRNA vaccine was able to reduce well-area coverage by VZV virus plaques at 1:80 dilution.

TABLE 10
In vitro neutralization assay

| | 10 ug | | 2 ug | |
|---|---|---|---|---|
| | Replicate | Replicate | Replicate | Replicate |
| Antigen | 1 | 2 | 1 | 2 |
| SE-VZV-GE-Full_with_ | 20 | 10 | 10 | 10 |
| AEAADA | | | | |
| SE-VZV-GE-Full_with_ | 40 | 20 | | 10 |
| AEAADA_and_Y582G | | | | |
| SE-VZV-GE-delete-562 | 40 | 20 | 20 | 10 |
| SE-VZV-GE-delete-562- | 40 | 40 | 20 | 40 |
| replacedSP-withIgKappa | | | | |
| SE-VZV-GE-delete_ | 80 | 40 | 40 | 20 |
| from_574 | | | | |
| SE-VZV-GE-delete_ | 20 | 80 | 10 | 10 |
| from_574-Y569A | | | | |
| KB_VZV_gE_Oka_ | 40 | <20 | 20 | 10 |
| hIgkappa | | | | |
| KB_VZV_gE_Oka | 160 | 10 | 10 | 10 |
| SE-VZV-GI-full | <10 | <10 | <20 | <20 |
| SE-VZV-GE-full_with_ | 20 | 10 | — | — |
| AEAADA + SE-VZV- | | | | |
| GI-full | | | | |
| SE-VZV-GE-truncated- | <10 | <10 | — | — |
| delete_from_574-Y569A | | | | |
| PBS | <20 | <20 | | |
| Positive Control | 20 | 20 | | |

### Example 20: Immunogenicity in Mice

Herpes zoster (HZ) or shingles is a debilitating disease characterized by a vesicular rash, with the most common complication being post-herpetic neuralgia (PHN). PHN is a constant and severe pain that develops after clearance of the cutaneous outbreak, and can last for several years, thereby contributing to the high morbidity of affected individuals. HZ is caused by reactivation of latent varicella-zoster virus (VZV) from the sensory ganglia. Immune responses generated during primary VZV infection (chickenpox) have been shown to prevent the reactivation of latent VZV. However, the incidence of HZ is strongly associated with advancing age. Several investigations have shown that T cell-mediated immune responses decline with increasing age and during immunosuppression, resulting in reactivation of VZV. Nonetheless, the levels of anti-VZV antibodies remain relatively stable with increasing age, demonstrating that the humoral immune response may not be sufficient for the prevention of HZ. Several studies have reported the induction of VZV-specific CD4+ and CD8⁺ T cells, with CD4⁺ T cells dominating the memory response.

The approved vaccine Zostavax demonstrates around 60-70% efficacy in 50-60 years adults and declines with age. Recently a subunit adjuvanted vaccine (Shingrix: gE protein+ASO1B) was shown to have ˜90% efficacy in all age group of adults 50+. However, this vaccine demonstrated grade 3 severe AE's in 10% of the vaccinated subjects. Shingrix demonstrated about a log fold better T and B cell response after two doses to Zostavax. In the present studies mRNA immunization with a gE construct was investigated for immunogenicity in mice and NHP.

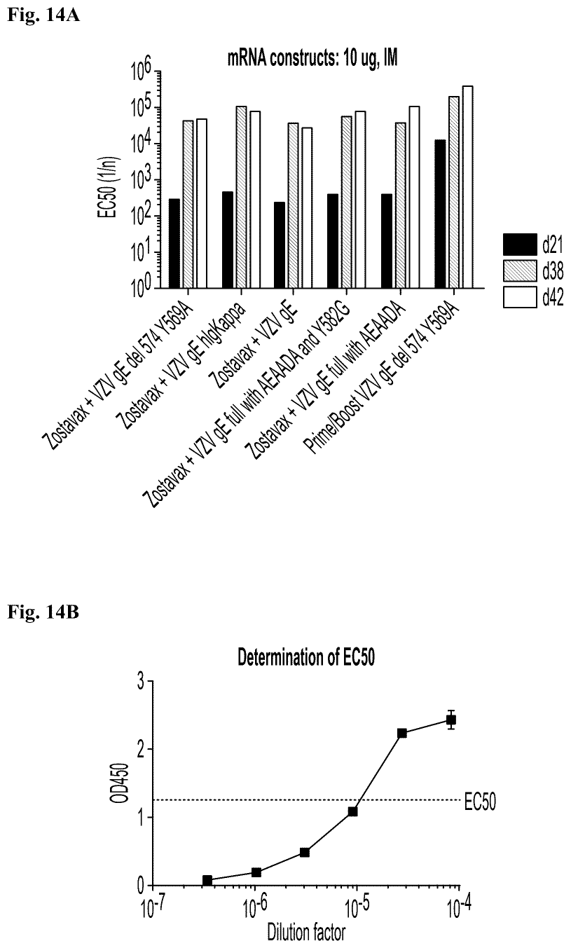

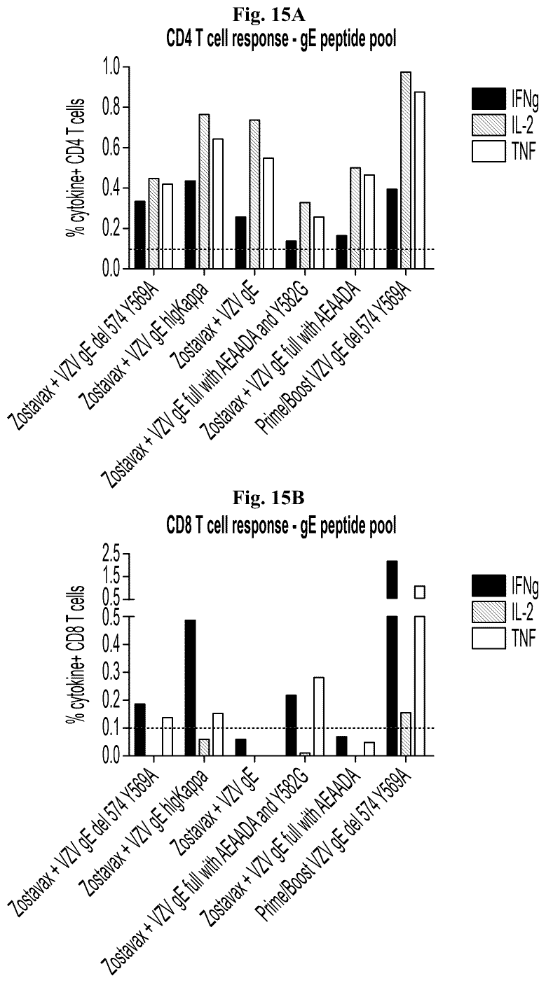

The instant study was defined to test the immunogenicity in BALB/C mice of candidate VZV vaccines comprising a mRNA polynucleotide encoding glycoprotein gE from VZV. Mice were immunized with various VZV mRNA vaccine formulations as set forth below in Table 11. Groups 1 to 5 were primed with ZOSTAVAX® to mimic a primary Varicella exposure and group 6 was primed with mRNA construct VZV-gE-del_574_Y569A. All groups (groups 1-6) were boosted on day 28, as shown in Table 11. The animals were bled on days-3, 21, 38 and 42. Blood and spleens were collected for serological and T cell analysis on days 38 and 42. As shown in FIG. 14A, all groups gave comparable anti-gE antibody responses to mRNA vaccination with the animal receiving mRNA for prime and boost (group 6) trending higher. As shown in FIG. 15A, all groups demonstrated CD4 T-cell responses with mRNA prime and boost (group 6) trending towards a higher response. As shown in FIG. 15B, variable CD8 T-cell responses were noted with the mRNA prime and boost (group 6) demonstrating highest CD8 T-cell frequencies.

TABLE 11
Injection Schedule

| | Primary | | Localization | |
|---|---|---|---|---|
| | Immunization | Boost | (VERO/MeWo) | Number |
| | (Day 0) | (Day 28) | of Boost | mice per |
| G# | 10 μg | 10 μg | Construct | group |
| 1 | ZOSTAVAX ® | VZV-gE-del_ | Golgi (high) | 10 |
| | | 574_Y569A | & cell | |
| | | | membrane | |
| | | | (high) | |
| 2 | ZOSTAVAX ® | VZV-gE-Oka- | Golgi (low) & | 10 |
| | | hIgKappa | cytoplasmic | |
| 3 | ZOSTAVAX ® | VZV-gE-Oka | Golgi/Golgi | 10 |
| 4 | ZOSTAVAX ® | VZV-gE | Organelles & | 10 |
| | | full_with_ | cytoplasmic/cell | |
| | | AEAADA | membrane | |
| | | (SEQ ID NO: | | |
| | | 58)_and_Y582G | | |
| 5 | ZOSTAVAX ® | VZV-gE-full with | Golgi & diffuse | 10 |
| | | AEAADA (SEQ | perinuclear/cell | |
| | | ID NO: 58) | membrane | |
| 6 | VZV-gE-del_ | VZV-gE-del_ | Golgi (high) | 10 |
| | 574_Y569A | 574_Y569A | & cell | |
| | | | membrane | |
| | | | (high) | |

### Example 21: Immunogenicity in Non-Human Primates

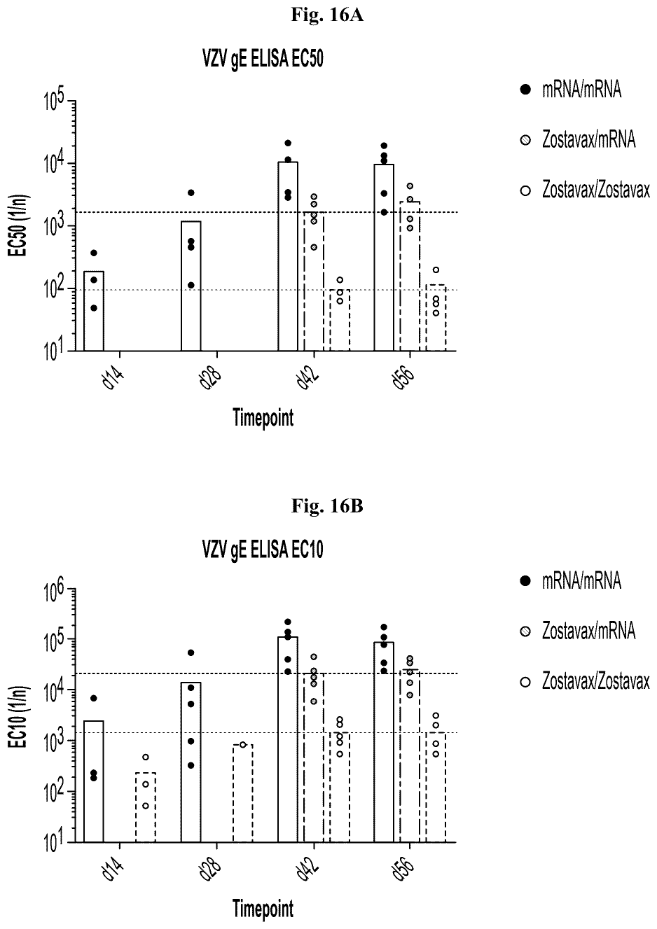

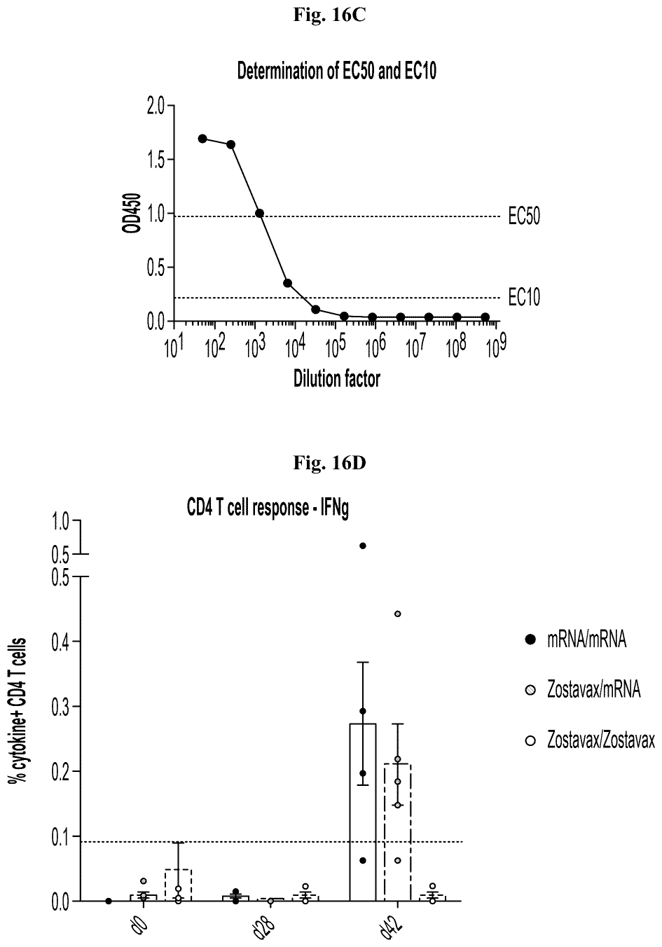

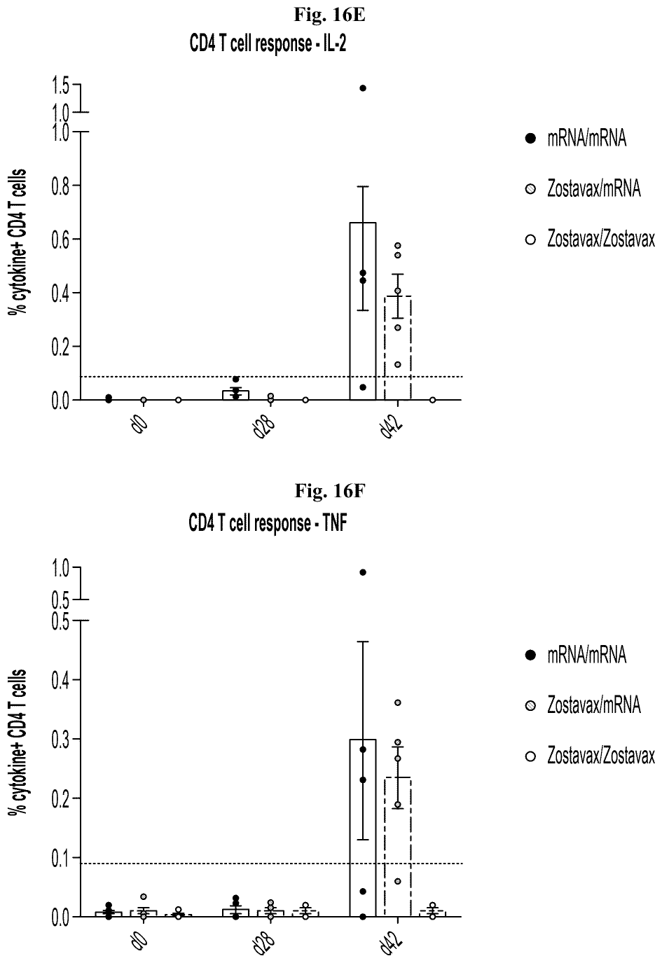

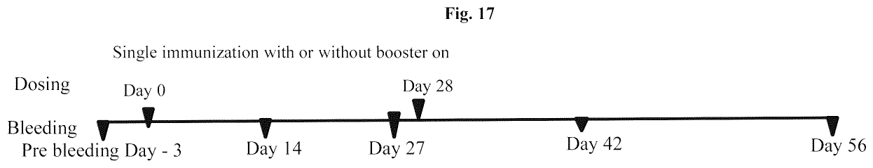

Based on the data in Example 20, the mRNA construct VZV-gE-del_574_Y569A was further evaluated for immunogenicity in non-human primates. Three groups of Rhesus monkeys were primed with mRNA (VZV-gE-del_574_Y569A) or ZOSTAVAX® and boosted as set forth below in Table 12. The animals were bled at days 0, 14, 28, and 42 for serological and T-cell analysis. T-cell analysis was performed on days 0, 28, and 42. As shown in FIGS. 16A and 16B, the mRNA prime and boost (group 1) gave the highest anti-gE titers which were followed by ZOSTAVAX® prime, mRNA boost (group 2). The latter group (group 2) anti-gE titers were approximately 10× better than the ZOSTAVAX® prime, ZOSTAVAX® boost (group 3). As shown in FIGS. 16C and 16D, no CD4-T cells producing IFNγ, IL-2 or TNFα were detected in the ZOSTAVAX® prime, ZOSTAVAX® boost group (group 3). In contrast, as shown in FIGS. 16C and 16D, reasonable frequency of CD4 T-cells producing IFNγ, IL-2 or TNFα were detected in the mRNA prime, mRNA boost group (group 1) and the ZOSTAVAX® prime, mRNA boost group (group 2) and were statistically undifferentiated. These data indicate that one dose of mRNA vaccination after Zostavax exposure was equivalent to two doses of mRNA vaccination in inducing comparable T-cell responses.

TABLE 12
Injection Schedule

| | Primary | | Number Rhesus |
|---|---|---|---|
| | Immunization | Boost | macaques |
| | (Day 0) | (Day 28) | (male and female) |
| G# | 10 μg | 10 μg | per group |
| 1 | VZV-gE-del_ | VZV-gE-del_ | 5 |
| | 574_Y569A | 574_Y569A | |
| 2 | ZOSTAVAX ® | VZV-gE-del_ | 5 |
| | | 574_Y569A | |
| 3 | ZOSTAVAX ® | ZOSTAVAX ® | 5 |

TABLE 13
Varicella zoster virus Amino Acid Sequences

| | | GenBank |
|---|---|---|
| Protein | Name | Accession |
| glycoprotein B | envelope glycoprotein B [Human | NP_040154.2 |
| | herpesvirus 3] | |
| glycoprotein B | ORF31 [Human herpesvirus 3] | AKG57704.1 |
| glycoprotein B | ORF 31 [Human herpesvirus 3] | AIT52967.1 |
| glycoprotein B | envelope glycoprotein B [Human | AFJ68532.1 |
| | herpesvirus 3] | |
| glycoprotein B | ORF31 [Human herpesvirus 3] | AKG57414.1 |
| glycoprotein B | ORF31 [Human herpesvirus 3] | AKG58507.1 |
| glycoprotein B | RecName: Full = Envelope | Q4JR05.2 |
| | glycoprotein B; Short = gB; AltName: | |
| | Full = Glycoprotein II; Flags: | |
| | Precursor [Human herpesvirus 3 strain | |
| | Oka vaccine] | |
| glycoprotein B | ORF31 [Human herpesvirus 3] | AEL30845.1 |
| glycoprotein B | glycoprotein B [Human herpesvirus 3] | AAK01041.1 |
| glycoprotein B | glycoprotein B [Human herpesvirus 3] | AEW89232.1 |
| glycoprotein B | glycoprotein B [Human herpesvirus 3] | AEW88728.1 |
| glycoprotein B | ORF31 [Human herpesvirus 3] | AAK19938.1 |
| glycoprotein B | glycoprotein B [Human herpesvirus 3] | AAP32845.1 |
| glycoprotein B | ORF 31 [Human herpesvirus 3] | AHJ08729.1 |
| glycoprotein B | ORF31 [Human herpesvirus 3] | AAY57715.1 |
| glycoprotein B | ORF31 [Human herpesvirus 3] | AGY33726.1 |
| glycoprotein B | ORF 31 [Human herpesvirus 3] | AHJ09321.1 |
| glycoprotein B | ORF31 [Human herpesvirus 3] | AAY57644.1 |
| glycoprotein B | ORF 31 [Human herpesvirus 3] | AHJ09025.1 |
| glycoprotein B | glycoprotein B [Human herpesvirus 3] | AEW88584.1 |
| glycoprotein B | ORF 31 [Human herpesvirus 3] | AHJ09099.1 |
| glycoprotein B | ORF31 [Human herpesvirus 3] | AGY33060.1 |
| glycoprotein B | ORF 31 [Human herpesvirus 3] | AHJ09395.1 |
| glycoprotein C | RecName: Full = Envelope | P09256.2 |
| | glycoprotein C; Short = gC; AltName: | |
| | Full = Glycoprotein V; Short = gpV | |
| glycoprotein C | envelope glycoprotein gC [Human | ABH08453.1 |
| | herpesvirus 3] | |
| glycoprotein C | ORF14 [Human herpesvirus 3] | AIH07125.1 |
| glycoprotein C | unknown protein [Human herpesvirus | AAA69563.1 |
| | 3] | |
| glycoprotein C | ORF14 [Human herpesvirus 3] | AIH07051.1 |
| glycoprotein C | ORF14 [Human herpesvirus 3] | AIJ28607.1 |
| glycoprotein C | ORF14 [Human herpesvirus 3] | AEL30828.1 |
| glycoprotein C | envelope glycoprotein gC [Human | ABE03032.1 |
| | herpesvirus 3] | |
| glycoprotein C | envelope glycoprotein gC [Human | ABE67122.1 |
| | herpesvirus 3] | |
| glycoprotein C | envelope glycoprotein C [Human | NP_040137.1 |
| | herpesvirus 3] | |
| glycoprotein C | membrane glycoprotein C [Human | AEW88351.1 |
| | herpesvirus 3] | |
| glycoprotein C | envelope glycoprotein C [Human | AFJ68515.1 |
| | herpesvirus 3] | |
| glycoprotein C | membrane glycoprotein C [Human | AAT07696.1 |
| | herpesvirus 3] | |
| glycoprotein C | envelope glycoprotein gC [Human | ABF22098.1 |
| | herpesvirus 3] | |
| glycoprotein C | membrane glycoprotein C [Human | AEW89287.1 |
| | herpesvirus 3] | |
| glycoprotein C | glycoprotein C [Human herpesvirus 3] | AGC94505.1 |
| glycoprotein C | envelope glycoprotein gC [Human | ABF21514.1 |
| | herpesvirus 3] | |
| glycoprotein C | envelope glycoprotein gC [Human | ABF21879.1 |
| | herpesvirus 3] | |
| glycoprotein C | envelope glycoprotein gC [Human | ABF21587.1 |
| | herpesvirus 3] | |
| glycoprotein C | ORF 14 [Human herpesvirus 3] | AIT53315.1 |
| glycoprotein C | membrane glycoprotein C [Human | AEW89215.1 |
| | herpesvirus 3] | |
| glycoprotein C | envelope glycoprotein gC [Human | ABF21660.1 |
| | herpesvirus 3] | |
| glycoprotein C | membrane glycoprotein C [Human | AEW88567.1 |
| | herpesvirus 3] | |
| glycoprotein C | envelope glycoprotein gC [Human | CAI44857.1 |
| | herpesvirus 3] | |
| glycoprotein C | envelope glycoprotein C [Human | AHB80244.1 |
| | herpesvirus 3] | |
| glycoprotein C | ORF14 [Human herpesvirus 3] | AAY57702.1 |
| glycoprotein C | glycoprotein c [Human herpesvirus 3] | AGS32072.1 |
| glycoprotein C | envelope glycoprotein gC [Human | AGL50971.1 |
| | herpesvirus 3] | |
| glycoprotein C | membrane glycoprotein C [Human | AAT07772.1 |
| | herpesvirus 3] | |
| glycoprotein C | membrane glycoprotein C [Human | AEW88495.1 |
| | herpesvirus 3] | |
| glycoprotein C | ORF 14 [Human herpesvirus 3] | AIT53461.1 |
| glycoprotein C | ORF 14 [Human herpesvirus 3] | AIT52950.1 |
| glycoprotein C | ORF14 [Human herpesvirus 3] | AAY57631.1 |
| glycoprotein C | envelope glycoprotein gC [Human | ABF21952.1 |
| | herpesvirus 3] | |
| glycoprotein C | membrane glycoprotein C [Human | AEW89143.1 |
| | herpesvirus 3] | |
| glycoprotein C | membrane glycoprotein C [Human | AEW88783.1 |
| | herpesvirus 3] | |
| glycoprotein C | membrane glycoprotein C [Human | AEW88999.1 |
| | herpesvirus 3] | |
| glycoprotein C | membrane glycoprotein C [Human | AEW88063.1 |
| | herpesvirus 3] | |
| glycoprotein C | membrane glycoprotein C [Human | AEW89071.1 |
| | herpesvirus 3] | |
| glycoprotein C | membrane glycoprotein C [Human | AEW88639.1 |
| | herpesvirus 3] | |
| glycoprotein C | membrane glycoprotein C [Human | AEW87991.1 |
| | herpesvirus 3] | |
| glycoprotein C | ORF 14 [Human herpesvirus 3] | AIT53753.1 |
| glycoprotein C | ORF 14 [Human herpesvirus 3] | AIT53096.1 |
| glycoprotein C | envelope glycoprotein gC [Human | ABF22025.1 |
| | herpesvirus 3] | |
| glycoprotein C | envelope glycoprotein gC [Human | AFO85518.1 |
| | herpesvirus 3] | |
| glycoprotein C | envelope glycoprotein gC [Human | ABF21733.1 |
| | herpesvirus 3] | |
| glycoprotein C | envelope glycoprotein gC [Human | ABF21806.1 |
| | herpesvirus 3] | |
| glycoprotein C | membrane glycoprotein C [Human | AEW89359.1 |
| | herpesvirus 3] | |
| glycoprotein C | membrane glycoprotein C [Human | AEW88855.1 |
| | herpesvirus 3] | |
| glycoprotein C | envelope glycoprotein gC [Human | AFO85591.1 |
| | herpesvirus 3] | |
| glycoprotein C | membrane glycoprotein C [Human | AEW89431.1 |
| | herpesvirus 3] | |
| glycoprotein C | membrane glycoprotein C [Human | AEW88711.1 |
| | herpesvirus 3] | |
| glycoprotein C | membrane glycoprotein C [Human | AEW88135.1 |
| | herpesvirus 3] | |
| glycoprotein C | membrane glycoprotein C [Human | AEW88927.1 |
| | herpesvirus 3] | |
| glycoprotein C | ORF14 [Human herpesvirus 3] | AKG56156.1 |
| glycoprotein C | ORF14 [Human herpesvirus 3] | AKG57178.1 |
| glycoprotein C | ORF14 [Human herpesvirus 3] | AKG58125.1 |
| glycoprotein C | ORF14 [Human herpesvirus 3] | AGY32970.1 |
| glycoprotein C | ORF14 [Human herpesvirus 3] | AKG56229.1 |
| glycoprotein C | ORF14 [Human herpesvirus 3] | AGY32896.1 |
| glycoprotein C | ORF14 [Human herpesvirus 3] | AKG56521.1 |
| glycoprotein C | ORF 14 [Human herpesvirus 3] | AHJ08712.1 |
| glycoprotein E | unknown [Human herpesvirus 3] | ABE03086.1 |
| glycoprotein E | glycoprotein E [Human herpesvirus 3] | AAK01047.1 |
| glycoprotein E | RecName: Full = Envelope | Q9J3M8.1 |
| | glycoprotein E; Short = gE; Flags: | |
| | Precursor | |
| glycoprotein E | membrane glycoprotein E [Human | AEW88548.1 |
| | herpesvirus 3] | |
| glycoprotein E | ORF68 [Human herpesvirus 3] | AGY33616.1 |
| glycoprotein E | membrane glycoprotein E [Human | AEW89124.1 |
| | herpesvirus 3] | |
| glycoprotein E | ORF 68 [Human herpesvirus 3] | AIT53150.1 |
| glycoprotein E | unnamed protein product [Human | CAA25033.1 |
| | herpesvirus 3] | |
| glycoprotein E | envelope glycoprotein E [Human | NP_040190.1 |
| | herpesvirus 3] | |
| glycoprotein E | ORF68 [Human herpesvirus 3] | AKG56356.1 |
| glycoprotein E | membrane glycoprotein E [Human | AEW89412.1 |
| | herpesvirus 3] | |
| glycoprotein E | membrane glycoprotein gE [Human | ABF21714.1 |
| | herpesvirus 3] | |
| glycoprotein E | membrane glycoprotein E [Human | AAT07749.1 |
| | herpesvirus 3] | |
| glycoprotein E | membrane glycoprotein E [Human | AEW88764.1 |
| | herpesvirus 3] | |
| glycoprotein E | glycoprotein E [Human herpesvirus 3] | AAG48520.1 |
| glycoprotein E | membrane glycoprotein E [Human | AEW88980.1 |
| | herpesvirus 3] | |
| glycoprotein H | envelope glycoprotein H [Human | NP_040160.1 |
| | herpesvirus 3] | |
| glycoprotein H | glycoprotein H [Human herpesvirus | AEW89454.1 |
| | 3] | |
| glycoprotein H | ORF37 [Human herpesvirus 3 VZV- | AAK19252.1 |
| | 32] | |
| glycoprotein H | RecName: Full = Envelope | Q775J3.1 |
| | glycoprotein H; Short = gH; AltName: | |
| | Full = Glycoprotein III; Short = | |
| | GPIII; Flags: Precursor | |
| glycoprotein H | glycoprotein H [Human herpesvirus | AAK01042.1 |
| | 3] | |
| glycoprotein H | ORF37 [Human herpesvirus 3 | AKG58587.1 |
| glycoprotein H | ORF37 [Human herpesvirus 3] | AGY33215.1 |
| glycoprotein H | glycoprotein H [Human herpesvirus | AAP32857.1 |
| | 3] | |
| glycoprotein H | envelope glycoprotein gH [Human | ABE03056.1 |
| | herpesvirus 3] | |
| glycoprotein H | ORF 37 [Human herpesvirus 3] | AHJ09328.1 |
| glycoprotein H | glycoprotein H [Human herpesvirus | AAP32862.1 |
| | 3] | |
| glycoprotein H | ORF37 [Human herpesvirus 3] | AKG57421.1 |
| glycoprotein H | ORF37 [Human herpesvirus 3] | AKG56618.1 |
| glycoprotein H | ORF37 [Human herpesvirus 3] | AKG56545.1 |
| glycoprotein H | glycoprotein H [Human herpesvirus | AEW89382.1 |
| | 3] | |
| glycoprotein H | glycoprotein H [Human herpesvirus | AGC94548.1 |
| | 3] | |
| glycoprotein I | envelope glycoprotein I [Human | NP_040189.1 |
| | herpesvirus 3] | |
| glycoprotein I | membrane glycoprotein I [Human | AEW89195.1 |
| | herpesvirus 3] | |
| glycoprotein I | ORF67 [Human herpesvirus 3] | AKG58616.1 |
| glycoprotein I | ORF67 [Human herpesvirus 3] | AGY34059.1 |
| glycoprotein I | membrane glycoprotein I [Human | AEW89051.1 |
| | herpesvirus 3] | |
| glycoprotein I | ORF67 [Human herpesvirus 3 VZV- | AAK19249.1 |
| | 32] | |
| glycoprotein I | membrane glycoprotein I [Human | AEW89483.1 |
| | herpesvirus 3] | |
| glycoprotein K | envelope glycoprotein K [Human | NP_040128.1 |
| | herpesvirus 3] | |
| glycoprotein K | glycoprotein K [Human herpesvirus | AEW88773.1 |
| | 3] | |
| glycoprotein K | ORF 5 [Human herpesvirus 3] | AHJ09368.1 |
| glycoprotein K | ORF5 [Human herpesvirus 3] | AKG58699.1 |
| glycoprotein K | glycoprotein K [Human herpesvirus | AEW88701.1 |
| | 3] | |
| glycoprotein K | ORF5 [Human herpesvirus 3] | AKG56803.1 |
| glycoprotein K | glycoprotein K [Human herpesvirus | AEW88053.1 |
| | 3] | |
| glycoprotein L | RecName: Full = Envelope | Q9J3N1.1 |
| | glycoprotein L; Short = gL; Flags: | |
| | Precursor | |
| glycoprotein L | virion glycoprotein gL [Human | ABE03078.1 |
| | herpesvirus 3] | |
| glycoprotein L | glycoprotein L [Human herpesvirus 3] | AGM33094.1 |
| glycoprotein L | ORF60 [Human herpesvirus 3] | AKG56786.1 |
| glycoprotein L | envelope glycoprotein L [Human | NP_040182.1 |
| | herpesvirus 3] | |
| glycoprotein L | virion glycoprotein gL [Human | ABF21706.1 |
| | herpesvirus 3] | |
| glycoprotein M | envelope glycoprotein M [Human | NP_040172.1 |
| | herpesvirus 3] | |
| glycoprotein M | ORF 50 [Human herpesvirus 3] | AIT53351.1 |
| glycoprotein M | ORF50 [Human herpesvirus 3] | AKG56119.1 |
| glycoprotein M | ORF50 [Human herpesvirus 3] | AGY33080.1 |
| glycoprotein M | envelope glycoprotein gM [Human | ABE03068.1 |
| | herpesvirus 3] | |
| glycoprotein M | virion membrane glycoprotein M | AEW88530.1 |
| | [Human herpesvirus 3] | |
| glycoprotein M | virion membrane glycoprotein M | AEW88674.1 |
| | [Human herpesvirus 3] | |
| glycoprotein N | envelope glycoprotein N [Human | YP_068406.1 |
| | herpesvirus 3] | |
| glycoprotein N | ORF9a [Human herpesvirus 3] | AGY33038.1 |
| glycoprotein N | membrane protein [Human | AAT07690.1 |
| | herpesvirus 3] | |
| glycoprotein N | membrane protein [Human | AEW88273.1 |
| | herpesvirus 3] | |
| glycoprotein N | membrane protein [Human | AEW88489.1 |
| | herpesvirus 3] | |

TABLE 14
VZV Polypeptide Sequences

| Name | Sequence | SEQ ID NO: |
|---|---|---|
| gi\|443500676\|gb\| | MGTVNKPVVGVLMGFGIITGTLRITNPVRASVLRYDDF | 45 |
| AGC94542.1\| | HIDEDKLDTNSVYEPYYHSDHAESSWVNRGESSRKAY | |
| glycoprotein E | DHNSPYIWPRNDYDGFLENAHEHHGVYNQGRGIDSGE | |
| [Human | RLMQPTQMSAQEDLGDDTGIHVIPTLNGDDRHKIVNV | |
| herpesvirus 3] | DQRQYGDVFKGDLNPKPQGQRLIEVSVEENHPFTLRAP | |
| | IQRIYGVRYTETWSFLPSLTCTGDAAPAIQHICLKHTTC | |
| | FQDVVVDVDCAENTKEDQLAEISYRFQGKKEADQPWI | |
| | VVNTSTLFDELELDPPEIEPGVLKVLRTEKQYLGVYIW | |
| | NMRGSDGTSTYATFLVTWKGDEKTRNPTPAVTPQPRG | |
| | AEFHMWNYHSHVFSVGDTFSLAMHLQYKIHEAPFDLL | |
| | LEWLYVPIDPTCQPMRLYSTCLYHPNAPQCLSHMNSG | |
| | CTFTSPHLAQRVASTVYQNCEHADNYTAYCLGISHME | |
| | PSFGLILHDGGTTLKFVDTPESLSGLYVFVVYFNGHVE | |
| | AVAYTVVSTVDHFVNAIEERGFPPTAGQPPATTKPKEIT | |
| | PVNPGTSPLLRYAAWTGGLAAVVLLCLVIFLICTAKRM | |
| | RVKAYRVDKSPYNQSMYYAGLPVDDFEDSESTDTEEE | |
| | FGNAIGGSHGGSSYTVYIDKTR | |
| gi\|443500675\|gb\| | MFLIQCLISAVIFYIQVTNALIFKGDHVSLQVNSSLTSILI | 46 |
| AGC94541.1\| | PMQNDNYTEIKGQLVFigEQLPTGTNYSGTLELLYADT | |
| glycoprotein I | VAFCFRSVQVIRYDGCPRIRTSAFISCRYKHSWHYGNST | |
| [Human | DRISTEPDAGVMLKITKPGINDAGVYVLLVRLDHSRST | |
| herpesvirus 3] | DGFILGVNVYTAGSHHNIHGVIYTSPSLQNGYSTRALF | |
| | QQARLCDLPATPKGSGTSLFQHMLDLRAGKSLEDNPW | |
| | LHEDVVTTETKSVVKEGIENHVYPTDMSTLPEKSLNDP | |
| | PENLLIIIPIVASVMILTAMVIVIVISVKRRRIKKHPIYRPN | |
| | TKTRRGIQNATPESDVMLEAAIAQLATIREESPPHSVVN | |
| | PFVK | |
| VZV-GE-delete- | MGTVNKPVVGVLMGFGIITGTLRITNPVRASVLRYDDF | 47 |
| 562 | HIDEDKLDTNSVYEPYYHSDHAESSWVNRGESSRKAY | |
| | DHNSPYIWPRNDYDGFLENAHEHHGVYNQGRGIDSGE | |
| | RLMQPTQMSAQEDLGDDTGVIPTLNGDDRHKIVNVDQ | |
| | RQYGDVFKGDLNPKPQGQRLIEVSVEENHPFTLRAPIQ | |
| | RIYGVRYTETWSFLPSLTCTGDAAPAIQHICLKHTTCFQ | |
| | DVVVDVDCAENTKEDQLAEISYRFQGKKEADQPWIVV | |
| | NTTLFDELELDPPEIEPGVLKVLRTEKQYLGVYIWNMR | |
| | GSDGTSTYATFLVTWKGDEKTRNPTPAVTPQPRGAEF | |
| | HMWNYHSHVFSVGDTFSLAMHLQYKIHEAPFDLLLEW | |
| | LYVPIDPTCQPMRLYSTCLYHPNAQCLSHMNSGCTFTS | |
| | PHLAQRVASTVYQNCEHADNYTAYCLGISHMEPSFGLI | |
| | LHDGGTTLKFVDTPESLSGLYVFVVYFNGHVEAVAYT | |
| | VVSTVDHFVNAIEERGFPPTAGQPPATTKPKEITPVNPG | |
| | TSPLLRYAWTGGLAAVVLLCLVIFLICTA | |
| gi\|46981496\|gb\| | MKRIQINLILTIACIQLSTESQPTPVSITELYTSAATRKPD | 48 |
| AAT07772.1\| | PAVAPTSAATRKPDPAVAPTSAATRKPDPAVAPTSAAT | |
| membrane | RKPDPAVAPTSAATRKPDPAVAPTSAATRKPDPAVAPT | |
| glycoprotein C | SAASRKPDPAVAPTSAASRKPDPAVAPTSAASRKPDPA | |
| [Human | ANTQHSQPPFLYENIQCVHGGIQSIPYFHTFIMPCYMRL | |
| herpesvirus 3] | TTGQQAAFKQQQKTYEQYSLDPEGSNITRWKSLIRPDL | |
| | HIEVWFTRHLIDPHRQLGNALIRMPDLPVMLYSNSADL | |
| | NLINNPEIFTHAKENYVIPDVKTTSDFSVTILSMDATTE | |
| | GTYIWRVVNTKTKNVISEHSITVTTYYRPNITVVGDPVL | |
| | TGQTYAAYCNVSKYYPPHSVRVRWTSRFGNIGKNFITD | |
| | AIQEYANGLFSYVSAVRIPQQKQMDYPPPAIQCNVLWI | |
| | RDGVSNMKYSAVVTPDVYPFPNVSIGIIDGHIVCTAKC | |
| | VPRGVVHFVWWVNDSPINHENSEITGVCDQNKRFVNM | |
| | QSSCPTSELDGPITYSCHLDGYPKKFPPFSAVYTYDAST | |
| | YATTFSVVAVIIGVISILGTLGLIAVIATLCIRCCS | |
| gi\|9625934\|ref\| | MASHKWLLQIVFLKTITIAYCLHLQDDTPLFFGAKPLSD | 49 |
| NP_040182.1\| | VSLIITEPCVSSVYEAWDYAAPPVSNLSEALSGIVVKTK | |
| envelope | CPVPEVILWFKDKQMAYWTNPYVTLKGLAQSVGEEH | |
| glycoprotein L | KSGDIRDALLDALSGVWVDSTPSSTNIPENGCVWGAD | |
| [Human | RLFQRVCQ | |
| herpesvirus 3] | | |
| gi\|9625925\|ref\| | MGTQKKGPRSEKVSPYDTTTPEVEALDHQMDTLNWRI | 50 |
| NP_040172.1\| | WIIQVMMFTLGAVMLLATLIAASSEYTGIPCFYAAVVD | |
| envelope | YELFNATLDGGVWSGNRGGYSAPVLFLEPHSVVAFTY | |
| glycoprotein M | YTALTAMAMAVYTLITAAIIHRETKNQRVRQSSGVAW | |
| [Human | LVVDPTTLFWGLLSLWLLNAVVLLLAYKQIGVAATLY | |
| herpesvirus 3] | LGHFATSVIFTTYFCGRGKLDETNIKAVANLRQQSVFL | |
| | YRLAGPTRAVFVNLMAALMAICILFVSLMLELVVANH | |
| | LHTGLWSSVSVAMSTFSTLSVVYLIVSELILAHYIHVLI | |
| | GPSLGTLVACATLGTAAHSYMDRLYDPISVQSPRLIPTT | |
| | RGTLACLAVFSVVMLLLRLMRAYVYHRQKRSRFYGA | |
| | VRRVPERVRGYIRKVKPAHRNSRRTNYPSQGYGYVYE | |
| | NDSTYETDREDELLYERSNSGWE | |
| gi\|9625912\|ref\| | MFALVLAVVILPLWTTANKSYVTPTPATRSIGHMSALL | 51 |
| NP_040160.1\| | REYSDRNMSLKLEAFYPTGFDEELIKSLHWGNDRKHV | |
| envelope | FLVIVKVNPTTHEGDVGLVIFPKYLLSPYHFKAEHRAPF | |
| glycoprotein H | PAGRFGFLSHPVTPDVSFFDSSFAPYLTTQHLVAFTTFP | |
| [Human | PNPLVWHLERAETAATAERPFGVSLLPARPTVPKNTILE | |
| herpesvirus 3] | HKAHFATWDALARHTFFSAEAIITNSTLRIHVPLFGSV | |
| | WPIRYWATGSVLLTSDSGRVEVNIGVGFMSSLISLSSGP | |
| | PIELIVVPHTVKLNAVTSDTTWFQLNPPGPDPGPSYRVY | |
| | LLGRGLDMNFSKHATVDICAYPEESLDYRYHLSMAHT | |
| | EALRMTTKADQHDINEESYYHIAARIATSIFALSEMGRT | |
| | TEYFLLDEIVDVQYQLKFLNYILMRIGAGAHPNTISGTS | |
| | DLIFADPSQLHDELSLLFGQVKPANVDYFISYDEARDQ | |
| | LKTAYALSRGQDHVNALSLARRVIMSIYKGLLVKQNL | |
| | NATERQALFFASMILLNFREGLENSSRVLDGRTTLLLM | |
| | TSMCTAAHATQAALNIQEGLAYLNPSKHMFTIPNVYSP | |
| | CMGSLRTDLTEEIHVMNLLSAIPTRPGLNEVLHTQLDES | |
| | EIFDAAFKTMMIFTTWTAKDLHILHTHVPEVFTCQDAA | |
| | ARNGEYVLILPAVQGHSYVITRNKPQRGLVYSLADVD | |
| | VYNPISVVYLSRDTCVSEHGVIETVALPHPDNLKECLY | |
| | CGSVFLRYLTTGAIMDIIIIDSKDTERQLAAMGNSTIPPF | |
| | NPDMHGDDSKAVLLFPNGTVVTLLGFERRQAIRMSGQ | |
| | YLGASLGGAFLAVVGFGIIGWMLCGNSRLREYNKIPLT | |
| gi\|584403829\|gb\| | MFYEALKAELVYTRAVHGFRPRANCVVLSDYIPRVAC | 52 |
| AHB80298.1\| | NMGTVNKPVVGVLMGFGIITGTLRITNPVRASVLRYDD | |
| envelope | FHIDEDKLDTNSVYEPYYHSDHAESSWVNRGESSRKA | |
| glycoprotein E | YDHNSPYIWPRNDYDGFLENAHEHHGVYNQGRGIDSG | |
| [Human | ERLMQPTQMSAQEDLGDDTGIHVIPTLNGDDRHKIVN | |
| herpesvirus 3] | VDQRQYGDVFKGDLNPKPQGQRLIEVSVEENHPFTLR | |
| | APIQRIYGVRYTETWSFLPSLTCTGDAAPAIQHICLKHT | |
| | TCFQDVVVDVDCAENTKEDQLAEISYRFQGKKEADQP | |
| | WIVVNTSTLFDELELDPPEIEPGVLKVLRTEKQYLGVYI | |
| | WNMRGSDGTSTYATFLVTWKGDEKTRNPTPAVTPQPR | |
| | GAEFHMWNYHSHVFSVGDTFSLAMHLQYKIHEAPFDL | |
| | LLEWLYVPIDPTCQPMRLYSTCLYHPNAPQCLSHMNSG | |
| | CTFTSPHLAQRVASTVYQNCEHADNYTAYCLGISHME | |
| | PSFGLILHDGGTTLKFVDTPESLSGLYVFVVYFNGHVE | |
| | AVAYTVVSTVDHFVNAIEERGFPPTAGQPPATTKPKEIT | |
| | PVNPGTSPLLRYAAWTGGLAAVVLLCLVIFLICTAKRM | |
| | RVKAYRVDKSPYNQSMYYAGLPVDDFEDSESTDTEEE | |
| | FGNAIGGSHGGSSYTVYIDKTR | |
| gi\|46981513\|gb\| | MFVTAVVSVSPSSFYESLQVEPTQSEDITRSAHLGDGDE | 53 |
| AAT07789.1\| | IREAIHKSQDAETKPTFYVCPPPTGSTIVRLEPTRTCPDY | |
| glycoprotein B | HLGKNFTEGIAVVYKENIAAYKFKATVYYKDVIVSTA | |
| [Human | WAGSSYTQITNRYADRVPIPVSEITDTIDKFGKCSSKAT | |
| herpesvirus 3] | YVRNNHKVEAFNEDKNPQDMPLIASKYNSVGSKAWH | |
| | TTNDTYMVAGTPGTYRTGTSVNCIIEEVEARSIFPYDSF | |
| | GLSTGDIIYMSPFFGLRDGAYREHSNYAMDRFHQFEGY | |
| | RQRDLDTRALLEPAARNFLVTPHLTVGWNWKPKRTEV | |
| | CSLVKWREVEDVVRDEYAHNFRFTMKTLSTTFISETNE | |
| | FNLNQIHLSQCVKEEARAIINRIYTTRYNSSHVRTGDIQ | |
| | TYLARGGFVVVFQPLLSNSLARLYLQELVRENTNHSPQ | |
| | KHPTRNTRSRRSVPVELRANRTITTTSSVEFAMLQFTYD | |
| | HIQEHVNEMLARISSSWCQLQNRERALWSGLFPINPSA | |
| | LASTILDQRVKARILGDVISVSNCPELGSDTRIILQNSMR | |
| | VSGSTTRCYSRPLISIVSLNGSGTVEGQLGTDNELIMSR | |
| | DLLEPCVANHKRYFLFGHHYVYYEDYRYVREIAVHDV | |
| | GMISTYVDLNLTLLKDREFMPLQVYTRDELRDTGLLD | |
| | YSEIQRRNQMHSLRFYDIDKVVQYDSGTAIMQGMAQF | |
| | FQGLGTAGQAVGHVVLGATGALLSTVHGFTTFLSNPF | |
| | GALAVGLLVLAGLVAAFFAYRYVLKLKTSPMKALYPL | |
| | TTKGLKQLPEGMDPFAEKPNATDTPIEEIGDSQNTEPSV | |
| | NSGFDPDKFREAQEMIKYMTLVSAAERQESKARKKNK | |
| | TSALLTSRLTGLALRNRRGYSRVRTENVTGV | |
| gi\|46981487\|gb\| | MQALGIKTEHFIIMCLLSGHAVFTLWYTARVKFEHECV | 54 |
| AAT07763.1\| | YATTVINGGPVVWGSYNNSLIYVTFVNHSTFLDGLSGY | |
| glycoprotein K | DYSCRENLLSGDTMVKTAISTPLHDKIRIVLGTRNCHA | |
| [Human | YFWCVQLKMIFFAWFVYGMYLQFRRIRRMFGPFRSSC | |
| herpesvirus 3] | ELISPTSYSLNYVTRVISNILLGYPYTKLARLLCDVSMR | |
| | RDGMSKVFNADPISFLYMHKGVTLLMLLEVIAHISSGCI | |
| | VLLTLGVAYTPCALLYPTYIRILAWVVVCTLAIVELISY | |
| | VRPKPTKDNHLNHINTGGIRGICTTCCATVMS | |
| | GLAIKCFYIVIFAIAVVIFMHYEQRVQVSLFGESENSQK | |
| | H | |
| gi\|443500633\|gb\| | MGSITASFILITMQILFFCEDSSGEPNFAERNFWHASCSA | 55 |
| AGC94499.1\| | RGVYIDGSMITTLFFYASLLGVCVALISLAYHACFRLFT | |
| glycoprotein N | RSVLRSTW | |
| [Human | | |
| herpesvirus 3] | | |
| Ig heavy chain | MDWTWILFLVAAATRVHS | 56 |
| epsilon-1 signal | | |
| peptide (IgE HC | | |
| SP) | | |
| IgGk chain V-III | METPAQLLFLLLLWLPDTTG | 57 |
| region HAH | | |
| signal peptide | | |
| (IgGk SP) | | |
| Japanese | MLGSNSGQRVVFTILLLLVAPAYS | 109 |
| encephalitis | | |
| PRM signal | | |
| sequence | | |
| VSVg protein | MKCLLYLAFLFIGVNCA | 110 |
| signal sequence | | |
| Japanese | MWLVSLAIVTACAGA | 111 |
| encephalitis JEV | | |
| signal sequence | | |

TABLE 15
Flagellin Nucleic Acid Sequences

| | | SEQ ID |
|---|---|---|
| Name | Sequence | NO: |
| NT (5′ | TCAAGCTTTTGGACCCTCGTACAGAAGCTAATACGACTCACTAT | 112 |
| UTR, ORF, | AGGGAAATAAGAGAGAAAAGAAGAGTAAGAAGAAATATAAG | |
| 3′ UTR) | AGCCACCATGGCACAAGTCATTAATACAAACAGCCTGTCGCTG | |
| | TTGACCCAGAATAACCTGAACAAATCCCAGTCCGCACTGGGCA | |
| | CTGCTATCGAGCGTTTGTCTTCCGGTCTGCGTATCAACAGCGCG | |
| | AAAGACGATGCGGCAGGACAGGCGATTGCTAACCGTTTTACCG | |
| | CGAACATCAAAGGTCTGACTCAGGCTTCCCGTAACGCTAACGA | |
| | CGGTATCTCCATTGCGCAGACCACTGAAGGCGCGCTGAACGAA | |
| | ATCAACAACAACCTGCAGCGTGTGCGTGAACTGGCGGTTCAGT | |
| | CTGCGAATGGTACTAACTCCCAGTCTGACCTCGACTCCATCCAG | |
| | GCTGAAATCACCCAGCGCCTGAACGAAATCGACCGTGTATCCG | |
| | GCCAGACTCAGTTCAACGGCGTGAAAGTCCTGGCGCAGGACAA | |
| | CACCCTGACCATCCAGGTTGGTGCCAACGACGGTGAAACTATC | |
| | GATATTGATTTAAAAGAAATCAGCTCTAAAACACTGGGACTTG | |
| | ATAAGCTTAATGTCCAAGATGCCTACACCCCGAAAGAAACTGC | |
| | TGTAACCGTTGATAAAACTACCTATAAAAATGGTACAGATCCT | |
| | ATTACAGCCCAGAGCAATACTGATATCCAAACTGCAATTGGCG | |
| | GTGGTGCAACGGGGGTTACTGGGGCTGATATCAAATTTAAAGA | |
| | TGGTCAATACTATTTAGATGTTAAAGGCGGTGCTTCTGCTGGTG | |
| | TTTATAAAGCCACTTATGATGAAACTACAAAGAAAGTTAATAT | |
| | TGATACGACTGATAAAACTCCGTTGGCAACTGCGGAAGCTACA | |
| | GCTATTCGGGGAACGGCCACTATAACCCACAACCAAATTGCTG | |
| | AAGTAACAAAAGAGGGTGTTGATACGACCACAGTTGCGGCTCA | |
| | ACTTGCTGCAGCAGGGGTTACTGGCGCCGATAAGGACAATACT | |
| | AGCCTTGTAAAACTATCGTTTGAGGATAAAAACGGTAAGGTTA | |
| | TTGATGGTGGCTATGCAGTGAAAATGGGCGACGATTTCTATGC | |
| | CGCTACATATGATGAGAAAACAGGTGCAATTACTGCTAAAACC | |
| | ACTACTTATACAGATGGTACTGGCGTTGCTCAAACTGGAGCTGT | |
| | GAAATTTGGTGGCGCAAATGGTAAATCTGAAGTTGTTACTGCT | |
| | ACCGATGGTAAGACTTACTTAGCAAGCGACCTTGACAAACATA | |
| | ACTTCAGAACAGGCGGTGAGCTTAAAGAGGTTAATACAGATAA | |
| | GACTGAAAACCCACTGCAGAAAATTGATGCTGCCTTGGCACAG | |
| | GTTGATACACTTCGTTCTGACCTGGGTGCGGTTCAGAACCGTTT | |
| | CAACTCCGCTATCACCAACCTGGGCAATACCGTAAATAACCTG | |
| | TCTTCTGCCCGTAGCCGTATCGAAGATTCCGACTACGCAACCGA | |
| | AGTCTCCAACATGTCTCGCGCGCAGATTCTGCAGCAGGCCGGT | |
| | ACCTCCGTTCTGGCGCAGGCGAACCAGGTTCCGCAAAACGTCC | |
| | TCTCTTTACTGCGTTGATAATAGGCTGGAGCCTCGGTGGCCATG | |
| | CTTCTTGCCCCTTGGGCCTCCCCCCAGCCCCTCCTCCCCTTCCTG | |
| | CACCCGTACCCCCGTGGTCTTTGAATAAAGTCTGAGTGGGCGG | |
| | C | |
| ORF | ATGGCACAAGTCATTAATACAAACAGCCTGTCGCTGTTGACCC | 113 |
| Sequence, | AGAATAACCTGAACAAATCCCAGTCCGCACTGGGCACTGCTAT | |
| NT | CGAGCGTTTGTCTTCCGGTCTGCGTATCAACAGCGCGAAAGAC | |
| | GATGCGGCAGGACAGGCGATTGCTAACCGTTTTACCGCGAACA | |
| | TCAAAGGTCTGACTCAGGCTTCCCGTAACGCTAACGACGGTAT | |
| | CTCCATTGCGCAGACCACTGAAGGCGCGCTGAACGAAATCAAC | |
| | AACAACCTGCAGCGTGTGCGTGAACTGGCGGTTCAGTCTGCGA | |
| | ATGGTACTAACTCCCAGTCTGACCTCGACTCCATCCAGGCTGAA | |
| | ATCACCCAGCGCCTGAACGAAATCGACCGTGTATCCGGCCAGA | |
| | CTCAGTTCAACGGCGTGAAAGTCCTGGCGCAGGACAACACCCT | |
| | GACCATCCAGGTTGGTGCCAACGACGGTGAAACTATCGATATT | |
| | GATTTAAAAGAAATCAGCTCTAAAACACTGGGACTTGATAAGC | |
| | TTAATGTCCAAGATGCCTACACCCCGAAAGAAACTGCTGTAAC | |
| | CGTTGATAAAACTACCTATAAAAATGGTACAGATCCTATTACA | |
| | GCCCAGAGCAATACTGATATCCAAACTGCAATTGGCGGTGGTG | |
| | CAACGGGGGTTACTGGGGCTGATATCAAATTTAAAGATGGTCA | |
| | ATACTATTTAGATGTTAAAGGCGGTGCTTCTGCTGGTGTTTATA | |
| | AAGCCACTTATGATGAAACTACAAAGAAAGTTAATATTGATAC | |
| | GACTGATAAAACTCCGTTGGCAACTGCGGAAGCTACAGCTATT | |
| | CGGGGAACGGCCACTATAACCCACAACCAAATTGCTGAAGTAA | |
| | CAAAAGAGGGTGTTGATACGACCACAGTTGCGGCTCAACTTGC | |
| | TGCAGCAGGGGTTACTGGCGCCGATAAGGACAATACTAGCCTT | |
| | GTAAAACTATCGTTTGAGGATAAAAACGGTAAGGTTATTGATG | |
| | GTGGCTATGCAGTGAAAATGGGCGACGATTTCTATGCCGCTAC | |
| | ATATGATGAGAAAACAGGTGCAATTACTGCTAAAACCACTACT | |
| | TATACAGATGGTACTGGCGTTGCTCAAACTGGAGCTGTGAAAT | |
| | TTGGTGGCGCAAATGGTAAATCTGAAGTTGTTACTGCTACCGAT | |
| | GGTAAGACTTACTTAGCAAGCGACCTTGACAAACATAACTTCA | |
| | GAACAGGCGGTGAGCTTAAAGAGGTTAATACAGATAAGACTG | |
| | AAAACCCACTGCAGAAAATTGATGCTGCCTTGGCACAGGTTGA | |
| | TACACTTCGTTCTGACCTGGGTGCGGTTCAGAACCGTTTCAACT | |
| | CCGCTATCACCAACCTGGGCAATACCGTAAATAACCTGTCTTCT | |
| | GCCCGTAGCCGTATCGAAGATTCCGACTACGCAACCGAAGTCT | |
| | CCAACATGTCTCGCGCGCAGATTCTGCAGCAGGCCGGTACCTC | |
| | CGTTCTGGCGCAGGCGAACCAGGTTCCGCAAAACGTCCTCTCTT | |
| | TACTGCGT | |
| mRNA | G*GGGAAAUAAGAGAGAAAAGAAGAGUAAGAAGAAAUAUAA | 114 |
| Sequence | GAGCCACCAUGGCACAAGUCAUUAAUACAAACAGCCUGUCGC | |
| (assumes | UGUUGACCCAGAAUAACCUGAACAAAUCCCAGUCCGCACUGG | |
| T100 tail) | GCACUGCUAUCGAGCGUUUGUCUUCCGGUCUGCGUAUCAACA | |
| | GCGCGAAAGACGAUGCGGCAGGACAGGCGAUUGCUAACCGUU | |
| | UUACCGCGAACAUCAAAGGUCUGACUCAGGCUUCCCGUAACG | |
| | CUAACGACGGUAUCUCCAUUGCGCAGACCACUGAAGGCGCGC | |
| | UGAACGAAAUCAACAACAACCUGCAGCGUGUGCGUGAACUGG | |
| | CGGUUCAGUCUGCGAAUGGUACUAACUCCCAGUCUGACCUCG | |
| | ACUCCAUCCAGGCUGAAAUCACCCAGCGCCUGAACGAAAUCG | |
| | ACCGUGUAUCCGGCCAGACUCAGUUCAACGGCGUGAAAGUCC | |
| | UGGCGCAGGACAACACCCUGACCAUCCAGGUUGGUGCCAACG | |
| | ACGGUGAAACUAUCGAUAUUGAUUUAAAAGAAAUCAGCUCU | |
| | AAAACACUGGGACUUGAUAAGCUUAAUGUCCAAGAUGCCUAC | |
| | ACCCCGAAAGAAACUGCUGUAACCGUUGAUAAAACUACCUAU | |
| | AAAAAUGGUACAGAUCCUAUUACAGCCCAGAGCAAUACUGAU | |
| | AUCCAAACUGCAAUUGGCGGUGGUGCAACGGGGGUUACUGG | |
| | GGCUGAUAUCAAAUUUAAAGAUGGUCAAUACUAUUUAGAUG | |
| | UUAAAGGCGGUGCUUCUGCUGGUGUUUAUAAAGCCACUUAU | |
| | GAUGAAACUACAAAGAAAGUUAAUAUUGAUACGACUGAUAA | |
| | AACUCCGUUGGCAACUGCGGAAGCUACAGCUAUUCGGGGAAC | |
| | GGCCACUAUAACCCACAACCAAAUUGCUGAAGUAACAAAAGA | |
| | GGGUGUUGAUACGACCACAGUUGCGGCUCAACUUGCUGCAGC | |
| | AGGGGUUACUGGCGCCGAUAAGGACAAUACUAGCCUUGUAA | |
| | AACUAUCGUUUGAGGAUAAAAACGGUAAGGUUAUUGAUGGU | |
| | GGCUAUGCAGUGAAAAUGGGCGACGAUUUCUAUGCCGCUACA | |
| | UAUGAUGAGAAAACAGGUGCAAUUACUGCUAAAACCACUAC | |
| | UUAUACAGAUGGUACUGGCGUUGCUCAAACUGGAGCUGUGA | |
| | AAUUUGGUGGCGCAAAUGGUAAAUCUGAAGUUGUUACUGCU | |
| | ACCGAUGGUAAGACUUACUUAGCAAGCGACCUUGACAAACAU | |
| | AACUUCAGAACAGGCGGUGAGCUUAAAGAGGUUAAUACAGA | |
| | UAAGACUGAAAACCCACUGCAGAAAAUUGAUGCUGCCUUGGC | |
| | ACAGGUUGAUACACUUCGUUCUGACCUGGGUGCGGUUCAGAA | |
| | CCGUUUCAACUCCGCUAUCACCAACCUGGGCAAUACCGUAAA | |
| | UAACCUGUCUUCUGCCCGUAGCCGUAUCGAAGAUUCCGACUA | |
| | CGCAACCGAAGUCUCCAACAUGUCUCGCGCGCAGAUUCUGCA | |
| | GCAGGCCGGUACCUCCGUUCUGGCGCAGGCGAACCAGGUUCC | |
| | GCAAAACGUCCUCUCUUUACUGCGUUGAUAAUAGGCUGGAGC | |
| | CUCGGUGGCCAUGCUUCUUGCCCCUUGGGCCUCCCCCCAGCC | |
| | CCUCCUCCCCUUCCUGCACCCGUACCCCCGUGGUCUUUGAAU | |
| | AAAGUCUGAGUGGGCGGCAAAAAAAAAAAAAAAAAAAAAAA | |
| | AAAAAAAAAAAAAAAAAAAAAAAAAAAAAAAAAAAAAAAAA | |
| | AAAAAAAAAAAAAAAAAAAAAAAAAAAAAAAAAAAAUCUAG | |

TABLE 16
Flagellin Amino Acid Sequences

| | | SEQ ID |
|---|---|---|
| Name | Sequence | NO: |
| ORF | MAQVINTNSLSLLTQNNLNKSQSALGTAIERLSSGLRINSAKDDAA | 115 |
| Sequence, | GQAIANRFTANIKGLTQASRNANDGISIAQTTEGALNEINNNLQRV | |
| AA | RELAVQSANGTNSQSDLDSIQAEITQRLNEIDRVSGQTQFNGVKVL | |
| | AQDNTLTIQVGANDGETIDIDLKEISSKTLGLDKLNVQDAYTPKET | |
| | AVTVDKTTYKNGTDPITAQSNTDIQTAIGGGATGVTGADIKFKDG | |
| | QYYLDVKGGASAGVYKATYDETTKKVNIDTTDKTPLATAEATAI | |
| | RGTATITHNQIAEVTKEGVDTTTVAAQLAAAGVTGADKDNTSLV | |
| | KLSFEDKNGKVIDGGYAVKMGDDFYAATYDEKTGAITAKTTTYT | |
| | DGTGVAQTGAVKFGGANGKSEVVTATDGKTYLASDLDKHNFRT | |
| | GGELKEVNTDKTENPLQKIDAALAQVDTLRSDLGAVQNRFNSAIT | |
| | NLGNTVNNLSSARSRIEDSDYATEVSNMSRAQILQQAGTSVLAQA | |
| | NQVPQNVLSLLR | |
| Flagellin- | MAQVINTNSLSLLTQNNLNKSQSALGTAIERLSSGLRINSAKDDAA | 116 |
| GS linker- | GQAIANRFTANIKGLTQASRNANDGISIAQTTEGALNEINNNLQRV | |
| circum- | RELAVQSANSTNSQSDLDSIQAEITQRLNEIDRVSGQTQFNGVKVL | |
| sporozoite | AQDNTLTIQVGANDGETIDIDLKQINSQTLGLDTLNVQQKYKVSD | |
| protein | TAATVTGYADTTIALDNSTFKASATGLGGTDQKIDGDLKFDDTTG | |
| (CSP) | KYYAKVTVTGGTGKDGYYEVSVDKTNGEVTLAGGATSPLTGGLP | |
| | ATATEDVKNVQVANADLTEAKAALTAAGVTGTASVVKMSYTDN | |
| | NGKTIDGGLAVKVGDDYYSATQNKDGSISINTTKYTADDGTSKTA | |
| | LNKLGGADGKTEVVSIGGKTYAASKAEGHNFKAQPDLAEAAATT | |
| | TENPLQKIDAALAQVDTLRSDLGAVQNRFNSAITNLGNTVNNLTS | |
| | ARSRIEDSDYATEVSNMSRAQILQQAGTSVLAQANQVPQNVLSLL | |
| | RGGGGSGGGGSMMAPDPNANPNANPNANPNANPNANPNANPNA | |
| | NPNANPNANPNANPNANPNANPNANPNANPNANPNANPNANPN | |
| | ANPNANPNKNNQGNGQGHNMPNDPNRNVDENANANNAVKNNN | |
| | NEEPSDKHIEQYLKKIKNSISTEWSPCSVTCGNGIQVRIKPGSANKP | |
| | KDELDYENDIEKKICKMEKCSSVFNVVNS | |
| Flagellin- | MMAPDPNANPNANPNANPNANPNANPNANPNANPNANPNANPN | 117 |
| RPVT | ANPNANPNANPNANPNANPNANPNANPNANPNANPNANPNKNN | |
| linker | QGNGQGHNMPNDPNRNVDENANANNAVKNNNNEEPSDKHIEQY | |
| circum- | LKKIKNSISTEWSPCSVTCGNGIQVRIKPGSANKPKDELDYENDIEK | |
| sporozoite | KICKMEKCSSVFNVVNSRPVTMAQVINTNSLSLLTQNNLNKSQSA | |
| protein | LGTAIERLSSGLRINSAKDDAAGQAIANRFTANIKGLTQASRNAND | |
| (CSP) | FISIAQTTEGALNEINNNLQRVRELAVQSANSTNSQSDLDSIQAEIT | |
| | QRLNEIDRVSGQTQFNGVKVLAQDNTLTIQVGANDGETIDIDLKQI | |
| | NSQTLGLDTLNVQQKYKVSDTAATVTGYADTTIALDNSTFKASAT | |
| | GLGGTDQKIDGDLKFDDTTGKYYAKVTVTGGTGKDGYYEVSVD | |
| | KTNGEVTLAGGATSPLTGGLPATATEDVKNVQVANADLTEAKAA | |
| | LTAAGVTGTASVVKMSYTDNNGKTIDGGLAVKVGDDYYSATQN | |
| | KDGSISINTTKYTADDGTSKTALNKLGGADGKTEVVSIGGKTYAA | |
| | SKAEGHNFKAQPDLAEAAATTTENPLQKIDAALAQVDTLRSDLG | |
| | AVQNRFNSAITNLGNTVNNLTSARSRIEDSDYATEVSNMSRAQILQ | |
| | QAGTSVLAQANQVPQNVLSLLR | |

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1-12. (canceled)

13. A messenger ribonucleic acid (mRNA) vaccine comprising;
(a) an mRNA polynucleotide comprising from 5′ to 3′ a 5′UTR, an open reading frame (ORF) encoding a varicella zoster virus (VZV) gE protein, and a 3′ UTR, wherein 100% of uracil nucleosides in the ORF of the mRNA polynucleotide are N1-methylpseudouridine; and
(b) a lipid nanoparticle comprising an ionizable cationic lipid, a neutral lipid, a sterol, and a polyethylene glycol (PEG)-modified lipid.

14. The mRNA vaccine of claim 13, wherein the VZV gE protein comprises the amino acid sequence of SEQ ID NO: 38.

15. The mRNA vaccine of claim 13, wherein the ORF comprises a nucleotide sequence comprising at least 90% sequence identity to a mRNA sequence corresponding to SEQ ID NO: 62.

16. The mRNA vaccine of claim 13, wherein the ORF comprises a nucleotide sequence of SEQ ID NO: 62.

17. The mRNA vaccine of claim 14, wherein the ORF comprises a nucleotide sequence comprising at least 90% sequence identity to a mRNA sequence corresponding to SEQ ID NO: 62.

18. The mRNA vaccine of claim 13, wherein the VZV gE protein comprises a Y569A mutation, relative to an amino acid sequence of SEQ ID NO: 10.

19. The mRNA vaccine of claim 13, wherein the VZV gE protein comprises a Y582G mutation, relative to an amino acid sequence of SEQ ID NO: 10.

20. The mRNA vaccine of claim 13, wherein the VZV gE polypeptide is a truncated polypeptide lacking a carboxy terminal tail domain.

21. The mRNA vaccine of claim 13, wherein the VZV gE polypeptide is a truncated polypeptide lacking an anchor domain.

22. The mRNA vaccine of claim 13, wherein the VZV gE polypeptide has at least one mutation in at least one phosphorylated acidic motif(s).

23. The mRNA vaccine of claim 13, wherein the lipid nanoparticle comprises 1,2-distearoyl-sn-glycero-3-phosphocholine (DSPC).

24. The mRNA vaccine of claim 13, wherein the lipid nanoparticle comprises PEG-distearoyl glycerol (PEG DMG).

25. The mRNA vaccine of claim 13, wherein the lipid nanoparticle comprises a cholesterol.

26. The mRNA vaccine of claim 13, wherein the lipid nanoparticle comprises 1,2-distearoyl-sn-glycero-3-phosphocholine (DSPC), cholesterol, and PEG-distearoyl glycerol (PEG DMG).

27. The mRNA vaccine of claim 13, wherein the mRNA vaccine comprises a second mRNA polynucleotide comprising a second ORF encoding a VZV antigen.

28. The mRNA vaccine of claim 27, wherein the second ORF encodes a VZV gI antigen.

29. The mRNA vaccine of claim 13, wherein the mRNA vaccine comprises a total dose of 25 μg-50 μg mRNA polynucleotide.

30. A method of inducing an antigen specific immune response in a subject, comprising:
administering to the subject to produce an antigen-specific immune response in the subject an mRNA vaccine comprising:
(a) an mRNA polynucleotide comprising from 5′ to 3′ a 5′UTR, an open reading frame (ORF) encoding a varicella zoster virus (VZV) gE protein, and a 3′ UTR, wherein 100% of uracil nucleosides in the ORF of the mRNA polynucleotide are N1-methylpseudouridine; and
(b) a lipid nanoparticle comprising an ionizable cationic lipid, a neutral lipid, a sterol, and a polyethylene glycol (PEG)-modified lipid.

31. The method of claim 30, wherein the mRNA vaccine is administered in a total dose of 25 μg-100 μg mRNA polynucleotide.
